(19) United States
(12) Patent Application Publication
(10) Pub. No.: US 2007/0232673 A1
(43) Pub. Date: Oct. 4, 2007

(54) 2-Imino-benzimidazoles

(76) Inventors: Gregory P. Roth, Woodstock, CT (US); Grier A. Wallace, Sterling, MA (US); Dawn M. George, Charlton, MA (US); Pintipa Grongsaard, Shrewsbury, MA (US); Martin Hayes, Lowell, MA (US); Eric C. Breinlinger, Charlton, MA (US)

Correspondence Address: ABBOTT BIORESEARCH, 100 RESEARCH DRIVE, WORCESTER, MA, 01605-4314, US

(21) Appl. No.: 11/655,661

(22) Filed: Jan. 19, 2007

Related U.S. Application Data
(60) Provisional application No. 60/760,199, filed on Jan. 19, 2006.

(51) Int. Cl. A61K 31/4184; C07D 235/30

(52) U.S. Cl. 514/388; 548/307.4

(57) ABSTRACT

Novel compounds of Formula (I) or pharmaceutically acceptable salts, prodrugs and biologically active metabolites thereof of Formula (I) 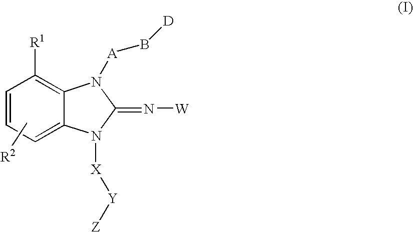 wherein the substituents are as defined herein, which are useful as therapeutic agents.

### BACKGROUND OF THE INVENTION

Chemokines are chemotactic cytokines that are released by a wide variety of cells to attract leukocytes, as illustrated by macrophages, T cells, B cells, eosinophils, basophils, and neutrophils to and from sites of inflammation or within specific compartments, as illustrated by lymph nodes (reviewed in Schall, Cytokine, 3:165-183 (1991), Schall, et al., Curr. Opin. Immunol., 6:865-873 (1994) and Murphy, Rev. Immun., 12:593-633 (1994)). In addition to stimulating chemotaxis, other changes can be selectively induced by chemokines in responsive cells, including changes in cell shape, transient rises in the concentration of intracellular free calcium ions ([Ca²⁺]), granule exocytosis, integrin upregulation, formation of bioactive lipids (e.g., leukotrienes), and respiratory burst, associated with leukocyte activation. Thus, the chemokines are early modulators of inflammatory response, effecting inflammatory mediator release, chemotaxis and extravasation to sites of infection or inflammation.

There are four classes of chemokines, CXC (α), CC (β), C (γ), and CX₃C (δ), depending on whether the first two cysteines are separated by a single amino acid (C—X—C), are adjacent (C—C), have a missing cysteine pair (C), or are separated by three amino acids (CXC₃). The α-chemokines, such as interleukin-8 (IL-8), melanoma growth stimulatory activity protein (MGSA), and stromal cell derived factor 1 (SDF-1) are chemotactic primarily for neutrophils and lymphocytes, whereas β-chemokines, such as RANTES, MIP-1α, MIP-1β, monocyte chemotactic protein-1 (MCP-1), MCP-2, MCP-3 and eotaxin are chemotactic for macrophages, T-cells, eosinophils and basophils (Deng, et al., Nature, 381:661-666 (1996)). The C chemokine lymphotactin shows specificity for lymphocytes (Kelner, et al., Science, 266:1395-1399 (1994)) while the CX₃C chemokine fractalkine shows specificity for lymphocytes and monocytes (Bazan, et al., Nature, 385:640-644 (1997)).

Chemokines bind specific cell-surface receptors belonging to the family of G-protein-coupled seven-transmembrane-domain proteins (reviewed in Horuk, Trends Pharm. Sci., 15:159-165 (1994)) termed “chemokine receptors.” On binding their cognate ligands, chemokine receptors transduce an intracellular signal through the associated heterotrimeric G protein, resulting in a rapid increase in intracellular calcium concentration. There are at least twelve human chemokine receptors that bind or respond to β-chemokines with the following characteristic pattern: CCR1 (or “CKR-1” or “CC—CKR-1”) MIP-1α, MIP-1β, MCP-3, RANTES (Ben-Barruch, et al., J. Biol. Chem., 270:22123-22128 (1995); Neote, et al., Cell, 72:415425 (1993)); CCR2A and CCR2B (or “CKR-2A”/“CKR-2A” or “CC—CKR-2A”/“CC-CKR2A”) MCP-1, MCP-3, MCP-4; CCR3 (or “CKR-3” or “CC—CKR-3”) eotaxin, RANTES, MCP; (Ponath, et al., J. Exp. Med., 183:2437-2448 (1996)); CCR4 (or “CKR4” or “CC—CKR4”) TARC, MDC (Imai, et al., J. Biol. Chem., 273:1764-1768 (1998)); CCR5 (or “CKR-5” or “CC—CKR-5”) MIP-1α, RANTES, MIP-1β; (Sanson, et al., Biochemistry, 35:3362-3367 (1996)); CCR6 MIP-3α (Greaves, et al., J. Exp. Med., 186:837-844 (1997)); CCR7 MIP-3β and 6Ckine (Campbell, et al., J. Cell. Biol., 141:1053-1059(1998)); CCR8 I-309, HHV8 vMIP-I, HHV-8 vMIP-II, MCV vMCC-I (Dairaghi, et al., J. Biol. Chem., 274:21569-21574 (1999)); CCR9 TECK (Zaballos, et al., J. Immunol., 162:5671-5675 (1999)), D6 MIP-1 beta, RANTES, and MCP-3 (Nibbs, et al., J. Biol. Chem., 272:32078-32083 (1997)), and the Duffy blood-group antigen RANTES, MCP-1 (Chaudhun, et al., J. Biol. Chem., 269:7835-7838 (1994)).

Chemokine receptors, such as CCR1, CCR2, CCR2A, CCR2B, CCR3, CCR4, CCR5, CCR6, CCR7, CCR8, CCR9, CXCR1, CXCR2, CXCR3, CXCR4, CXCR5, CX₃CR1, and XCR1 have been implicated as being important mediators of inflammatory and immunoregulatory disorders and diseases, including asthma and allergic diseases, as well as autoimmune pathologies such as rheumatoid arthritis and atherosclerosis.

The CXCR3 chemokine receptor is expressed primarily in T lymphocytes, and its functional activity can be measured by cytosolic calcium elevation or chemotaxis. The receptor was previously referred to as GPR9 or CKR-L2. Its chromosomal location is unusual among the chemokine receptors in being localized to Xq13. Ligands that have been identified that are selective and of high affinity are the CXC chemokines, IP10, MIG and ITAC.

The highly selective expression of CXCR3 makes it an ideal target for intervention to interrupt inappropriate T cell trafficking. The clinical indications for such intervention are in T-cell mediated autoimmune diseases such as multiple sclerosis, rheumatoid arthritis, asthma, allergy, and type I diabetes. Inappropriate T-cell infiltration also occurs in psoriasis and other pathogenic skin inflammation conditions, although the diseases may not be true autoimmune disorders. In this regard, up-regulation of IP-10 expression in keratinocytes is a common feature in cutaneous immunopathologies. Inhibition of CXCR3 can be beneficial in reducing rejection in organ transplantation. Ectopic expression of CXCR3 in certain tumors, especially subsets of B cell malignancies indicate that selective inhibitors of CXCR3 will have value in tumor immunotherapy, particularly attenuation of metastasis.

In view of the clinical importance of CXCR3, the identification of compounds that modulate CXCR3 function represents an attractive avenue into the development of new therapeutic agents. Such compounds are provided herein.

### SUMMARY OF THE INVENTION

The present invention provides a compound of Formula (I) 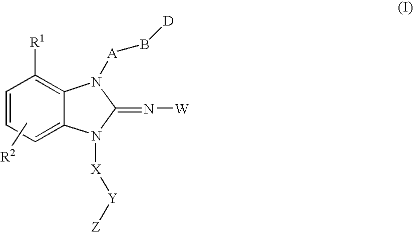
and pharmaceutically acceptable salts, prodrugs and biologically active metabolites thereof wherein
A is selected from the group consisting of a bond, —C(O)—, optionally substituted (C₁-C₆)alkyl and optionally substituted (C₂-C₆)alkenyl;
B is selected from the group consisting of a bond, O, C(O), N(R^{a}), —C(O)—N(R^{a})—, —N(R^{a})—C(O), —CH₂—C(O)—N(R^{a})—, —N(R^{a})—C(O)—CH₂—, —CH₂—N(R^{a})—C(O)—, —C(O)—N(R^{a})—CH₂ and optionally substituted (C₁-C₃)alkyl;
   wherein R^{a} is H, CHF₂, (C₁-C₄)alkyl or (C₃-C₆)cycloalkyl;
D is selected from the group consisting of H, halo, OH, CF₃, COOH, (C₁-C₄)alkoxy and dimethylamino; or
   D is selected from the optionally substituted group consisting of (C₁-C₆)alkyl, (C₂-C₆)alkenyl, (C₃-C₆)cycloalkyl, —C(O)—OR^{b}, aryl, aryl(C₁-C₄)alkyl, amino, heteroaryl and heterocyclyl;
wherein R^{b} is (C₁-C₄)alkyl, aryl(C₁-C₄)alkyl or aryl;
X is selected from the group consisting of a bond or an optionally substituted (C₁-C₆)alkyl and (C₂-C₄)alkenyl;
Y is selected from the group consisting of a bond, —C(O)—, —NR^{c}, —N(R^{c})—C(O)—, —C(O)—N(R^{c})—, S, optionally substituted (C₃-C₆)alkenyl, —C(O)—N(Q¹)-(CH₂)ₐ, or —N(Q¹)-(CH₂)ₐ or S(O)_{b};
   wherein R^{c} is H or (C₁-C₄)alkyl;
   wherein Q¹ is H or (C₁-C₄)alkyl;
   a is 0, 1 or 2;
   b is 1 or 2;
Z is H, or —N(Q²)₂ wherein Q² is (C₁-C₃)alkyl or optionally substituted benzyl; or
Z is selected from the optionally substituted group consisting of (C₂-C₆)alkenyl, (C₁-C₆)alkyl, (C₃-C₆)cycloalkyl, heterocyclyl, aryl, heteroaryl, phenylcarbonylheterocyclyl and phenylcarbonylheteroaryl;
R¹ is selected from the group consisting of H, halo, CF₃, —CH₂—CH₂-optionally substituted phenyl, —C(O)—OCH₃, (C₁-C₃)alkoxycarbonyl, (C₁-C₂)alkyl-O-phenyl, and (C₁-C₆)alkoxy; or
R¹ is selected from the optionally substituted group consisting of (C₁-C₆)alkyl, (C₂-C₆)alkenyl, (C₃-C₆)cycloalkyl, aryl, aryl(C₁-C₃)alkyl, heteroaryl and heterocyclyl;
R² is one or more substitutents selected from the group consisting of H, CF₃, halo, CN, OCF₃, —C(O)-Optionally substituted phenyl, (C₁-C₆)alkoxy and optionally substituted (C₁-C₆)alkyl;
W is H or CN; or
W is selected from the optionally substituted group consisting of (C₁-C₃)alkoxy(C₁-C₃)alkyl, aryl, aryl(C₁-C₄)alkyl, cycloalkyl(C₁-C₄)alkyl, heterocyclyl(C₁-C₄)alkyl, heteroaryl(C₁-C₄)alkyl, —C(O)—(C₁-C₆)alkoxy, —C(O)—NH-phenyl, —C(O)—(C₁-C₆)alkyl, (C₁-C₆)alkyl and —(CH₂)_{d}-Q³;
   wherein d is 1, 2, 3 or 4; and
   Q³ is selected from the optionally substituted group consisting of (C₃-C₆)cycloalkyl, dimethylamino and phenyl;
provided that the compound of Formula (I) is not 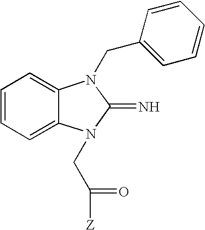
   wherein Z is an optionally substituted phenyl;
provided that the compound of Formula (I) is not 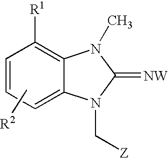
   wherein Z is optionally substituted phenyl; and
R¹, R² and W are as defined for Formula (I);
provided that the compound of Formula (I) is not 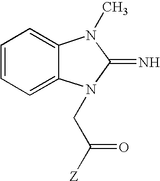
   wherein Z is phenyl substituted with OH, t-butyl and —O—CH₂—CH₂—CH₂—CO—NH₂; OH, t-butyl and —O—CH₂—CH₂—CH₂—CN; OH, t-butyl and —OCH₂—CH₂—CH₂—C(O)—NH₂; pyrrolidinyl, t-butyl and —OCH₂—CH₂—CH₂—COOH; pyrrolidinyl, t-butyl and —OCH₂—COOH; or t-butyl and dimethylamino; provided that the compound of Formula (I) is not 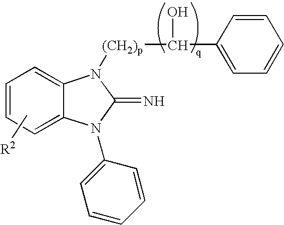
   wherein
p is 1 or 2;
q is 0 or 1; and
R² is as defined for Formula (I);
provided that the compound of Formula (I) is not 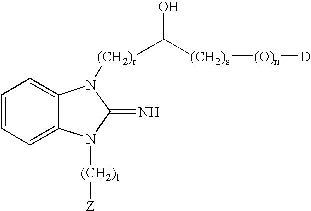
wherein D is CH₃, —CH═CH₂, propyl, butyl, t-butyl, furanyl, naphthyl, optionally substituted thienyl or optionally substituted phenyl;
Z is selected from the group consisting of H, CH₃, CH₂F, ethyl, morpholinyl, dimethylamino, diethylamino, —CH═CH₂, pentyl, dibenzylamino, naphthyl, piperidinyl and optionally substituted phenyl;
n is 0 or 1;
r is 1, 2 or 3;
s is 0, 1 or 2; and
t is 0, 1, 2 or 3;
provided that the compound of Formula (I) is not 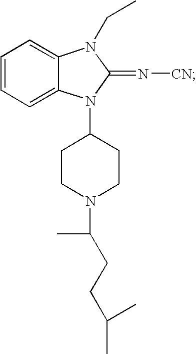
   provided that the compound of Formula (I) is not 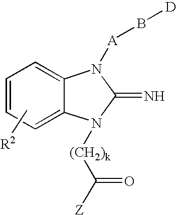
   wherein
A-B is a bond or an optionally substituted (C₁-C₅)alkyl;
D is selected from the group consisting of H, COOH, OH, NH₂, propyl, isopropyl, t-butyl, biphenyl, furanyl, pyridinyl, thiazolyl, quinolinyl, morpholinyl, cyclohexyl optionally substituted with NH₂, —C(O)NH₂, COOH or —C(O)—OCH₂CH₃, phenyl optionally substituted with OH, t-butyl and —S(O)₂—CH₃, phenyl substituted with OH and two t-butyl or phenyl substituted with substituted propyl and COOH; or phenyl substituted with a substituent selected from the group consisting of Cl, F, CH₃, CN, COOH, CH₂—CH₂—COOH, —CH₂—C(CH₃)₂—COOH, —CH₂—CH₂—C(O)—O—CH₂—CH₃, —NH—CH₂—COOH, —C(O)—O—CH₂—CH₃, —C(O)OH, dimethylamino, —S(O)₂—NH₂, —NH—CH₂—C(O)—NH₂, —NH—CH₂—C(O)—OH, —NH—C(O)—OH, —NH—C(O)—CH₂—CH₃, —NH—CH₂—C(O)—CH₂—CH₃, —NH₂, —CH₂—NH₂, NO₂, one or two OCH₃, —O—CH(CH₃)—C(O)—CH₂—CH₃, —O—CH(CH₃)—C(O)—OH, OH, —O—CH₂—CH₂—CH₃, CF₃, and t-butyl;
k is 1 or 2;
Z is NH₂ or phenyl substituted with OH and two t-butyl; and
R² is H or CF₃;
provided that in the compound of Formula (I) A-B-D and X-Y-Z are not both simultaneously bromobenzyl, —CH₂—CH₂-phenyl, —CH₂—CH₂-bromophenyl, —CH₂—CH₂—CH₂-phenyl or —CH₂—CH₂—CH₂-bromophenyl;
provided that the compound of Formula (I) is not 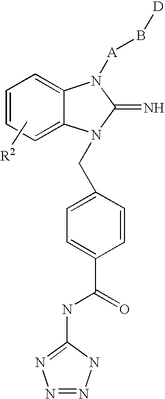
   wherein A-B-D is ethyl or isopropyl;
provided that the compound of Formula (I) is not 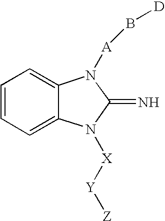
   wherein
A is selected from the group consisting of a bond, optionally substituted methyl, ethyl, and —CH₂—CH(OH)—CH₂;
B is selected from the group consisting of a bond, —C(O)—, —NH—C(O)— and O;
D is selected from the group consisting of H, OH, COOH, methyl, dimethylamino, furanyl, biphenyl, 3,5-di-t-butyl-4-hydroxyphenyl and phenyl wherein the phenyl is optionally substituted with Br, F, Cl, or —CH₂—OCH₂CH₃;
X is selected from the group consisting of a bond, CH₂ and pentyl;
Y is selected from the group consisting of a bond and —C(O); and
Z is selected from the group consisting of H, OH, butyl, biphenyl, heptyl, and morpholinyl, or
Z is selected from the group consisting of
   benzo[1,3]dioxazinyl substituted with two methyls;
   benzimidazolyl substituted with methyl and t-butyl;
   benz[1,3,4]oxathiazin is optionally substituted with one or more CH₃, t-butyl and oxo;
   cyclohexyl substituted with propyl;
   indolyl substituted with OCH₃;
   phenyl optionally substituted with Br, Cl or —C(O)NH-tetrazolyl;
   phenyl substituted with OH and two t-butyls;
   phenyl is optionally substituted with pyrrolidinyl substituted with two —CH₂—O—C(CH₃)₃; the dihydrobenz[1,4]oxazinyl substituted with CH₃ and t-butyl;
   piperazinyl is optionally substituted with diphenylmethyl;
   piperidinyl substituted with OH and four CH₃;
   pyrimidinyl substituted with OH and two t-butyl;
   pyrrolidinyl substituted with two —H₂—O—CH(CH₃)₃;
   pyrrolyl substituted with —C(O)—CH(CH₃)₂ and two CH₃;
provided that the compound of Formula (I) is not 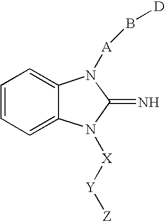
   wherein
A is selected from the group consisting of a bond, CH₂, ethyl and propyl;
B is selected from a bond, and —C(O)—NH—CH₂;
D is selected from H, COOH, ethyl, propyl, (C₁-C₂)alkoxy, pentyl, and phenyl wherein the phenyl is optionally substituted with Br, —CH₂—OCH₂CH₃ or —O—CH₂CH(CH₃)₂;
X is selected from the group consisting of a bond, —CH(CH₃), CH₂, —CH₂—CH(OCH₃), —CH(OH), ethyl and pentyl;
Y is selected from the group consisting of a bond, —C(O), —C(O)—NH and NH; and
Z is selected from the group consisting of H, CH₃, ethyl, propyl, butyl, and morpholinyl; or
Z is selected from the group consisting of H, CH₃, CH₂OH, benzyloxy, cyclohexyl substituted with propyl and phenyl substituted with Br, and phenyl substituted with Br and 3,5-di-t-butyl-4-hydroxyphenyl; and
provided that the compound of Formula (I) is not 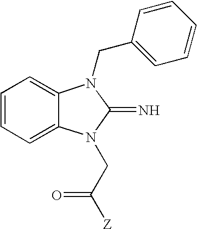
   wherein Z is selected from the group consisting of
benz[1,3,4]oxathiazin substituted with t-butyl and two oxo,
benzo[1,4]oxazinyl substituted with one or more CH₃, oxo, t-butyl or —C(O)—CH₃,
benzimidazolyl substituted with CH₃ and t-butyl,
benzo[1,3]dioxazinyl substituted with one or more CH₃,
benzo[1,3]dioxazolyl substituted with one or more CH₃,
benzofuranyl substituted with one or more t-butyl, CH₃, ethyl, NO₂, and oxo,
benzoxazolyl substituted with one or more CH₃, oxo and t-butyl, biphenyl,
dihydrobenz[1,4]oxazinyl substituted with two CH₃,
dihydrobenzo[b]thiophenyl substituted with one or more t-butyl or CH₃,
dihydrobenzofuranyl substituted with one or more —N(CH₃)—C(O)—CH₃ or CH₃, indolyl substituted with one or more Br, CH₃ or —CH₂—C(CH₃)₃,
naphthyl substituted with OH, or
phenyl substituted with one or more OH, CH₃, t-butyl or —CH₂—OCH₃.

In a second embodiment the invention provides compounds or pharmaceutically acceptable salts thereof according to any of the foregoing inventions wherein the compound is 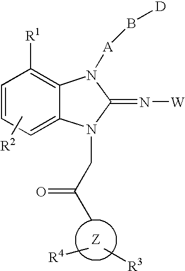
wherein
R¹ is selected from the group consisting of H, Br, Cl, CF₃, —C(O)OCH₃, pyridinyl, OCH₃, (C₂-C₅)alkenyl, phenyl, phenylethyl, biphenyl, imidazolyl, naphthyl, pyrazolyl and optionally substituted (C₁-C₅)alkyl;
Z is selected from the group consisting of benzo[1,3]dioxazolyl, benzo[d]isoxazolyl, 2,3-dihydrobenzo[1,4]dioxine, naphthyl, benzoxazolyl, furanyl, thienyl, phenyl, 4-morpholin-4-yl-phenyl and 4-pyrrolidin-1-yl-phenyl;
R³ is selected from the group consisting of H, Br, Cl, CH₃, CF₃, t-butyl and phenyl;
R⁴ is selected from the group consisting of H, Br, Cl, NO₂, CH₃, CF₃ and phenyl;
R² is selected from the group consisting of H, one or two CH₃, CN, (C₁-C₅)alkoxy, CF₃, OCF₃ and —C(O)-phenyl;
R³ is selected from the group consisting of H, Br, Cl, CH₃, pyrrolidinyl, morpholinyl, CF₃, t-butyl and phenyl;
R⁴ is selected from the group consisting of H, Br, Cl, NO₂, CH₃, CF₃ and phenyl;
A is selected from the group consisting of a bond or (C₁-C₃)alkyl;
B is selected from the group consisting of a bond, —C(O)—N(R^{a})₂—, —N(R^{a})—C(O)—, C(O) and O;
   R^{a} is H or (C₁-C₄)alkyl;
D is selected from the group consisting of H, OH, CH₃, COOH, (C₃)alkenyl, (C₂-C₄)alkoxy, (C₃-C₅)cycloalkyl, and dimethylamino, or is selected from the optionally substituted group consisting of morpholinyl, piperidinyl, benzyl, phenyl, piperazinyl, pyridyl, quinolinyl, amino, thienyl, pyridylcarbonyl, phenylcarbonylmorpholinyl, phenylcarbonylpiperizinyl and phenylcarbonylpyrrolidinyl;
W is selected from the group consisting of H, CN, (C₁-C₄)alkyl, —CH₂—CH₂—CH₂OH, CH₂CH₂OH, —CH₂—CH₂—OCH₃, —CH₂-Cyclopropyl, benzyl, dimethylaminobutyl, dimethylaminoethyl, dimethylaminopropyl, —C(O)—(C₁-C₂)alky, —CH₂-pyridinyl and —C(O)NH-phenyl wherein the phenyl is substituted with Br.

In a third embodiment the invention provides compounds or pharmaceutically acceptable salts thereof according to any of the foregoing inventions wherein R¹ is selected from the group consisting of Br, Cl, CH₂OH, CF₃, —C(O)OCH₃, pyridinyl, OCH₃, (C₂-C₅)alkenyl, phenyl, phenylethyl, biphenyl, imidazolyl, naphthyl, pyrazolyl and optionally substituted (C₁-C₅)alkyl.

In a fourth embodiment the invention provides compounds or pharmaceutically acceptable salts thereof according to any of the foregoing inventions wherein R¹ is H, Z is biphenyl or Z is phenyl optionally substituted with CN, NO₂, OCHF₂, OCF₃, CF₃, one or more F, one or more OCH₃ or one or more methyl and A-B-D is not benzyl.

In a fifth embodiment the invention provides compounds or pharmaceutically acceptable salts thereof according to any of the foregoing first through third embodiments of the invention wherein the compound is 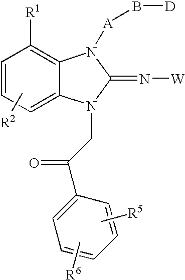
wherein
R¹ is selected from the group consisting of H, —C(O)—OCH₃, Br, Cl, OCH₃, CH₂OH, —C(═CH₂)CH₃, —CH═CH₂, —CH═CH—CH₃, —CH₂CH₂—O-phenyl, —CH₂CH₂CH₂OCH₃, CF₃, phenylethyl, CH₃, ethyl, isopropyl, butyl, propyl and cyclopropyl;
R² is selected from the group consisting of H, Cl, CN, OCH₃, CF₃, CH₃ and —C(O)-phenyl;
A is selected from the group consisting of a bond and optionally substituted (C₁-C₄)alkyl;
B is selected from the group consisting of a bond, —N(R^{a})—C(O)—, —C(O)—N(R^{a})C(O)—, —C(O)N(R^{a})—, C(O) and O;
   wherein R^{a} is H or CH₃;
D is selected from the group consisting of H, (C₁-C₂)alkoxy, COOH, optionally substituted (C₁-C₂)alkyl, (C₃-C₆)cycloalkyl, dibenzylamino, thienyl, morpholinyl, optionally substituted benzyl, CF₃, Cl, and optionally substituted phenyl;
   wherein the benzyl or the phenyl is optionally substituted with Br, CH₃, NO₂, CF₃ or OCH₃;
W is selected from the group consisting of H, —CH(CH₃)₂ and optionally substituted (C₁₋C₄)alkyl;
R⁵ is selected from the group consisting of H, Br, Cl, F, NO₂, OCF₃, OCH₃, ethyl and CH₃;
R⁶ is selected from the group consisting of H, Br, Cl, F, OCH₃, CH₃ and phenyl.

In a sixth embodiment the invention provides for the following compounds:

1-(4-bromophenyl)-2-[2-imino-3-(2-morpholin-4-yl)-ethyl)-2,3-dihydro-benzoimidazol-1-yl]-ethanone

2-(4-bromophenyl)-1-{3-[2(4-bromophenyl)-2-oxo-ethyl]-2-imino-2,3-dihydro-benzoimidazol-1-yl}-ethanone

1-(4-chlorophenyl)-2-[2-imino-3-methyl-4-((E)-propenyl)-2,3-dihydro-benzoimidazol-1-yl-ethanone

2-(4-chloro-2-imino-3-propyl-2,3-dihydrobenzoimidazol-1-yl)-1-(4-chlorphenyl)-ethanone

2-(2-imino-3-methyl-2,3-dihydrobenzoimidazol-1-yl)-1-4-trifluormethoxyphenyl)-ethanone

2-(2-imino-3-methyl-2,3-dihydrobenzoimidazol-1-yl)-1-p-tolyl-ethanone

2-(3-benzyl-2-imino-2,3-dihydrobenzoimidazol-1-yl)-1-(4-bromophenyl)-ethanone

1-(4-bromophenyl)-2-(3-ethyl-2-imino-2,3-dihydrobenzoimidazol-1-yl)-ethanone

1-(4-bromophenyl)-2-[4-chloro-2-imino-3-(2-phenoxyethyl)-2,3-dihydrobenzoimidazol-1-yl]-ethanone

3-{3-[2-(4-chlorophenyl)-2-oxo-ethyl]-2-imino-2,3-dihydrobenzoimidazol-1-yl}—N-methyl-N-(3-trifluoromethylbenzyl)-propionamide

1-(4-bromophenyl)-2-{2-[(Z)-isopropylimino]-3-methyl-2,3-dihydrobenzoimidazol-1-yl}-ethanone

1-(4-bromophenyl)-2-{2-[(Z)-methylimino]-3-phenethyl-2,3-dihydrobenzoimidazol-1-yl}-ethanone

1-(4-bromophenyl)-2-{2[Z)-methylimino]-3-phenethyl-2,3-dihydrobenzoimidazol-1-yl}-ethanone

1-(2,4-dimethylphenyl)-2-(2-imino-3-methyl-2,3-dihydrobenzoimidazol-1-yl)-ethanone

1-(2,4-dimethylphenyl)-2-(2-imino-3-(2-methoxy-ethyl)-2,3-dihydrobenzoimidazol-1-yl)-ethanone

2-(4-chloro-3-ethyl-2-imino-2,3-imino-2,3-dihydrobenzimidazol-1-yl)-(2,4-dichlorophenyl)-ethanone

1-(4-chlorophenyl)-2(2-imino-3,5-dimethyl-2,3-dihydrobenzoimdazol-1-yl)-ethanone

3-{3-[2-(4-chlorophenyl)-2-oxoethyl]-2-imino-6-methyl-2,3-dihydrobenzoimidazol-1-yl}-propionic acid ethyl ester

2-(3-butyl-4-chlor-2-imino-2,3-dihydrobenzoimidazol-1-yl)-1-(4-chlorophenyl)-ethanone

1-(4-bromophenyl)-2-[2-imino-3-(2,2,2-trifluoroethyl)-2,3-dihydrobenzoimidazol-1-yl]-ethanone

2-[4-chloro-2-imino-3-(3-methoxypropyl)-2,3dihydrobenzoimidazol-1-yl]-1-(2,4-dichlorophenyl)-ethanone

1-(3-bromophenyl)-2-(2-imino-3-methyl-2,3-dhydrobenzoimidazol-1-yl)-ethanone

1-(2-chlorphenyl)-2-(2-imino-3-methyl-2,3-dihydrobenzoimidazol-1-yl)-ethanone

2-(4-chloro-2-imino-3-propyl-2,3-dihydrobenzoimidazol-1-yl)-1phenyl-ethanone

1-(2-Nitrophenyl)-2-(4-chloro-2-imino-3-methyl-2,3-dihydrobenzoimidazol-1-yl)-ethanone

1-(4-bromophenyl)-2-(4-ethyl-2-imino-3-methyl-2,3-dihydrobenzoimidazol-1-yl)-ethanone

1-(4-bromophenyl)-2-(2-imino-3-propyl-2,3-dihydrobenzoimidazol-1-yl)-ethanone

N-(2-{3-[2-(4-chlorophenyl)-2-oxoethyl]-2-imino-2,3-dihydrobenzoimidazol-1-yl]-ethyl)-N-methyl-2-phenyl acetamide

N-(2-{3-[2-(4-chlorophenyl)-2-oxoethyl]-2-imino-2,3-dihydrobenzoimidazol-1-yl]-ethyl)-N-methyl-benzamide

3-{3-[2-(4-chlorphenyl)-2-oxo-ethyl]-2-imino-2,3-dihydrobenzoimidazol-1-yl]—N-methyl-benzyl)-propionamide

N-(3-{3-2-(4-bromophenyl)-2-oxoethyl]-7-ethyl-2-imino2,3-dihydrobenzoimidazol-1-yl}-propyl)-N-methyl-2-phenyl-acetamide

1-(4-bromophenyl)-2-(3-cyclopropyl)-2-imino-2,3-dihydrobenzoimidazol-1-yl)-ethanone

1-(4-chlorophenyl)-2-(2-imino-3-methyl-2,3-dihydrobenzoimidazol-1-yl)-ethanone

1-(4-chlorophenyl)-2-[3-(-dibenzylaminoethyl)-2-imino-2,3-dihydrobenzoimidazol-1-yl]-ethanone

3-{3-[2-(4-chlorophenyl)-2oxo-ethyl]-2-imino-2,3-dihydrobenzoimidazol-1-yl}—N-emthyl-N-(3-methylbenzyl)-propionamide

N-(2-{3-[2-(4-chlorophenyl)-2-oxo-ethyl]-2-imino-2,3-dihydrobenzoimidazol-1-yl}-ethyl)-2-cyclohexyl-N-methyl-acetamide

N-(3-chlorobenzyl)-3-{3-[2-(4-chlorphenyl)-2-oxo-ethyl]-2-imino-2,3-dihydrobenzoimidazol-1-yl}—N-methyl-propionamide

3-{3-[2-(4-bromophenyl)-2-oxo-ethyl]-2-imino-2,3-dihydrobenzoimidazol-1-yl}-propionic acid ethyl ester

1-(4-bromophenyl)-2-(3-butyl-2-imino-2,3-dihydrobenzoimidazol-1-yl)ethanone

2-[3-(2-benzylmethylamino)-ethyl]-2-imino-2,3-dihydrobenzoimdazol-1-yl]-1-(4-chlorophenyl)-ethanone

1-(4-Nitrophenyl)-2-(2-imino-3-methyl-2,3-dihydrobenzoimidazol-1-yl)-ethanone

N-(2-{3-[2-(2,4-dichlorophenyl§-2-oxo-ethyl]-2-imino-2,3-dihydrobenzoimidazol-1-yl}-ethyl)-N-methyl-2-phenyl-acetamide

1-(4-bromophenyl)-2-(3-ethyl-2-imino-2,3-dihydrobenzoimidazol-1-yl)-ethanone

1-(4-bromophenyl)-2-(3-cyclopropylmethyl-2-imino-2,3-dihydrobenzoimidazol-1-yl)-ethanone

1-(4-bromophenyl)-2-(3-methyl-2-(3-hydroxypropylimino-2,3-dihydrobenzoimidazol-1-yl)-ethanone.

In a seventh embodiment the invention provides compounds or pharmaceutically acceptable salts thereof according to the first through third embodiments and the fifth embodiment of the invention wherein R¹ is selected from the group consisting of —C(O)—OCH₃, Br, Cl, OCH₃, CH₂OH, —C(═CH₂)CH₃, —CH═CH₂, —CH═CH—CH₃, —CH₂CH₂—O-phenyl, —CH₂CH₂CH₂OCH₃, CF₃, phenylethyl, CH₃, ethyl, isopropyl, butyl and propyl.

In an eighth embodiment the invention provides compounds or pharmaceutically acceptable salts thereof according to the first through third embodiments and the fifth and seventh embodiments of the invention wherein
R¹ is selected from the group consisting of H, Br, Cl, CF₃, OCH₃, CH₂OH, —C(═CH₂)CH₃, —CH═CH₂, —CH₂═CH—CH₃, —CH₂CH₂—O-phenyl, —CH₂CH₂CH₂OCH₃, CH₃, ethyl, isopropyl, propyl and butyl;
A is a bond or optionally substituted (C₁-C₄)alkyl;
   wherein the alkyl is optionally substituted by OH;
      B is selected from the group consisting of a bond, —N(CH₃)—C(O), C(O)—N(CH₃), C(O) and O;
D is selected from the group consisting of H, COOH, CH₂OH, (C₁-C₂)alkoxy, cyclopropyl, cyclohexyl, dibenzylamino, phenyl and optionally substituted benzyl;
   wherein the benzyl is optionally substituted with CH₃ or NO₂;
W is selected from the group consisting of H, CH₃, ethyl, CH₂CH₂OH and —CH₂CH₂CH₂OH;
R⁵ is selected from the group consisting of H, Br, Cl, OCH₃, ethyl and NO₂; and
R⁶ is selected from the group consisting of H, Br, Cl and OCH₃.

In a ninth embodiment the invention provides compounds or pharmaceutically acceptable salts thereof according to the first through third and the fifth, seventh and eighth embodiments of the invention wherein R¹ is selected from the group consisting of Br, Cl, OCH₃, CH₂OH, —C(═CH₂)CH₃, —CH═CH₂, —CH₂═CH—CH₃, CH₃, ethyl, isopropyl and propyl.

In a tenth embodiment the invention provides compounds or pharmaceutically acceptable salts thereof according to the first through third, fifth and seventh through ninth embodiments of the invention wherein
R¹ is selected from the group consisting of H, Br, Cl, CH₂OH, —C(═CH₂)CH₃, —CH═CH₂, —CH₂CH₂—O-phenyl, —CH₂CH₂CH₂OCH₃, CF₃, CH₃, ethyl, isopropyl, butyl and propyl;
A is a bond or (C₁-C₄)alkyl;
B is selected from the group consisting of a bond, C(O), N(CH₃)—C(O), C(O)N(CH₃), and O;
D is selected from the group consisting of H, ethoxy, cyclopropyl, cyclohexyl, dibenzylamino, optionally substituted phenyl and optionally substituted benzyl;
W is H or ethyl; R⁵ is Br or Cl; and R⁶is H or Cl.

In an eleventh embodiment the invention provides compounds or pharmaceutically acceptable salts thereof according to the first through third, fifth and seventh through tenth embodiments of the invention wherein R¹ is Br, Cl, CH₂OH, —C(═CH₂)CH₃, —CH═CH₂, —CH₂CH₂—O-phenyl, —CH₂CH₂CH₂OCH₃, CF₃, CH₃, ethyl, isopropyl, butyl or propyl.

In a twelfth embodiment the invention provides compounds or pharmaceutically acceptable salts thereof according to the first through third, fifth and seventh through eleventh embodiments of the invention wherein R¹ is selected from the group consisting of H, Br, Cl, CH₂OH, —C(═CH₂)CH₃, CH₃, ethyl, isopropyl and propyl; A is CH₂; B is a bond; D is H; and W is H.

In a thirteenth embodiment the invention provides compounds or pharmaceutically acceptable salts thereof according to the first through third, fifth and seventh through twelfth embodiments of the invention wherein R¹ is selected from the group consisting of H, Cl, CH₃, ethyl, isopropyl, propyl and —C(═CH₂)CH₃.

In a fourteenth embodiment the invention provides compounds or pharmaceutically acceptable salts thereof according to the first through third, fifth and seventh through thirteenth embodiments of the invention wherein
R¹ is selected from the group consisting of H, Cl, CH₃, and ethyl.

In a fifteenth embodiment the invention provides compounds or pharmaceutically acceptable salts thereof according to the first embodiment of the invention wherein the compound is 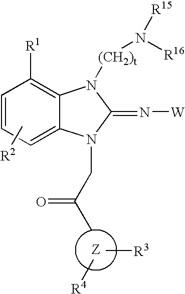
wherein
t is 0, 1, 2 or 3;
Z is phenyl or thienyl,
R¹ is selected from the group consisting of H, Cl and ethyl;
R² is H;
R¹⁵ is selected from the group consisting of H, (C₁-C₂)alkyl, phenyl, benzyl and —C(O)—OC(CH₃)₃;
R¹⁶ is selected from the group consisting of (C₁-C₂)alkyl, (C₃-C₆)cycloalkyl, optionally substituted phenylcarbonyl, optionally substituted benzyl, optionally substituted benzylcarbonyl, methylcarbonyl, and thienylcarbonyl;
R³ is selected from the group consisting of H, Br, Cl and CH₃; and
R⁴ is H or Cl.

In a sixteenth embodiment the invention provides compounds or pharmaceutically acceptable salts thereof according to the first or fifteen embodiment of the invention wherein Z is phenyl; R¹⁵ is CH₃ or benzyl; R¹⁶ is selected from the group consisting of thienylcarbonyl, benzylcarbonyl, benzyl and cyclohexyl; and R³ is selected from the group consisting of Br, Cl and CH₃.

In a seventeenth embodiment the invention provides compounds or pharmaceutically acceptable salts thereof according to the first, fifteenth or sixteenth embodiments of the invention wherein t is 2 or 3; R¹ is H or ethyl; R¹⁵ is CH₃; R¹⁶ is thienylcarbonyl or benzylcarbonyl; and R³ is Cl.

In an eighteenth embodiment the invention provides compounds or pharmaceutically acceptable salts thereof according to the first or fifteenth through seventeenth embodiment of the invention wherein the compound is 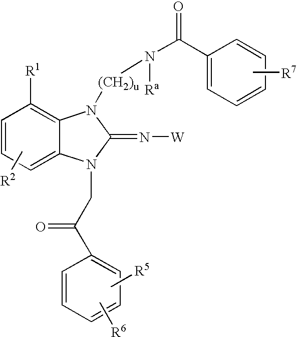
wherein
R¹ is selected from the group consisting of methyl, ethyl and Cl;
R²is H or Cl;
u is 2, 3 or 4;
R⁵ is selected from the group consisting of H, Br, Cl and OCH₃;
R⁶ is selected from the group consisting of H, Cl and OCH₃;
R⁷ is selected from the group consisting of H, CH₃, Cl and F;
R^{a} is H or CH₃; and
W is H.

In a nineteenth embodiment the invention provides compounds or pharmaceutically acceptable salts thereof according to the first or fifteenth through eighteenth embodiments of the invention wherein the compound is 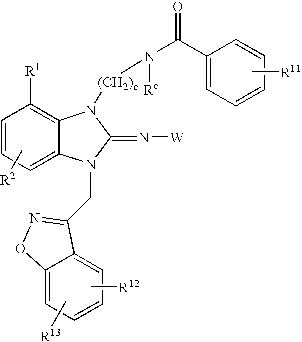
wherein
e is 0, 1 or 2;
R¹¹ is one or more substituents selected from the group consisting of H, CH₃, OH, CN, NO₂, CO₂H, CO₂(C₁-C₃)alkyl, CF₃ and halo;
R¹² and R¹³ are independently selected from the group consisting of H, CH₃, OH, CN, NO₂, CF₃ and halo; and
R^{c} is H, CH₃, NO₂, or CF₃.

In a twentieth embodiment the invention provides compounds or pharmaceutically acceptable salts thereof according to the first or fifteenth through nineteenth embodiments of the invention wherein the compound is 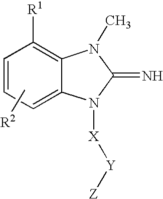
wherein
R¹ is H;
R² is H;
X is CH₂;
Y is S(O) or S; and
Z is phenyl optionally substituted with Cl.

### DETAILED DESCRIPTION OF THE INVENTION

In a related aspect the invention provides a method for modulating activity of CXCR3 in a human subject suffering from a disorder in which CXCR3 functional activity is detrimental, comprising administering to the human subject a compound of Formula (I) such that CXCR3 activity in the human subject is inhibited and treatment is achieved.

A compound of formula (I) or a salt thereof or pharmaceutical compositions containing a therapeutically effective amount thereof is useful in the treatment of a disorder selected from the group comprising rheumatoid arthritis, osteoarthritis, juvenile chronic arthritis, Lyme arthritis, psoriatic arthritis, reactive arthritis, and septic arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection (including but not limited to bone marrow and solid organ rejection), acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjögren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, and cancers such as lung, breast, stomach, bladder, colon, pancreas, ovarian, prostate and rectal cancer and hematopoietic malignancies (leukemia and lymphoma), and hematopoietic malignancies (leukemia and lymphoma), and diseases involving inappropriate vascularization for example diabetic retinopathy, retinopathy of prematurity, choroidal neovascularization due to age-related macular degeneration, and infantile hemangiomas in human beings. In addition, such compounds may be useful in the treatment of disorders such as, edema, ascites, effusions, and exudates, including for example macular edema, cerebral edema, acute lung injury, adult respiratory distress syndrome (ARDS), proliferative disorders such as restenosis, fibrotic disorders such as hepatic cirrhosis and atherosclerosis, mesangial cell proliferative disorders such as glomerulonephritis, diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, Crow-Fukase syndrome (POEMS), preeclampsia, menometrorrhagia, cat scratch fever, rubeosis, neovascular glaucoma and retinopathies such as those associated with diabetic retinopathy, retinopathy of prematurity, or age-related macular degeneration. In addition, these compounds can be used as active agents against solid tumors, malignant ascites, von Hippel Lindau disease, hematopoietic cancers and hyperproliferative disorders such as thyroid hyperplasia (especially Grave's disease), and cysts (such as hypervascularity of ovarian stroma characteristic of polycystic ovarian syndrome (Stein-Leventhal syndrome) and polycystic kidney disease since such diseases require a proliferation of blood vessel cells for growth and/or metastasis.

Compounds of formula (I) of the invention can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the compound of the present invention. The additional agent also can be an agent that imparts a beneficial attribute to the therapeutic composition e.g., an agent which effects the viscosity of the composition.

It should further be understood that the combinations which are to be included within this invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

For example, in the treatment or prevention of inflammation, the present compounds may be used in conjunction or combination with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codiene, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextromethorphan; a diuretic; and a sedating or non-sedating antihistamine. Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention. Examples of other active ingredients that may be combined with a compound of the present invention, either administered separately or in the same pharmaceutical compositions, include, but are not limited to: (a) VLA-4 antagonists, (b) steroids such as beclomethasone, methylprednisolone, betamethasone, prednisone, dexamethasone, and hydrocortisone; (c) immunosuppressants such as cyclosporine (cyclosporine A, Sandimmune®, Neoral®), tacrolimus (FK-506, Prograf®), rapamycin (sirolimus, Rapamune®) and other FK-506 type immunosuppressants, and mycophenolate, e.g., mycophenolate mofetil (CellCept®); (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as .beta.2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors such as celecoxib (Celebrex®) and rofecoxib (Vioxx®); (h) inhibitors of phosphodiesterase type IV (PDE-IV); (i) gold compounds such as auranofin and aurothioglucose, (j) inhibitors of phosphodiesterase type IV (PDE-IV); (k) other antagonists of the chemokine receptors, especially CCR1, CCR2, CCR3, CCR5, CCR6, CCR8 and CCR10; (l) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (m) anti-diabetic agents such as insulin, sulfonylureas, biguanides (metformin), α-glucosidase inhibitors (acarbose) and glitazones (troglitazone and pioglitazone); (n) preparations of interferon beta (interferon β-1α; interferon β-1b); (o) etanercept (Enbrel®), (p) antibody therapies such as orthoclone (OKT3), daclizumab (Zenapax®), infliximab (Remicade®), basiliximab (Simulect®) and anti-CD40 ligand antibodies (e.g., MRP-1); and (q) other compounds such as 5-aminosalicylic acid and prodrugs thereof, hydroxychloroquine, D-penicillamine, antimetabolites such as azathioprene and 6-mercaptopurine, and cytotoxic cancer chemotherapeutic agents. The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

Immunosuppressants within the scope of the present invention further include, but are not limited to, leflunomide, RAD001, ERL080, FTY720, CTLA-4, antibody therapies such as orthoclone (OKT3), daclizumab (Zenapax®) and basiliximab (Simulect®), and antithymocyte globulins such as thymoglobulins.

In particularly preferred embodiments, the present methods are directed to the treatment or prevention of multiple sclerosis using a compound of the invention either alone or in combination with a second therapeutic agent selected from betaseron, avonex, azathioprene (Imurek®, Imuran®), capoxone, prednisolone and cyclophosphamide. When used in combination, the practitioner can administer a combination of the therapeutic agents, or administration can be sequential.

In still other particularly preferred embodiments, the present methods are directed to the treatment or prevention of rheumatoid arthritis, wherein the compound of the invention is administered either alone or in combination with a second therapeutic agent selected from the group consisting of methotrexate, sulfasalazine, hydroxychloroquine, cyclosporine A, D-penicillamine, infliximab (Remicade®), etanercept (Enbrel®), adalimumab (Humira®), auranofin and aurothioglucose.

In yet other particularly preferred embodiments, the present methods are directed to the treatment or prevention of an organ transplant condition wherein the compound of the invention is used alone or in combination with a second therapeutic agent selected from the group consisting of cyclosporine A, FK-506, rapamycin, mycophenolate, prednisolone, azathioprene, cyclophosphamide and an antilymphocyte globulin.

A compound of formula (I) of the invention may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors, TNFα converting enzyme (TACE) inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™ and p55TNFRIgG (Lenercept)), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol Hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl, misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, and Mesopram. Preferred combinations include methotrexate or leflunomide and in moderate or severe rheumatoid arthritis cases, cyclosporine and anti-TNF antibodies as noted above.

Non-limiting examples of therapeutic agents for inflammatory bowel disease with which a compound of formula (I) of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-12, IL-15, IL-16, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signalling by proinflammatory cytokines such as TNFα or IL-1 (e.g. IRAK, NIK, IKK, p38 or MAP kinase inhibitors); IL-1β converting enzyme inhibitors; TNFα converting enzyme inhibitors; T-cell signalling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p⁵⁵ or p75 TNF receptors, sIL-1RI, sIL-1RII, sIL-6R) and antiinflammatory cytokines (e.g. IL-4, IL-10, L-11, IL-13 and TGFβ). Preferred examples of therapeutic agents for Crohn's disease in which a compound of formula (I) can be combined include the following: TNF antagonists, for example, anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (LENERCEPT™)) inhibitors and PDE4 inhibitors. A compound of formula (I) can be combined with corticosteroids, for example, budenoside and dexamethasone; sulfasalazine, 5-aminosalicylic acid; olsalazine; and agents which interfere with synthesis or action of proinflammatory cytokines such as IL-1, for example, IL-1β converting enzyme inhibitors and IL-1ra; T cell signaling inhibitors, for example, tyrosine kinase inhibitors 6-mercaptopurines; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone hcl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam HCl; cyanocobalamin; folic acid; levofloxacin; methylprednisolone; natalizumab and interferon-gamma.

Non-limiting examples of therapeutic agents for multiple sclerosis with which a compound of formula (I) can be combined include the following: corticosteroids; prednisolone; methylprednisolone; azathioprine; cyclophosphamide; cyclosporine; methotrexate; 4-aminopyridine; tizanidine; interferon-β1a (AVONEX; Biogen); interferon-β1b (BETASERON; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE; Teva Pharmaceutical industries, Inc.); hyperbaric oxygen; intravenous immunoglobulin; clabribine; antibodies to or antagonists of other human cytokines or growth factors and their receptors, for example, TNF, LT, IL-1, IL-2, IL-6, IL-7, IL-8, IL-12, IL-23, IL-15, IL-16, EMAP-II, GM-CSF, FGF, and PDGF. A compound of formula (I) can be combined with antibodies to cell surface molecules such as CD2, CD3, CD4, CD8, CD19, CD20, CD25, CD28, CD30, CD40, CD45, CD69, CD80, CD86, CD90 or their ligands. A compound of formula (I) may also be combined with agents, such as methotrexate, cyclosporine, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-13 and TGFβ).

Preferred examples of therapeutic agents for multiple sclerosis in which a compound of formula (I) can be combined to include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

A compound of formula (I) may also be combined with agents, such as alemtuzumab, dronabinol, Unimed, daclizumab, mitoxantrone, xaliproden hydrochloride, fampridine, glatiramer acetate, natalizumab, sinnabidol, α-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist) MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL-4 agonists.

Non-limiting examples of therapeutic agents for Angina with which a compound of formula (I) of the invention can be combined include the following: aspirin, nitroglycerin, isosorbide mononitrate, metoprolol succinate, atenolol, metoprolol tartrate, amlodipine besylate, diltiazem hydrochloride, isosorbide dinitrate, clopidogrel bisulfate, nifedipine, atorvastatin calcium, potassium chloride, furosemide, simvastatin, verapamil HCl, digoxin, propranolol hydrochloride, carvedilol, lisinopril, spironolactone, hydrochlorothiazide, enalapril maleate, nadolol, ramipril, enoxaparin sodium, heparin sodium, valsartan, sotalol hydrochloride, fenofibrate, ezetimibe, bumetanide, losartan potassium, lisinopril/hydrochlorothiazide, felodipine, captopril and bisoprolol fumarate.

Non-limiting examples of therapeutic agents for Ankylosing Spondylitis with which a compound of formula (I) can be combined include the following: ibuprofen, diclofenac and misoprostol, naproxen, meloxicam, indomethacin, diclofenac, celecoxib, rofecoxib, Sulfasalazine, Methotrexate, azathioprine, minocyclin, prednisone, etanercept, infliximab and adalimumab (Humira®).

Non-limiting examples of therapeutic agents for Asthma with which a compound of formula (I) can be combined include the following: albuterol, salmeterol/fluticasone, montelukast sodium, fluticasone propionate, budesonide, prednisone, salmeterol xinafoate, levalbuterol HCl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, methylprednisolone, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin HCl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine hcl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone and metaproterenol sulfate.

Non-limiting examples of therapeutic agents for COPD with which a compound of formula (I) can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol HCl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, Cilomilast and Roflumilast.

Non-limiting examples of therapeutic agents for HCV with which a compound of formula (I) can be combined include the following: Interferon-alpha-2a, Interferon-alpha-2b, Interferon-alpha con1, Interferon-alpha-n1, Pegylated interferon-alpha-2a, Pegylated interferon-alpha-2b, ribavirin, Peginterferon alfa-2b+ribavirin, Ursodeoxycholic Acid, Glycyrrhizic Acid, Thymalfasin, Maxamine, VX-497 and any compounds that are used to treat HCV through intervention with the following targets: HCV polymerase, HCV protease, HCV helicase and HCV IRES (internal ribosome entry site).

Non-limiting examples of therapeutic agents for Idiopathic Pulmonary Fibrosis with which a compound of formula (I) can be combined include the following: prednisone, azathioprine, albuterol, colchicine, albuterol sulfate, digoxin, gamma interferon, methylprednisolone sod succ, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone HCl, potassium chloride, triamcinolone acetonide, tacrolimus anhydrous, calcium, interferon-alpha, methotrexate, mycophenolate mofetil and Interferon-gamma-1β.

Non-limiting examples of therapeutic agents for Myocardial Infarction with which a compound of formula (I) can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril HCl/mag carb, bumetanide, alteplase, enalaprilat, amiodarone hydrochloride, tirofiban HCl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutamine HCl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe and cariporide.

Non-limiting examples of therapeutic agents for Psoriasis with which a compound of formula (I) can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB and sulfasalazine.

Non-limiting examples of therapeutic agents for Psoriatic Arthritis with which a compound of formula (I) can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, efalizumab and adalimumab (Humira®).

Non-limiting examples of therapeutic agents for Restenosis with which a compound of formula (I) can be combined include the following: sirolimus, paclitaxel, everolimus, tacrolimus, ABT-57 and acetaminophen.

Non-limiting examples of therapeutic agents for Sciatica with which a compound of formula (I) can be combined include the following: hydrocodone bitartrate/apap, rofecoxib, cyclobenzaprine HCl, methylprednisolone, naproxen, ibuprofen, oxycodone HCl/acetaminophen, celecoxib, valdecoxib, methylprednisolone acetate, prednisone, codeine phosphate/apap, tramadol hcl/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone HCl, tizanidine HCl, diclofenac sodium/misoprostol, propoxyphene napsylate/apap, asa/oxycod/oxycodone ter, ibuprofen/hydrocodone bit, tramadol HCl, etodolac, propoxyphene HCl, amitriptyline HCl, carisoprodol/codeine phos/asa, morphine sulfate, multivitamins, naproxen sodium, orphenadrine citrate and temazepam.

Preferred examples of therapeutic agents for SLE (Lupus) in which a compound of formula (I) include the following: NSAIDS, for example, diclofenac, naproxen, ibuprofen, piroxicam, indomethacin; COX2 inhibitors, for example, Celecoxib, rofecoxib, valdecoxib; anti-malarials, for example, hydroxychloroquine; Steroids, for example, prednisone, prednisolone, budenoside, dexamethasone; Cytotoxics, for example, azathioprine, cyclophosphamide, mycophenolate mofetil, methotrexate; inhibitors of PDE4 or purine synthesis inhibitor, for example Cellcept. A compound of formula (I) may also be combined with agents such as sulfasalazine, 5-aminosalicylic acid, olsalazine, Imuran and agents which interfere with synthesis, production or action of proinflammatory cytokines such as IL-1, for example, caspase inhibitors like IL-1β converting enzyme inhibitors and IL-1ra. A compound of formula (I) may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of formula (I) can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of formula (I) may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, adalimumab (HUMIRA™), CA2 (REMICADE™), CDP 571, TNFR-Ig constructs, (p75TNFRIgG (ENBREL™) and p55TNFRIgG (LENERCEPT™)).

In this invention, the following definitions are applicable:

“Pharmaceutically acceptable salts” refers to those salts which retain the biological effectiveness and properties of the free bases and which are obtained by reaction with inorganic acids, for example, hydrochloric acid, hydrobromic acid, hydroiodic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, acetic acid, trifluoracetic acid, tartaric acid (e.g. (+) or (−)-tartaric acid or mixtures thereof), amino acids (e.g. (+) or (−)-amino acids or mixtures thereof), and the like. These salts can be prepared by methods known to those skilled in the art.

Certain compounds of formula I which have acidic substituents may exist as salts with pharmaceutically acceptable salts with bases. The present invention includes such salts. Examples of such salts include sodium salts, potassium salts, lysine salts and arginine salts. These salts may be prepared by methods known to those skilled in the art.

Certain compounds of formula I and their salts may exist in more than one crystal form and the present invention includes each crystal form and mixtures thereof.

Certain compounds of formula I and their salts may also exist in the form of solvates, for example hydrates, and the present invention includes each solvate and mixtures thereof.

Certain compounds of formula I may contain one or more chiral centers, and exist in different optically active forms. When compounds of formula I contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of formula I contains more than one chiral center it may exist in diastereoisomeric forms. The diastereoisomeric pairs may be separated by methods known to those skilled in the art, for example chromatography or crystallization and the individual enantiomers within each pair may be separated as described above. The present invention includes each diastereoisomer of compounds of formula I and mixtures thereof.

Certain compounds of formula I may exist in different tautomeric forms or as different geometric isomers, and the present invention includes each tautomer and/or geometric isomer of compounds of formula I and mixtures thereof.

Certain compounds of formula I may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of formula I and mixtures thereof.

Certain compounds of formula I may exist in zwitterionic form and the present invention includes each zwitterionic form of compounds of formula I and mixtures thereof.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The prodrug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial

Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to carboxylic acid substituents (e.g., —(CH₂)C(O)H or a moiety that contains a carboxylic acid) wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of Formula I wherein the free hydrogen of the hydroxyl substituent (e.g., R¹ contains hydroxyl) is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylamino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, P(O)(OH)₂, —P(O)(O(C₁-C₆)alkyl)₂ or glycosyl (the radical resulting from detachment of the hydroxyl of the hemiacetal of a carbohydrate).

The term “heterocyclic” or “heterocyclyl” as used herein, include non-aromatic, ring systems, including, but not limited to, monocyclic, bicyclic and tricyclic rings, which can be completely saturated or which can contain one or more units of unsaturation and have 3 to 12 atoms including at least one heteroatom, such as nitrogen, oxygen, or sulfur. For purposes of exemplification, which should not be construed as limiting the scope of this invention: azetidinyls, morpholinos, piperazines, piperidines, pyrans, triazoles, tetrazoles, thiadiazoles, thiomorpholinos or triazoles.

The term “heteroaryl”, as used herein include include aromatic and non-aromatic, ring systems, including, but not limited to, monocyclic, bicyclic and tricyclic rings, which can be completely saturated or which can contain one or more units of unsaturation and have 3 to 12 atoms including at least one heteroatom, such as nitrogen, oxygen, or sulfur. For purposes of exemplification, which should not be construed as limiting the scope of this invention: azaindole, benzo(b)thienyl, benzimidazolyl, benzo[1,3]dioxazinyl, benz[1,3,4]oxathiazinyl, dihydrobenz[1,4]oxazinyl, benzo[1,4]oxazinyl, benzo[d]isoxazolyl, benzoxazolyl, benzothiazolyl, benzothiadiazolyl, benzoxadiazolyl, furans, imidazoles, imidazopyridine, indole, indazoles, isoxazoles, isoquinolines, isothiazoles, oxadiazoles, oxazoles, naphthyridines, purine, pyrazines, pyrazoles, pyridines, pyrimidines, pyrroles, pyrrolidines, pyrrolo[2,3-d]pyrimidine, pyrazolo[3,4-d]pyrimidine), quinazolines, quinolines, quinazolines, thiazoles, tetrahydroindole, or thienyls.

As used herein, many moieties or substituents are termed as being either “substituted” or “optionally substituted”. When a moiety is modified by one of these terms, it denotes that any portion of the moiety that is known to one skilled in the art as being available for substitution can be substituted, which includes one or more substituents, where if more than one substituent then each substituent is independently selected. Such means for substitution are well-known in the art and/or taught by the instant disclosure. For purposes of exemplification, which should not be construed as limiting the scope of this invention, some examples of groups that are substituents are: alkenyl groups, alkoxy group (which itself can be substituted, such as —O—C₁-C₆-alkyl-OR, —O—C₁-C₆-alkyl-N(R)₂, and OCF₃), alkoxyalkoxy, alkoxycarbonyl, alkoxycarbonylpiperidinyl-alkoxy, alkyl groups (which itself can also be substituted, such as —C₁-C₆-alkyl-OR, —C₁-C₆-alkyl-N(R)₂, and —CF₃), alkylamino, alkylcarbonyl, alkylester, alkylnitrile, alkylsulfonyl, amino, aminoalkoxy, benzyl, CF₃, COH, COOH, CN, cycloalkyl, dialkylamino, dialkylaminoalkoxy, dialkylaminocarbonyl, dialkylaminocarbonylalkoxy, dialkylaminosulfonyl, esters (—C(O)—OR, where R is groups such as alkyl, heterocycloalkyl (which can be substituted), heterocyclyl, etc., which can be substituted), halogen or halo group (F, Cl, Br, I), hydroxy, morpholinoalkoxy, morpholinoalkyl, nitro, oxo, OCF₃, optionally substituted phenyl, S(O)₂CH₃, S(O)₂CF₃, and sulfonyl, N-alkylamino or N,N-dialkylamino (in which the alkyl groups can also be substituted).

For purposes of exemplification, which should not be construed as limiting the scope of this invention, some examples of groups that are substituents of amine groups are: alkenyl groups, alkyl groups (which itself can also be substituted, such as —C₁-C₆-alkyl-OR, —C₁-C₆-alkyl-N(R)₂, and —CF₃), —C(O)—O-alkyl, cycloalkyl, phenylcarbony (which itself can also be substituted) 1, benzylcarbonyl (which itself can also be substituted), thienylcarbonyl (which itself can also be substituted) and alkylcarbonyl (which itself can also be substituted), benzyl (which itself can also be substituted) and phenyl (which itself can also be substituted).

When the term “substituted heterocyclic” (or heterocyclyl), “substituted heteroaryl (or heteroaryl) or “substituted aryl” (or aryl) is used, what is meant is that the heterocyclic group is substituted with one or more substituents that can be made by one of ordinary skill in the art and results in a molecule that is a kinase inhibitor. For purposes of exemplification, which should not be construed as limiting the scope of this invention, preferred substituents for the heterocyclyls of this invention are each independently selected from the optionally substituted group consisting of alkenyl, alkoxy, alkoxyalkoxy, alkoxyalkyl, alkoxycarbonyl, alkoxycarbonylheterocycloalkoxy, alkyl, alkylcarbonyl, alkylester, alkyl-O—C(O)—, alkyl-heterocyclyl, alkyl-cycloalkyl, alkyl-cycloalkenyl, alkyl-nitrile, alkynyl, amido groups, amino, aminoalkyl, aminocarbonyl, benzyl, carbonitrile, carbonylalkoxy, carboxamido, CF₃, CN, —C(O)OH, —C(O)H, —C(O)—(O)(CH₃)₃, —OH, —C(O)O-alkyl, —C(O)O-cycloalkyl, —C(O)O-heterocyclyl, —C(O)-alkyl, —C(O)-amino, —C(O)—Cycloalkyl, —C(O)-heterocyclyl, cycloalkyl, dialkylaminoalkoxy, dialkylaminocarbonylalkoxy, dialkylaminocarbonyl, halogen, heterocyclyl, a heterocycloalkyl group, heterocyclyloxy, hydroxy, hydroxyalkyl, morpholinyl, nitro, NO₂, OCF₃, oxo, phenyl, phenylcarbonyl, pyrrolidinyl, —SO₂CH₃, —SO₂CR₃, tetrazolyl, thienylalkoxy, trifluoromethylcarbonylamino, trifluoromethylsulfonamido, heterocyclylalkoxy, heterocyclyl-S(O)ₚ, cycloalkyl-S(O)ₚ, alkyl-S-, heterocyclyl-S, heterocycloalkyl, cycloalkylalkyl, heterocycolthio, cycloalkylthio, -Z¹⁰⁵-C(O)N(R)₂, -Z¹⁰⁵-N(R)—C(O)-Z²⁰⁰, -Z¹⁰⁵-N(R)—S(O)₂-Z²⁰⁰, -Z¹⁰⁵-N(R)—C(O)—N(R)-Z²⁰⁰, —N(R)—C(O)R, —N(R)—C(O) OR, or —C(O)-heterocyclyl-OR, R_{c} and —CH₂OR;
where R_{c} for each occurrence is independently hydrogen, optionally substituted alkyl, optionally substituted aryl, —(C₁-C₆)—NR_{d}Rₑ, -E-(CH₂)ₜ—NR_{d}Rₑ, -E-(CH₂)ₜ—O-alkyl, -E-(CH₂),-S-alkyl, or -E-(CH₂),-OH
   wherein t is an integer from about 1 to about 6;
Z¹⁰⁵ for each occurrence is independently a covalent bond, alkyl, alkenyl or alkynyl; and
Z²⁰⁰ for each occurrence is independently selected from an optionally substituted group selected from the group consisting of alkyl, alkenyl, alkynyl, phenyl, alkyl-phenyl, alkenyl-phenyl or alkynyl-phenyl;
E is a direct bond, O, S, S(O), S(O)₂, or NR_{f}, wherein R_{f} is H or alkyl and R_{d} and Rₑ are independently H, alkyl, alkanoyl or SO₂-alkyl; or R_{d}, Rₑ and the nitrogen atom to which they are attached together form a five- or six-membered heterocyclic ring.

When the term “substituted phenyl” is used, what is meant is that the phenyl group is substituted with one or more substituents that can be made by one of ordinary skill in the art and results in a molecule that is a kinase inhibitor. For purposes of exemplification, which should not be construed as limiting the scope of this invention, preferred substituents for the phenyls of this invention are each independently selected from the optionally substituted group consisting of alkenyl, alkoxy, alkoxyalkyl, alkoxycarbonyl, alkyl, alkylcarbonyl, alkylester, alkyl-heterocyclyl, alkyl-cycloalkyl, alkyl-cycloalkenyl, alkynyl, amido groups, amino, aminoalkyl, aminocarbonyl, benzyl, carbonitrile, carbonylalkoxy, CF₃, CHF₂, CN, —C(O)OH, —C(O)H, —C(O)—(O)(CH₃)₃, —OH, —C(O)-alkyl, —C(O)-amino, —C(O)—Cycloalkyl, —C(O)-heterocyclyl, —C(O)—NH-heterocyclyl, especially —C(O)—NH-tetrazolyl, cycloalkyl, dialkylaminoalkoxy, dialkylaminocarbonyl, halogen, heterocyclyl, a heterocycloalkyl group, heterocyclyloxy, hydroxy, hydroxyalkyl, morpholinyl, nitro, NO₂, OCF₃, oxo, phenyl, pyrrolidinyl, —SO₂CH₃, —SO₂CR₃, tetrazolyl, trifluoromethylsulfonamido, heterocyclylalkoxy, heterocyclyl-S(O)p, cycloalkyl-S(O)ₚ, alkyl-S-, heterocyclyl-S, heterocycloalkyl, cycloalkylalkyl, heterocycolthio, cycloalkylthio, -Z¹⁰⁵-C(O)N(R)₂, -Z¹⁰⁵-N(R)—C(O)-Z²⁰⁰, -Z¹⁰⁵-N(R)—S(O)₂-Z²⁰⁰, -Z¹⁰⁵-N(R)—C(O)—N(R)-Z²⁰⁰, —N(R)—C(O)R, —N(R)—C(O)OR, or —C(O)-heterocyclyl-OR, R^{c} and —CH₂OR_{c};
where R_{c} for each occurrence is independently hydrogen, optionally substituted alkyl, optionally substituted aryl, —(C₁-C₆)—NR_{d}Rₑ, -E-(CH₂)₁—NR_{d}Rₑ, -E-(CH₂)ₜ—O-alkyl, -E-(CH₂)ₜ-S-alkyl, or -E-(CH₂)ₜ—OH
   wherein t is an integer from about 1 to about 6;
Z¹⁰⁵ for each occurrence is independently a covalent bond, alkyl, alkenyl or alkynyl; and
Z²⁰⁰ for each occurrence is independently selected from an optionally substituted group selected from the group consisting of alkyl, alkenyl, alkynyl, phenyl, alkyl-phenyl, alkenyl-phenyl or alkynyl-phenyl;
E is a direct bond, O, S, S(O), S(O)₂, or NR_{f}, wherein R_{f} is H or alkyl and R_{d} and Rₑ are independently H, alkyl, alkanoyl or SO₂-alkyl; or R_{d}, Rₑ and the nitrogen atom to which they are attached together form a five- or six-membered heterocyclic ring.

An “heterocycloalkyl” group, as used herein, is a heterocyclic group that is linked to a compound by an aliphatic group having from one to about eight carbon atoms. For example, imidazolylethyl group is an example of a heterocycloalkyl group.

As used herein, “aliphatic” or “an aliphatic group” or notations such as “(C₀-C₈)” include straight chained or branched hydrocarbons which are completely saturated or which contain one or more units of unsaturation, and, thus, includes alkyl, alkenyl, alkynyl and hydrocarbons comprising a mixture of single, double and triple bonds. When the group is a C₀ it means that the moiety is not present or in other words, it is a bond. As used herein, “alkyl” means C₁-C₈ and includes straight chained or branched hydrocarbons which are completely saturated. Preferred alkyls are methyl, ethyl, propyl, butyl, pentyl, hexyl and isomers thereof. As used herein, “alkenyl” and “alkynyl” means C₂-C₈ and includes straight chained or branched hydrocarbons which contain one or more units of unsaturation, one or more double bonds for alkenyl and one or more triple bonds for alkynyl.

As used herein, aromatic groups (or aryl groups) include aromatic carbocyclic ring systems (e.g. phenyl and cyclopentyldienyl) and fused polycyclic aromatic ring systems (e.g. naphthyl, biphenylenyl and 1,2,3,4-tetrahydronaphthyl).

As used herein, cycloalkyl means C₃-C₁₂ monocyclic or multicyclic (e.g., bicyclic, tricyclic, etc.) hydrocarbons which is completely saturated or has one or more unsaturated bonds but does not amount to an aromatic group. Preferred examples of a cycloalkyl group are cyclopropyl, cyclobutyl, cyclopentyl, cyclopentenyl, cyclohexyl and cyclohexenyl.

As used herein, amido group means —NHC(═O)—.

As used herein, acyloxy groups are —OC(O)R.

Pharmaceutical Formulations

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Routes of Administration.

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

Composition/Formulation

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition of a given protein kinase activity). In some cases it is appropriate to determine the IC₅₀ in the presence of 3 to 5% serum albumin since such a determination approximates the binding effects of plasma protein on the compound. Such information can be used to more accurately determine useful doses in humans. Further, the most preferred compounds for systemic administration effectively inhibit protein kinase signaling in intact cells at levels that are safely achievable in plasma.

A therapeutically effective dose refers to that amount of a compound of Formula I or a combination of two or more such compounds, which inhibits, totally or partially, the progression of a condition or alleviates, at least partially, one or more symptoms of the condition. A therapeutically effective amount can also be an amount which is prophylactically effective. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art. The exact formulation, route of administration and dosage can be chosen by the individual physician in view of the patient's condition. (See e.g. Fingl et al., 1975, in “The Pharmacological Basis of Therapeutics”, Ch. 1 p 1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g. the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the preceding Examples.

a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

b) Tablets

Tablets can be prepared, for example, from the following ingredients.

Parts by weight

| | |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated -with a solution of the polyvinyl-pyrrolidone in ethanol. The dry granulate can be blended with the magnesium stearate and the rest of the starch. The mixture is then compressed in a tabletting machine to give tablets each containing a unit dose or a part of a unit dose of active compound.

c) Enteric Coated Tablets

Tablets can be prepared by the method described in (b) above: The tablets can be enteric coated in a conventional manner using a solution of 20% cellulose acetate phthalate and 3% diethyl phthalate in ethanol:dichloromethane (1:1).

d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

In the compositions of the present invention the active compound may, if desired, be associated with other compatible pharmacologically active ingredients. For example, the compounds of this invention can be administered in combination with another therapeutic agent that is known to treat a disease or condition described herein. For example, with one or more additional pharmaceutical agents that inhibit or prevent the production of VEGF or angiopoietins, attenuate intracellular responses to VEGF or angiopoietins, block intracellular signal transduction, inhibit vascular hyperpermeability, reduce inflammation, or inhibit or prevent the formation of edema or neovascularization. The compounds of the invention can be administered prior to, subsequent to or simultaneously with the additional pharmaceutical agent, whichever course of administration is appropriate. The additional pharmaceutical agents include, but are not limited to any of the agents, for examples, described in pages 20-28. The compounds of the invention and the additional pharmaceutical agents act either additively or synergistically. Thus, the administration of such a combination of substances that inhibit angiogenesis, vascular hyperpermeability and/or inhibit the formation of edema can provide greater relief from the deletrious effects of a hyperproliferative disorder, angiogenesis, vascular hyperpermeability or edema than the administration of either substance alone. In the treatment of malignant disorders combinations with antiproliferative or cytotoxic chemotherapies or radiation are included in the scope of the present invention.

The present invention also comprises the use of a compound of formula I as a medicament.

A further aspect of the present invention provides the use of a compound of formula I or a salt thereof in the manufacture of a medicament for treating vascular hyperpermeability, angiogenesis-dependent disorders, proliferative diseases and/or disorders of the immune system in mammals, particularly human beings.

The present invention also provides a method of treating vascular hyperpermeability, inappropriate neovascularization, proliferative diseases and/or disorders of the immune system which comprises the administration of a therapeutically effective amount of a compound of formula I to a mammal, particularly a human being, in need thereof.

The contents of all references, patents and published patent applications, in their entirety, cited throughout this application are incorporated herein by reference.

Assays for Screening Compounds of Formula (I)

The in vitro potency of compounds in inhibiting CXCR3 discussed herein or described in the art may be determined by the procedures detailed below.

Inhibition of IP-10 Binding to hCXCR3

Radioligand binding assays were performed in CHO cells expressing human CXCR3 or anti-CD3/anti-CD28 activated murine splenocytes. All compounds were dissolved in DMSO and assays run at a final DMSO concentration of 1% (v/v). [¹²⁵-labeled IP-10 was purchased from PerkinElmer and used at 50 pM per assay.

Compounds are serially diluted in DMSO before diluting into assay buffer (25 mM HEPES, pH 7.4, 5 mM MgCl₂, 1 mM CaCl₂, 0.5% BSA) with CHO cell membranes expressing hCXCR3 (3 μg/well) and [¹²⁵I]-IP-10. The reaction was incubated at room temperature for 90 minutes before transferring to GF/C filter plates (PerkinElner) pre-treated with 0.3% polyethyleneimine for 2 hours at 4 C. The filter plates are washed six times with ice cold wash buffer (25 mM HEPES, pH 7.4, 5 mM MgCl₂, 1 mM CaCl₂, 500 mM NaCl) and dried before adding 50 ul/well Microscint to each well. Plates are counted on Packard Topcount scintillation counter where background binding is determined 100 nM IP-10 and control total binding determined by addition of DMSO in place of the test compound. The radioactivity values (cpm) were used to calculate the per cent inhibition at a given compound concentration and the data fit to a sigmoidal curve in a semi-log plot to determine IC₅₀ values.

Inhibition of IP-10-Induced Intracellular Calcium Release in Cells Expressing hCXCR3

Calcium flux assays were performed in CHO cells expressing human CXCR3 and the Gα₁₆ coupling protein. All compounds were dissolved in DMSO and assays run at a final DMSO concentration of 1% (v/v). Human IP-10 was purchased from Peprotech and used at a final assay concentration of 30 nM.

Briefly, cells were suspended in a microtiter plate at 50,000 per well in assay buffer (20 mM HEPES pH.7.4, 0.1% bovine serum albumin, and 2.5 mM Probenocid in Hank's Buffered Saline Solution) containing 2.5 μM Fluo-4 dye (Molecular Probes) and incubated at room temperature for 60-90 min before resuspending in assay buffer without dye. Calcium flux assays were performed on a FLIPR instrument (Molecular Devices) by adding compound to the cells followed by addition of IP-10 and measuring the change in fluorescence as a function of time. Maximal and minimal values for fluorescence were determined using 300 nM IP-10 and buffer addition, respectively. Fluorescence values were used to calculate the per cent inhibition at a given compound concentration and the data fit to a sigmoidal curve in a semi-log plot to determine IC₅₀ values.

Chemotaxis Assay in Transwell Format

Chemotaxis assays were run with chemokine receptor transfected BA/F3 cells, PHA activated human peripheral blood mononuclear cells or anti-CD3/anti-CD28 activated murine splenocytes. All compounds were dissolved in DMSO. All assays were run at a final DMSO concentration of 1% (v/v). Human IP-10 was purchased from Peprotech and used at a concentration of 125 nanograms per ml.

Briefly, cells were resuspended in RPMI 1640 and preincubated at room temperature with test article or DMSO for 10 minutes prior to being used in chemotaxis assay. The lower chambers of the Transwell plates were filled with 600 μLs of PBS 0.1% BSA (w/v) with or without chemotactic ligand. Concentrations of test articles corresponding to those used above during preincubation were added to ligand containing wells. Maximal chemotaxis was measured in the presence of DMSO only. Filter units were placed in the wells and the pretreated cells were added in a 100 μL volume. Following a 2 hour incubation at 37° C. the assay was scored by hemacytometer, particle counter or optical imaging system.

### ABBREVIATIONS

DMF N,N-Dimethylformamide
SEM 2-(Trimethylsilyl)ethoxymethyl chloride
PMB p-methoxybenzyl
Cbz benzyloxycarbonyl
TMS trimethylsilyl
BOC t-butoxycarbonyl
HPLC High Performance Liquid Chromatography
Rₜ Retention time
TBDMS tert-butyl-dimethyl-silane
THF tetrahydrofurane
HOAc acetic acid
i-PrOH 2-propanol
t-BuOH tert-butyl alcohol
t-BuOK potassium tert-butoxide
Et₂O diethyl ether
EtOAc ethyl acetate
DME 1, 2 -dimethoxyethane
Racemic-BINAP (±)-2,2′-bis(diphenylphosphino)-1,1′-binaphthalene
(R)-BINAP (R)-(+)-2,2′-bis(diphenylphosphino)-1,1′-binaphthalene
(S)-BINAP (S)-(−)-2,2′-bis(diphenylphosphino)-1,1′-binaphthalene
DPPF 1,1′-bis(diphenylphosphino)ferrocene
TFA trifluoroacetic acid
DCC N,N′-dicyclohexylcarbodiimide
DIC N,N′-diisopropylcarbodiimide
EDC 1-(3-dimethylaminopropyl)-3-ethylcarbodiimide
HBTU O-benzotriazo-1-yl-N,N,N′,N′-tetramethyluronium hexafluorophosaphate
HATU O-(7-Azabenzotriazo-1-yl)-N,N,N′,N′-tetramethyluronium hexafluorophosaphate.
TFFH fluoro-n,n,n′,n′-tetramethylformamidinium hexafluorophosphate
HOBT 1-hydroxybenzotriazole.
HOAT 1-hydroxy-7-azabenzotriazole
DIEA N,N-diisopropylethylamine
XANTPHOS 9,9-dimethyl-4,5-bis(diphenylphosphino)xanthene
SEMCl 2-(trimethylsilyl)ethoxymethyl chloride
KOAc potassium acetate
DMSO dimethyl sulfoxide
LDA lithium diisopropylamide
PPh₃ triphenylphosphine
Et₃N triethyl amine
PPTS pyridinium p-toluenesulfonate
DMFDMA N,N-dimethylformamide dimethyl acetal
TBAF tetra-n-butylammonium fluoride
AcCN acetonitrile

The following examples are ordered according to the ultimate final general procedure used in their preparation. The synthetic routes to any novel intermediates are detailed by sequentially listing the general procedure (letter codes) in parentheses after their name. A worked example of this protocol is given below. Analytical data is defined either within the general procedures or in the tables of examples. Unless otherwise stated, all 1H or 13C NMR data were collected on a Varian Mercury Plus 400 MHz or a Bruker DRX 400 MHz instrument; chemical shifts are quoted in parts per million (ppm). High pressure liquid chromatography (HPLC) analytical data are either detailed within the experimental or referenced to the table of HPLC conditions using the lower case method letter in parentheses provided in Table 1.

Table 1. List of HPLC Methods:

| Method | Conditions |
|---|---|
| a | RP-HPLC (10% to 100% acetonitrile/0.05M aqueous ammonium |
| | acetate, buffered to pH 4.5, over 5 min; 100% acetonitrile for 2 min |
| | at 1.7 mL/min; λ = 254 nm; Hypersil C18, 100 Å, 5 μm, 150 × 4.6 mm |
| | column). |
| b | RP-HPLC (10% to 100% acetonitrile/0.05M aqueous ammonium |
| | acetate, buffered to pH 4.5, over 5 min; 100% acetonitrile for 7 min at 1.7 mL/ |
| | min; λ = 254 nm; Hypersil C18, 100 Å, 5 μm, 150 × 4.6 mm |
| | column). |
| c | LC/MS (30% to 95% acetonitrile/0.01M aqueous ammonium |
| | acetate over 2.0 min; 95% acetonitrile/0.01M aqueous ammonium |
| | acetate for 1.5 min at 1.0 mL/min; UV λ = 210-360 nm; Genesis C8, 4 μm, |
| | 30 × 4.6 mm column; ESI +ve/−ve) |
| d | LC/MS (5% acetonitrile/0.01M aqueous ammonium acetate for |
| | 0.25 min; 5% to 95% acetonitrile/0.01M aqueous ammonium |
| | acetate over 2.5 min; 95% acetonitrile/0.01M aqueous ammonium |
| | acetate for 0.85 min at 1.4 mL/min; UV λ = 210-360 nm; Genesis C8, 4 μm, |
| | 30 × 4.6 mm column; ESI +ve/−ve) |
| e | LC/MS (30% to 95% acetonitrile/0.01M aqueous ammonium |
| | acetate over 2.0 min; 95% acetonitrile/0.01M aqueous ammonium |
| | acetate for 3.5 min at 1.0 mL/min; UV λ = 210-360 nm; Genesis C8, 4 μm, |
| | 30 × 4.6 mm column; ESI +ve/−ve) |
| f | LC/MS (5% to 35% acetonitrile/0.01M aqueous ammonium acetate |
| | over 4.0 min; 35% to 95% acetonitrile/0.01M aqueous ammonium |
| | acetate over 2.0 min; 95% acetonitrile/0.01M aqueous ammonium |
| | acetate for 1 min at 1.0 mL/min; UV λ = 210-360 nm; Genesis C8, 4 μm, |
| | 30 × 4.6 mm column; ESI +ve/−ve) |
| g | LC/MS (5% to 25% acetonitrile/0.1% formic acid in water over 1.4 min; |
| | 25% to 95% acetonitrile/0.1% formic acid in water over 1.1 min; |
| | 95% acetonitrile/0.1% formic acid in water for 1 min at 1.4 mL/min; |
| | UV λ = 210-360 nm; Genesis C8, 4 μm, 30 × 4.6 mm column; ESI +ve/−ve) |
| h | LC/MS (5% to 70% acetonitrile/0.1% formic acid in water over 2 min; |
| | 70% to 95% acetonitrile/0.1% formic acid in water over 3 min; |
| | 95% acetonitrile/0.1% formic acid in water for 2 min at 1.4 mL/min; |
| | UV λ = 210-360 nm; Genesis C8, 4 μm, 30 × 4.6 mm column; ESI +ve/−ve) |

The general synthetic schemes that were utilized to construct the majority of compounds disclosed in this application are described below in (Schemes 1-18).

The general procedure letter codes constitute a synthetic route to the final product. A worked example of how the route is determined is given below using Example #D15 as a non-limiting illustration. The synthesis of Example #D15 was completed using general procedure N (scheme 5), i.e.

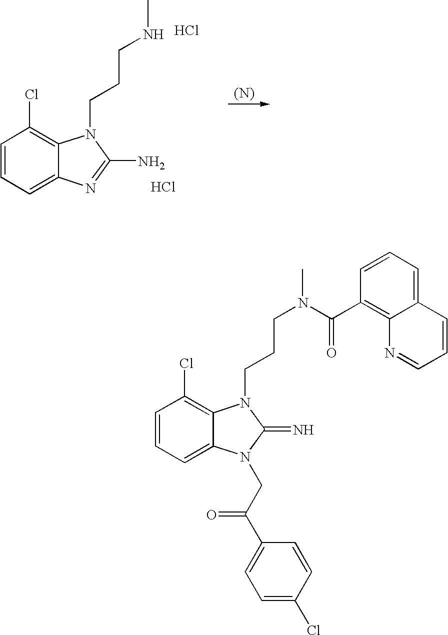

The amine starting material for this reaction was prepared using, the route (B, C, D, M) (schemes 1 and 5). This translates into the following sequence where the amine starting material used in general procedure N is the product of following the procedures B, C, D and M, in the given order.

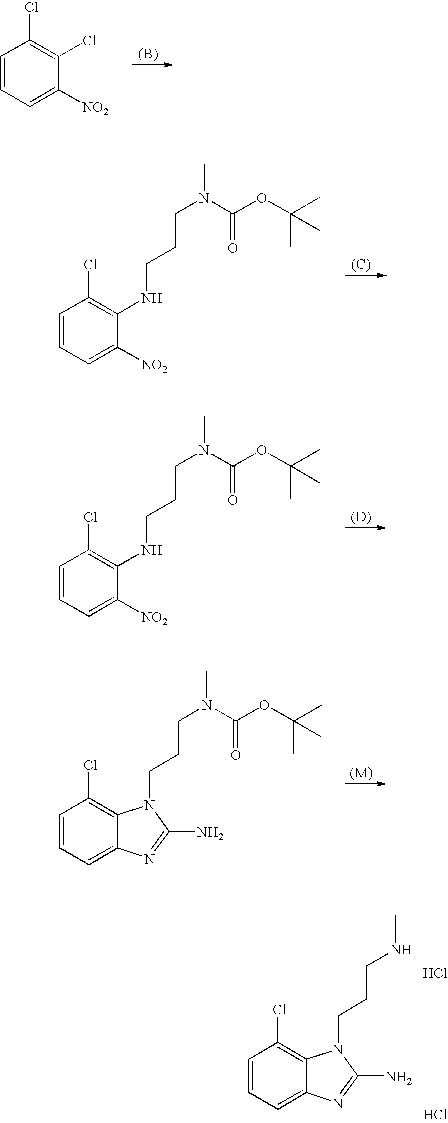

### Scheme 1. General synthetic routes to 2-iminobenzimidazoles (General procedures A, B, C, E, F, J or G)

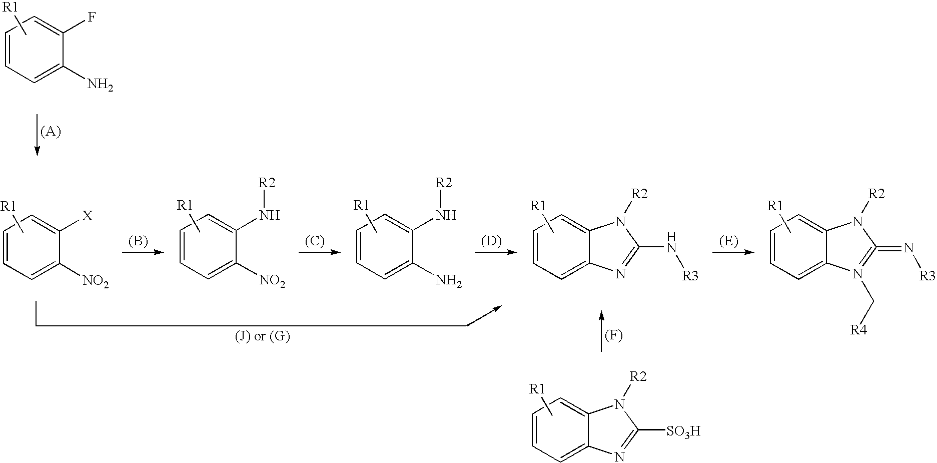

### Scheme 2. General synthetic routes to 2-iminobenzimidazoles (General procedures H, I)

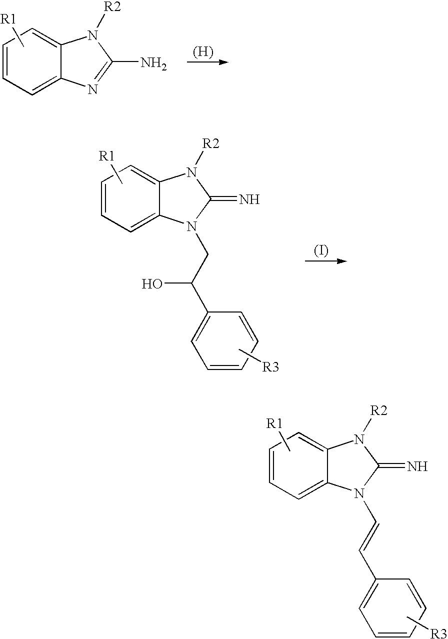

### Scheme 3. General synthetic routes to 2-iminobenzimidazoles (General procedure K)

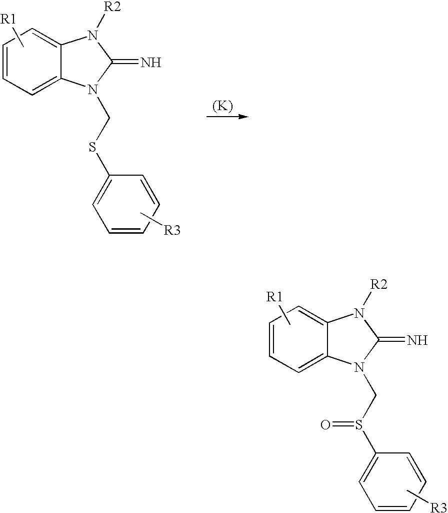

### Scheme 4. General synthetic routes to 2-iminobenzimidazoles (General procedure L)

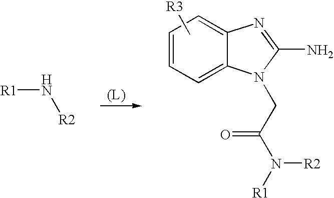

### Scheme 5. Deprotection of BOC protected amines. (General procedures M, N, R)

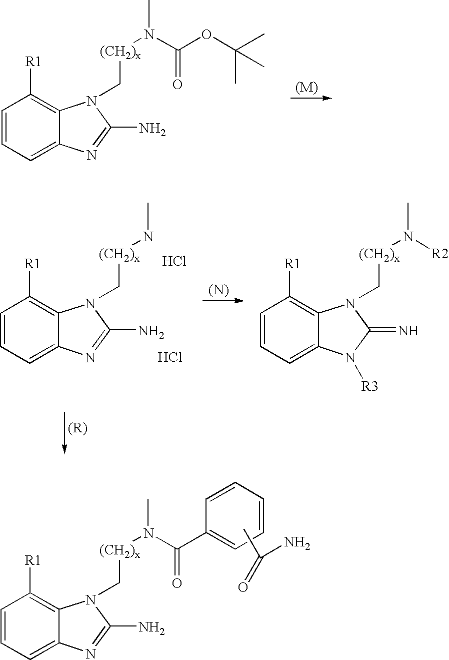

### Scheme 6. General synthetic routes to 2-iminobenzimidazoles (General procedure P)

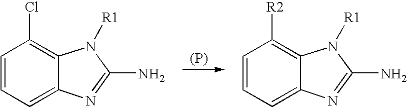

### Scheme 7. General synthetic routes to 2-iminobenzimidazoles (General procedure Q)

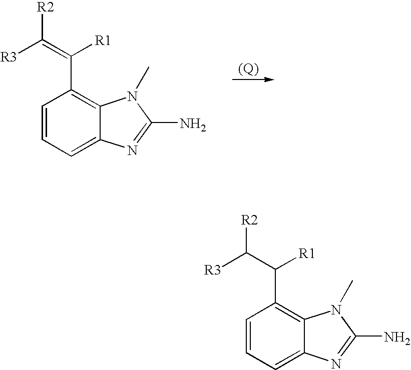

### Scheme 8. General synthetic routes to 2-iminobenzimidazoles (General procedures T, U)

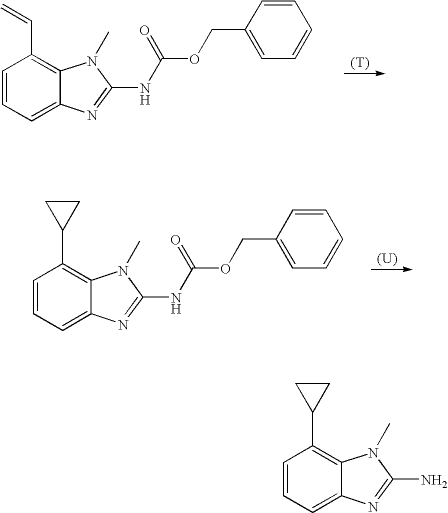

### Scheme 9. General synthetic routes to 2-iminobenzimidazoles (General procedures C, D, V, W)

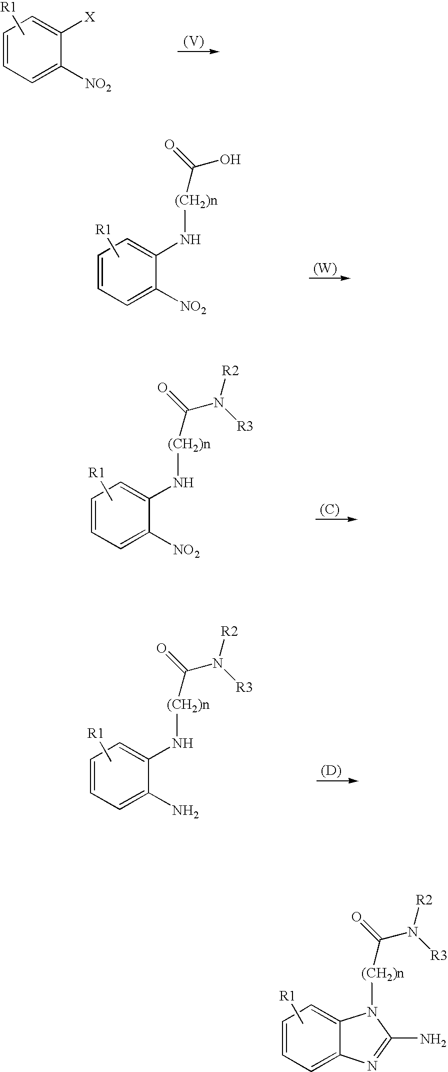

### Scheme 10. General synthetic routes to 2-iminobenzimidazoles (General procedure X)

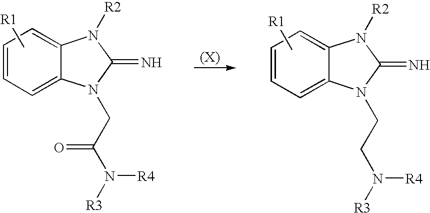

### Scheme 11. General synthetic routes to 2-iminobenzimidazoles (General procedure Y)

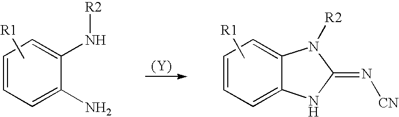

### Scheme 12. General synthetic routes to 2-iminobenzimidazoles (General procedure Z)

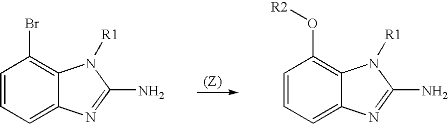

### Scheme 13. Formation of a protected 2-aminobenzimidazole (General procedures AA, BB)

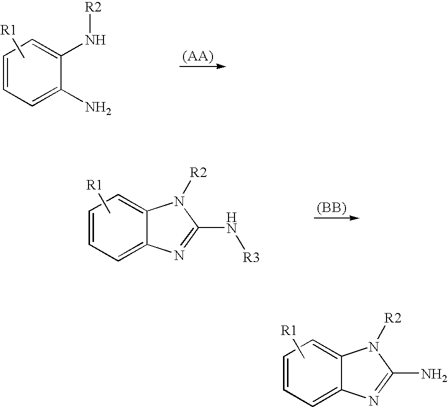

### Scheme 14. General synthetic routes to benzyl-(2-chloroethyl)alkylamine (General procedures CC, DD)

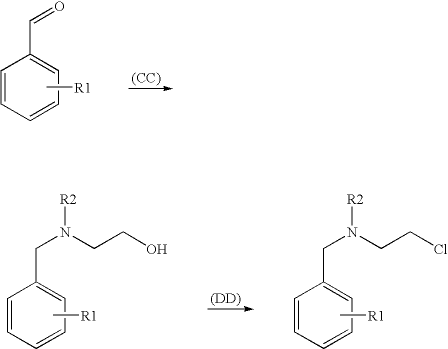

### Scheme 15. General synthetic routes to oxime formation (General procedure EE)

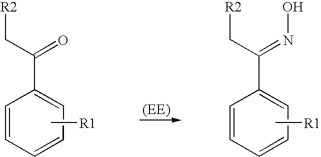

### Scheme 16. Reduction of a carbonyl to a carbanol (General procedure FF)

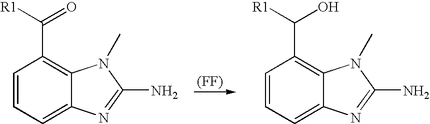

### Scheme 17. General synthetic routes to 2-iminobenzimidazoles (General procedure FF)

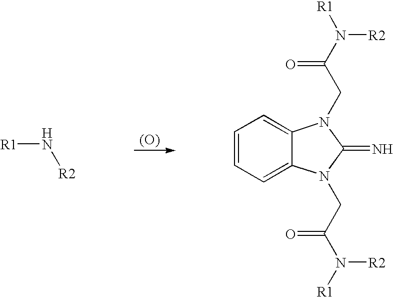

### Scheme 18. Acylation of an amine (General procedure S)

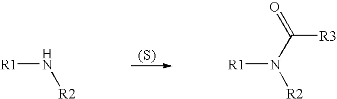

### LIST OF GENERAL PROCEDURES

General Procedure A: Oxidation of an aniline to a nitrobenzene.
General Procedure B: Addition of an amine to a halo-nitrobenzene.
General Procedure C: Reduction of a nitrobenzene ring to an aniline.
General Procedure D: Formation of a 2-aminobenzimidazole.
General Procedure E: Alkylation of a 2-aminobenzimidazole to form a 2-iminobenzimidazole.
General Procedure F: Sulfonic acid displacement with an amine.
General Procedure G: Conversion of a nitrobenzene to a 2-aminobenzimidazole
General Procedure H: Alkylation of a 2-aminobenzimidazole followed by reduction of a ketone.
General Procedure I: Conversion of a secondary alcohol to an alkene.
General Procedure J: Formation of a 2-aminobenzimidazole.
General Procedure K: Oxidation of a sulfide to the sulfoxide.
General Procedure L: Formation of a N-1-acetamide-substituted 2-aminobenzimidazole.
General Procedure M: Deprotection of BOC protected amines.
General Procedure N: Acylation or Sulfonylation of an amine followed by alkylation of a 2-aminobenzimidazole.
General Procedure O: Formation of a N-1,N-3-diacetamide-substituted 2-aminobenzimidazole.
General Procedure P: Cross coupling of a 7-Halo-2-aminobenzimidazole.
General Procedure Q: Reduction of an alkene.
General Procedure R: Acylation of an amine followed by hydrolysis of a nitrile.
General Procedure S: Acylation of an amine.
General Procedure T: Cyclopropanation of an alkene.
General Procedure U: Removal of a CBz group.
General Procedure V: Synthesis of a nitro-phenylamino-propionic acid.
General Procedure W: Conversion of a carboxylic acid to a carboxamide.
General Procedure X: Reduction of an amide to an amine.
General Procedure Y: Formation of a cyanoguanidine.
General Procedure Z: Ullmann coupling of an aryl bromide.
General Procedure AA: Formation of a protected 2-aminobenzimidazole.
General Procedure BB: Deprotection of a Carbamate Protected 2-Aminobenzimidazole.
General Procedure CC: Reductive Amination of an Amine with an Aldehyde.
General Procedure DD: Conversion of a primary alcohol to a chloride.
General Procedure EE: Oxime formation.
General Procedure FF: Reduction of a carbonyl to a carbanol.

The general procedure letter codes constitute a synthetic route to the final product. A worked example of how the route is determined is given below using Example #17 as a non-limiting illustration. The synthesis of Example #17 was completed using general procedure G as detailed in Table 5, i.e.

General Procedure A: Oxidation of an aniline to a nitrobenzene. A solution of the aniline (preferably 1 equivalent) in a polar protic solvent (preferably acetic acid) is added to a solution of an oxidant (preferably sodium perborate tetrahydrate) in a polar protic solvent (preferably acetic acid). The reaction mixture is maintained at temperatures of about 0° C. to 100° C. (preferably 55° C.). After about 30 min following the addition of the oxidant the reaction mixture is allowed to reach room temperature and an organic solvent (preferably diethyl ether and ethyl acetate) and water is added. The organic layer is separated and washed repeatedly with a basic aqueous solution (preferably saturated sodium bicarbonate). The organic layer is dried, filtered and concentrated.

Illustration of General Procedure A

### Preparation #1: 2-Fluoro-1=Nitro-3-trifluoromethyl-benzene

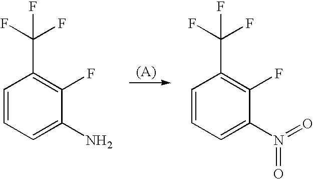

A solution of 2-Fluoro-3-trifluoromethyl-phenylamine (645 mg, 3.6 mmol) in acetic acid (7 mL) was added drop-wise to a solution of sodium perborate tetrahydrate in acetic acid (7 mL) heated to about 55° C. After about 30 minutes following the addition the reaction mixture was cooled to room temperature diluted with diethyl ether and ethyl acetate (10:1) and water. The organic layer was separated and washed repeatedly with saturated sodium bicarbonate until neutral pH was achieved. The organic layer was dried, filtered and concentrated to provide 560 mg of the crude product that was used in subsequent reactions without further purification. RP-HPLC Rₜ 6.46 min. (table 1, method a).

General Procedure B: Addition of an amine to a halo-nitrobenzene. To about 1 to 20 equivalents amine (preferably 2.25 equivalents), about 0 to 5 equivalents of an organic base (preferably diisopropylethylamine, preferably 2 equivalents) and a halo-nitrobenzene (preferably 1 equivalent) are combined in an organic solvent or neat (preferably neat). The reaction mixture is stirred at about 0° C. to 200° C. (preferably 100° C.) for about 1 to 10 days (preferably 3 days). The reaction mixture is concentrated, diluted with an organic solvent (preferably diethyl ether) and washed with an acidic aqueous solution (preferably 1N HCl) followed by brine. The organic layer is dried, filtered and concentrated in vacuo.

Illustration of General Procedure B

### Preparation #2: [3-(2-Chloro-6-Nitro-phenylamino)-propyl]-methyl-carbamic acid tert-butyl ester

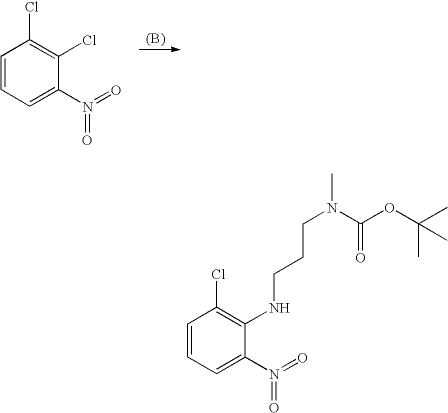

1,2 -Dichloro-3-Nitro-benzene (1.0 g, 5.2 mmol), (3-Amino-propyl)-methyl-carbamic acid tert-butyl ester (2.25 g, 11.9 mmol) and diisopropylethylamine (1.8 mL, 10.4 mmol) were combined and heated to about 100° C. After about 3 days the reaction mixture was diluted with diethyl ether (200 mL) and 1N HCl (200 mL). The organic layer was separated, washed with brine, dried (Na₂SO₄), filtered and concentrated in vacuo to provide 1.8 g of [3-(2-Chloro-6-Nitro-phenylamino)-propyl]-methyl-carbamic acid tert-butyl ester as an oil which was used in subsequent reactions without further purification. RP-HPLC Rₜ 7.51 min. (table 1, method a); m/z: (M+H)⁺ 244.0.

General Procedure C: Reduction of a nitrobenzene ring to an aniline. To a solution of the nitrobenzene compound in a polar protic solvent (preferably acetic acid) is added a metallic reducing agent (preferably iron) (about 1 to 10 equivalents, preferably 4 equivalents). The reaction mixture is stirred at about 0° C. to 100° C. (preferably 25° C.) for about 2 to about 48 (preferably 15 hours). The reaction mixture is filtered and concentrated in vacuo. The crude product is dissolved in an organic solvent (preferably diethyl ether) and washed with a basic aqueous solution (preferably 2N NaOH) that had been saturated with an iron-chelating agent (preferably EDTA). The organic layer is further washed with brine, filtered and concentrated in vacuo.

Illustration of General Procedure C

### Preparation #3: [3-(2-Amino-6-chloro-phenylamino)-propyl]-methyl-carbamic acid tert-butyl ester

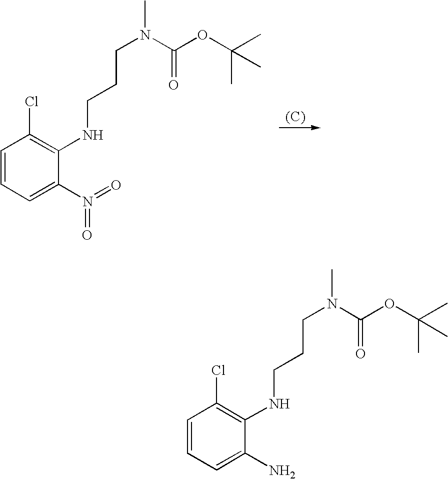

To a solution of [3-(2-Chloro-6-Nitro-phenylamino)-propyl]-methyl-carbamic acid tert-butyl ester (2.85 g, 8.3 mmol) in acetic acid (160 mL) at room temperature was added iron powder (3.7 g, 66.8 mmol). After stirring for 20 hours the reaction mixture was filtered and concentrated in vacuo. The crude product was dissolved in diethyl ether and washed with a solution of 2N NaOH that had been saturated with EDTA. The organic layer was further washed with brine, dried with Na₂SO₄, filtered and concentrated in vacuo to provide [3-(2-Amino-6-chloro-phenylamino)-propyl]-methyl-carbamic acid tert-butyl ester as a brown oil that was used in subsequent reactions without further purification. RP-HPLC Rₜ 6.05 min. (table 1, method a).

General Procedure D: Formation of a 2-aminobenzimidazole. To a solution of the phenylenediamine in an organic solvent (preferably ethanol) is added about 0 to 10 equivalents of an organic base (preferably sodium acetate, preferably 5 equivalents) followed by a solution of about 1 to 3 equivalents of cyanogen bromide in an organic solvent (preferably acetonitrile, preferably 1.5 equivalents). After stirring for about 20 hours at about 25° C. the reaction mixture is concentrated in vacuo. The crude mixture is diluted with an organic solvent (preferably ethyl acetate) and the product is isolated by filtration or taken through an aqueous work up and purified by chromatography.

Illustration of General Procedure D

### Preparation #4: [3-(2-Amino-6-chloro-phenylamino)-propyl]-methyl-carbamic acid tert-butyl ester

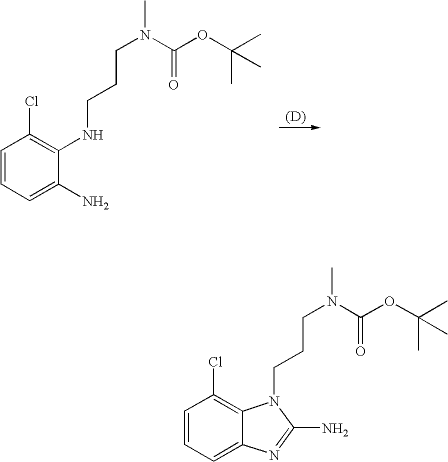

To a solution of [3-(2-Amino-6-chloro-phenylamino)-propyl]-methyl-carbamic acid tert-butyl ester (4.57 g, 13.3 mmol) in EtOH (260 mL) was added NaOAc (5.5 g, 67 mmol) followed by a 5 N solution of cyanogen bromide in acetonitrile (4 mL, 20 mmol). After stirring for about 20 hours at room temperature the reaction mixture was concentrated in vacuo. The crude reaction mixture was diluted with Et₂O (200 mL) and 2 N NaOH (200 mL). The organic layer was separated, washed with brine, dried (Na₂SO₄), filtered and concentrated. The crude product was purified by column chromatography on silica gel (gradient elution 5-10% MeOH/CH₂Cl₂, containing 1% Et₃N) to provide 2.9 g of [3-(7-Chloro-2-imino-2,3-dihydro-benzoimidazol-1-yl)-propyl]-methyl-carbamic acid tert-butyl ester as a brown oil. RP-HPLC Rₜ 6.05 min 6.07 (table 1, method a); m/z: (M+H)⁺ 339.0, 341.1 (3:1).

TABLE A
Examples prepared using general procedure D

| | | | | m/z |
|---|---|---|---|---|
| Starting Material | Product | Example # | Rₜ/min (method) | or ¹H NMR (d₆ DMSO, 400 MHz) |
| 1-Bromo-2-fluoro-3-nitro- benzene | 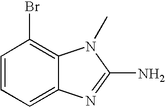 | A1 | 4.50 min (a) | 226.3, 228.3 (1:1) (M + H)⁺ |
| 1,2-Dichloro-3-nitro- benzene | 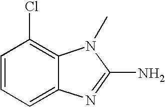 | A2 | 4.42 min (a) | 182.3, 184.3 (3:1) (M + H)⁺ |

General Procedure E: Alkylation of a 2-aminobenzimidazole to form a 2-iminobenzimidazole. To a solution of the 2-aminobenzimidazole in an organic solvent (preferably DMF) is added about 1 to 5 equivalents of an alkylating agent (preferably 1 equivalent). The reaction mixture is stirred at about 0° C. to 150° C. (preferably room temperature) for about 1 to 5 days (preferably 1 day). The reaction mixture is diluted with an organic solvent (preferably ethyl acetate) and filtered or purified by chromatography.

Illustration of General Procedure E

### EXAMPLE #1

### N-(3-{3-[2-(4-Bromo-phenyl)-2-oxo-ethyl]-7-chloro-2-imino-2,3-dihydro-benzoimidazol-1-yl}-propyl)-N-methyl-benzamide hydrobromide

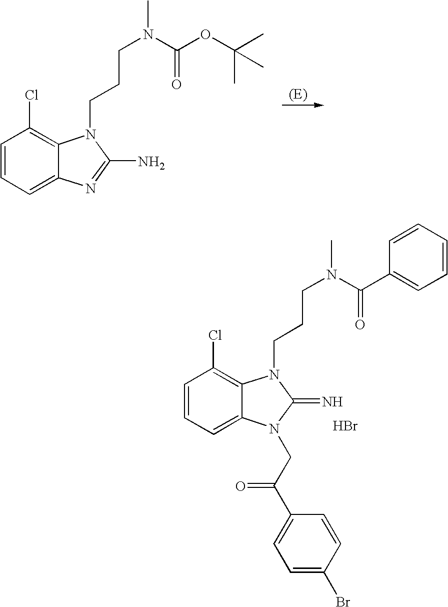

To a solution of N-[3-(7-Chloro-2-imino-2,3-dihydro-benzoimidazol-1-yl)-propyl]—N-methyl-benzamidine (104 mg, 0.30 mmol) in DMF (3 mL) was added 2-Bromo-1-(4-bromo-phenyl)-ethanone (83 mg, 0.30 mmol). The reaction mixture was stirred at room temperature for about 20 hours, diluted with ethyl acetate and filtered to provide 120 mg of N-(3-{3-[2-(4-Bromo-phenyl)-2-oxo-ethyl]-7-chloro-2-imino-2,3-dihydro-benzoimidazol-1-yl -propyl)-N-methyl-benzamide hydrobromide as a white solid. RP-HPLC Rₜ 6.05 min (table 1, method a), LC/MS (M+H)⁺ 540.0.

Table B. Examples Prepared Using General Procedure E

| | | | | | m/z |
|---|---|---|---|---|---|
| | | | | | or ¹H |
| | | | | | NMR (d₆ |
| | | | | Rₜ/min | DMSO, |
| Starting Material | Alkylating Reagent | Product | Example # | (method) | 400 MHz) |
| 1-Phenethyl-1,3-dihydro- benzoimidazol-2-yl- ideneamine (B, C, D) | 2-Bromo-1-(4-bromo- phenyl)-etha- none | 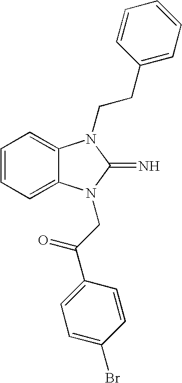 | B1 | 2.19 min (c) | (M + H)⁺434.4, 436.2 |
| 1-Cyclopropyl- meth- yl-1,3-dihydro- benzoimi- dazol-2-ylidene- amine (B, C, D) | 2-Bromo-1-(4-bromo- phenyl)-etha- none | 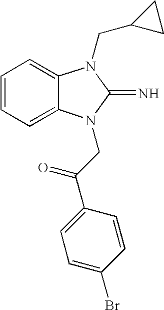 | B2 | 1.97 min (c) | (M + H)⁺384.2, 386.2 |
| 1-Cyclopropyl-1,3-di- hydro-benzoimi- dazol-2-ylidene- amine (B, C, D) | 2-Bromo-1-(4-bromo- phenyl)-etha- none | 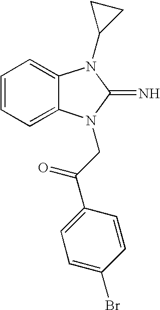 | B3 | 1.86 min (c) | (M + H)⁺370.2, 372.2 |
| 1-Cyclopentyl-1,3-di- hydro-benzo- imidazol-2-yl- ideneamine (B, C, D) | 2-Bromo-1-(4-bromo- phenyl)-etha- none | 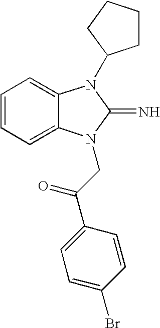 | B4 | 2.08 min (c) | (M + H)⁺398.2, 400.2 |
| 1-Benzyl-1,3-dihydro- benzoimidazole-2-yl- ideneamine (B, C, D) | 2-Bromo-1-(4-bromo- phenyl)-etha- none | 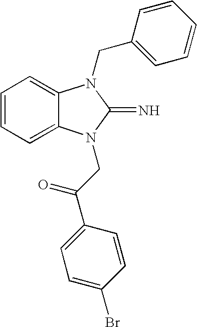 | B5 | 2.16 min (c) | (M + H)⁺420.3, 422.3 |
| 1-Isobutyl-1,3-dihydro- benzoimidazol-2-yl- ideneamine (B, C, D) | 2-Bromo-1-(4-bromo- phenyl)-etha- none | 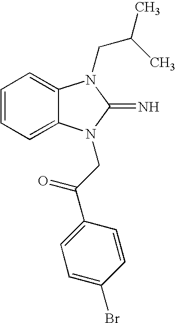 | B6 | 1.98 min (c) | (M + H)⁺386.3, 388.3 |
| 3-(2-Imino-2,3-dihydro- benzoimidazol-1-yl)-pro- pionic acid ethyl ester (B, C, D) | 2-Bromo-1-(4-bromo- phenyl)-etha- none | 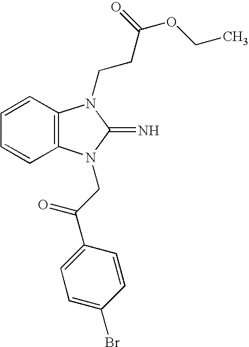 | B7 | 2.07 min (c) | (M + H)⁺430.3, 432.3 |
| 1-(2,2,2-Trifluoro- ethyl)-1,3-di- hydro-benzo- imidazol-2-yl- ideneamine (B, C, D) | 2-Bromo-1-(4-bromo- phenyl)-etha- none | 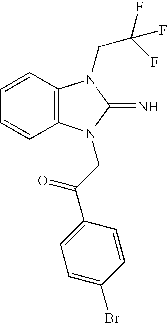 | B8 | 2.06 min (c) | (M + H)⁺412.4, 414.2 |
| 1,3-Dihydro-benzo- imidazol-2-yl- ideneamine | 2-Bromo-1-(4-bromo- phenyl)-etha- none | 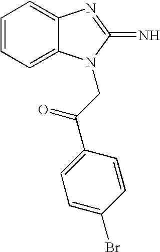 | B9 | 1.74 min (c) | (M + H)⁺330, 332 |
| 1,3-Dihydro-benzo- imidazol-2-ylidene- amine | 2-Bromo-1-(4-bromo- phenyl)-etha- none | 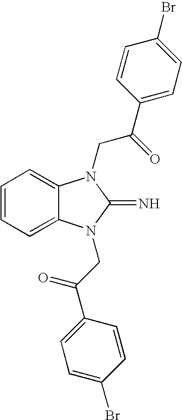 | B10 | 2.57 min (c) | (M + H)⁺526.4, 528.3, 530.2 |
| 1-(4-Bromo- phenyl)-2-(2-imi- no-2,3-dihydro- benzoimidazol-1-yl)-eth- anone (E) | 2-Chloro-N,N-di- methyl-aceta- mide | 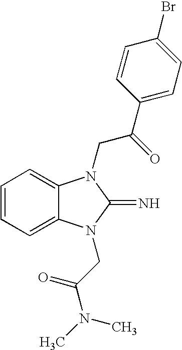 | B11 | 1.83 min (c) | (M + H)⁺413.1, 415.1 |
| 3-(2-Imino-2,3-di- hydro-benzo- imidazol-1-yl)-N-meth- yl-N-(3-methyl- benzyl)-pro- pionamide (B, V, W, C, D) | 2-Bromo-1-(4-chloro- phenyl)-etha- none | 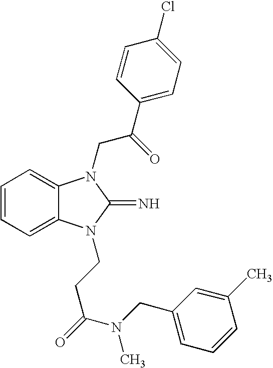 | B12 | 3.63 min (c) | (M + H)⁺475.0, 476.9 |
| 3-(2-Imino-2,3-di- hydro-benzo- imidazol-1-yl)-N-meth- yl-N-(3-tri- fluoromethyl- benzyl)-pro- pionamide (B, V, W, C, D) | 2-Bromo-1-(4-chloro- phenyl)-etha- none | 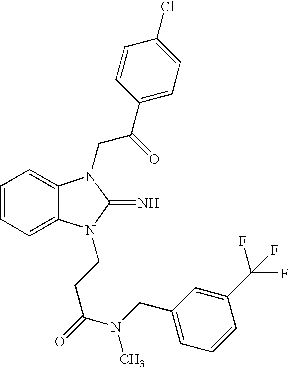 | B13 | 3.92 min (c) | (M + H)⁺528.9, 530.9 |
| 3-(2-Imino-2,3-di- hydro-benzo- imidazol-1-yl)-N-(3-meth- oxy-benzyl)-N-methyl- propionamide (B, V, W, C, D) | 2-Bromo-1-(4-chloro- phenyl)-etha- none | 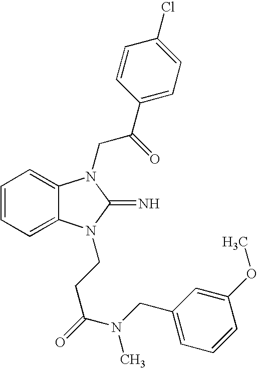 | B14 | 3.05 min (c) | (M + H)⁺491.0, 493.0 |
| 3-(2-Imino-2,3-di- hydro-benzo- imidazol-1-yl)-N-meth- yl-N-(4-methyl- benzyl)-pro- pionamide (B, V, W, C, D) | 2-Bromo-1-(4-chloro- phenyl)-etha- none | 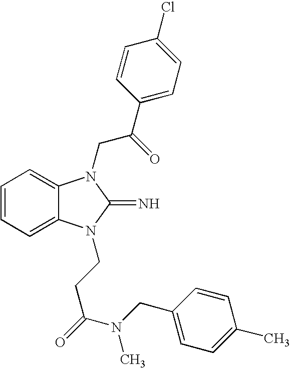 | B15 | 3.67 min (c) | (M + H)⁺475.0, 476.9 |
| 3-(2-Imino-2,3-di- hydro-benzo- imidazol-1-yl)-N-meth- yl-N-(4-tri- fluoromethyl- benzyl)-pro- pionamide (B, V, W, C, D) | 2-Bromo-1-(4-chloro- phenyl)-etha- none | 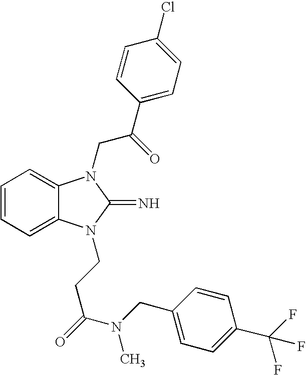 | B16 | 3.96 min (c) | (M + H)⁺528.9, 530.9 |
| 3-(2-Imino-2,3-di- hydro-benzo- imidazol-1-yl)-N-(4-meth- oxy-benzyl)-N-meth- ylpropionamide (B, V, W, C, D) | 2-Bromo-1-(4-chloro- phenyl)-etha- none | 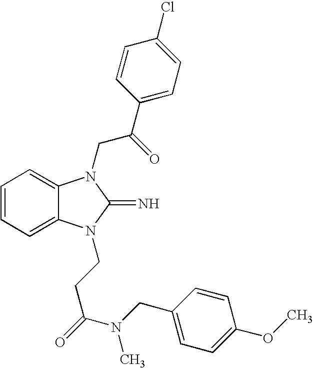 | B17 | 6.67 min (e) | (M + H)⁺491.0, 493.0 |
| N-(3-Chloro- benzyl)-3-(2-imi- no-2,3-dihydro- benzo- imidazol-1-yl)-N-methyl- propionamide (B, V, W, C, D) | 2-Bromo-1-(4-chloro- phenyl)-etha- none | 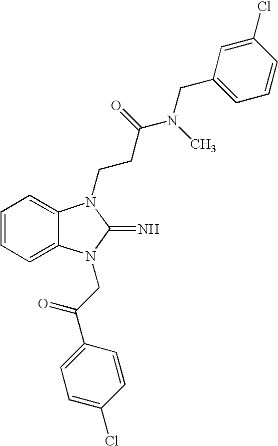 | B18 | 3.21 min (c) | (M + H)⁺494.4, 495.4, 496.4, 497.3 |
| N-(4-Chloro- benzyl)-3-(2-imi- no-2,3-dihydro- benzo- imidazol-1-yl)-N-methyl- propionamide (B, V, W, C, D) | 2-Bromo-1-(4-chloro- phenyl)-etha- none | 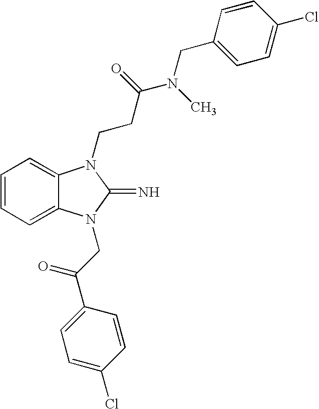 | B19 | 3.24 min (c) | (M + H)⁺494.5, 495.3, 496.3, 497.2 |
| N-Benzyl-3-(2-imi- no-2,3-dihydro- benzoimi- dazol-1-yl)-N-methyl- propionamide (B, V, W, C, D) | 2-Bromo-1-(4-chloro- phenyl)-etha- none | 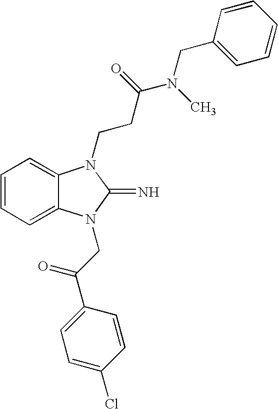 | B20 | 2.79 min (c) | (M + H)⁺461.3, 463.3 |
| 1,7-Dimethyl-1,3-di- hydro- benzoimidazol-2-yl- ideneamine (G) | 2-Bromo-1-phenyl- ethanone | 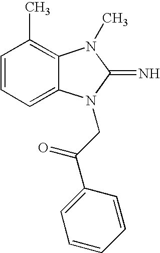 | B21 | 1.64 min (c) | (M + H)⁺280.3 |
| 1,7-Dimethyl-1,3-di- hydro- benzoimidazol-2-yl- ideneamine (G) | 2-Bromo-1-(2,4-dichloro- phenyl)-etha- none | 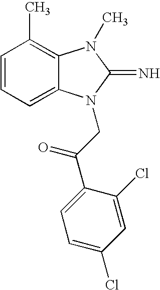 | B22 | 2.05 min (c) | (M + H)⁺348.3, 350.3 |
| 3-(2-Imino-2,3-dihydro- benzoimidazol-1-yl)-pro- pionic acid ethyl ester (B, C, D) | 2-Bromo-1-(4-chloro- phenyl)- ethanone | 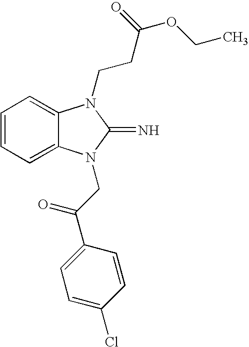 | B23 | 2.12 min (c) | (M + H)⁺386.3, 388.2 |
| N-Benz- yl-4-(7-chloro-2-imi- no-2,3-di- hydro-benzo- imidazol-1-yl)-N-methyl- butyramide (B, V, W, C, D) | 2-Bromo-1-(4-chloro- phenyl)-etha- none | 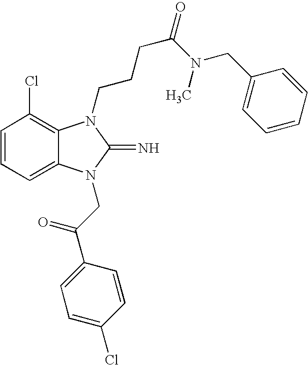 | B24 | 2.17 min (c) | (M + H)⁺509.5, 510, 511, 512.5 |
| N-[3-(2-Imino-3-meth- yl-2,3-dihydro-1H-benzo- imidazol-4-yl)-prop-2-yn- yl]-N-methyl- benzamide (B, C, D, P) | 2-Bromo-1-(4-chloro- phenyl)-etha- none | 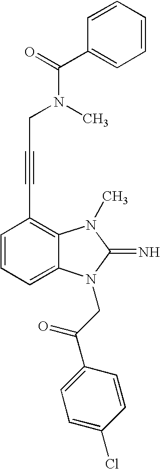 | B25 | 1.91 min (c) | (M + H)⁺471.3, 472.2 |
| 3-(2-Imino-3-meth- yl-2,3-di- hydro-1H-benzo- imidazol-4-yl)-pro- pionic acid (B, C, D, P, Q) | 2-Bromo-1-(4-bromo- phenyl)-eth- anone | 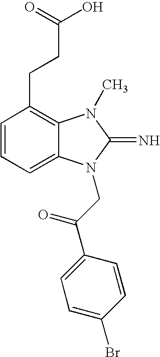 | B26 | 1.34 min (c) | (M + H)⁺416.3, 418.3 |
| 3-(2-Imino-3-meth- yl-2,3-di- hydro-1H-benzo- imidazol-4-yl)-pro- pionic acid (B, C, D, P, Q) | 2-Bromo-1-(4-bromo- phenyl)-eth- anone | 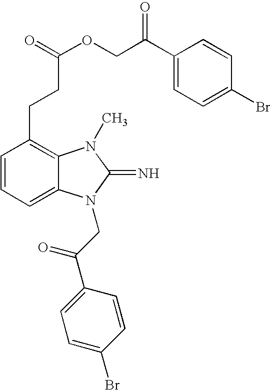 | B27 | 2.25 min (c) | (M + H)⁺612.4, 613.3, 614.3, 615.3 |
| 3-(2-Imino-3-meth- yl-2,3-di- hydro-1H-benzo- imidazol-4-yl)-pro- pionitrile (B, C, D, P, Q) | 2-Bromo-1-(4-chloro- phenyl)-etha- none | 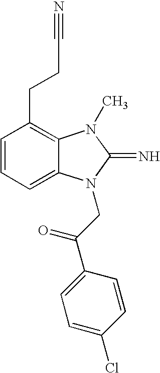 | B28 | 1.55 min (c) | (M + H)⁺353.2, 355.2 |
| 1-Methyl-7-vin- yl-1H-benzo- imidazol-2-yl- amine (J, P) | 2-Bromo-1-(4-bromo- phenyl)-etha- none | 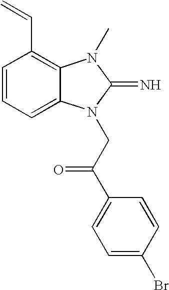 | B29 | 5.75 min (a) | 370, 372 (1:1) (M + H)⁺ |
| 7-Ethyl-1-meth- yl-1H-benzo- imidazol-2-yl- amine (J, P, Q) | 2-Bromo-1-(4-bromo- phenyl)-etha- none | 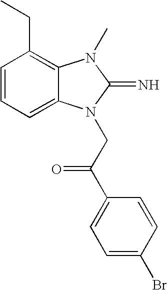 | B30 | 5.82 min (a) | 372.4, 374.4 (1:1) (M + H)⁺ |
| 7-Ethyl-1-meth- yl-1H-benzo- imidazol-2-yl- amine (J, P, Q) | 2-Chloro-1-(4-bromo- phenyl)-etha- none | 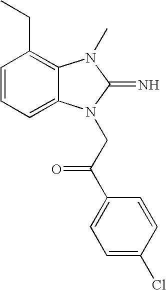 | B31 | 5.70 min (a) | 328.3, 330.3 (3:1) (M + H)⁺ |
| 7-Bromo-1-meth- yl-1H-benzo- imidazol-2-yl- amine (J) | 2-Bromo-1-(4-bromo- phenyl)-etha- none | 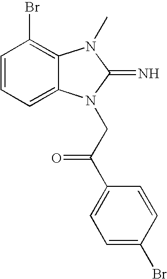 | B32 | 5.72 min (a) | (M + H)⁺422.1, 424.1, 426.1 (1:2:1) |
| 1-Methyl-7-phe- nyl-1H-benzo- imidazol-2-yl- amine (J, P) | 2-Bromo-1-(4-bromo- phenyl)-eth- anone | 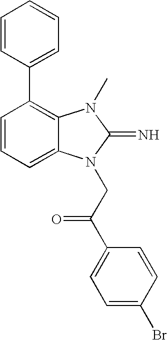 | B33 | 6.23 min (a) | (M + H)⁺420.3, 422.3 (1:1) |
| 7-Biphenyl-4-yl-1-meth- yl-1H-benzo- imidazol-2-ylamine (J, P) | 2-Bromo-1-(4-bromo- phenyl)-etha- none | 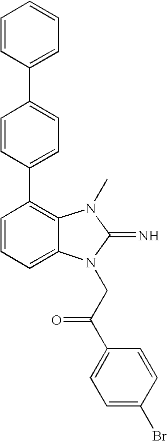 | B34 | 7.03 min (a) | (M + H)⁺496.4, 498.3 (1:1) |
| 7-Biphenyl-3-yl-1-meth- yl-1H-benzo- imidazol-2-ylamine (J, P) | 2-Bromo-1-(4-bromo- phenyl)-etha- none | 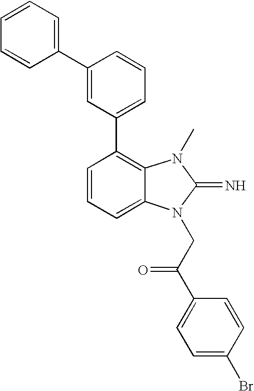 | B35 | 6.96 min (a) | (M + H)⁺496.3, 498.3 (1:1) |
| 1-Methyl-7-pyri- midin-5-yl-1H-benzo- imidazol-2-yl- amine (J, P) | 2-Bromo-1-(4-bromo- phenyl)-etha- none | 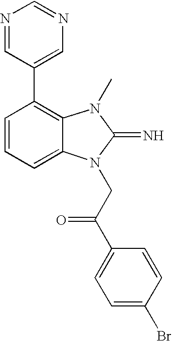 | B36 | 5.09 min (a) | (M + H)⁺422.4, 424.3 (1:1) |
| 1-Methyl-7-naph- thalen-1-yl-1H-benzo- imidazol-2-yl- amine (J, P) | 2-Bromo-1-(4-bromo- phenyl)-eth- anone | 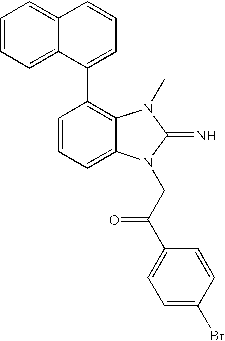 | B37 | 6.67 min (a) | (M + H)⁺470.4, 472.3 (1:1) |
| 1-Methyl-7-((E)-sty- ryl)-1H-benzo- imidazol-2-ylamine (J, P) | 2-Bromo-1-(4-bromo- phenyl)-etha- none | 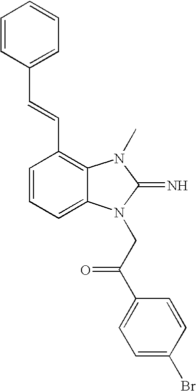 | B39 | 6.55 min (a) | (M + H)⁺446.4, 448.3 (1:1) |
| 1,7-Dimethyl-1H-benzo- imidazol-2-yla- mine (G) | 2-Bromo-1-phenyl- ethanone | 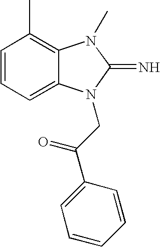 | B39 | 5.18 (a) | (M + H)⁺280.2 |
| 1,7-Dimethyl-1H-benzo- imidazol-2-yla- mine (G) | 2-Bromo-1-(2,4-dichloro- phenyl)-etha- none | 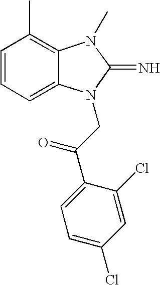 | B40 | 5.77 (a) | (M + H)⁺348.2, 350.2 (3:2) |
| 1-Methyl-7-phen- ethyl-1H-benzo- imidazol-2-yl- amine (J, P, Q) | 2-Bromo-1-(4-bromo- phenyl)-eth- anone | 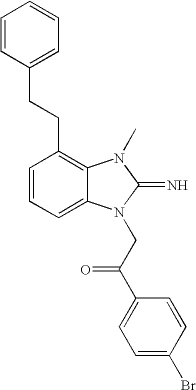 | B41 | 6.51 min (a) | (M + H)⁺448, 450 (1:1) |
| 1-Methyl-7-phen- ethyl-1H-benzo- imidazol-2-yl- amine (J, P, Q) | 2-Bromo-1-(4-chloro- phenyl)-eth- anone | 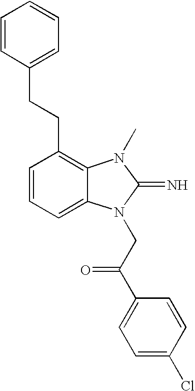 | B42 | 6.45 min (a) | (M + H)⁺404.2, 406.2 (3:1) |
| 1-Methyl-7-phen- ethyl-1H-benzo- imidazol-2-yl- amine (J, P, Q) | 2-Bromo-1-phenyl- ethanone | 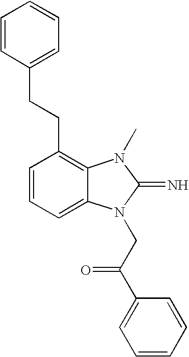 | B43 | 6.20 min (a) | (M + H)⁺370.2 |
| 1-Methyl-7-phen- ethyl-1H-benzo- imidazol-2-yl- amine (J, P, Q) | 2-Bromo-1-(2,4-dichloro- phenyl)-eth- anone | 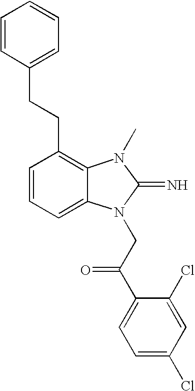 | B44 | 6.63 min (a) | (M + H)⁺438.2, 440.2 (3:2) |
| 1-Methyl-7-tri- fluoromethyl-1H-benzo- imidazol-2-yl- amine (A, G) | 2-Bromo-1-phenyl- ethanone | 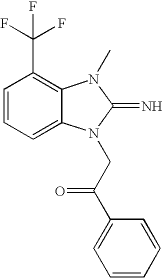 | B45 | 5.44 min (a) | (M + H)⁺334.2 |
| 1-Methyl-7-tri- fluoromethyl-1H-benzo- imidazol-2-yl- amine (A, G) | 2-Bromo-1-(4-bromo- phenyl)-eth- anone | 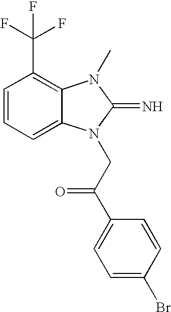 | B46 | 5.79 min (a) | (M + H)⁺412.2, 414.1 (1:1) |
| 1-Methyl-7-tri- fluoromethyl-1H-benzo- imidazol-2-yl- amine (A, G) | 2-Bromo-1-(4-chloro- phenyl)-etha- none | 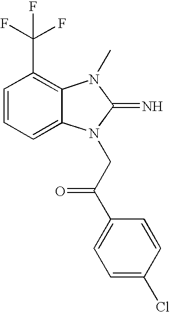 | B47 | 5.72 min (a) | (M + H)⁺368.2, 370.2 (3:1) |
| 1-Methyl-7-tri- fluoromethyl-1H-benzo- imidazol-2-yl- amine (A, G) | 2-Bromo-1-(2,4-di- chloro-phenyl)-eth- anone | 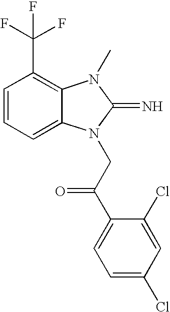 | B48 | 5.93 min (a) | (M + H)⁺402.1, 404.1 (3:2) |
| 7-Chloro-1-meth- yl-1H-benzo- imidazol-2-yl- amine (D) | 2-Bromo-1-phenyl- ethanone | 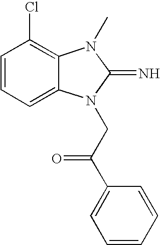 | B49 | 5.23 min (a) | (M + H)⁺300.2, 302.2 (3:1) |
| 7-Chloro-1-meth- yl-1H-benzo- imidazol-2-yl- amine (D) | 2-Bromo-1-(2,4-di- chloro-phenyl)-eth- anone | 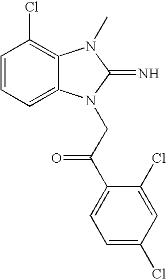 | B50 | 5.77 min (a) | (M + H)⁺368.1, 370.1, 372.1 (3:3:1) |
| 7-Chloro-1-meth- yl-1H-benzo- imidazol-2-yl- amine (D) | 3-Bromomethyl- benzo[d]isoxazole | 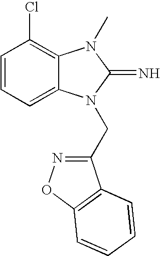 | B51 | 5.17 min (a) | (M + H)⁺313.1, 315.1 (3:1) |
| 7-Isopropyl-1-meth- yl-1H-benzo- imidazol-2-yl- amine (D, P, Q) | 2-Bromo-1-phenyl- ethanone | 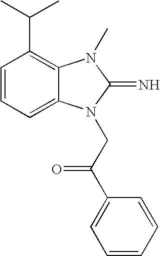 | B52 | 5.68 min (a) | (M + H)⁺308.3 |
| 7-Isopropyl-1-meth- yl-1H-benzo- imidazol-2-yl- amine (D, P, Q) | 2-Bromo-1-(4-bromo- phenyl)-eth- anone | 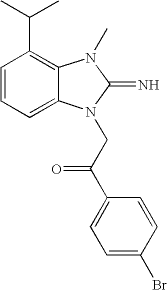 | B53 | 6.08 min (a) | (M + H)⁺368.1, 388.1 (1:1) |
| 7-Isopropyl-1-meth- yl-1H-benzo- imidazol-2-yl- amine (D, P, Q) | 2-Bromo-1-(4-chloro- phenyl)-eth- anone | 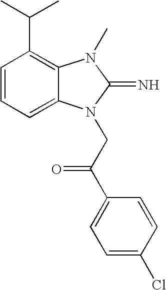 | B54 | 5.99 min (a) | (M + H)⁺342.2, 344.2 (1:1) |
| 7-Isopropyl-1-meth- yl-1H-benzo- imidazol-2-yl- amine (D, P, Q) | 2-Bromo-1-(2,4-di- chloro-phenyl)-eth- anone | 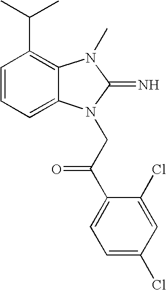 | B55 | 6.17 min (a) | (M + H)⁺376.2, 378.2 (3:2) |
| 7-Isopropenyl-1-meth- yl-1H-benzoimida- zol-2-ylamine (D, P) | 2-Bromo-1-phenyl- ethanone | 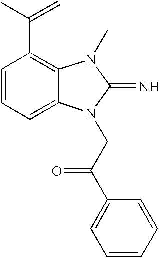 | B56 | 5.67 min (a) | (M + H)⁺306.2 |
| 7-Isopropenyl-1-meth- yl-1H-benzo- imidazol-2-yl- amine (D, P) | 2-Bromo-1-(4-bromo- phenyl)-etha- none | 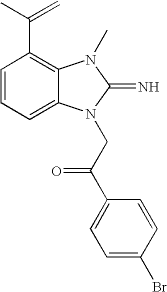 | B57 | 5.98 min (a) | (M + H)⁺384.1, 386.1 (1:1) |
| 7-Isopropenyl-1-meth- yl-1H-benzo- imida- zol-2-ylamine (D, P) | 2-Bromo-1-(4-chloro- phenyl)-etha- none | 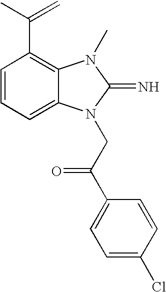 | B58 | 5.95 min (a) | (M + H)⁺340.1, 342.1 (3:1) |
| 7-Isopropenyl-1-meth- yl-1H-benzo- imida- zol-2-ylamine (D, P) | 2-Bromo-1-(2,4-di- chloro-phenyl)-eth- anone | 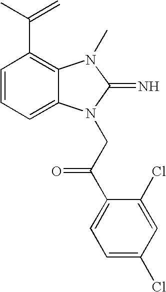 | B59 | 6.16 min (a) | (M + H)⁺374.2, 376.2 (3:2) |
| 2-Amino-3-meth- yl-3H-benzo- imidazole-4-carboxylic acid methyl ester (G) | 2-Bromo-1-phenyl- ethanone | 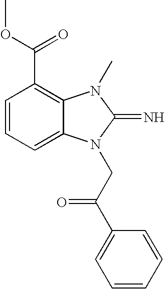 | B60 | 5.08 min (a) | (M + H)⁺324.2 |
| 2-Amino-3-meth- yl-3H-benzo- imidazole-4-carboxylic acid methyl ester (G) | 2-Bromo-1-(4-bromo- phenyl)-etha- none | 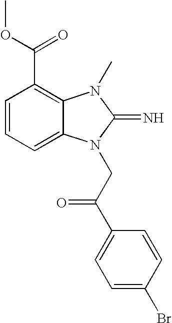 | B61 | 5.50 min (a) | (M + H)⁺402.1, 404.2 (1:1) |
| 2-Amino-3-meth- yl-3H-benzo- imidazole-4-carboxylic acid methyl ester (G) | 2-Bromo-1-(4-chloro- phenyl)-etha- none | 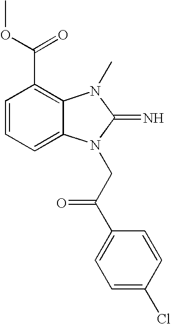 | B62 | 5.43 min (a) | (M + H)⁺358.1, 360.1 (3:1) |
| 2-Amino-3-meth- yl-3H-benzo- imidazole-4-carboxylic acid methyl ester (G) | 2-Bromo-1-(2,4-di- chloro-phenyl)-eth- anone | 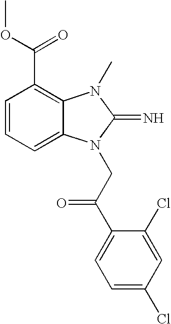 | B63 | 5.63 min (a) | (M + H)⁺392.1, 394.2 (3:2) |
| 1-Methyl-7-((E)-pro- penyl)-1H-benzoimi- dazol-2-yl- amine (D, P) | 2-Bromo-1-phenyl- ethanone | 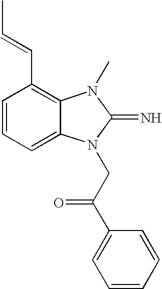 | B64 | 5.72 min (a) | (M + H)⁺306.3 |
| 1-Methyl-7-((E)-pro- penyl)-1H-benzoimi- dazol-2-yl- amine (D, P) | 2-Bromo-1-(4-bromo- phenyl)-eth- anone | 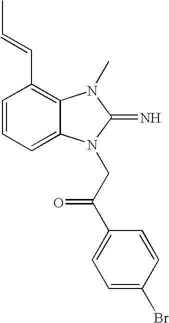 | B65 | 6.08 min (a) | (M + H)⁺384.1, 386.1 (1:1) |
| 1-Methyl-7-((E)-pro- penyl)-1H-benzoimi- dazol-2-yl- amine (D, P) | 2-Bromo-1-(4-chloro- phenyl)-eth- anone | 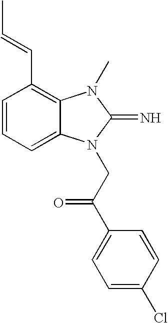 | B66 | 5.99 min (a) | (M + H)⁺340.2, 342.1 (3:1) |
| 1-Methyl-7-((E)-pro- penyl)-1H-benzoimi- dazol-2-yl- amine (D, P) | 2-Bromo-1-(2,4-di- chloro-phenyl)-eth- anone | 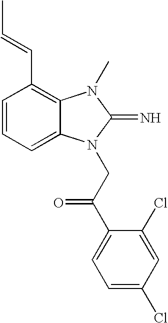 | B67 | 6.20 min (a) | (M + H)⁺374.2, 376.2 (3:2) |
| 1-Methyl-7-pro- pyl-1,3-di- hydro-benzo- imidazol-2-yl- ideneamine (D, P, Q) | 2-Bromo-1-phenyl- ethanone | 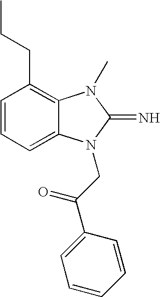 | B68 | 5.75 min (a) | (M + H)⁺308.2 |
| 1-Methyl-7-pro- pyl-1,3-di- hydro-benzo- imidazol-2-yl- ideneamine (D, P, Q) | 2-Bromo-1-(4-bromo- phenyl)-etha- none | 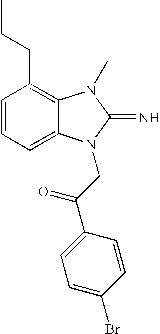 | B69 | 6.11 min (a) | (M + H)⁺386.2, 388.2 (1:1) |
| 1-Methyl-7-pro- pyl-1,3-dihydro- benzo- imidazol-2-yl- ideneamine (D, P, Q) | 2-Bromo-1-(4-chloro- phenyl)-eth- anone | 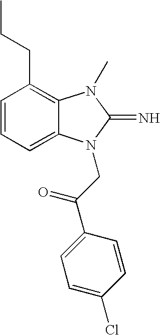 | B70 | 6.03 min (a) | (M + H)⁺342.2, 344.2 (3:1) |
| 1-Methyl-7-pro- pyl-1,3-di- hydro-benzo- imidazol-2-yl- ideneamine (D, P, RQ) | 2-Bromo-1-(2,4-di- chloro-phenyl)-eth- anone | 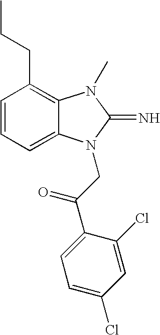 | B71 | 6.27 min (a) | (M + H)⁺376.2, 378.2 (3:2) |
| (2-Amino-3-meth- yl-3H-benzo- imidazol-4-yl)-meth- anol (G, H) | 2-Bromo-1-phenyl- ethanone | 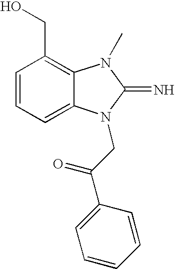 | B72 | 4.57 min (a) | (M + H)⁺296.2 |
| (2-Amino-3-meth- yl-3H-benzo- imidazol-4-yl)-meth- anol (G, H) | 2-Bromo-1-(4-bromo- phenyl)-eth- anone | 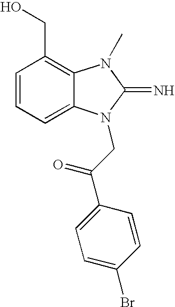 | B73 | 5.07 min (a) | (M + H)⁺374.2, 376.2 |
| (2-Amino-3-meth- yl-3H-benzo- imidazol-4-yl)-meth- anol (G, H) | 2-Bromo-1-(4-chloro- phenyl)-eth- anone | 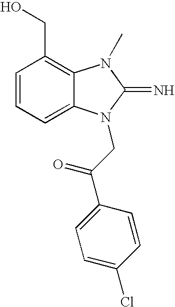 | B74 | 4.96 min (a) | (M + H)⁺330.1, 332.3 |
| (2-Amino-3-meth- yl-3H-benzo- imidazol-4-yl)-meth- anol (G, H) | 2-Bromo-1-(2,4-di- chloro-phenyl)-eth- anone | 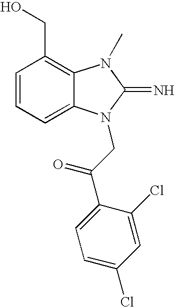 | B75 | 5.17 min (a) | (M + H)⁺364.2, 366.2 (3:2) |
| N-[3-(2-Amino-7-chloro- benzoimidazol-1-yl)-pro- pyl]-N-meth- yl-2-phenyl-a- cetamide (D, M, N) | 2-Bromo-1-(4-bromo- phenyl)-eth- anone | 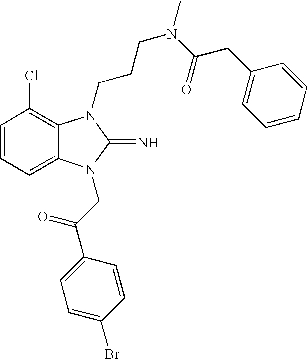 | B76 | 6.03 min (a) | (M + H)⁺553.1, 555.1, 557.0 (2:3:1) |
| N-[3-(2-Amino-7-chloro- benzoimidazol-1-yl)-pro- pyl]-N-methyl- benzamide (D, M, N) | 2-Bromo-1-(4-bromo- phenyl)-ethanone | 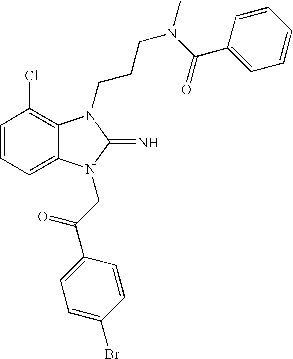 | B77 | | (M + H)⁺539.0, 541.0, 543.0 (2:3:1) |
| 1-Methyl-7-(1H-pyra- zol-4-yl)-1H-ben- zoimidazol-2-yl- amine (D, P) | 2-Bromo-1-phenyl- ethanone | 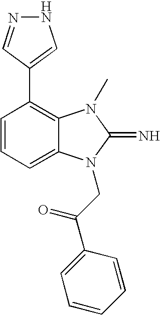 | B78 | 4.79 min (a) | (M + H)⁺332.3 |
| 1-Methyl-7-(1H-pyra- zol-4-yl)-1H-ben- zoimidazol-2-yl- amine (D, P) | 2-Bromo-1-(4-bromo- phenyl)-eth- anone | 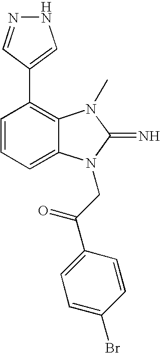 | B79 | 5.21 min (a) | (M + H)⁺410.2, 412.2 (1:1) |
| 1-Methyl-7-(1H-pyra- zol-4-yl)-1H-ben- zoimidazol-2-yl- amine (D, P) | 2-Bromo-1-(4-chloro- phenyl)-eth- anone | 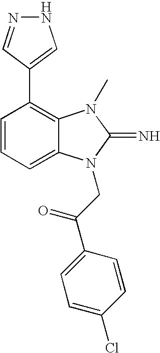 | B80 | 5.14 min (a) | (M + H)⁺366.3, 368.2 (3:1) |
| 1-Methyl-7-(1H-pyra- zol-4-yl)-1H-ben- zoimidazol-2-yl- amine (D, P) | 2-Bromo-1-(2,4-di- chloro-phenyl)-eth- anone | 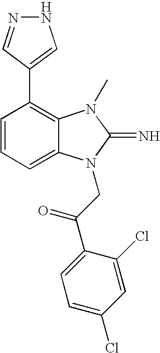 | B81 | 5.30 min (a) | (M + H)⁺400.2, 402.2 (3:2) |
| 7-Bromo-1-meth- yl-1H-benzo- imidazol-2-yl- amine (D) | 2-Bromo-1-phenyl- ethanone | 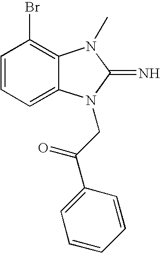 | B82 | 5.30 min (a) | (M + H)⁺344.2, 346.2 (1:1) |
| 7-Bromo-1-meth- yl-1H-benzo- imidazol-2-yl- amine (D) | 2-Bromo-1-(4-chloro- phenyl)-eth- anone | 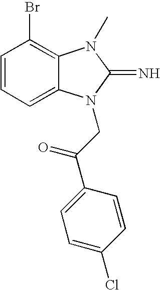 | B83 | 5.64 min (a) | (M + H)⁺378.1, 380.1 (1:1) |
| 7-Bromo-1-meth- yl-1H-benzo- imidazol-2-yl- amine (D) | 2-Bromo-1-(2,4-di- chloro-phenyl)-eth- anone | 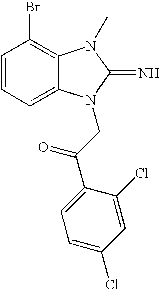 | B84 | 5.85 min (a) | (M + H)⁺412.1, 414.1, 416.0 (2:3:2) |
| 1-Methyl-7-((E)-pent-1-en- yl)-1H-ben- zoimidazol-2-yl- amine (D, P) | 2-Bromo-1-phenyl- ethanone | 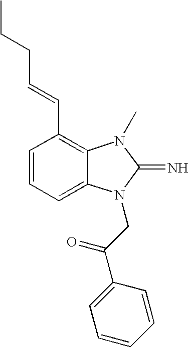 | B85 | 6.32 min (a) | (M + H)⁺334.3 |
| 1-Methyl-7-((E)-pent-1-en- yl)-1H-ben- zoimidazol-2-yl- amine (D, P) | 2-Bromo-1-(4-bromo- phenyl)-eth- anone | 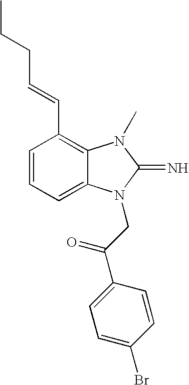 | B86 | 6.64 min (a) | (M + H)⁺412.3, 414.3 (1:1) |
| 1-Methyl-7-((E)-pent-1-en- yl)-1H-ben- zoimidazol-2-yl- amine (D, P) | 2-Bromo-1-(4-chloro- phenyl)-eth- anone | 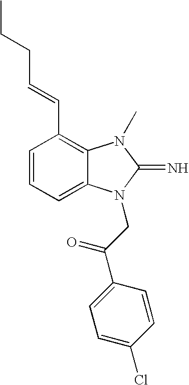 | B87 | 6.63 min (a) | (M + H)⁺368.2, 370.2 (3:1) |
| 1-Methyl-7-((E)-pent-1-en- yl)-1H-ben- zoimidazol-2-yl- amine (D, P) | 2-Bromo-1-(2,4-dichloro- phenyl)-eth- anone | 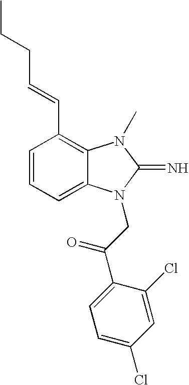 | B88 | 6.83 min (a) | (M + H)⁺402.3, 404.3 (3:2) |
| 5-[(Z)-1-Meth- oxy-but-2-en-(Z)-yl- idene]-1-meth- yl-4-meth- ylene-4,5-di- hydro-1H-imi- dazol-2-yl- amine (D, Z) | 2-Bromo-1-(4-bromo- phenyl)-eth- anone | 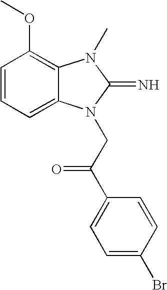 | B89 | 5.63 min (a) | (M + H)⁺374.1, 376.1 (1:1) |
| 1-Methyl-7-pen- tyl-1H-benzo- imidazol-2-yl- amine (D, P, Q) | 2-Bromo-1-phenyl- ethanone | 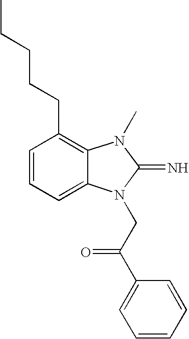 | B90 | 6.41 min (a) | (M + H)⁺336.3 |
| 1-Methyl-7-pen- tyl-1H-benzo- imidazol-2-yl- amine (D, P, Q) | 2-Bromo-1-(4-bromo- phenyl)-eth- anone | 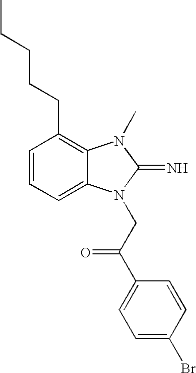 | B91 | 6.70 min (a) | (M + H)⁺414.3, 416.2 (1:1) |
| 1-Methyl-7-pen- tyl-1H-benzo- imidazol-2-yl- amine (D, P, Q) | 2-Bromo-1-(4-chloro- phenyl)-eth- anone | 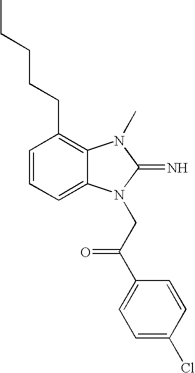 | B92 | 6.59 min (a) | (M + H)⁺370.2, 372.3 (3:1) |
| 1-Methyl-7-pen- tyl-1H-benzo- imidazol-2-yl- amine (D, P, Q) | 2-Bromo-1-(2,4-di- chloro-phenyl)-eth- anone | 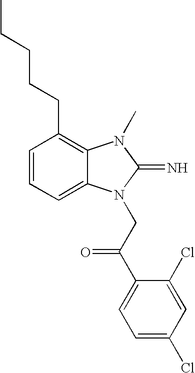 | B93 | 6.83 min (a) | (M + H)⁺404.2, 406.2 (3:2) |
| 2-(2-Amino- benzo- imidazol-1-yl)-N-meth- yl-N-phenyl- acetamide (L) | 2-Bromo-1-phenyl- ethanone | 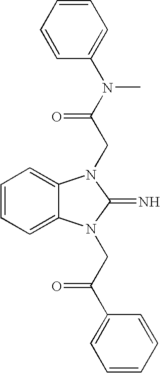 | B94 | 5.62 min (a) | (M + H)⁺399.1 |
| 2-(2-Amino- benzo- imidazol-1-yl)-N-meth- yl-N-phenyl- acetamide (L) | 2-Bromo-1-(4-bromo- phenyl)-eth- anone | 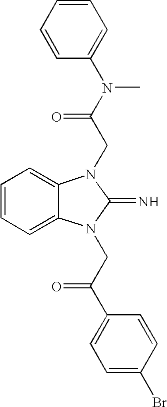 | B95 | 5.99 min (a) | (M + H)⁺477.1, 479.1 (1:1) |
| 2-(2-Amino- benzo- imidazol-1-yl)-N-meth- yl-N-phenyl- acetamide (L) | 2-Bromo-1-(4-chloro- phenyl)-eth- anone | 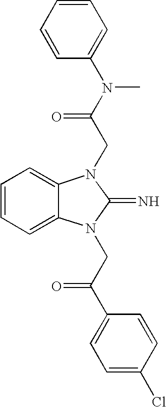 | B96 | 5.88 min (a) | (M + H)⁺433.1, 435.2 (3:1) |
| 2-(2-Amino-benzo- imidazol-1-yl)-N-meth- yl-N-phenyl- acetamide (L) | 2-Bromo-1-(2,4-di- chloro-phenyl)-eth- anone | 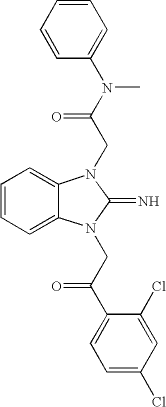 | B97 | 6.05 min (a) | (M + H)⁺467.1, 469.2 (3:2) |
| 2-(2-Amino- benzoimidazol-1-yl)-N-ben- zyl-N-methyl- acetamide (L) | 2-Bromo-1-phenyl- ethanone | 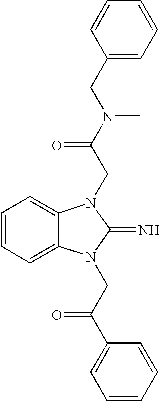 | B98 | 5.67 min (a) | (M + H)⁺413.2 |
| 2-(2-Amino- benzoimidazol-1-yl)-N-ben- zyl-N-methyl- acetamide (L) | 2-Bromo-1-(4-bromo- phenyl)-eth- anone | 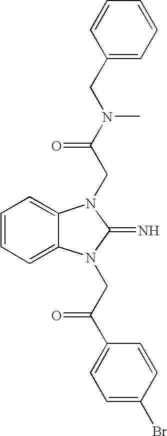 | B99 | 6.01 min (a) | (M + H)⁺491.1, 493.1 (1:1) |
| 2-(2-Amino- benzoimidazol-1-yl)-N-ben- zyl-N-methyl- acetamide (L) | 2-Bromo-1-(4-chloro- phenyl)-eth- anone | 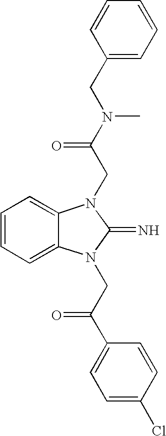 | B100 | 5.97 min (a) | (M + H)⁺447.1, 449.2 (3:1) |
| 2-(2-Amino- benzoimidazol-1-yl)-N-ben- zyl-N-methyl- acetamide (L) | 2-Bromo-1-(2,4-dichloro- phenyl)-eth- anone | 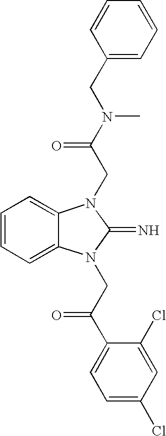 | B101 | 6.14 min (a) | (M + H)⁺481.1, 483.1 (3:2) |
| 7-Cyclopropyl-1-meth- yl-1H-benzo- imidazol-2-ylamine (D, P, S, T, U) | 2-Bromo-1-(4-bromo- phenyl)-eth- anone | 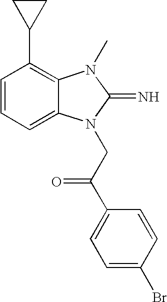 | B102 | 5.95 min (a) | (M + H)⁺384.2, 386.2 (1:1) |
| 1,7-Dimethyl-1H-benzo- imidazol-2-yla- mine (G) | 2-Bromo-1-(4-bromo- phenyl)-eth- anone | 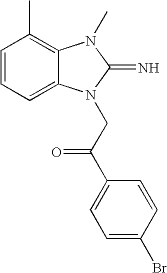 | B103 | 5.61 min (a) | (M + H)⁺358.3, 360.3 (1:1) |
| 1,7-Dimethyl-1H-benzo- imidazol-2-yla- mine (G) | 3-Bromomethyl- benzo[d]isoxazole | 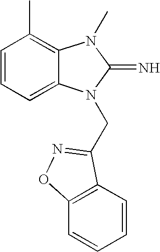 | B104 | 5.16 min (a) | (M + H)⁺293.1 |
| N-[3-(2-Amino-7-chloro- benzoimidazol-1-yl)-pro- pyl]-N-methyl- isophthala- mide (D, M, R) | 2-Bromo-1-(4-chloro- phenyl)-eth- anone | 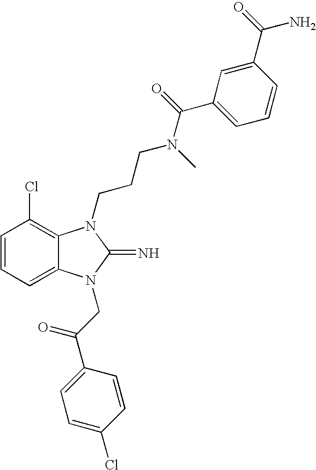 | B105 | 5.21 min (a) | (M + H)⁺538.1, 540.1 (3:2) |
| 1,7-Dimethyl-1H-benzo- imidazol-2-yla- mine (G) | 1-Bromomethyl- isoquinoline | 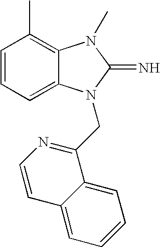 | B106 | 5.26 min (a) | (M + H)⁺303.2 |
| 7-Chloro-1-meth- yl-1H-benzo- imidazol-2-yl- amine (B, C, D) | 2-Bromo-1-(5-chlor- o-2-hydroxy- phenyl)-ethanone oxime (AI) | 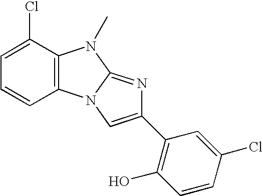 | B107 | 8.1 (a) | 332.1 (M + H)⁺ |
| 7-Chloro-1-meth- yl-1H-benzo- imidazol-2-yl- amine (B, C, D) | 2-Bromo-1-(5-chlor- o-2-hydroxy- phenyl)-ethanone oxime (AI) | 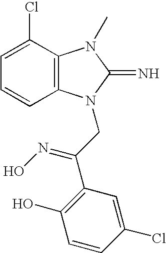 | B108 | 4.8 (a) | 365 (M + H)⁺ |
| 7-Chloro-1-meth- yl-1H-benzo- imidazol-2-yl- amine (B, C, D) | 2-Bromo-1-(5-chlor- o-2-hydroxy- phenyl)-ethanone oxime (AI) | 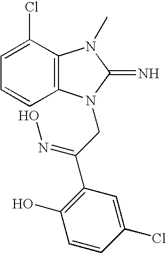 | B109 | 4.9 (a) | 365 (M + H)⁺ |
| N-[2-(2-Amino- benzoimidazol-1-yl)-eth- yl]-N-methyl- acetamide (B, BB, CC, C, D, E) | 2-Bromo-1-(4-chloro- phenyl)-eth- anone | 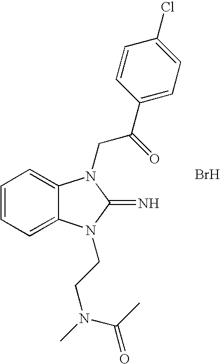 | B110 | 5.11 min (a) | 384.9, 386.9 (M + H)⁺ |
| N-[2-(2-Amino- benzoimidazol-1-yl)-eth- yl]-N-methyl- benzamide (B, BB, CC, C, D, E) | 2-Bromo-1-(4-chloro- phenyl)-eth- anone | 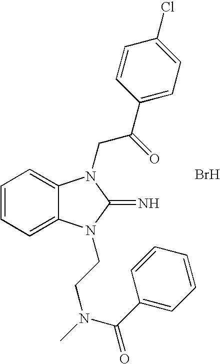 | B111 | 5.62 min (a) | 446.9, 448.9 (M + H)⁺ |
| N-[2-(2-Amino- benzoimidazol-1-yl)-eth- yl]-N-meth- yl-2-phenyl- acetamide (B, BB, CC, C, D, E) | 2-Bromo-1-(4-chloro- phenyl)-eth- anone | 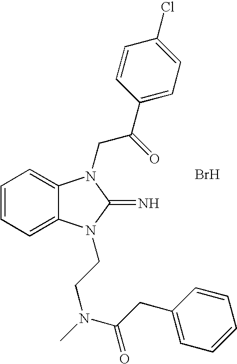 | B112 | 5.86 min (a) | 460.9, 463.0 (M + H)⁺ |
| Thiophene-2-carboxy- lic acid [2-(2-amino- benzoimidazol-1-yl)-eth- yl]-methyl- amide (B, BB, CC, C, D, E) | 2-Bromo-1-(4-chloro- phenyl)-etha- none | 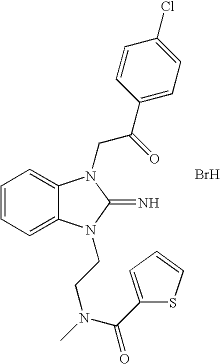 | B113 | 5.62 min (a) | 452.9, 454.9 (M + H)⁺ |
| Cyclohexanecarboxy- lic acid [2-(2-amino- benzoimidazol-1-yl)-eth- yl]-methyl- amide (B, BB, CC, C, D, E) | 2-Bromo-1-(4-chloro- phenyl)-etha- none | 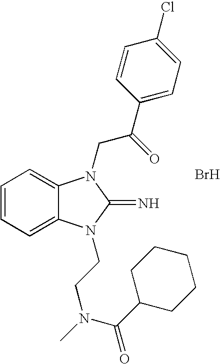 | B114 | 6.04 min (a) | 453.0, 454.3 (M + H)⁺ |
| N-[2-(2-Amino-7-chloro- benzoimidazol-1-yl)-eth- yl]-N-methyl- benzamide (B, BB, CC, C, D, E) | 2-Bromo-1-(4-chloro- phenyl)-eth- anone | 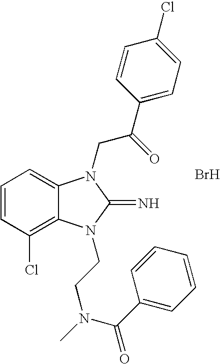 | B115 | 5.86 min (a) | 481.2, 483.1 (M + H)⁺ |
| N-[2-(2-Amino-7-chloro- benzoimidazol-1-yl)-eth- yl]-N-meth- yl-2-phenyl- acetamide (B, BB, CC, C, D, E) | 2-Bromo-1-(4-chloro- phenyl)-etha- none | 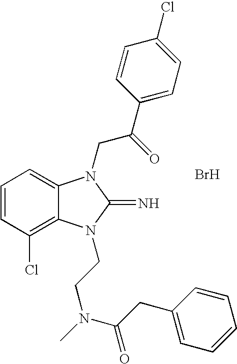 | B116 | 6.09 min (a) | 495.2, 497.1 (M + H)⁺ |
| N-[2-(2-Amino-7-chloro- benzoimidazol-1-yl)-eth- yl]-N-meth- yl-2-phenyl- acetamide (B, BB, CC, C, D, E) | 2-Bromo-1-(4-bromo- phenyl)-eth- anone | 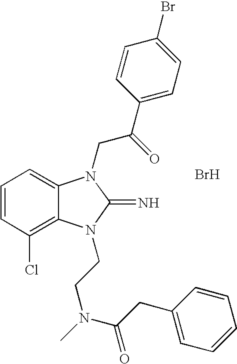 | B117 | 6.19 min (a) | 539.0, 541.0, 543.0 (M + H)⁺ |
| N-[2-(2-Amino-7-chloro- benzoimidazol-1-yl)-eth- yl]-N-meth- yl-2-phenyl- acetamide (B, BB, CC, C, D, E) | 2-Bromo-1-phenyl- ethanone | 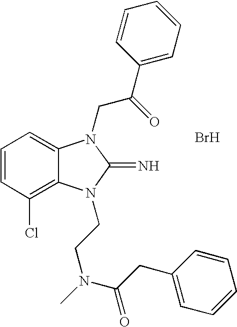 | B118 | 5.86 min (a) | 461.1, 463.1 (M + H)⁺ |
| N-[2-(2-Amino-7-chloro- benzoimidazol-1-yl)-eth- yl]-N-meth- yl-2-phenyl- acetamide (B, BB, CC, C, D, E) | 2-Bromo-1-(4-chloro- phenyl)-etha- none | 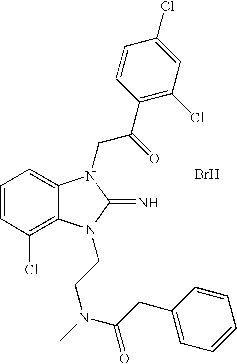 | B119 | 6.33 min (a) | 529.0, 531.0, 532.0 (M + H)⁺ |
| N-[3-(2-Amino-7-chloro- benzoimidazol-1-yl)-pro- pyl]-N-meth- yl-2-phenyl- acetamide (B, BB, CC, C, D, E) | 2-Bromo-1-(4-chloro- phenyl)-etha- none | 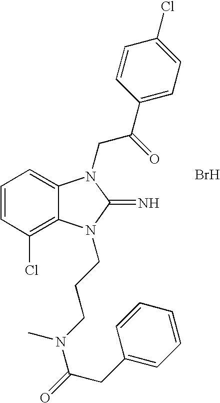 | B120 | 6.17 min (a) | 509.1, 510.2, 511.1 (M + H)⁺ |
| N-[3-(2-Amino-7-chloro- benzoimidazol-1-yl)-pro- pyl]-N-meth- yl-2-phenyl- acetamide (B, BB, CC, C, D, E) | 2-Bromo-1-(4-bromo- phenyl)-eth- anone | 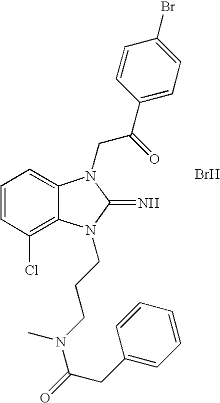 | B121 | 6.21 min (a) | 553.2, 555.1, 557.1 (M + H)⁺ |
| N-[3-(2-Amino-7-chloro- benzoimidazol-1-yl)-pro- pyl]-N-meth- yl-2-phenyl- acetamide (B, BB, CC, C, D, E) | 2-Bromo-1-phenyl- ethanone | 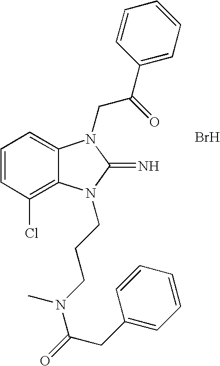 | B122 | 5.90 min (a) | 475.1, 476.1 (M + H)⁺ |
| N-[3-(2-Amino-7-chloro- benzoimidazol-1-yl)-pro- pyl]-N-meth- yl-2-phenyl- acetamide (B, BB, CC, C, D, E) | 2-Bromo-1-(2,4-dichloro- phenyl)-etha- none | 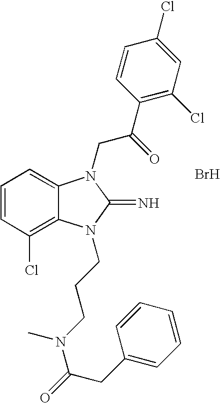 | B123 | 6.34 min (a) | 543.0, 545.1, 546.0 (M + H)⁺ |
| N-[3-(2-Amino-7-chloro- benzoimidazol-1-yl)-pro- pyl]-N-methyl- benzamide (B, BB, CC, C, D, E) | 2-Bromo-1-(4-chloro- phenyl)-etha- none | 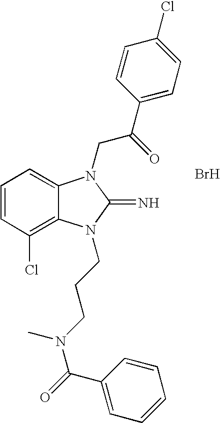 | B124 | 5.98 min (a) | 495.1, 497.1, 498.1 (M + H)⁺ |
| N-[3-(2-Amino-7-chloro- benzoimidazol-1-yl)-pro- pyl]-N-methyl- benzamide (B, BB, CC, C, D, E) | 2-Bromo-1-(4-bromo- phenyl)-etha- none | 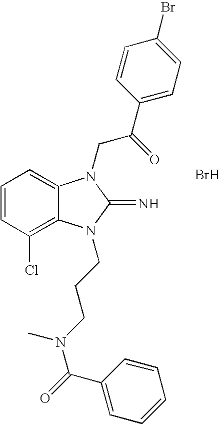 | B125 | 6.03 min (a) | 539.0, 541.0, 543.0 (M + H)⁺ |
| N-[3-(2-Amino-7-chloro- benzoimidazol-1-yl)-pro- pyl]-N-methyl- benzamide (B, BB, CC, C, D, E) | 2-Bromo-1-phenyl- ethanone | 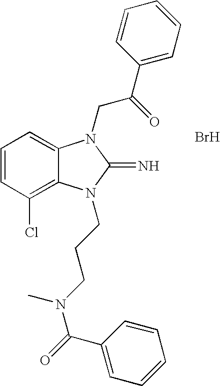 | B126 | 5.70 min (a) | 461.1, 463.1 (M + H)⁺ |
| N-[3-(2-Amino-7-chloro- benzoimidazol-1-yl)-pro- pyl]-N-methyl- benzamide (B, BB, CC, C, D, E) | 2-Bromo-1-(2,4-dichloro- phenyl)-etha- none | 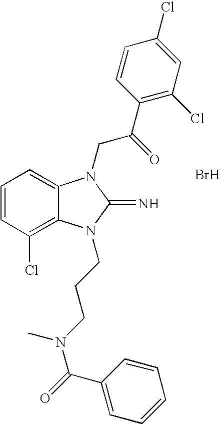 | B127 | 6.15 min (a) | 529.0, 631.0, 532.0, 533.0 (M + H)⁺ |
| N-[3-(2-Amino-7-chloro- benzoimidazol-1-yl)-pro- pyl]-N-methyl- acetamide (B, BB, CC, C, D, E) | 2-Bromo-1-(4-chloro- phenyl)-eth- anone | 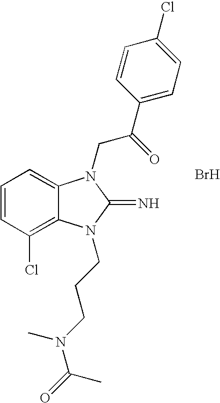 | B128 | 5.43 min (a) | 433.1, 435.1, 436.1 (M + H)⁺ |
| N-[3-(2-Amino-7-chloro- benzoimidazol-1-yl)-pro- pyl]-N-methyl- acetamide (B, BB, CC, C, D, E) | 2-Bromo-1-(4-bromo- phenyl)-etha- none | 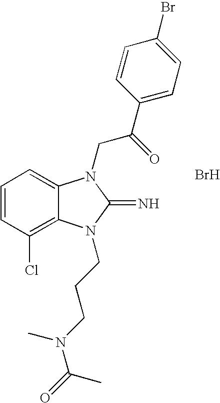 | B129 | 5.49 min (a) | 477.2, 479.0, 481.1 (M + H)⁺ |
| N-[3-(2-Amino-7-chloro- benzoimidazol-1-yl)-pro- pyl]-N-methyl- acetamide (B, BB, CC, C, D, E) | 2-Bromo-1-phenyl- ethanone | 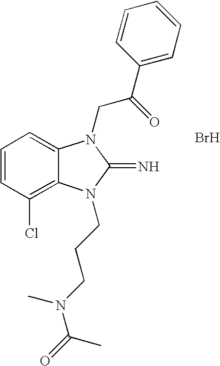 | B130 | 5.11 min (a) | 399.1, 401.1 (M + H)⁺ |
| N-[3-(2-Amino-7-chloro- benzoimidazol-1-yl)-pro- pyl]-N-methyl- acetamide (B, BB, CC, C, D, E) | 2-Bromo-1-(2,4-dichloro- phenyl)-etha- none | 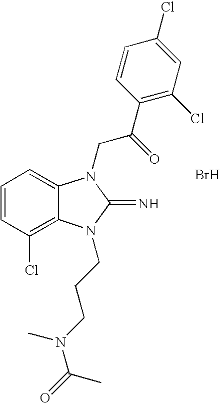 | B131 | 5.59 min (a) | 467.1, 469.1, 471.1 (M + H)⁺ |
| N-[2-(2-Amino-7-chloro- benzoimidazol-1-yl)-eth- yl]-N-methyl- benzamide (B, BB, CC, C, D, E) | 2-Bromo-1-(4-bromo- phenyl)-eth- anone | 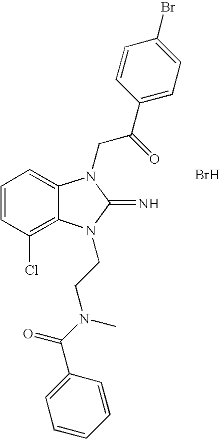 | B132 | 5.97 min (a) | 525.1, 527.0, 529.0 (M + H)⁺ |
| N-[2-(2-Amino-7-ethyl- benzoimidazol-1-yl)-eth- yl]-N-methyl- benzamide (B, BB, CC, C, D, P, Q, E) | 2-Bromo-1-(4-chloro- phenyl)-etha- none | 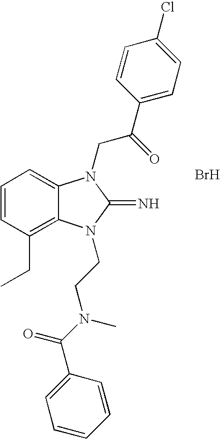 | B133 | 6.16 min (a) | 475.3, 477.3 (M + H)⁺ |
| N-[2-(2-Amino-7-ethyl- benzoimidazol-1-yl)-eth- yl]-N-meth- yl-2-phenyl- acetamide (B, BB, CC, C, D, P, Q, E) | 2-Bromo-1-(4-chloro- phenyl)-etha- none | 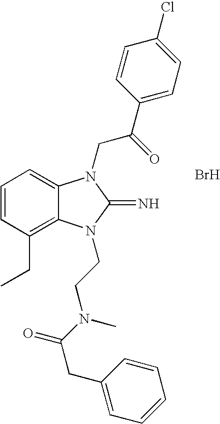 | B134 | 2.41 (d) | 489.1, 491.1 (M + H)⁺ |
| N-[3-(2-Amino-7-ethyl- benzoimidazol-1-yl)-pro- pyl]-N-meth- yl-2-phenyl- acetamide (B, BB, CC, C, D, P, Q, E) | 2-Bromo-1-(4-chloro- phenyl)-eth- anone | 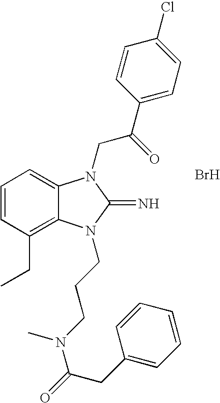 | B135 | 6.31 min (a) | 503.2, 505.2 (M + H)⁺ |
| N-[3-(2-Amino-7-ethyl- benzoimidazol-1-yl)-pro- pyl]-N-meth- yl-2-phenyl- acetamide (B, BB, CC, C, D, P, Q, E) | 2-Bromo-1-(4-bromo- phenyl)-eth- anone | 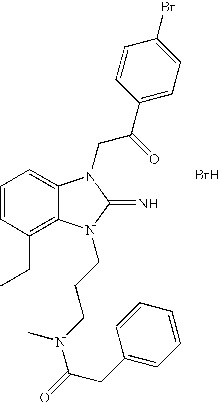 | B136 | 6.40 min (a) | 547.2, 549.1 (M + H)⁺ |
| N-[3-(2-Amino-7-ethyl- benzoimidazol-1-yl)-pro- pyl]-N-meth- yl-2-phenyl- acetamide (B, BB, CC, C, D, P, Q, E) | 2-Bromo-1-phenyl- ethanone | 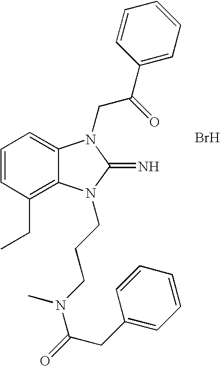 | B137 | 6.08 min (a) | 469.3 (M + H)⁺ |
| N-[3-(2-Amino-7-ethyl- benzoimidazol-1-yl)-pro- pyl]-N-meth- yl-2-phenyl- acetamide (B, BB, CC, C, D, P, Q, E) | 2-Bromo-1-(2,4-dichloro- phenyl)-etha- none | 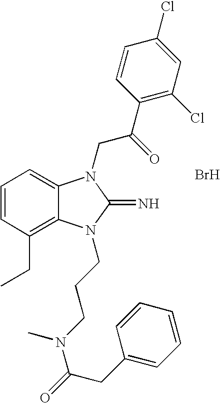 | B138 | 6.49 min (a) | 537.1, 539.1 (M + H)⁺ |
| N-[3-(2-Amino-7-ethyl- benzoimidazol-1-yl)-pro- pyl]-N-methyl- benzamide (B, BB, CC, C, D, P, Q, E) | 2-Bromo-1-(4-chloro- phenyl)-etha- none | 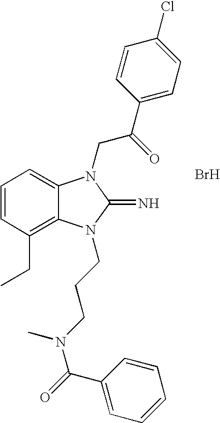 | B139 | 6.13 min (a) | 489.1, 491.2 (M + H)⁺ |
| N-[3-(2-Amino-7-ethyl- benzoimidazol-1-yl)-pro- pyl]-N-methyl- benzamide (B, BB, CC, C, D, P, Q, E) | 2-Bromo-1-(4-bromo- phenyl)-etha- none | 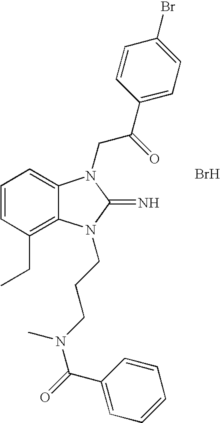 | B140 | 6.21 min (a) | 533.2, 535.1, 536.2 (M + H)⁺ |
| N-[3-(2-Amino-7-ethyl- benzoimidazol-1-yl)-pro- pyl]-N-methyl- benzamide (B, BB, CC, C, D, P, Q, E) | 2-Bromo-1-phenyl- ethanone | 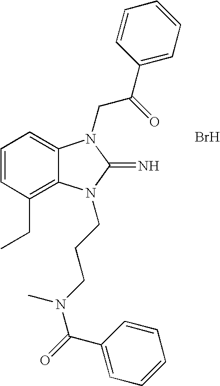 | B141 | 5.87 min (a) | 455.2 (M + H)⁺ |
| N-[3-(2-Amino-7-ethyl- benzoimidazol-1-yl)-pro- pyl]-N-methyl- benzamide (B, BB, CC, C, D, P, Q, E) | 2-Bromo-1-(2,4-dichloro- phenyl)-etha- none | 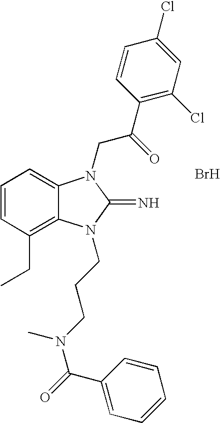 | B142 | 6.31 min (a) | 523.2, 525.1 (M + H)⁺ |
| N-[3-(2-Amino-7-ethyl- benzoimidazol-1-yl)-pro- pyl]-N-methyl- acetamide (B, BB, CC, C, D, P, Q, E) | 2-Bromo-1-(4-chloro- phenyl)-etha- none | 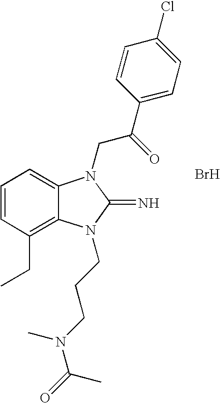 | B143 | 5.61 min (a) | 427.2, 429.1 (M + H)⁺ |
| N-[3-(2-Amino-7-ethyl- benzoimidazol-1-yl)-pro- pyl]-N-methyl- acetamide (B, BB, CC, C, D, P, Q, E) | 2-Bromo-1-(4-bromo- phenyl)-etha- none | 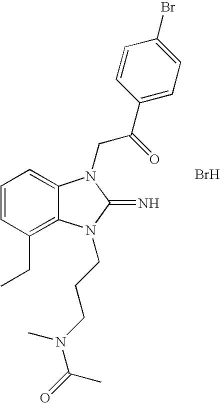 | B144 | 5.67 min (a) | 471.1, 473.1 (M + H)⁺ |
| N-[3-(2-Amino-7-ethyl- benzoimidazol-1-yl)-pro- pyl]-N-methyl- acetamide (B, BB, CC, C, D, P, Q, E) | 2-Bromo-1-phenyl- ethanone | 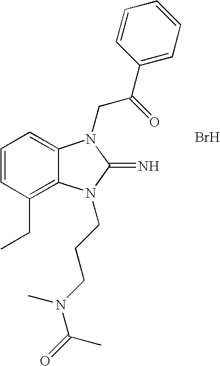 | B145 | 5.30 min (a) | 393.2 (M + H)⁺ |
| N-[3-(2-Amino-7-ethyl- benzoimidazol-1-yl)-pro- pyl]-N-methyl- acetamide (B, BB, CC, C, D, P, Q, E) | 2-Bromo-1-(2,4-dichloro- phenyl)-etha- none | 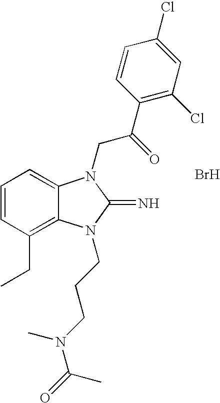 | B146 | 5.78 min (a) | 461.1, 463.1 (M + H)⁺ |
| 7-Chloro-1-(2-di- benzylamino- ethyl)-1H-benzo- imidazol-2-yl- amine (B, C, DD, EE, E) | 2-Bromo-1-(4-chloro- phenyl)-etha- none | 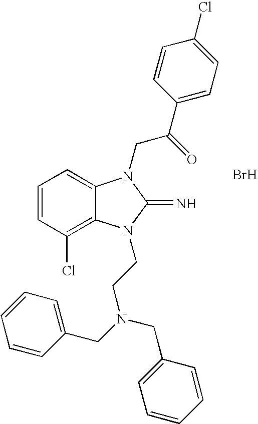 | B147 | 6.20 min (a) | 543.5, 545.4, 546.5, 547.5 (M + H)⁺ |
| 7-Ethyl-1-(2-di- benzylamino- ethyl)-1H-benzo- imidazol-2-yl- amine (B, C, DD, EE, P, R, E) | 2-Bromo-1-(4-chloro- phenyl)-etha- none | 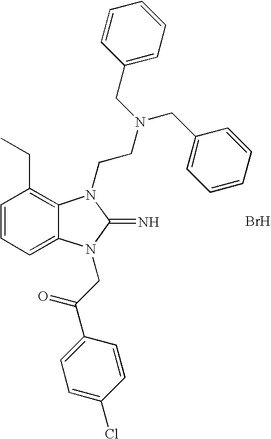 | B148 | 3.88 min (e) | 537.2, 539.2, 540.3 (M + H)⁺ |
| [3-(2-Amino-7-chloro- benzoimidazol-1-yl)-pro- pyl]-carbamic acid tert-bu- tyl ester (B, C, D, E) | 2-Bromo-1-(4-chloro- phenyl)-etha- none | 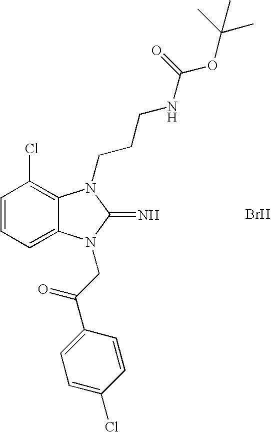 | B149 | 6.21 min (a) | 477.1, 479.0, 480.1, 481.1 (M + H)⁺ |
| [3-(2-Amino-7-chloro- benzoimidazol-1-yl)-pro- pyl]-carbamic acid tert-bu- tyl ester (B, C, D, E) | 2-Bromo-1-(4-bromo- phenyl)-etha- none | 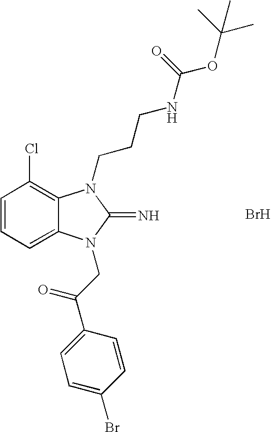 | B150 | 6.29 min (a) | 521.0, 523.0, 524.1, 525.1 (M + H)⁺ |
| 3-(2-Amino-7-chloro- benzoimidazol-1-yl- methyl)-pipe- ridine-1-carboxylic acid tert-butyl ester (B, C, D, E) | 2-Bromo-1-(4-chloro- phenyl)-etha- none | 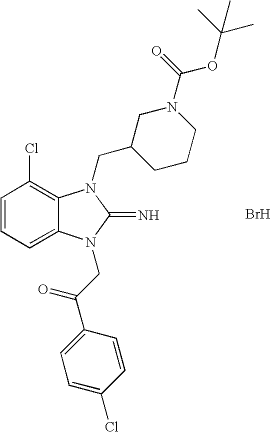 | B151 | 6.65 min (a) | 517.1, 519.1, 521.2 (M + H)⁺ |
| 3-(2-Amino-7-chloro- benzoimidazol-1-yl- methyl)-pipe- ridine-1-carboxylic acid tert-butyl ester (B, C, D, E) | 2-Bromo-1-(4-bromo- phenyl)-etha- none | 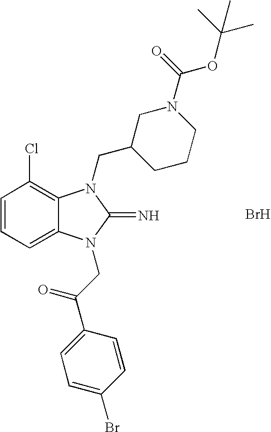 | B152 | 6.71 min (a) | 561.1, 563.2, 564.0 (M + H)⁺ |
| (2-Amino-1-meth- yl-1H-benzo- imidazol-5-yl)-phenyl- methanone (G) | 2-Bromo-1-(4-chloro- phenyl)-etha- none | 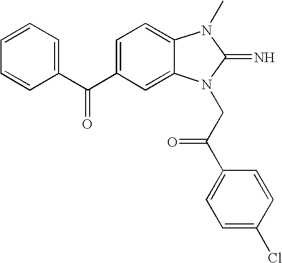 | B153 | 2.49 (d) | M+ 404 |
| 1,5-Dimethyl-1H-benzo- imidazol-2-yla- mine (G) | 2-Bromo-1-(4-chloro- phenyl)-etha- none | 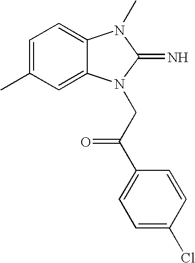 | B154 | 2.25 (d) | M+ 314 |
| 1-Methyl-5-tri- fluoromethyl-1H-benzo- imidazol-2-yl- amine (G) | 2-Bromo-1-(4-chloro- phenyl)-eth- anone | 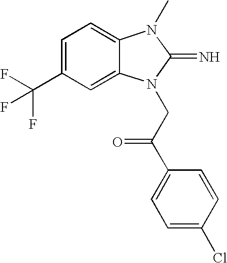 | B155 | 2.56 (d) | M+ 2.56 |
| 5-Methoxy-1-meth- yl-1H-benzo- imidazol-2-yl- amine (G) | 2-Bromo-1-(4-chloro- phenyl)-etha- none | 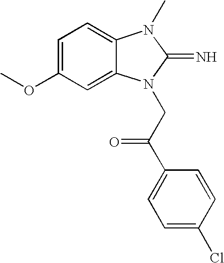 | B156 | 2.18 (d) | M+ 330 |
| 5-Methoxy-1H-benzo- imidazol-2-ylamine (G) | 2-Bromo-1-(4-chloro- phenyl)-eth- anone | 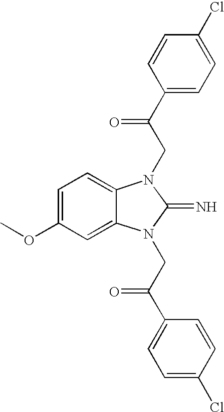 | B157 | 2.80 (d) | M+ 468/470 |
| 5-Ethoxy-1-meth- yl-1H-benzo- imidazol-2-yl- amine (G) | 2-Bromo-1-(4-chloro- phenyl)-eth- anone | 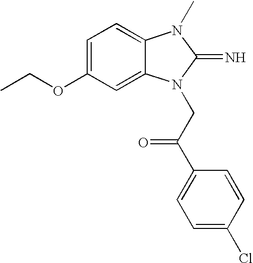 | B158 | 2.45 (d) | M+ 344 |
| 1-Methyl-5-tri- fluoromethoxy-1H-benzo- imidazol-2-yl- amine (G) | 2-Bromo-1-(4-chloro- phenyl)-eth- anone | 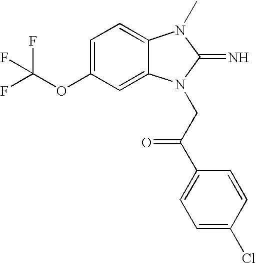 | B159 | 2.69 (d) | M+ 384 |
| 1,6-Dimethyl-1H-benzo- imidazol-2-yla- mine (G) | 2-Bromo-1-(4-chloro- phenyl)-etha- none | 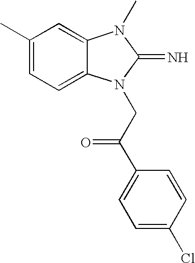 | B160 | 1.83 (d) | (M + H)⁺314 |
| 2-Amino-3-meth- yl-3H-benzo- imidazole-5-carbo- nitrile (G) | 2-Bromo-1-(4-chloro- phenyl)-eth- anone | 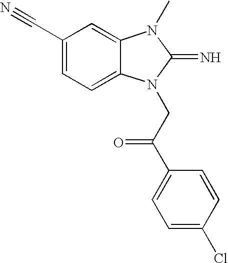 | B161 | 1.88 (d) | (M + H)⁺325 |
| 6-Methoxy-1-meth- yl-1H-benzo- imidazol-2-yl- amine (G) | 2-Bromo-1-(4-chloro- phenyl)-eth- anone | 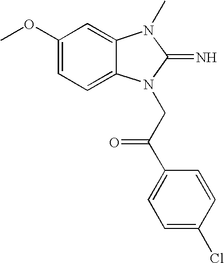 | B162 | 1.80 (d) | (M + H)⁺330 |
| 1,7-Dimethyl-1H-benzo- imidazol-2-yla- mine (G) | 2-Bromo-1-(4-chloro- phenyl)-etha- none | 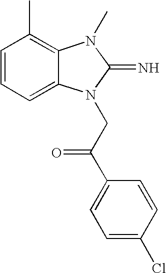 | B163 | 1.97 (d) | (M + H)⁺314 |
| Methyl-(1-meth- yl-1H-benzo- imidazol-2-yl)-a- mine (F) | 2-Bromo-1-(4-chloro- phenyl)-etha- none | 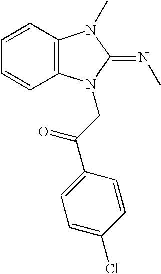 | B164 | 5.45 (a) | 314.3 (M + H)⁺ |
| Methyl-(1-meth- yl-1H-benzo- imidazol-2-yl)-a- mine (F) | 2-Bromo-1-(4-bromo- phenyl)-etha- none | 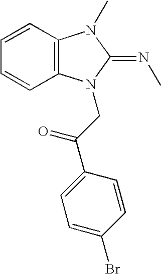 | B165 | 5.50 (a) | 359 (M + H)⁺ |
| Ethyl-(1-methyl-1H-benzo- imidazol-2-yl)-a- mine (F) | 2-Bromo-1-(4-bromo- phenyl)-etha- none | 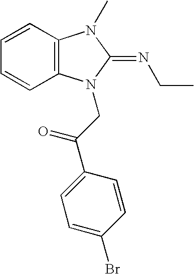 | B166 | 5.75 (a) | 373 (M + H)⁺ |
| (1-Methyl-1H-benzo- imidazol-2-yl)-propyl- amine (F) | 2-Bromo-1-(4-bromo- phenyl)-etha- none | 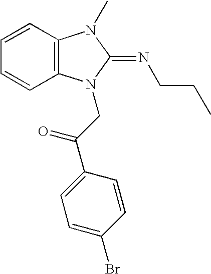 | B167 | 5.97 (a) | 387 (M + H)⁺ |
| Butyl-(1-methyl-1H-benzo- imidazol-2-yl)-a- mine (F) | 2-Bromo-1-(4-bromo- phenyl)-etha- none | 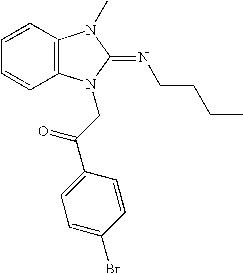 | B168 | 6.22 (a) | 401 (M + H)⁺ |
| Isopropyl-(1-meth- yl-1H-benzo- imidazol-2-yl)-a- mine (F) | 2-Bromo-1-(4-bromo- phenyl)-etha- none | 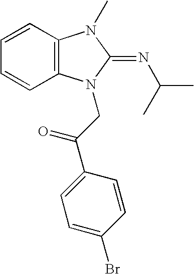 | B169 | 5.95 (a) | 387 (M + H)⁺ |
| Cyclopropylmeth- yl-(1-methyl-1H-benzo- imidazol-2-yl)-a- mine (F) | 2-Bromo-1-(4-bromo- phenyl)-etha- none | 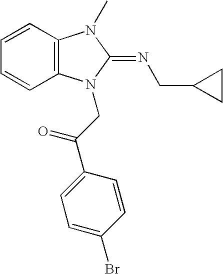 | B170 | 6.03 (a) | 399 (M + H)⁺ |
| (2-Methoxy- ethyl)-(1-methyl-1H-benzo- imidazol-2-yl)-a- mine (F) | 2-Bromo-1-(4-bromo- phenyl)-eth- anone | 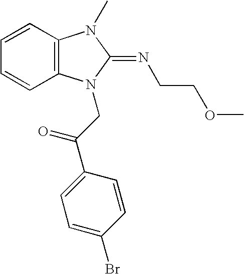 | B171 | 5.68 (a) | 403 (M + H)⁺ |
| 2-(1-Methyl-1H-benzo- imidazol-2-yl- amino)-ethanol (F) | 2-Bromo-1-(4-bromo- phenyl)-eth- anone | 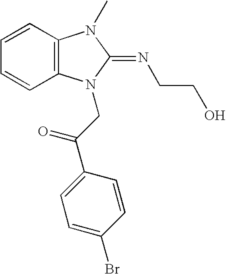 | B172 | 5.2 (a) | 389 (M + H)⁺ |
| 3-(1-Methyl-1H-benzo- imidazol-2-yl- amino)-propan-1-ol (F) | 2-Bromo-1-(4-bromo- phenyl)-etha- none | 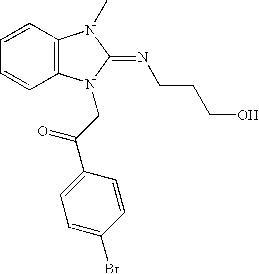 | B173 | 5.26 (a) | 403 (M + H)⁺ |
| Benzyl-(1-meth- yl-1H-benzo- imidazol-2-yl)-a- mine (F) | 2-Bromo-1-(4-bromo- phenyl)-etha- none | 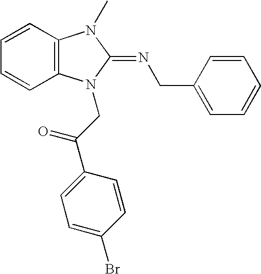 | B174 | 6.1 (a) | 435 (M + H)⁺ |
| 1-[2-(Benzyl- methyl-amino)-eth- yl]-1H-benzo- imidazol-2-yl- amine (E) | 2-Bromo-1-(4-chloro- phenyl)-etha- none | 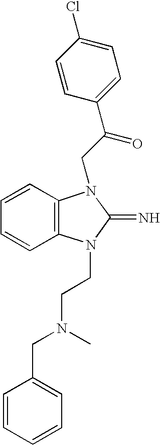 | B175 | 6.4 (a) | 434 (M + H)⁺ |
| 1-(2-Di- benzylamino- ethyl)-1H-benzo- imidazol-2-yl- amine (E) | 2-Bromo-1-(4-chloro- phenyl)-etha- none | 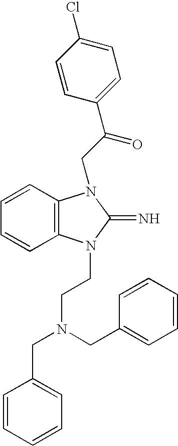 | B176 | 7.1 (a) | 510 (M + H)⁺ |
| 1-Methyl-1H-benzo- imidazol-2-yl- amine | 2-Bromo-1-(3,4-di- chlorophenyl)-etha- none | 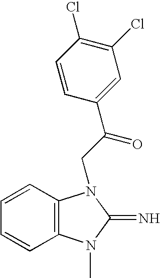 | B177 | 2.50 (c) | 335.3 (M + H)⁺ |
| 1-Methyl-1H-benzo- imidazol-2-yl- amine | 2-Bromo-1-(3-bromo- phenyl)-etha- none | 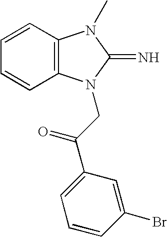 | B178 | 2.29 (c) | 345.3 (M + H)⁺ |
| 1-Methyl-1H-benzo- imidazol-2-yl- amine | 5-(2-Bromo- acetyl)-2-hydroxy- benzamide | 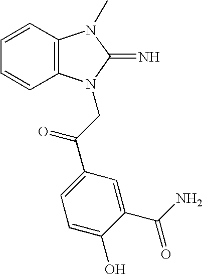 | B179 | 1.24(c) | 325.1 (M + H)⁺ |
| 1-Methyl-1H-benzo- imidazol-2-yl- amine | 2-Bromo-1-(2,5-di- methoxyphenyl)-etha- none | 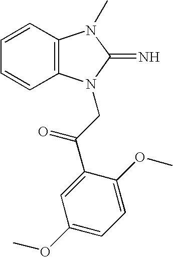 | B180 | 2.15 (c) | 326.2 (M + H)⁺ |
| 1-Methyl-1H-benzo- imidazol-2-yl- amine | 2-Bromo-1-(4-chlor- o-3-methyl- phenyl)-etha- none | 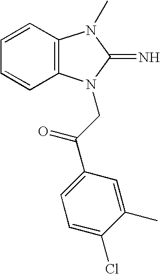 | B181 | 2.42 (c) | 314.3 (M + H)⁺ |
| 1-Methyl-1H-benzo- imidazol-2-yl- amine | 2-Bromo-1-(2-nitro- phenyl)-etha- none | 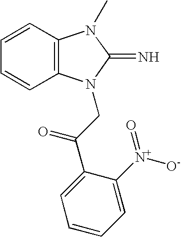 | B182 | 1.94 (c) | 311.2 (M + H)⁺ |
| 1-Methyl-1H-benzo- imidazol-2-yl- amine | 2-Bromo-1-(2,4-di- methylphenyl)-etha- none | 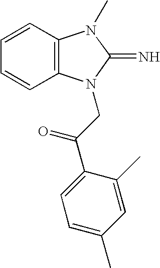 | B183 | 2.33 (c) | 294.3 (M + H)⁺ |
| 1-Methyl-1H-benzo- imidazol-2-yl- amine | 2-Bromo-1-(4-fluoro- phenyl)-etha- none | 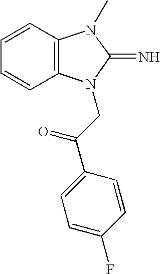 | B184 | 2.02 (c) | 284.3 (M + H)⁺ |
| 1-Methyl-1H-benzo- imidazol-2-yl- amine | 2-Bromo-1-(4-tri- fluoromethoxy- phenyl)-ethanone | 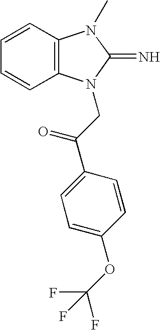 | B185 | 2.47 (c) | 350.3 (M + H)⁺ |
| 1-Methyl-1H-benzo- imidazol-2-yl- amine | 1-(3,5-Bis- trifluoromethylphe- nyl)-2-bromo- ethanone | 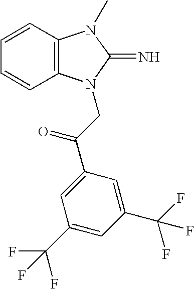 | B186 | 2.63 (c) | 402.3 (M + H)⁺ |
| 1-Methyl-1H-benzo- imidazol-2-yl- amine | 2-Bromo-1-(3-chloro- phenyl)-eth- anone | 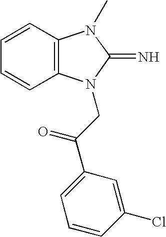 | B187 | 2.25 (c) | 300.3 (M + H)⁺ |
| 1-Methyl-1H-benzo- imidazol-2-yl- amine | 2-Bromo-1-(2-chloro- phenyl)-etha- none | 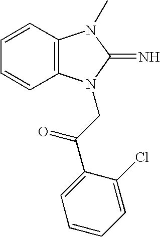 | B188 | 2.12 (c) | 300.3 (M + H)⁺ |
| 1-Methyl-1H-benzo- imidazol-2-yl- amine | 1-Ben- zo[1,3]dioxol-5-yl-2-bro- moethanone | 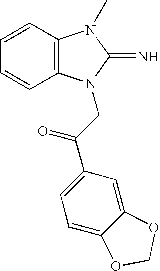 | B189 | 1.98 (c) | 310.2 (M + H)⁺ |
| 1-Methyl-1H-benzo- imidazol-2-yl- amine | 2-Bromo-1-(4-di- fluoromethoxy- phenyl)-ethanone | 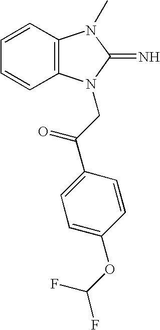 | B190 | 2.26 (c) | 332.3 (M + H)⁺ |
| 1-Methyl-1H-benzo- imidazol-2-yl- amine | 2-Bromo-1-(3,4-di- fluorophenyl)-etha- none | 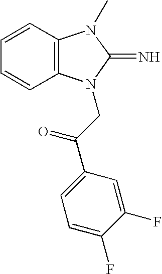 | B191 | 2.13 (c) | 302.2 (M + H)⁺ |
| 1-Methyl-1H-benzo- imidazol-2-yl- amine | 2-Bromo-1-(2,3-di- hydrobenzo[1,4]di- oxin-6-yl)-eth- anone | 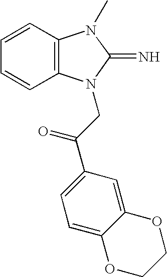 | B192 | 2.02 (c) | 324.3 (M + H)⁺ |
| 1-Methyl-1H-benzo- imidazol-2-yl- amine | 2-Bromo-1-(3-fluoro- phenyl)-etha- none | 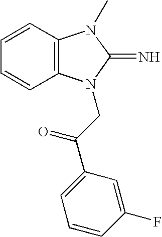 | B193 | 2.10 (c) | 284.3 (M + H)⁺ |
| 1-Methyl-1H-benzo- imidazol-2-yl- amine | 2-Bromo-1-(3-ni- tro-4-chloro- phenyl)-etha- none | 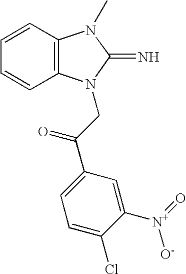 | B194 | 2.29 (c) | 345.2 (M + H)⁺ |
| 1-Methyl-1H-benzo- imidazol-2-yl- amine | 2-Bromo-1-(4-tri- fluoromethylphe- nyl)-ethanone | 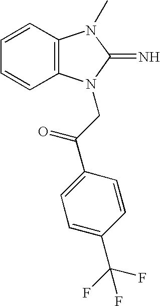 | B195 | 2.41 (c) | 334.3 (M + H)⁺ |
| 1-Methyl-1H-benzo- imidazol-2-yl- amine | 2-Bromo-1-(3-tri- fluoromethylphe- nyl)-ethanone | 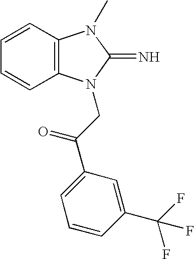 | B196 | 2.39 (c) | 334.3 (M + H)⁺ |
| 1-Methyl-1H-benzo- imidazol-2-yl- amine | 2-Bromo-1-(2-tri- fluoromethylphe- nyl)-ethanone | 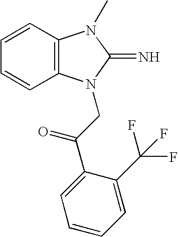 | B197 | 2.24 (c) | 334.2 (M + H)⁺ |
| 1-Methyl-1H-benzo- imidazol-2-yl- amine | N-[4-(2-Bromo- acetyl)-phe- nyl]-acet- amide | 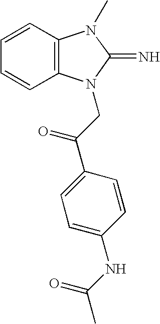 | B198 | 1.65 (c) | 323.3 (M + H)⁺ |
| 1-Methyl-1H-benzo- imidazol-2-yl- amine | 2-Bromo-1-(2,4-di- fluorophenyl)-etha- none | 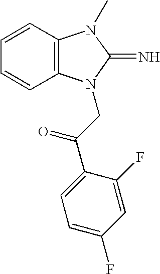 | B199 | 2.04 (c) | 302.3 (M + H)⁺ |
| 1-Methyl-1H-benzo- imidazol-2-yl- amine | 2-Bromo-1-(3-chlor- o-4-fluoro- phenyl)-etha- none | 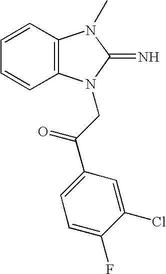 | B200 | 2.31 (c) | 318.2 (M + H)⁺ |
| 1-Methyl-1H-benzo- imidazol-2-yl- amine | 2-Bromo-1-(2-fluoro- phenyl)-etha- none | 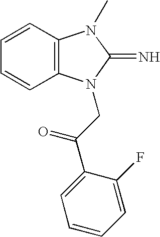 | B201 | 1.97 (c) | 284.2 (M + H)⁺ |
| 1-Methyl-1H-benzo- imidazol-2-yl- amine | 2-Bromo-1-(2,4-di- chlorophenyl)-etha- none | 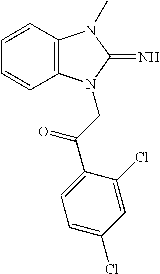 | B202 | 2.41 (c) | 335.2 (M + H)⁺ |
| 1-Methyl-1H-benzo- imidazol-2-yl- amine | 2-Bromo-1-(3,5-di- methoxyphenyl)-etha- none | 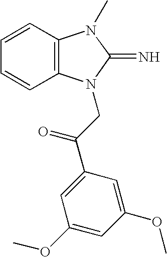 | B203 | 2.19 (c) | 326.3 (M + H)⁺ |
| 1-Methyl-1H-benzo- imidazol-2-yl- amine | 2-Bromo-1-(3,4-di- methoxyphenyl)-eth- anone | 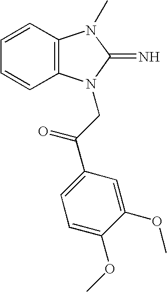 | B204 | 1.91 (c) | 326.3 (M + H)⁺ |
| 1-Methyl-1H-benzo- imidazol-2-yl- amine | 2-Bromo-1-(4-meth- anesulfonyl- phenyl)-ethanone | 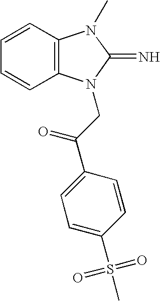 | B205 | 1.73 (c) | 344.2 (M + H)⁺ |
| 1-Methyl-1H-benzo- imidazol-2-yl- amine | 2-Bromo-1-(4-morpho- lin-4-yl- phenyl)-ethanone | 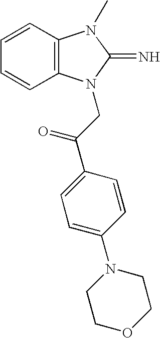 | B206 | 1.98 (c) | 351.3 (M + H)⁺ |
| 1-Methyl-1H-benzo- imidazol-2-yl- amine | 6-(2-Bromo- acetyl)-3H-benzo- oxazol-2-one | 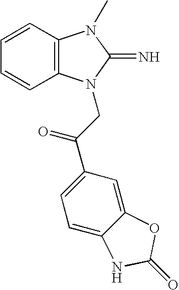 | B207 | 1.58 (c) | 321.2 (M − H)⁻ |
| 1-Methyl-1H-benzo- imidazol-2-yl- amine | 3-(2-Bromo- acetyl)-benzoic acid | 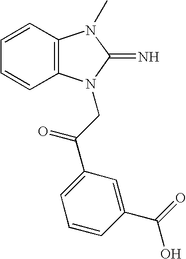 | B208 | 1.25 (c) | 308.2 (M − H)⁻ |
| 1-Methyl-1H-benzo- imidazol-2-yl- amine | 2-Bromo-1-(4-tert- butylphenyl)-eth- anone | 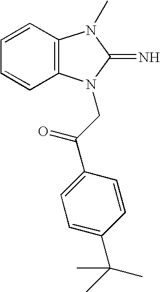 | B209 | 2.62 (d) | 322.3 (M + H)⁺ |
| 1-Methyl-1H-benzo- imidazol-2-yl- amine | 2-Bromo-1-(2,5-di- chlorophenyl)-eth- anone | 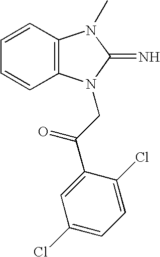 | B210 | 2.23 (d) | 335.3 (M + H)⁺ |
| 7-Chloro-1-meth- yl-1H-benzo- imidazol-2-yl- amine (B, C, D) | 2-Bromomethyl-1-meth- yl-1H-benzoimi- dazole | 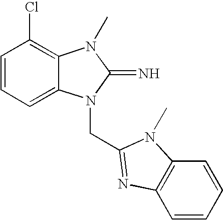 | B211 | 4.9 (a) | 326 (M + H)⁺ |
| 7-Chloro-1-meth- yl-1H-benzo- imidazol-2-yl- amine (B, C, D) | 2-Bromomethyl-benzo- thiazole | 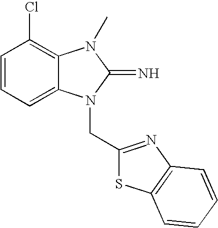 | B212 | 5.2 (a) | 329 (M + H)⁺ |
| 7-Chloro-1-meth- yl-1H-benzo- imidazol-2-yl- amine (B, C, D) | 2-Bromomethyl-3H-quina- zolin-4-one | 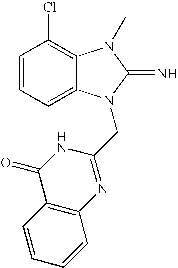 | B213 | 4.8 (a) | 340.2 (M + H)⁺ |
| 7-Chloro-1-meth- yl-1H-benzo- imidazol-2-yl- amine (B, C, D) | 2-Bromometh- yl-5-(4-chloro- phenyl)-oxa- zole | 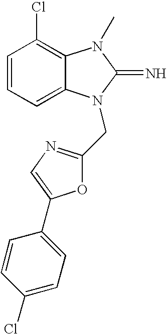 | B214 | 5.7 (a) | 374.0 (M + H)⁺ |
| 7-Chloro-1-meth- yl-1H-benzo- imidazol-2-yl- amine (B, C, D) | 5-Bromometh- yl-3-(4-chloro- phenyl)-[1,2,4]oxa- diazole | 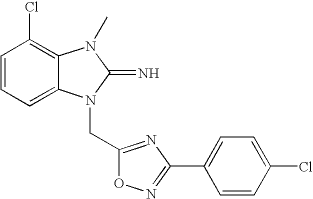 | B215 | 5.8 (a) | 375.1 (M + H)⁺ |
| 7-Chloro-1-meth- yl-1H-benzo- imidazol-2-yl- amine (B, C, D) | 1-Bromomethyl-iso- quinoline | 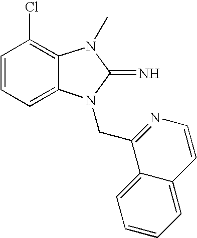 | B216 | 5.2 (a) | 323 (M + H)⁺ |
| 7-Chloro-1-meth- yl-1H-benzo- imidazol-2-yl- amine (B, C, D) | 4-Bromometh- yl-2-(4-chloro- phenyl)-thia- zole | 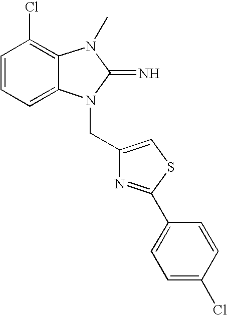 | B217 | 6.2 (a) | 390.1 (M + H)⁺ |
| 7-Chloro-1-meth- yl-1H-benzo- imidazol-2-yl- amine (B, C, D) | 2-Bromometh- yl-5-(4-chloro- phenyl)-[1,3,4]oxa- diazole | 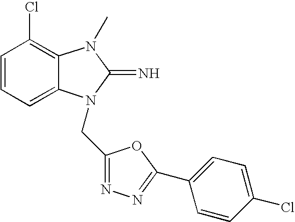 | B218 | 5.3 (a) | 375.1 (M + H)⁺ |
| 7-Chloro-1-meth- yl-1H-benzo- imidazol-2-yl- amine (B, C, D) | 4-Bromomethyl-2-phenyl- thiazole | 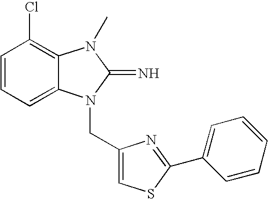 | B219 | 5.8 (a) | 355.1 (M + H)⁺ |
| 7-Chloro-1-meth- yl-1H-benzo- imidazol-2-yl- amine (B, C, D) | 4-Bromometh- yl-2-(3-chloro- phenyl)-thia- zole | 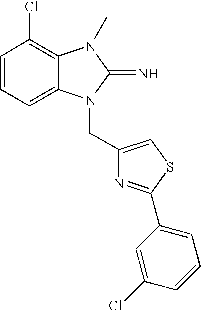 | B220 | 6.2 (a) | 390.1 (M + H)⁺ |
| 7-Chloro-1-meth- yl-1H-benzo- imidazol-2-yl- amine (B, C, D) | 3-(2-Bro- moethyl)-1H-quina- zoline-2,4-di- one | 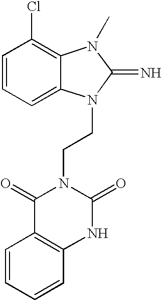 | B221 | 4.7 (a) | 370.1 (M + H)⁺ |
| 7-Chloro-1-meth- yl-1H-benzo- imidazol-2-yl- amine (B, C, D) | 2-Bromomethyl-2,3-di- hydrobenzo[1,4]di- oxine | 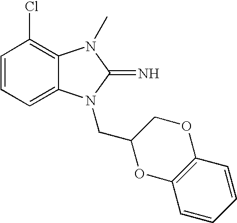 | B222 | 5.5 (a) | 330.2 (M + H)⁺ |
| 1,7-Diethyl-1H-benzo- imidazol-2-yl- amine (B, C, D, P, Q) | 2-Bromo-1-(4-chloro- phenyl)-etha- none | 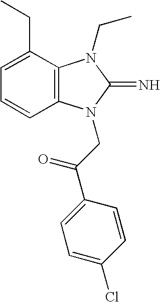 | B223 | 1.87 (c) | 342.2 (M + H)⁺ |
| 1,7-Diethyl-1H-benzo- imidazol-2-yl- amine (B, C, D, P, Q) | 2-Bromo-1-(3,4-di- chlorophenyl)-etha- none | 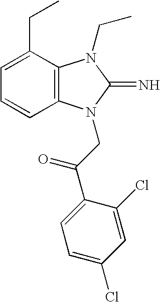 | B224 | 2.00 (c) | 377.2 (M + H)⁺ |
| 1-Ethyl-1H-benzo- imidazol-2-yl- amine (B, C, D) | 2-Bromo-1-(4-bromo- phenyl)-etha- none | 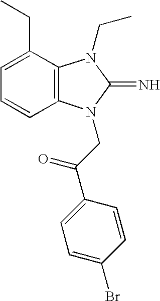 | B225 | 1.92 (c) | 387.1 (M + H)⁺ |
| 7-Ethyl-1-propyl-1H-benzo- imidazol-2-yl- amine (B, C, D, P, Q) | 2-Bromo-1-(4-chloro- phenyl)-etha- none | 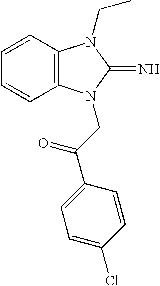 | B226 | 1.61 (c) | 314.2 (M + H)⁺ |
| 7-Ethyl-1-propyl-1H-benzo- imidazol-2-yl- amine (B, C, D, P, Q) | 2-Bromo-1-(4-chloro- phenyl)-etha- none | 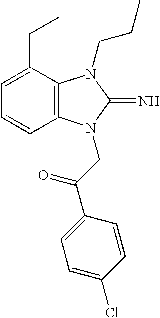 | B227 | 2.02 (c) | 356.2 (M + H)⁺ |
| 7-Ethyl-1-propyl-1H-benzo- imidazol-2-yl- amine (B, C, D, P, Q) | 2-Bromo-1-(3,4-di- chlorophenyl)-etha- none | 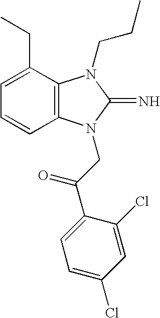 | B228 | 2.13 (c) | 391.2 (M + H)⁺ |
| 1-Butyl-7-ethyl-1H-benzo- imidazol-2-yl- amine (B, C, D, P, Q) | 2-Bromo-1-(4-bromo- phenyl)-etha- none | 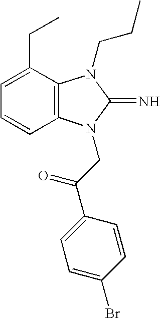 | B229 | 2.03 (c) | 401.1 (M + H)⁺ |
| 1-Butyl-7-ethyl-1H-benzo- imidazol-2-yl- amine (B, C, D, P, Q) | 2-Bromo-1-(4-chloro- phenyl)-eth- anone | 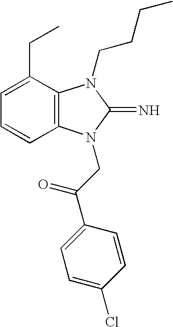 | B230 | 2.18 (c) | 370.2 (M + H)⁺ |
| 1-Butyl-7-ethyl-1H-benzo- imidazol-2-yl- amine (B, C, D, P, Q) | 2-Bromo-1-(4-bromo- phenyl)-eth- anone | 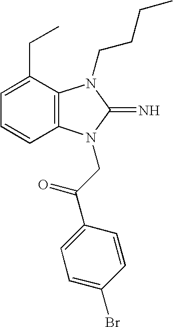 | B231 | 2.23 (c) | 415.2 (M + H)⁺ |
| 1-Butyl-7-ethyl-1H-benzo- imidazol-2-yl- amine | 2-Bromo-1-(4-chloro- phenyl)-eth- anone | 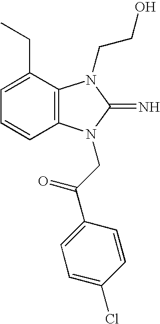 | B232 | 1.62 (c) | 358.2 (M + H)⁺ |
| 7-Ethyl-1-(2-methoxy- ethyl)-1,3-dihydro- benzoimidazol-2-yl- ideneamine (B, C, D, P, Q) | 2-Bromo-1-(4-bromo- phenyl)-eth- anone | 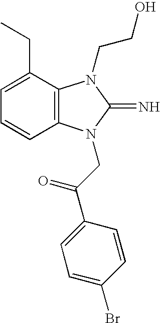 | B233 | 1.67 (c) | 403.2 (M + H)⁺ |
| 7-Ethyl-1-(2-meth- oxy-ethyl)-1,3-di- hydro-benzoimidazol-2-yl- ideneamine (B, C, D, P, Q) | 2-Bromo-1-phenyl- ethanone | 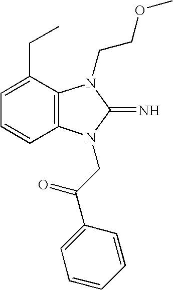 | B234 | 1.74 (c) | 338.2 (M + H)⁺ |
| 7-Ethyl-1-(2-methoxy- ethyl)-1,3-dihydro- benzoimidazol-2-yl- ideneamine | 2-Bromo-1-(4-chloro- phenyl)-eth- anone | 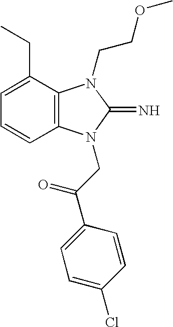 | B235 | 1.92 (c) | 372.2 (M + H)⁺ |
| 7-Ethyl-1-(3-methoxy- propyl)-1H-benzo- imidazol-2-yl- amine (B, C, D, P, Q) | 2-Bromo-1-(4-bromo- phenyl)-eth- anone | 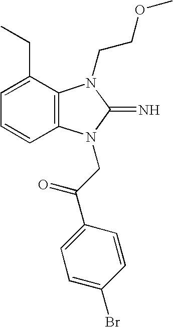 | B236 | 1.95 (c) | 417.1 (M + H)⁺ |
| 7-Ethyl-1-(3-methoxy- propyl)-1H-benzo- imidazol-2-yl- amine (B, C, D, P, Q) | 2-Bromo-1-phenyl- ethanone | 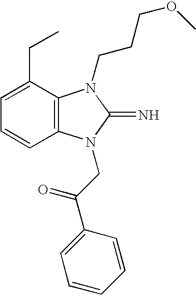 | B237 | 1.78 (c) | 352.2 (M + H)⁺ |
| 7-Ethyl-1-(3-methoxy- propyl)-1H-benzo- imidazol-2-yl- amine (B, C, D, P, Q) | 2-Bromo-1-(4-chloro- phenyl)-eth- anone | 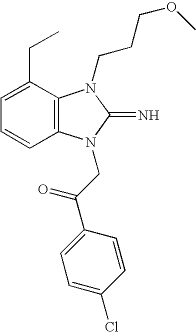 | B238 | 1.96 (c) | 386.2 (M + H)⁺ |
| 1-(3-Methoxy- propyl)-1H-benzo- imidazol-2-yl- amine (B, C, D) | 2-Bromo-1-(4-bromo- phenyl)-eth- anone | 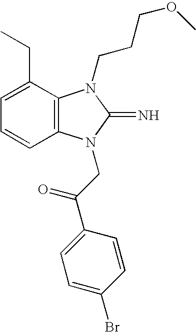 | B239 | 2.01 (c) | 431.2 (M + H)⁺ |
| 7-Ethyl-1-(2-phenoxy- ethyl)-1H-benzo- imidazol-2-yl- amine (B, C, D, P, Q) | 2-Bromo-1-(4-bromo- phenyl)-eth- anone | 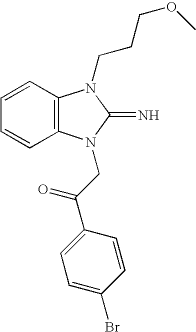 | B240 | 1.69 (c) | 403.2 (M + H)⁺ |
| 7-Ethyl-1-(2-phenoxy- ethyl)-1H-benzo- imidazol-2-yl- amine (B, C, D, P, Q) | 2-Bromo-1-(4-chloro- phenyl)-eth- anone | 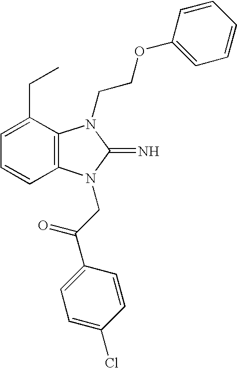 | B241 | 2.82 (c) | 434.2 (M + H)⁺ |
| 1-Propyl-1H-benzo- imidazol-2-yl- amine (B, C, D) | 2-Bromo-1-(4-bromo- phenyl)-eth- anone | 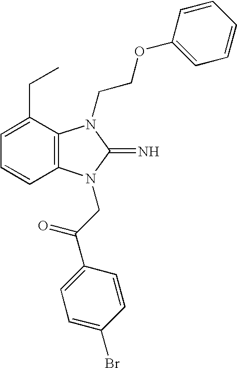 | B242 | 2.32 (c) | 479.2 (M + H)⁺ |
| 1-Butyl-1H-benzo- imidazol-2-yl- amine (B, C, D) | 2-Bromo-1-(3,4-di- chlorophenyl)-eth- anone | 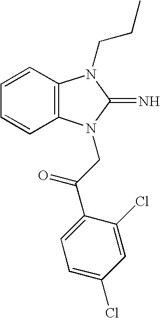 | B243 | 1.86 (c) | 363 (M + H)⁺ |
| 7-Ethyl-1-(2-methoxy- ethyl)-1H-benzo- imidazol-2-yl- amine (B, C, D) | 2-Bromo-1-(3,4-di- chlorophenyl)-eth- anone | 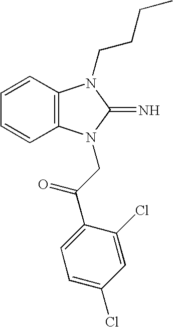 | B244 | 2.00 (c) | 377 (M + H)⁺ |
| 7-Ethyl-1-(2-methoxy- ethyl)-1H-benzo- imidazol-2-yl- amine (B, C, D, P, Q) | 2-Bromo-1-(3,4-di- chlorophenyl)-eth- anone | 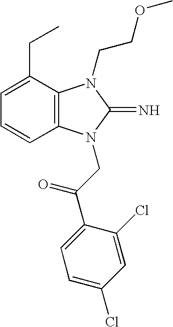 | B245 | 2.03 (c) | 407 (M + H)⁺ |
| 7-Chloro-1-ethyl-1H-benzo- imidazol-2-yl- amine (B, C, D) | 2-Bromo-1-phenyl- ethanone | 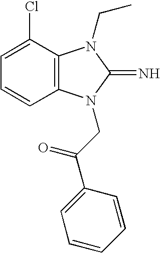 | B246 | 1.63 (c) | 315.2 (M + H)⁺ |
| 7-Chloro-1-ethyl-1H-benzo- imidazol-2-yl- amine (B, C, D) | 2-Bromo-1-(4-chloro- phenyl)-eth- anone | 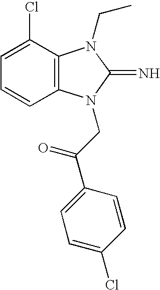 | B247 | 1.77 (c) | 349.2 (M + H)⁺ |
| 7-Chloro-1-ethyl-1H-benzo- imidazol-2-yl- amine (B, C, D) | 2-Bromo-1-(3,4-di- chlorophenyl)-eth- anone | 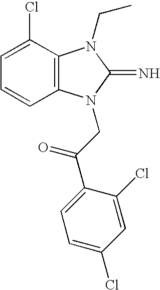 | B248 | 1.94 (c) | 384.1 (M + H)⁺ |
| 7-Chloro-1-ethyl-1H-benzo- imidazol-2-yl- amine (B, C, D) | 2-Bromo-1-(4-bromo- phenyl)-eth- anone | 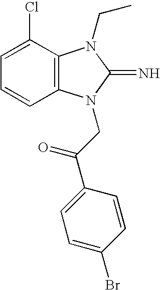 | B249 | 1.84 (c) | 394 (M + H)⁺ |
| 7-Chloro-1-pro- pyl-1H-benzo- imidazol-2-yl- amine (B, C, D) | 2-Bromo-1-phenyl- ethanone | 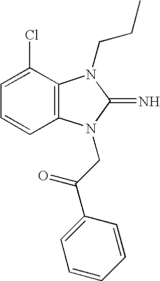 | B250 | 1.79 (c) | 328 (M + H)⁺ |
| 7-Chloro-1-pro- pyl-1H-benzo- imidazol-2-yl- amine (B, C, D) | 2-Bromo-1-(4-chloro- phenyl)-eth- anone | 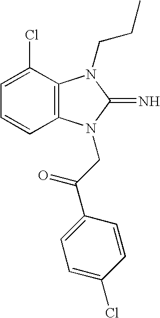 | B251 | 1.91 (c) | 363 (M + H)⁺ |
| 7-Chloro-1-pro- pyl-1H-benzo- imidazol-2-yl- amine (B, C, D) | 2-Bromo-1-(3,4-di- chlorophenyl)-eth- anone | 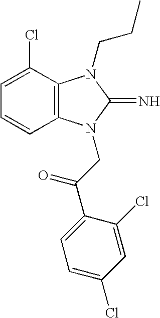 | B252 | 2.15 (c) | 397.1 (M + H)⁺ |
| 7-Chloro-1-pro- pyl-1H-benzo- imidazol-2-yl- amine (B, C, D) | 2-Bromo-1-(4-bromo- phenyl)-eth- anone | 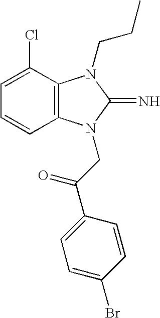 | B253 | 1.91 (c) | 408.2 (M + H)⁺ |
| 1-Butyl-7-chloro-1H-benzo- imidazol-2-yl- amine (B, C, D) | 2-Bromo-1-phenyl- ethanone | 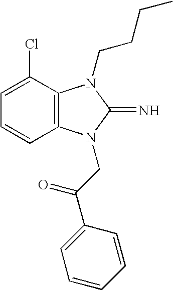 | B254 | 1.95 (c) | 343.3 (M + H)⁺ |
| 1-Butyl-7-chloro-1H-benzo- imidazol-2-yl- amine (B, C, D) | 2-Bromo-1-(4-chloro- phenyl)-eth- anone | 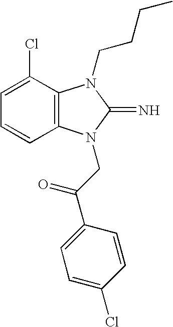 | B255 | 2.15 (c) | 377.2 (M + H)⁺ |
| 1-Butyl-7-chloro-1H-benzo- imidazol-2-yl- amine (B, C, D) | 2-Bromo-1-(3,4-di- chlorophenyl)-eth- anone | 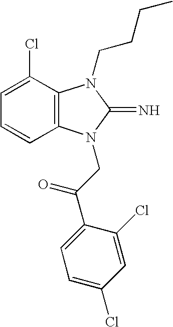 | B256 | 2.24 (c) | 411.2 (M + H)⁺ |
| 1-Butyl-7-chloro-1H-benzo- imidazol-2-yl- amine (B, C, D) | 2-Bromo-1-(4-bromo- phenyl)-eth- anone | 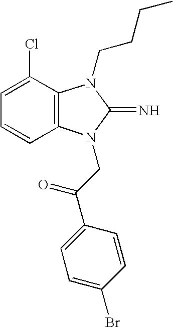 | B257 | 2.17 (c) | 422.1 (M + H)⁺ |
| 7-Chloro-1-(3-methoxy- propyl)-1H-benzo- imidazol-2-yl- amine (B, C, D) | 2-Bromo-1-phenyl- ethanone | 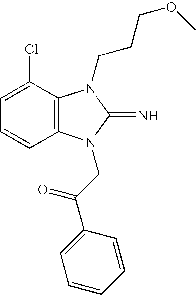 | B258 | 1.66 (c) | 358.2 (M + H)⁺ |
| 7-Chloro-1-(3-methoxy- propyl)-1H-benzo- imidazol-2-yl- amine (B, C, D) | 2-Bromo-1-(3,4-di- chlorophenyl)-eth- anone | 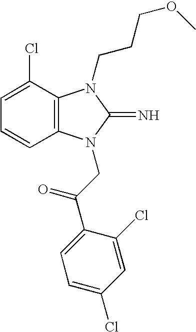 | B259 | 1.98 (c) | 427.2 (M + H)⁺ |
| 7-Chloro-1-(3-methoxy- propyl)-1H-benzo- imidazol-2-yl- amine (B, C, D) | 2-Bromo-1-(4-chloro- phenyl)-eth- anone | 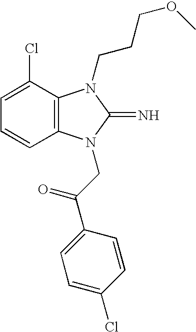 | B260 | 1.83 (c) | 393.2 (M + H)⁺ |
| 7-Chloro-1-(3-methoxy- propyl)-1H-benzo- imidazol-2-yl- amine (B, C, D) | 2-Bromo-1-(4-bromo- phenyl)-eth- anone | 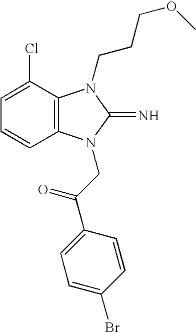 | B261 | 1.91 (c) | 438.1 (M + H)⁺ |
| 7-Chloro-1-(2-phenoxy- ethyl)-1H-benzo- imidazol-2-yl- amine (B, C, D) | 2-Bromo-1-phenyl- ethanone | 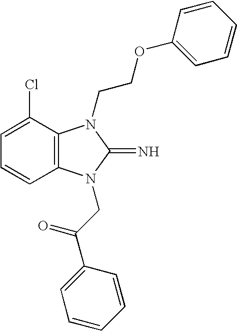 | B262 | 2.04 (c) | 406.3 (M + H)⁺ |
| 7-Chloro-1-(2-phenoxy- ethyl)-1H-benzo- imidazol-2-yl- amine (B, C, D) | 2-Bromo-1-(4-chloro- phenyl)-eth- anone | 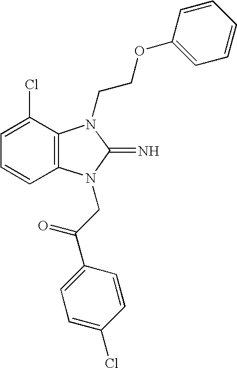 | B263 | 2.25 (c) | 441.2 (M + H)⁺ |
| 7-Chloro-1-(2-phenoxy- ethyl)-1H-benzo- imidazol-2-yl- amine (B, C, D) | 2-Bromo-1-(4-bromo- phenyl)-eth- anone | 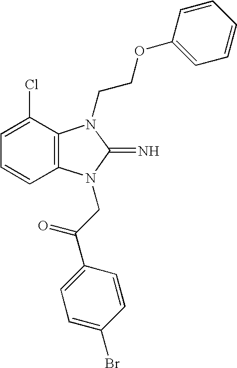 | B264 | 2.31 (c) | 486.2 (M + H)⁺ |
| 7-Chloro-1-(2-phenoxy- ethyl)-1H-benzo- imidazol-2-yl- amine (B, C, D) | 2-Bromo-1-(3,4-di- chlorophenyl)-eth- anone | 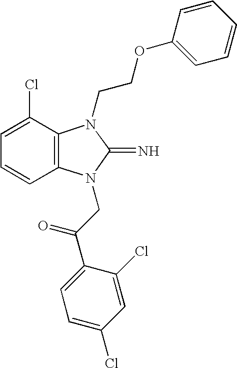 | B265 | 2.38 (c) | 476.1 (M + H)⁺ |
| 2-(2-Amino-7-chloro- benzoimidazol-1-yl)-eth- anol (B, C, D) | 2-Bromo-1-phenyl- ethanone | 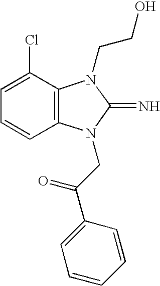 | B266 | 1.25 (c) | 331.2 (M + H)⁺ |
| 2-(2-Amino-7-chloro- benzoimidazol-1-yl)-eth- anol (B, C, D) | 2-Bromo-1-(4-chloro- phenyl)-eth- anone | 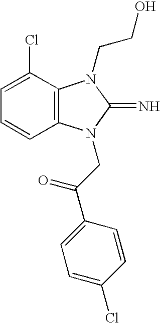 | B267 | 1.49 (c) | 365.1 (M + H)⁺ |
| 2-(2-Amino-7-chloro- benzoimidazol-1-yl)-eth- anol (B, C, D) | 2-Bromo-1-(3,4-di- chlorophenyl)-eth- anone | 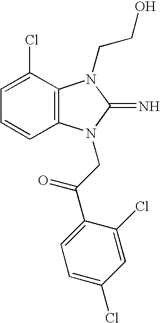 | B268 | 1.61 (c) | 399.1 (M + H)⁺ |
| 2-(2-Amino-7-chloro- benzoimidazol-1-yl)-eth- anol (B, C, D) | 2-Bromo-1-(4-bromo- phenyl)-eth- anone | 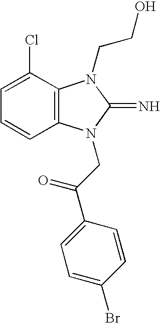 | B269 | 1.54 (c) | 409.1 (M + H)⁺ |
| 7-Chloro-1-(2-methoxy- ethyl)-1H-benzo- imidazol-2-yl- amine (B, C, D) | 2-Bromo-1-phenyl- ethanone | 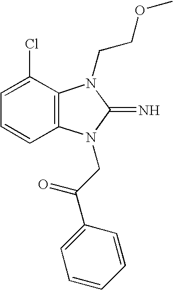 | B270 | 1.54 (c) | 345.2 (M + H)⁺ |
| 7-Chloro-1-(2-methoxy- ethyl)-1H-benzo- imidazol-2-yl- amine (B, C, D) | 2-Bromo-1-(4-chloro- phenyl)-eth- anone | 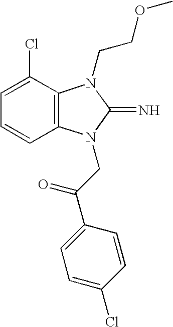 | B271 | 1.79 (c) | 379.1 (M + H)⁺ |
| 7-Chloro-1-(2-methoxy- ethyl)-1H-benzo- imidazol-2-yl- amine (B, C, D) | 2-Bromo-1-(3,4-di- chlorophenyl)-eth- anone | 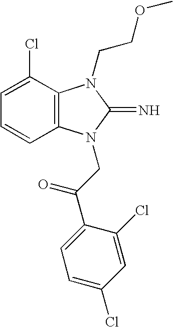 | B272 | 1.94 (c) | 414.1 (M + H)⁺ |
| 7-Chloro-1-(2-methoxy- ethyl)-1H-benzo- imidazol-2-yl- amine (B, C, D) | 2-Bromo-1-(4-bromo- phenyl)-eth- anone | 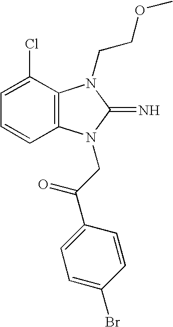 | B273 | 1.83 (c) | 424.0 (M + H)⁺ |
| N-(1-Methyl-1H-benzo- imidazol-2-yl)-acet- amide (S) | 2-Bromo-1-(4-nitro- phenyl)-eth- anone | 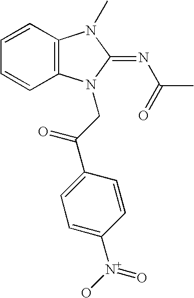 | B274 | 5.19 (a) | 353.2 (M + H)⁺ |
| 1-{2-[(4-Bromo- benzyl)-methyl- amino]-ethyl}-1H-benzo- imidazol-2-yl- amine (CC, DD, E) | 2-Bromo-1-(4-chloro- phenyl)-eth- anone | 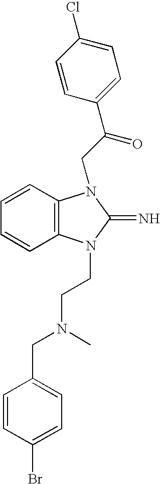 | B275 | 6.8 (a) | 513.1 (M + H)⁺ |
| 1-{2-[Methyl-(4-methyl- benzyl)-a- mino]-ethyl}-1H-benzo- imidazol-2-yl- amine (CC, DD, E) | 2-Bromo-1-(4-chloro- phenyl)-eth- anone | 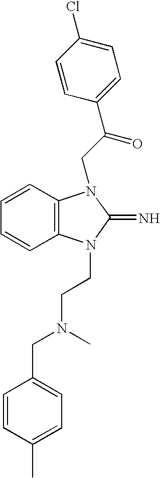 | B276 | 6.6 (a) | 447.3 (M + H)⁺ |
| 1-{2-[(4-Methoxy- benzyl)-methyl- amino]-ethyl}-1H-benzo- imidazol-2-yl- amine (CC, DD, E) | 2-Bromo-1-(4-chloro- phenyl)-eth- anone | 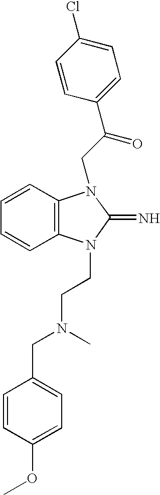 | B277 | 6.3 (a) | 463.3 (M + H)⁺ |
| 4-({[2-(2-Amino- benzoimidazol-1-yl)-eth- yl]-methyl- amino}-meth- yl)-benzo- nitrile (CC, DD, E) | 2-Bromo-1-(4-chloro- phenyl)-eth- anone | 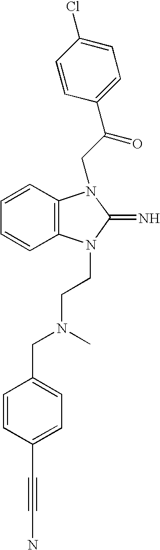 | B278 | 6.2 (a) | 456.2 (M + H)⁺ |
| 7-Chloro-2-imi- no-1-(3-meth- ylamino- propyl)-2,3-di- hydro-1H-benzo- imidazole (B, C, D, M) | 2-Bromo-1-(4-chloro- phenyl)-eth- anone | 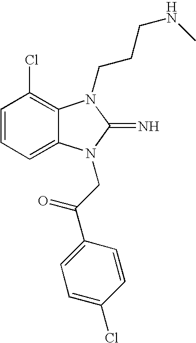 | B279 | 5.5 (a) | 391 (M + H)⁺ |

General Procedure F: Sulfonic acid displacement with an amine.

To a pressure tube equipped with a stir bar is added about 0.5 to 2 equivalents of the sulfonic acid (preferably 1 equivalent) and about 2 to 20 equivalents of the corresponding amine (preferably 20 equivalents). The resulting mixture is heated under pressure at about 120° C. to 150° C. (preferably 150° C.) for about 2 to5 hours (preferably 4 hours). After the reaction is cooled down to ambient temperature, the product is either precipitated out with appropriate solvent (for example water, dichloromethane, diethyl ether; preferably water) or is isolated via aqueous-organic extraction procedure (for example aqueous-dichloromethane extraction).

Illustration of General Procedure F

### Preparation #5: (1-Methyl-1H-benzoimidazol-2-yl)-propylamine

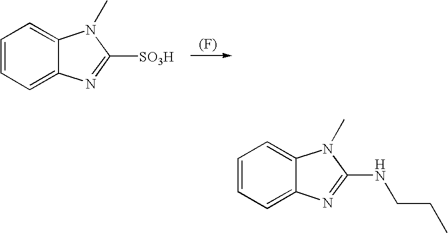

A mixture of 1-methyl-1H-benzoimidazole-2-Sulfonic acid (202 mg, 1.0 mmol) and N-propylamine (1.6 mL, 19.4 mmol) in a sealed tube equipped with a stir bar was heated at about 150° C. for about 4 hours. The resulting mixture was cooled down to ambient temperature and water (2 to 5 mL) was added while stirring. The precipitated solid was filtered, washed with least amount of water, and air dried to afford (1-Methyl-1H-benzoimidazol-2-yl)-propylamine (141 mg, 0.74 mmol): RP-HPLC Rₜ 4.3 min (Table 1, Method a), which was used in subsequent reactions without further purification.

General Procedure G: Conversion of a nitrobenzene to a 2-aminobenzimidazole. About 1 to 20 equivalents of an amine (preferably 10 equivalents) and about 1 to 5 equivalents of a halo-nitrobenzene (preferably 1 equivalent) are combined neat or in a protic organic solvent (preferably ethanol). The reaction mixture is stirred at about 0° C. to 200° C. (preferably 120° C.) for about 1 to 100 hours (preferably 4 hours). After cooling to about room temperature the reaction mixture is concentrated in vacuo. In the cases where there is a carboxylic acid present in the molecule, conversion to the ester can be accomplished by dissolving the crude carboxylic acid in an organic solvent (preferably DMF). About 1 to 5 equivalents inorganic base is added (preferably potassium carbonate, preferably 1.2 equivalents) followed by about 0.01 to about 10 equivalents of an alkylating agent (preferably 1.0 equivalents). After the reaction is complete an aqueous extraction provides the crude ester that is used directly in the next step. To a solution of the crude nitrobenzene compound dissolved in an organic solvent (preferably ethanol) is added about 0.01 to 10 equivalents of palladium on carbon (preferably 0.1 equivalents). Hydrogen gas is bubbled through the solution for about 5 minutes after which a hydrogen atmosphere was maintained by balloon. After about 1 to 5 days (preferably 2 days) the reaction mixture is either used directly in subsequent reactions or filtered and concentrated. When the reaction mixture is filtered and concentrated the crude product can be purified by chromatography. When the reaction mixture is used directly the reaction mixture is purged with nitrogen and about 1 to 5 equivalents of cyanogen bromide (preferably 2 equivalents) is added. After about 1 to 48 hours (preferably 15 hours) the reaction mixture is filtered and concentrated in vacuo to provide the crude product that can be purified by crystallization or chromatography.

Illustration of General Procedure G

### Preparation #6: 1,7-Dimethyl-1H-benzoimidazol-2-ylamine

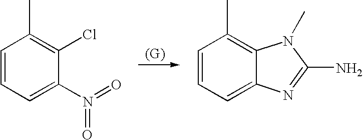

To a 33% solution of methylarnine in ethanol (10 mL, 80 mmol) was added 2-Chloro-1-methyl-3-Nitro-benzene (1.3 g, 7.6 mmol). The reaction mixture was heated at about 120° C. for about 4 hours. After cooling to about room temperature the reaction mixture was concentrated in vacuo. To a solution of the crude Methyl-(2-methyl-6-Nitro-phenyl)-amine in ethanol was added palladium on carbon (806 mg, 0.76 mmol). Hydrogen gas was bubbled through the solution for about 5 minutes after which a hydrogen atmosphere was maintained by balloon. After about 5 hours the reaction mixture was purged with nitrogen and a solution of cyanogen bromide in acetonile (3.0 mL, 15.2 mmol) was added. After about 15 hours the reaction mixture was filtered and concentrated in vacuo. The crude product was triturated with ethyl acetate to provide 1.3 g of 1,7-Dimethyl-1,3-dihydro-benzoimidazol-2-ylideneamine hydrobromide as a tan solid that could be used directly or further purified by RP-HPLC. RP-HPLC Rₜ 3.90 min (table 1, method a), m/z: (M+H)⁺ 162.2.

TABLE C
Examples prepared using general procedure G

| | | | | m/z |
|---|---|---|---|---|
| | | | | or ¹H NMR |
| Starting | | Example | R₁/min | (d₆ DMSO, |
| Material | Product | # | (method) | 400 MHz) |
| 2-Bromo- 3-nitro- benzoic acid | 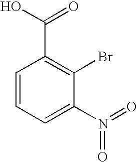 | Cl | 4.18 min (a) | 206.2 (M + H)⁺ |

General Procedure H: Alkylation of a 2-aminobenzimidazole followed by reduction of a ketone. A benzimidazole (preferably 1 equivalent) and about 1 to 5 equivalents of a haloacetophenone (preferably bromoacetophenone, preferably 1 equivalent) are combined in an organic solvent (preferably dimethylformamide). The reaction mixture is stirred at about 0° C. to 60° C. (preferably 25° C.) for about 1 to 72 hours (preferably 2 hours). To the reaction mixture is added about 1 to 10 equivalents of a hydride reducing agent (preferably sodium borohydride, preferably 1 equivalent) and an organic solvent (preferably isopropanol). The reaction mixture is stirred at about 0° C. to 80° C. (preferably 25° C.) for about 1 to 24 hours (preferably 2 hours). The reaction mixture is concentrated in vacuo and triturated ethanol to give a suspension that is collected by filtration and dried in vacuo.

Illustration of General Procedure H

### Preparation #7: 1-(4-Bromo-phenyl)-2-(2-imino-3-methyl-2,3-dihydro-benzimidazol-1-yl)-ethanol

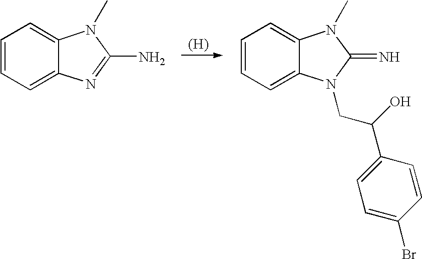

1-Methyl-1H-benzimidazol-2-ylamine (0.675 g, 4.59 mmol), 2-Bromo-1-(4-bromo-phenyl)-ethanone (1.28 g, 4.59 mmol), and dimethylformamide (4.6 mL) were combined and stirred at about 25° C. After about 2 hours, sodium borohydride (0.174 g, 4.59 mmols) and isopropanol (4.6 mL) were added to the reaction mixture and the resulting solution was stirred at about 25° C. After about 2 hours the mixture was concentrated and the residue was triturated with ethanol and filtered to give 1.92 g of 1-(4-Bromo-phenyl)-2-(2-imino-3-methyl-2,3-dihydro-benzimidazol-1-yl)-ethanol as a white solid. RP-HPLC Rₜ 1.57 min. (table 1, method d); m/z: (M+H)⁺ 346.2.

General Procedure I: Conversion of a secondary alcohol to an alkene. The alcohol (preferably 1 equivalent) and about 1 to 50 equivalents of a chlorinating reagent (preferably thionyl chloride, preferably 11 equivalents) are combined in an organic solvent (preferably dimethylformamide). The reaction mixture is stirred at about 0° C. to 200° C. (preferably 130° C.) for about 1 to 72 hours (preferably 6 hours). The reaction mixture is concentrated and dissolved in an organic solvent (preferably methylene chloride) and washed with water and aqueous basic solution, dried, and concentrated in vacuo. The residue is dissolved in an organic solvent (preferably dimethylformamide) and heated at about 100° C. to 250° C. (preferably 180° C.) for about 1 to 5 days (preferably 2 days); alternatively the solution is heated at about 100° C. to 250° C. (preferably 180° C.) using single-mode microwave irradiation for about 5 to 150 minutes (preferably 15 minutes). The reaction mixture is then purified by reverse-phase chromatography.

Illustration of General Procedure I

### Preparation #8: 1-[(E)-2-(4-Bromo-phenyl)-vinyl]-3-methyl-1,3-dihydro-benzimidazol-2-ylideneamine

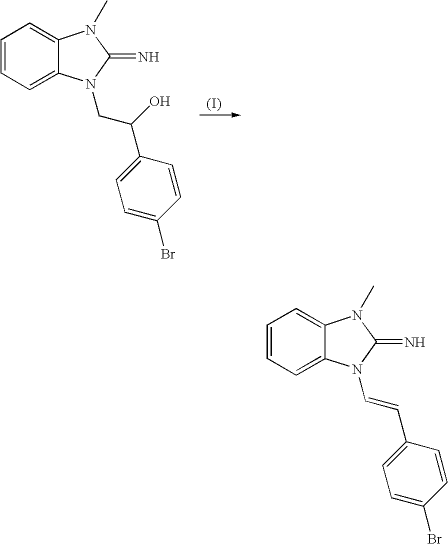

1-(4-Bromo-phenyl)-2-(2-imino-3-methyl-2,3-dihydro-benzimidazol-1-yl)-ethanol (0.100 g, 2.89 mmol), thionyl chloride (0.378 g, 3.18 mmol), and dimethylformamide (1.5 mL) were combined and stirred at about 25° C. After about 16 hours, the solution was heated to about 130° C. After about 6 hours, the reaction mixture was cooled at ambient temperature and concentrated in vacuo, then dissolved in methylene chloride and washed with water and saturated NaHCO₃ solution, dried over MgSO4, filtered and concentrated in vacuo. To the residue was added dimethylformamide (1.5 mL) and the mixture was heated at about 180° C. for about 15 minutes using single-mode microwave irradiation. The reaction mixture was purified by reverse-phase chromatography using 40-60% MeCN: water to provide 0.049 g of 1-[(E)-2-(4-Bromo-phenyl)-vinyl]-3-methyl-1,3-dihydro-benzimidazol-2-ylideneamine as a white solid. RP-HPLC Rₜ 1.88 min. (table 1, method d); m/z: (M+H)⁺ 328.4.

General Procedure J: Formation of a 2-aminobenzimidazole. About 1 to 20 equivalents of an amine (preferably 2.25 equivalents), about 0 to 5 equivalents of an organic base (preferably diisopropylethylamine, preferably 2 equivalents) and a halo-nitrobenzene (preferably 1 equivalent) are combined in an organic solvent or neat (preferably neat). The reaction mixture is stirred at about 0° C. to 200° C. (preferably 100° C.) for about 1 to 10 days (preferably 3 reaction mixture is concentrated, diluted with an organic solvent (preferably diethyl ether) and washed with an acidic aqueous solution (preferably 1N HCl) followed by brine. The organic layer is dried, filtered and concentrated in vacuo. To a solution of the crude nitrobenzene compound in a polar protic solvent (preferably acetic acid) is added about 1 to 10 equivalents of a reducing agent (preferably iron, preferably 4 equivalents). The reaction mixture is stirred at about 0° C. to 100° C. (preferably 25° C.) for about 2 to 48 hours (preferably 15 hours). The reaction mixture is filtered and concentrated in vacuo. The crude product is dissolved in an organic solvent (preferably diethyl ether) and washed with a basic aqueous solution (preferably 2N NaOH) that had been saturated with an iron-chelating agent (preferably EDTA). The organic layer is further washed with brine, filtered and concentrated in vacuo. To a solution of the crude phenylenediamine in an organic solvent (preferably ethanol) is added about 0 to 10 equivalents of an organic base (preferably sodium acetate, preferably 5 equivalents) followed by a solution of about 1 to 3 equivalents of cyanogen bromide in an organic solvent (preferably acetonitrile, preferably 1.5 equivalents). After stirring for about 20 hours at about 25° C. the reaction mixture is concentrated in vacuo. The crude mixture is diluted with an organic solvent (preferably ethyl acetate) and the product is isolated by filtration or taken through an aqueous work up and purified by chromatography.

Illustration of General Procedure J

### Preparation #9: [3-(2-Amino-6-chloro-phenylamino)-propyl]-methyl-carbamic acid tert-butyl ester

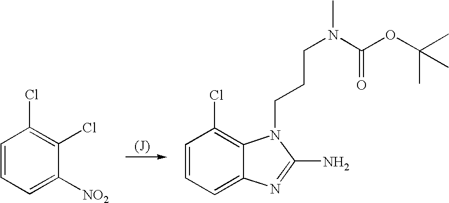

1, 2 -Dichloro-3-Nitro-benzene (1.0 g, 5.2 mmol), (3-Amino-propyl)-methyl-carbamic acid tert-butyl ester (2.25 g, 11.9 mmol) and diisopropylethylamine (1.8 mL, 10.4 mmol) were combined and heated to about 100° C. After about 3 days the reaction mixture was diluted with diethyl ether (200 mL) and 1N HCl (200 mL). The organic layer was separated, washed with brine, dried (Na₂SO₄), filtered and concentrated in vacuo to provide 1.8 g of [3-(2-Chloro-6-Nitro-phenylamino)-propyl]-methyl-carbamic acid tert-butyl ester as an oil which was used in subsequent reactions without further purification. RP-HPLC Rₜ 7.51 min. (table 1, method a); m/z: (M+H)⁺ 244.0. To a solution of the above nitro aniline in acetic acid (53 mL) at room temperature was added iron powder (1.2 g, 21.2 mmol). After stirring for about 15 hours the reaction mixture was filtered and concentrated in vacuo. The crude product was dissolved in diethyl ether and washed with a solution of 2N NaOH that had been saturated with EDTA. The organic layer was further washed with brine, dried with Na₂SO₄, filtered and concentrated in vacuo to provide [3-(2-Amino-6-chloro-phenylamino)-propyl]-methyl-carbamic acid tert-butyl ester as a brown oil that was used in subsequent reactions without further purification. RP-HPLC Rₜ 6.05 min. (table 1, method a). To a solution of the crude phenylene diamine in EtOH (98 mL) was added NaOAc (1.8 g, 22 mmol) followed by a 5 N solution of cyanogen bromide in acetonile (1.4 mL, 7.2 mmol). After stirring for about 20 hours at room temperature the reaction mixture was concentrated in vacuo. The crude reaction mixture was diluted with Et₂O (200 mL) and 2 N NaOH (200 mL). The organic layer was separated, washed with brine, dried (Na₂SO₄), filtered and concentrated. The crude product was purified by column chromatography on silica gel (gradient elution 5-10% MeOH/CH₂Cl₂, containing 1% Et₃N) to provide 1.3 g of [3-(7-Chloro-2-imino-2,3-dihydro-benzoimidazol-1-yl)-propyl]-methyl-carbamic acid tert-butyl ester as a brown oil. RP-HPLC Rₜ 6.05 min (table 1, method a). m/z: (M+H)⁺ 339.0, 341.1 (3:1).

General Procedure K: Oxidation of a sulfide to the sulfoxide. To a solution of the sulfide (preferably 1 equivalent) in an organic solvent (preferably methylene chloride) is added about 0.5 to 2.0 equivalents of an oxidant (preferably MCPBA, preferably 1.1 equivalents). The reaction mixture is stirred at about 0° C. to 100° C. (preferably, room temperature) for about 10 minutes to 15 hours (preferably 2 hours) and diluted with an organic solvent (preferably ethyl acetate). The product can be isolated by filtration or taken through an aqueous work up.

Illustration of General Procedure K

### Preparation #10: 1-(4-Chloro-benzenesulfinylmethyl)-3-methyl-1,3-dihydro-benzoimidazol-2-ylideneamine hydrochloride

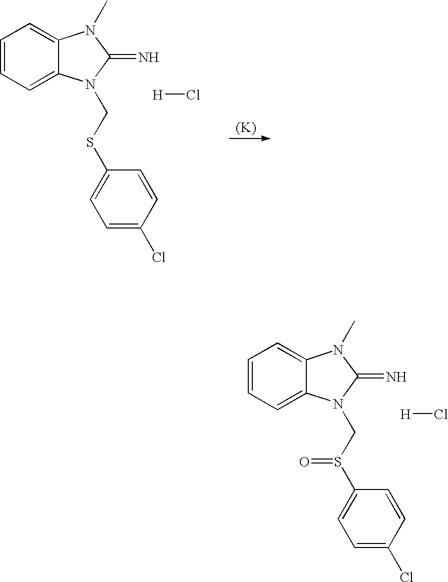

To a solution of 1-(4-Chloro-phenylsulfanylmethyl)-3-methyl-1,3-dihydro-benzoimidazol-2-ylideneamine hydrochloride (35 mg, 0.10 mmol) in methylene chloride (2 mL) at room temperature was added technical grade MCPBA (70%, 28 mg, 0.11 mmol). After about 2 hours the reaction mixture was diluted with ethyl acetate (4 mL) and filtered to provide 14 mg of 1-(4-Chloro-benzenesulfinylmethyl)-3-methyl-1,3-dihydro-benzoimidazol-2-ylideneamine hydrochloride as a white solid. RP-HPLC Rₜ 4.74 min. (table 1, method a). LC/MS (M+H)⁺ 320.1.

General Procedure L: Formation of a N-1-acetamide-substituted 2-aminobenzimidazole. To a solution of about 1 equivalent of an amine in an organic solvent (preferably ether) is added about 0.5 to 5.0 equivalents of an organic base (preferably diisopropylethylamine, preferably 1.0 equivalents) followed by about 0.5 to 2.0 equivalents of chloroacetyl chloride (preferably 1.0 equivalents). The reaction mixture is stirred at about 0° C. to 50° C. (preferably room temperature) for about 4 hours. The reaction mixture is filtered and concentrated in vacuo. The crude reaction mixture is dissolved in an organic solvent (preferably DMF) and about 0.5 to about 3.0 equivalents of 1H-benzoimidazol-2-ylamine (preferably 1.0 equivalents) is added. After about 20 hours an organic solvent (preferably ethyl acetate) is added and the reaction mixture is filtered and concentrated in vacuo. The crude product can be purified my chromatography.

Illustration of General Procedure L

Preparation #11: 2-(2-Amino-benzoimidazol-1-yl)-N-methyl-N-phenyl-acetamide

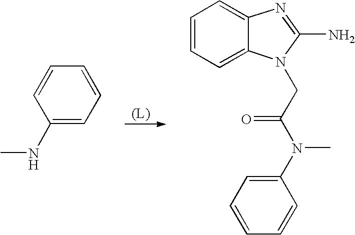

To a solution of methyl-phenyl-amine (536 mg, 5.0 mmol) and diisopropylethylamine (565 mg, 5.0 mmol) in diethyl ether (50 mL) at room temperature was added chloro-acetyl chloride (565 mg, 5.0 mmol) dropwise. After stirring for about 4 hours the reaction mixture was filtered and concentrated in vacuo. The crude reaction mixture was dissolved in DMF (25 mL) and 1H-benzoimidazol-2-ylamine (666 mg, 5.0 mmol) was added. After stirring for about 20 hours ethyl acetate (50 mL) was added and the reaction mixture was filtered and concentrated in vacuo. The crude product was purified by RP-HPLC. The fractions containing the product were concentrated in vacuo to remove acetonitrile, 2 N NaOH (50 mL) was added and the resulting precipitate was collected to provide 210 mg of 2-(2-amino-benzoimidazol-1-yl)-N-methyl-N-phenyl-acetamide as a white solid. RP-HPLC Rₜ 4.81 min. (table 1, method a); m/z: (M+H)⁺ 281.1.

General Procedure M: Deprotection of BOC protected amines. To a solution of the protected amine in a suitable solvent (preferably dichloromethane) is added an equal amount of trifluoroacetic acid. The reaction is stirred at ambient temperature for about 1 to 24 hours (preferably 1 hour) after which time the solvents are removed. The resulting residue is dissolved in a protic solvent (preferably methanol) and about 1 to 5 equivalents (preferably 3 equivalents) of resin bound scavenger base (preferably MP-carbonate) is added and the reaction stirred for about 30 minutes to 2 hours (preferably 1 hour). The reaction is filtered, concentrated, and redissolved in a solvent (preferably ethyl acetate). The product may be precipitated by the addition of HCl in an ethereal solvent (preferably 1.0 M HCl in diethyl ether). The resulting solid is collected by vacuum filtration, washed with ether, and dried.

Illustration of General Procedure M

### Preparation of #12: 7-Chloro-1-piperidin-3-ylmethyl-1H-benzoimidazol-2-ylamine

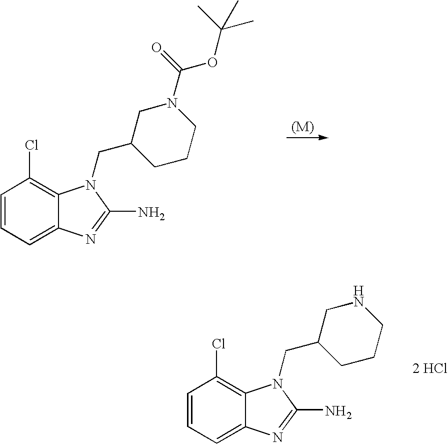

To a solution of 3-(2-Amino-7-chloro-benzoimidazol-1-ylmethyl)-piperidine-1carboxylic acid tert-butyl ester (1.12 g, 3.1 mmol) in methylene chloride (15 mL) was added trifluoroacetic acid (15 mL). The reaction was stirred at ambient temperature for about 2 hours. The solvents were removed in vacuo and the residue dissolved in methanol (10 mL). MP-carbonate resin (6.2 g, 18 mmol, 2.89 mmol/g) was added and the reaction stirred about 1 hour. The reaction was filtered and the resin washed with methanol. The solvents were removed under vacuum and the residue dissolved in ethyl acetate. A solution of 1M HCl in diethyl ether (9.5 mL, 9.5 mmol) was added. The resulting solid was collected by vacuum filtration and washed with ether. The solid was dried under vacuum to provide 7-Chloro-1-piperidin-3-ylmethyl-1H-benzoimidazol-2-ylamine (1.02 g, 82%) as a tan solid. RP-HPLC Rₜ 3.814 min. (table 1, method a).

General Procedure N: Acylation or Sulfonylation of an amine followed by alkylation of a 2-aminobenzimidazole. To a solution of a 2-aminobenzimidazole containing a tethered secondary amine in an organic solvent (preferably methylene chloride) at about 78° C. to 50° C. (preferably 0° C.) is added about 1.0 to 10.0 equivalents of diisopropylethylamine (preferably 5.0 equivalents) followed by about 0.5 to 2.0 equivalents of a solution of an acylating or sulfonylating reagent (preferably an acid chloride or an isocyanate or a solfonyl chloride) preferably 1.1 equivalents) in an organic solvent (preferably methylene chloride). After the addition the reaction mixture is allowed to warm to room temperature and stirred for about 1 hour. The reaction mixture is diluted in about half with a protic solvent (preferably methanol) and about 1.0 to 10 equivalents of MP-carbonate (preferably 5 equivalents) is added. After stirring for about 3 hours the reaction mixture is filtered and concentrated in vacuo. The crude reaction mixture is dissolved in an organic solvent (preferably DMF) and an electrophile is added. After about 15 hours the crude product can be purified by trituration with an organic solvent (preferably ethyl acetate) or by chromatography.

Illustration of General Procedure N

### EXAMPLE #2

### N-(3-{3-[2-(4-Bromo-phenyl)-2-oxo-ethyl]-7-chloro-2-imino-2,3-dihydro-benzoimidazol-1-yl}-propyl)-N-methyl-benzenesulfonamide hydrobromide

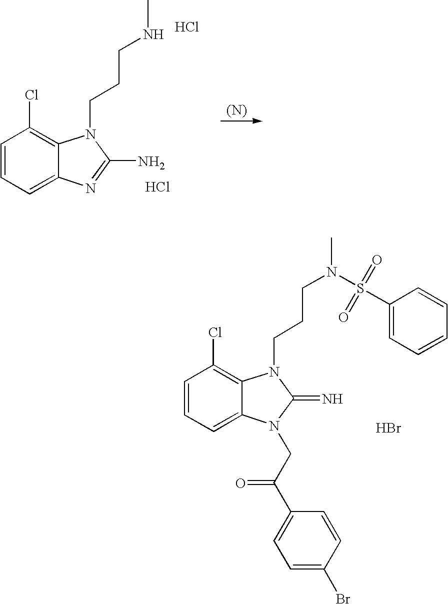

To a solution of 7-Chloro-1-(3-methylamino-propyl)-1H-benzoimidazol-2-ylamine bis HCl salt (43 mg, 0.14 mmol) in methylene chloride (2 mL) at about 0° C. was added diisopropylethylamine (0.12 mL, 0.69 mmol) followed by a 1.0M solution of benzensulfonyl chloride in methylene chloride (166 uL, 0.17 mmol). After the addition, the reaction mixture was allowed to warm to room temperature and stirred for about 1 hour. The reaction mixture was diluted in half with methanol and MP-carbonate (250 mg) was added. After stirring for about 3 hours the reaction mixture was filtered and concentrated in vacuo. The crude reaction mixture was dissolved in DMF (2 mL) and 2-bromo-1-(4-bromo-phenyl)-ethanone (38 mg, 0.14 mmol) was added. After 15 about hours the reaction mixture was concentrated in vacuo and the crude product was triturated with ethyl acetate to provide 8 mg of N-(3-{3-[2-(4-Bromo-phenyl)-2-oxo-ethyl]-7-chloro-2-imino-2,3-dihydro-benzoimidazol-1-yl}-propyl)-N-methyl-benzenesulfonamide hydrobromide RP-HPLC Rₜ 6.35 min. (table 1, method a). LC/MS (M+H)⁺ 576.4.

TABLE D
Examples prepared using general procedure N

| | | | | | | m/z |
|---|---|---|---|---|---|---|
| | | | | | | or ¹H |
| | Acylating | | | | | NMR |
| | or | | | | | (d₆ |
| | Sulfonyl- | | | Exam- | | DMSO, |
| Starting | ating | Alkylating | | ple | R₁/min | 400 |
| Material | Reagent | Reagent | Product | # | (method) | MHz) |
| 7-Chloro-2- imino-1-(3- methylamino- o-propyl)- 2,3-dihydro- 1H-benzo- imidazole (B, C, D, M) | Naphtha lene-2- carboxylic acid | 2-Bromo- 1-(4- chloro- phenyl)- ethanone | 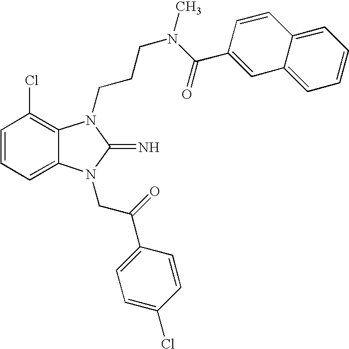 | D1 | 2.20 min (c) | (M + H)⁺545.1, 547.0 |
| 7-Chloro-2- imino-1-(3- methylamino- o-propyl)- 2,3-dihydro- 1H-benzo- imidazole (B, C, D, M) | 2-Cyano- benzoic acid | 2-Bromo- 1-(4- chloro- phenyl)- ethanone | 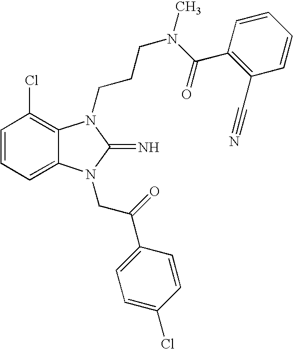 | D2 | 1.90 min (c) | (M + H)⁺520.1, 522.2 |
| 7-Chloro-2- imino-1-(3- methylamino- o-propyl)- 2,3-dihydro- 1H-benzo- imidazole (B, C, D, M) | Benzo [1,3]dioxole-5- carboxylic acid | 2-Bromo- 1-(4- chloro- phenyl)- ethanone | 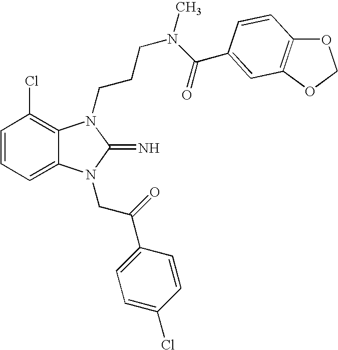 | D4 | 2.01 min (c) | (M + H)⁺539.0, 541.1 |
| 7-Chloro-2- imino-1-(3- methylamino- o-propyl)- 2,3-dihydro- 1H-benzo- imidazole (B, C, D, M) | 3-Methyl- benzoic acid | 2-Bromo- 1-(4- chloro- phenyl)- ethanone | 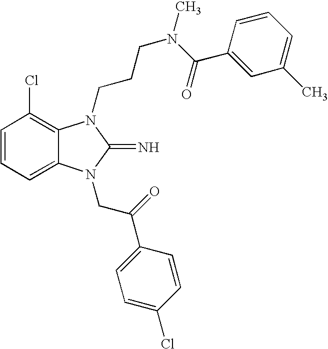 | D4 | 2.23 min (c) | (M + H)⁺509.2, 511.1 |
| 7-Chloro-2- imino-1-(3- methylamino- o-propyl)- 2,3-dihydro- 1H-benzo- imidazole (B, C, D, M) | Isophthalic acid monomethyl ester | 2-Bromo- 1-(4- chloro- phenyl)- ethanone | 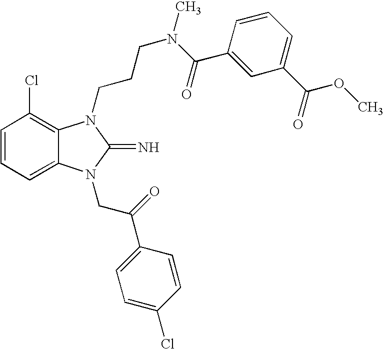 | D5 | 2.04 min (c) | (M + H)⁺553.1, 555.0 |
| 7-Chloro-2- imino-1-(3- methylamino- o-propyl)- 2,3-dihydro- 1H-benzo- imidazole (B, C, D, M) | Terephthalic acid monomethyl ester | 2-Bromo- 1-(4- chloro- phenyl)- ethanone | 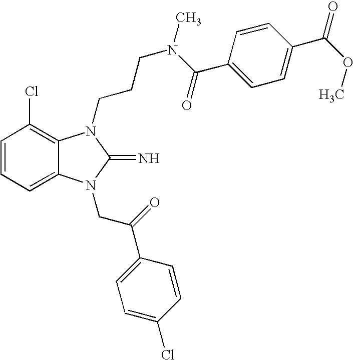 | D6 | 2.09 min (c) | (M + H)⁺553.1, 555.1 |
| 7-Chloro-2- imino-1-(3- methylamino- o-propyl)- 2,3-dihydro- 1H-benzo- imidazole (B, C, D, M) | 4- Isopropoxy- benzoic acid | 2-Bromo- 1-(4- chloro- phenyl)- ethanone | 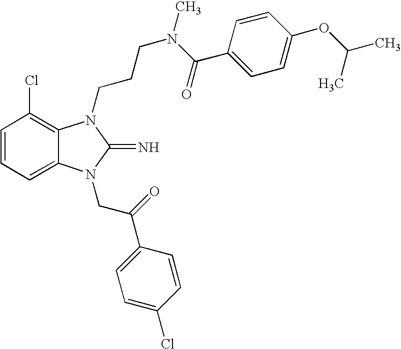 | D7 | 2.46 min (c) | (M + H)⁺553.2, 555.1 |
| 7-Chloro-2- imino-1-(3- methylamino- o-propyl)- 2,3-dihydro- 1H-benzo- imidazole (B, C, D, M) | 2,3- Dimethoxy- benzoic acid | 2-Bromo- 1-(4- chloro- phenyl)- ethanone | 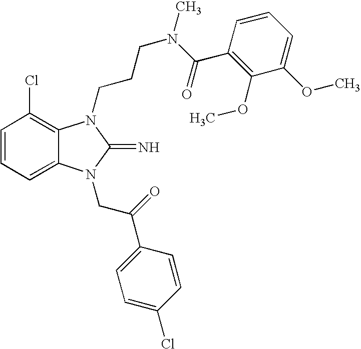 | D8 | 2.15 min (c) | (M + H)⁺555.2, 557.1 |
| 7-Chloro-2- imino-1-(3- methylamino- o-propyl)- 2,3-dihydro- 1H-benzo- imidazole (B, C, D, M) | 3,4- Dimethoxy- benzoic acid | 2-Bromo- 1-(4- chloro- phenyl)- ethanone | 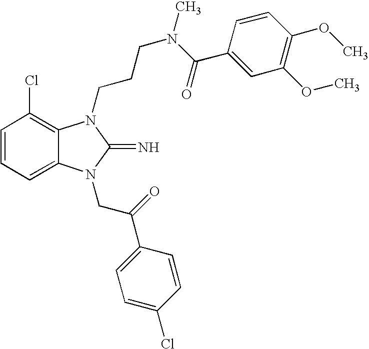 | D9 | 1.96 min (c) | (M + H)⁺555.2, 557.1 |
| 7-Chloro-2- imino-1-(3- methylamino- o-propyl)- 2,3-dihydro- 1H-benzo- imidazole (B, C, D, M) | 2,3- Dimethyl- benzoic acid | 2-Bromo- 1-(4- chloro- phenyl)- ethanone | 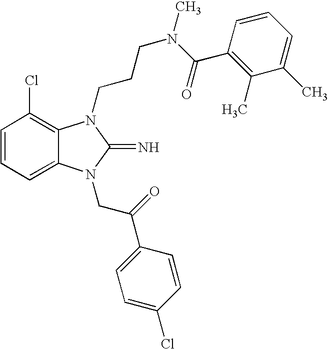 | D10 | 2.28 min (c) | (M + H)⁺523.1, 525.0 |
| 7-Chloro-2- imino-1-(3- methylamino- o-propyl)- 2,3-dihydro- 1H-benzo- imidazole (B, C, D, M) | 2,4- Dichloro- benzoic acid | 2-Bromo- 1-(4- chloro- phenyl)- ethanone | 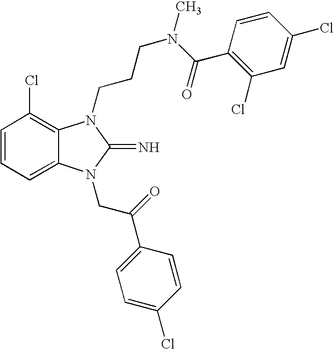 | D11 | 2.50 min (c) | (M + H)⁺563.1, 565.1, 567.1 |
| 7-Chloro-2- imino-1-(3- methylamino- o-propyl)- 2,3-dihydro- 1H-benzo- imidazole (B, C, D, M) | 3,4- Dichloro- benxoic acid | 2-Bromo- 1-(4- chloro- phenyl)- ethanone | 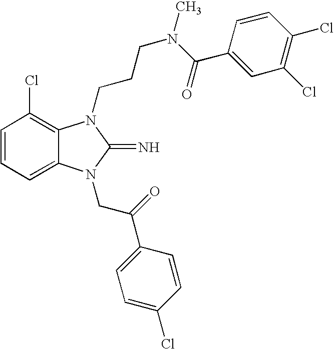 | D12 | 2.53 min (c) | (M + H)⁺563.0, 565.0, 566.9 |
| 7-Chloro-2- imino-1-(3- methylamino- o-propyl)- 2,3-dihydro- 1H-benzo- imidazole (B, C, D, M) | 3,5- Dichloro- benzoic acid | 2-Bromo- 1-(4- chloro- phenyl)- ethanone | 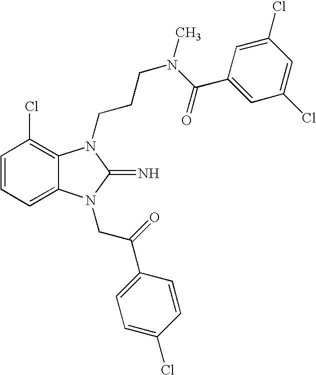 | D13 | 2.53 min (c) | (M + H)⁺563.1, 565.1, 566.9 |
| 7-Chloro-2- imino-1-(3- methylamino- o-propyl)- 2,3-dihydro- 1H-benzo- imidazole (B, C, D, M) | Naphthalene- 1- carboxylic acid | 2-Bromo- 1-(4- chloro- phenyl)- ethanone | 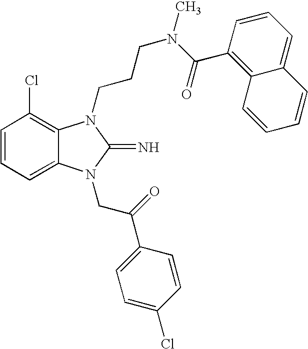 | D14 | 2.26 min (c) | (M + H)⁺545.1, 547.1 |
| 7-Chloro-2- imino-1-(3- methylamino- o-propyl)- 2,3-dihydro- 1H-benzo- imidazole (B, C, D, M) | Quinoline- 8- carboxylic acid | 2-Bromo- 1-(4- chloro- phenyl)- ethanone | 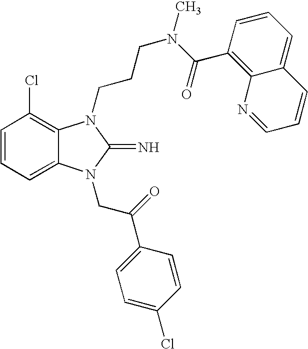 | D15 | 1.6-2.1 min (c) | (M + H)⁺546.1, 548.1 |
| 7-Chloro-2- imino-1-(3- methylamino- o-propyl)- 2,3-dihydro- 1H-benzo- imidazole (B, C, D, M) | Quinoline- 6-6 carboxylic acid | 2-Bromo- 1-(4- chloro- phenyl)- ethanone | 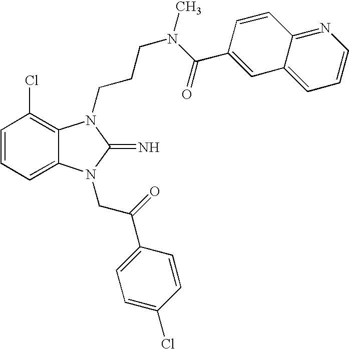 | D16 | 1.70 min (c) | (M + H)⁺546.1, 548.1 |
| 7-Chloro-2- imino-1-(3- methylamino- o-propyl)- 2,3-dihydro- 1H-benzo- imidazole (B, C, D, M) | Quinoline- 5- carboxylic acid | 2-Bromo- 1-(4- chloro- phenyl)- ethanone | 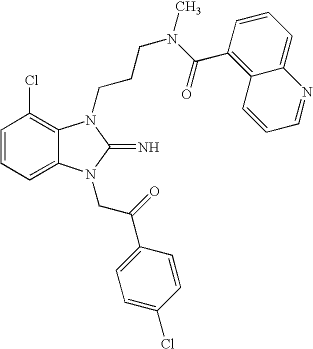 | D17 | 1.69 min (c) | (M + H)⁺546.1, 548.2 |
| 7-Chloro-2- imino-1-(3- methylamino- o-propyl)- 2,3-dihydro- 1H-benzo- imidazole (B, C, D, M) | Benzo[b]thiophene- 5- carboxylic acid | 2-Bromo- 1-(4- chloro- phenyl)- ethanone | 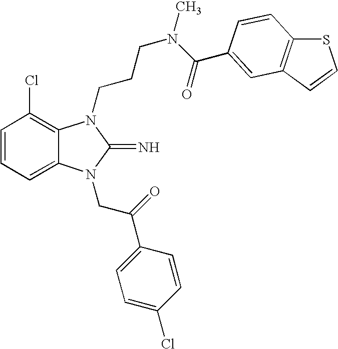 | D18 | 2.20 min (c) | (M + H)⁺551.0, 553.1 |
| 7-Chloro-2- imino-1-(3- methylamino- o-propyl)- 2,3-dihydro- 1H-benzo- imidazole (B, C, D, M) | 2-tert- Butoxycar- bonyl- amino- benzoic acid | 2-Bromo- 1-(4- chloro- phenyl)- ethanone | 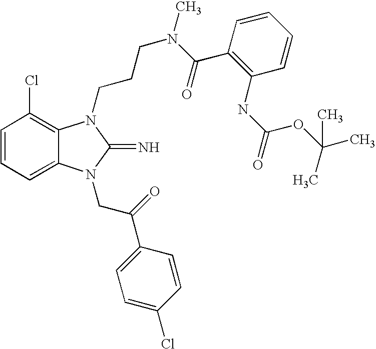 | D19 | 2.53 min (c) | (M + H)⁺610.2, 612.1 |
| 7-Chloro-2- imino-1-(3- methylamino- o-propyl)- 2,3-dihydro- 1H-benzo- imidazole (B, C, D, M) | 4-tert- Butoxycar- bonyl- amino- benzoic acid | 2-Bromo- 1-(4- chloro- phenyl)- ethanone | 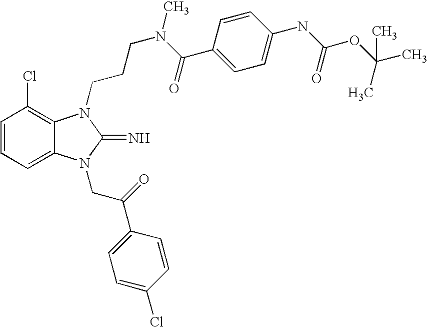 | D20 | 2.41 min (c) | (M + H)⁺610.2, 612.2 |
| 7-Chloro-2- imino-1-(3- methylamino- o-propyl)- 2,3-dihydro- 1H-benzo- imidazole (B, C, D, M) | 2,4- Dimethyl- benzoic acid | 2-Bromo- 1-(4- chloro- phenyl)- ethanone | 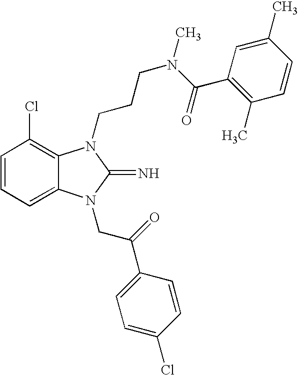 | D21 | 2.38 min (c) | (M + H)⁺523.2, 525.1 |
| 7-Chloro-2- imino-1-(3- methylamino- o-propyl)- 2,3-dihydro- 1H-benzo- imidazole (B, C, D, M) | 3,4- Dimethyl- benzoic acid | 2-Bromo- 1-(4- chloro- phenyl)- ethanone | 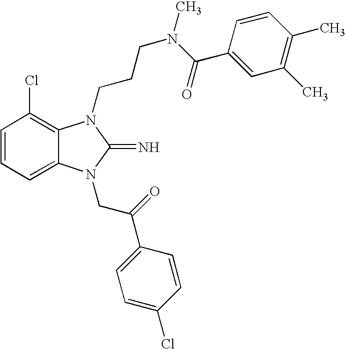 | D22 | 2.42 min (c) | (M + H)⁺523.2, 525.1 |
| 7-Chloro-2- imino-1-(3- methylamino- o-propyl)- 2,3-dihydro- 1H-benzo- imidazole (B, C, D, M) | 3,5- Dimethyl- benzoic acid | 2-Bromo- 1-(4- chloro- phenyl)- ethanone | 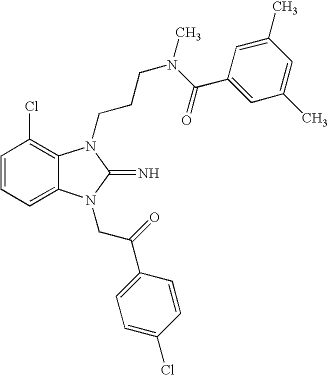 | D23 | 2.44 min (c) | (M + H)⁺523.2, 525.1 |
| 7-Chloro-2- imino-1-(3- methylamino- o-propyl)- 2,3-dihydro- 1H-benzo- imidazole (B, C, D, M) | 2,3- Dichloro- benzoic acid | 2-Bromo- 1-(4- chloro- phenyl)- ethanone | 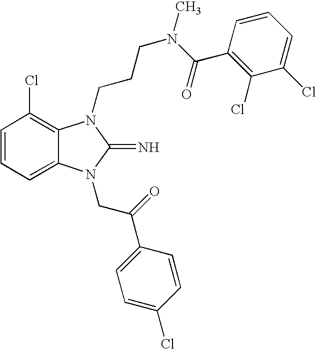 | D24 | 2.1-2.6 min (c) | (M + H)⁺563.0, 565.0, 567.0 |
| 7-Chloro-2- imino-1-(3- methylamino- o-propyl)- 2,3-dihydro- 1H-benzo- imidazole (B, C, D, M) | 2,5- Dichloro- benzoic acid | 2-Bromo- 1-(4- chloro- phenyl)- ethanone | 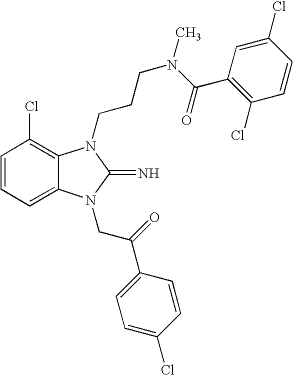 | D25 | 2.5 min (c) | (M + H)⁺563.0, 565.0, 567.0 |
| 7-Chloro-2- imino-1-(3- methylamino- o-propyl)- 2,3-dihydro- 1H-benzo- imidazole (B, C, D, M) | 2,5- Dichloro- benzoic acid | 2-Bromo- 1-(4- chloro- phenyl)- ethanone | 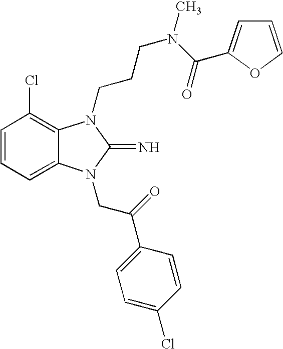 | D26 | 1.96 min (c) | (M + H)⁺485.1, 487.1 |
| 7-Chloro-2- imino-1-(3- methylamino- o-propyl)- 2,3-dihydro- 1H-benzo- imidazole (B, C, D, M) | Thiophene- 2- carboxylic acid | 2-Bromo- 1-(4- chloro- phenyl)- ethanone | 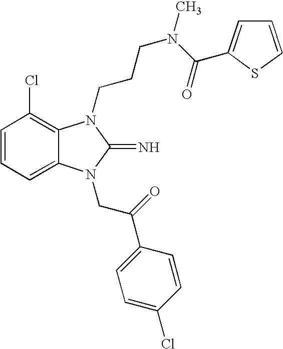 | D27 | 2.40 min (c) | (M + H)⁺501.1, 503.1 |
| 7-Chloro-2- imino-1-(3- methylamino- o-propyl)- 2,3-dihydro- 1H-benzo- imidazole (B, C, D, M) | 1-Methyl- 1H- pyrrole-2- carboxylic acid | 2-Bromo- 1-(4- chloro- phenyl)- ethanone | 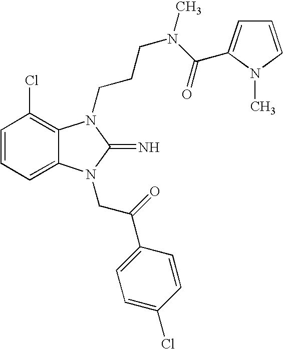 | D28 | 2.58 min (c) | (M + H)⁺498.2, 500.1 |
| 7-Chloro-2- imino-1-(3- methylamino- o-propyl)- 2,3-dihydro- 1H-benzo- imidazole (B, C, D, M) | Thiazole-4- carboxylic acid | 2-Bromo- 1-(4- chloro- phenyl)- ethanone | 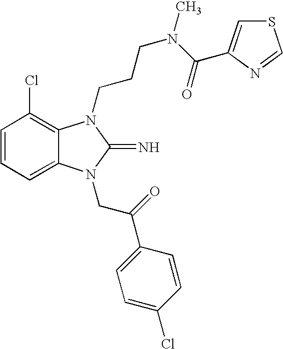 | D29 | 1.93 min (c) | (M + H)⁺502.1, 504.1, 506.1 |
| 7-Chloro-2- imino-1-(3- methylamino- o-propyl)- 2,3-dihydro- 1H-benzo- imidazole (B, C, D, M) | 3-Trifluoro methyl- benzoic acid | 2-Bromo- 1-(4- chloro- phenyl)- ethanone | 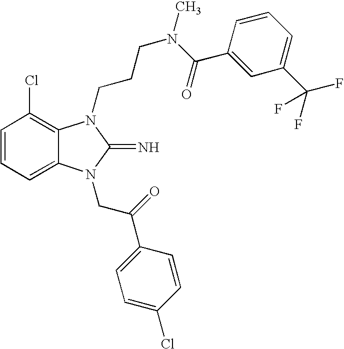 | D30 | 1.59 min (c) | (M + H)⁺585.2, 587.2 |
| 7-Chloro-2- imino-1-(3- methylamino- o-propyl)- 2,3-dihydro- 1H-benzo- imidazole (B, C, D, M) | 4-Trifluoro methyl- benzoic acid | 2-Bromo- 1-(4- chloro- phenyl)- ethanone | 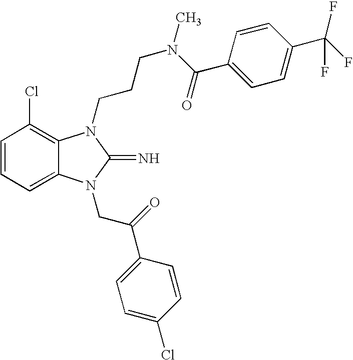 | D31 | 4.03 min (c) | (M + H)⁺563.2, 565.1 |
| 7-Chloro-2- imino-1-(3- methylamino- o-propyl)- 2,3-dihydro- 1H-benzo- imidazole (B, C, D, M) | (2- Methoxy- phenyl)- acetic acid | 2-Bromo- 1-(4- chloro- phenyl)- ethanone | 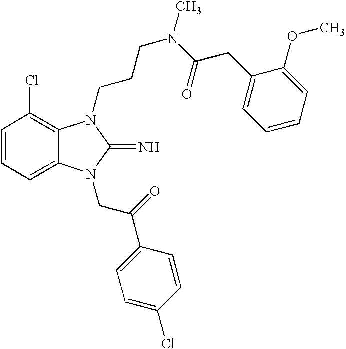 | D32 | 3.12 min (c) | (M + H)⁺539.2, 541.2 |
| 7-Chloro-2- imino-1-(3- methylamino- o-propyl)- 2,3-dihydro- 1H-benzo- imidazole (B, C, D, M) | Bicyclo [2.2.1]hept- 2-yl- acetic acid | 2-Bromo- 1-(4- chloro- phenyl)- ethanone | 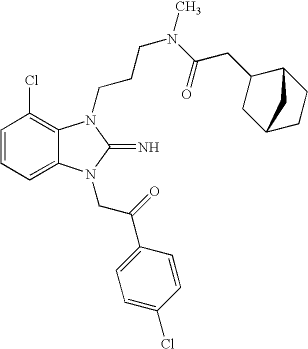 | D33 | 1.98 min (c) | (M + H)⁺502.1, 504.1 |
| 7-Chloro-2- imino-1-(3- methylamino- o-propyl)- 2,3-dihydro- 1H-benzo- imidazole (B, C, D, M) | (3- Phenoxy- phenyl)- acetic acid | 2-Bromo- 1-(4- chloro- phenyl)- ethanone | 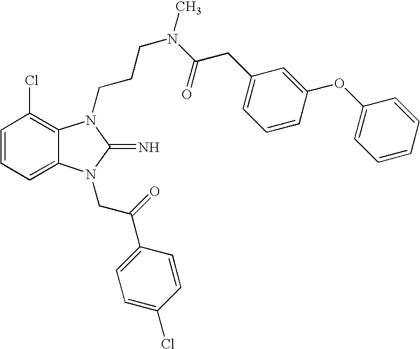 | D34 | 2.31 min (i) | (M + H)⁺601.2, 603.2 |
| 7-Chloro-2- imino-1-(3- methylamino- o-propyl)- 2,3-dihydro- 1H-benzo- imidazole (B, C, D, M) | 4- Benzyloxy- benzoic acid | 2-Bromo- 1-(4- chloro- phenyl)- ethanone | 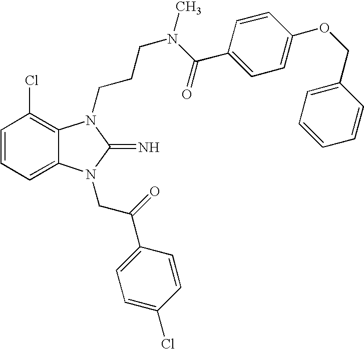 | D35 | 2.21 min (i) | (M + H)⁺601.2, 603.2 |
| 7-Chloro-2- imino-1-(3- methylamino- o-propyl)- 2,3-dihydro- 1H-benzo- imidazole (B, C, D, M) | Cyclopentyl- acetic acid | 2-Bromo- 1-(4- chloro- phenyl)- ethanone | 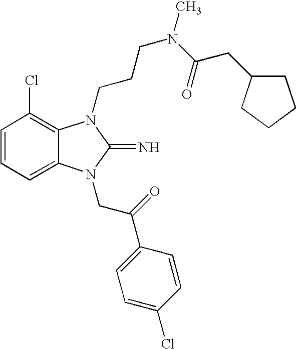 | D36 | 2.15 min (i) | (M + H)⁺501.2, 503.2 |
| 7-Chloro-2- imino-1-(3- methylamino- o-propyl)- 2,3-dihydro- 1H-benzo- imidazole (B, C, D, M) | 3,4,5- Trimethoxy- benzoic acid | 2-Bromo- 1-(4- chloro- phenyl)- ethanone | 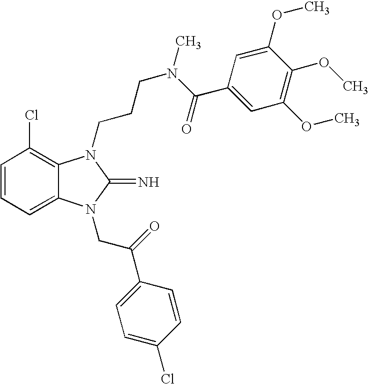 | D37 | 1.86 min (i) | (M + H)⁺583.1, 585.1 |
| 7-Chloro-2- imino-1-(3- methylamino- o-propyl)- 2,3-dihydro- 1H-benzo- imidazole (B, C, D, M) | 2,5- Dimethyl- furan-3- carboxylic acid | 2-Bromo- 1-(4- chloro- phenyl)- ethanone | 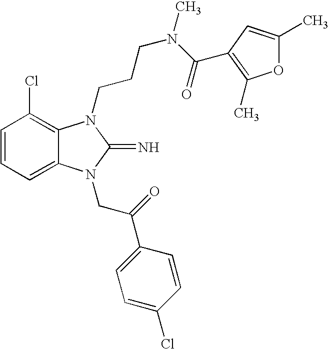 | D38 | 2.00 min (i) | (M + H)⁺511.1, 513.1 |
| 7-Chloro-2- imino-1-(3- methylamino- o-propyl)- 2,3-dihydro- 1H-benzo- imidazole (B, C, D, M) | 3-Methyl- thiophene-2- carboxylic acid | 2-Bromo- 1-(4- chloro- phenyl)- ethanone | 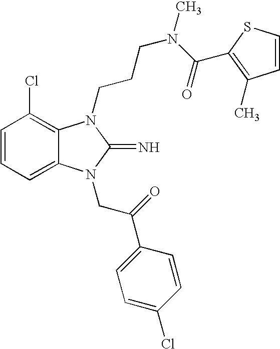 | D39 | 1.97 min (i) | (M + H)⁺513.1, 515.1 |
| 7-Chloro-2- imino-1-(3- methylamino- o-propyl)- 2,3-dihydro- 1H-benzo- imidazole (B, C, D, M) | 5-Methyl- thiophene-2- carboxylic acid | 2-Bromo- 1-(4- chloro- phenyl)- ethanone | 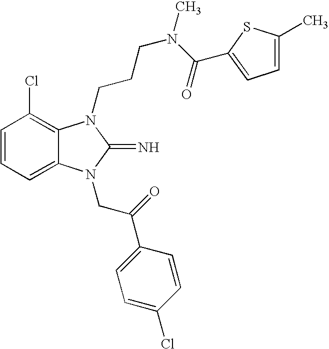 | D40 | 2.03 min (i) | (M + H)⁺515.1, 517.1 |
| 7-Chloro-2- imino-1-(3- methylamino- o-propyl)- 2,3-dihydro- 1H-benzo- imidazole (B, C, D, M) | 1-Phenyl- 1H-pyrazole- 4-carboxylic acid | 2-Bromo- 1-(4- chloro- phenyl)- ethanone | 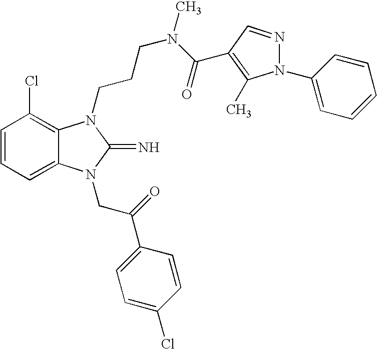 | D41 | 1.96 min (i) | (M + H)⁺575.2, 577.2 |
| 7-Chloro-2- imino-1-(3- methylamino- o-propyl)- 2,3-dihydro- 1H-benzo- imidazole (B, C, D, M) | 1-Methyl- 1H-indole-3- carboxylic acid | 2-Bromo- 1-(4- chloro- phenyl)- ethanone | 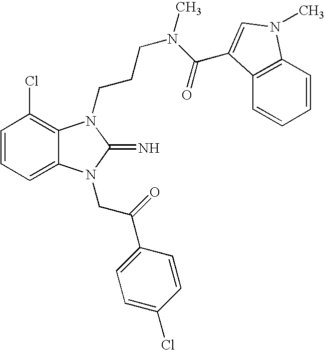 | D42 | 2.05 min (i) | (M + H)⁺548.2, 550.2 |
| 7-Chloro-2- imino-1-(3- methylamino- o-propyl)- 2,3-dihydro- 1H-benzo- imidazole (B, C, D, M) | (3,4,5- Trimethoxy- phenyl)- acetic acid | 2-Bromo- 1-(4- chloro- phenyl)- ethanone | 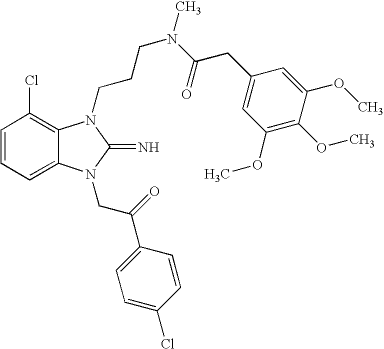 | D43 | 1.64 min (i) | (M + H)⁺599.1, 601.2 |
| 7-Chloro-2- imino-1-(3- methylamino- o-propyl)- 2,3-dihydro- 1H-benzo- imidazole (B, C, D, M) | (3,4- Dichloro- phenyl)- acetic acid | 2-Bromo- 1-(4- chloro- phenyl)- ethanone | 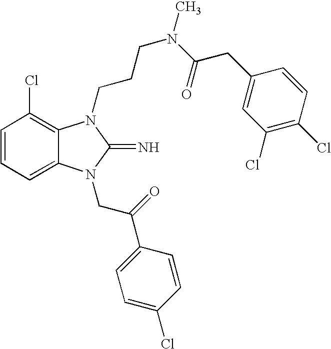 | D44 | 1.85 min (i) | (M + H)⁺577.0, 579.0, 581.0 |
| 7-Chloro-2- imino-1-(3- methylamino- o-propyl)- 2,3-dihydro- 1H-benzo- imidazole (B, C, D, M) | Naphthalen- 1-yl- acetic acid | 2-Bromo- 1-(4- chloro- phenyl)- ethanone | 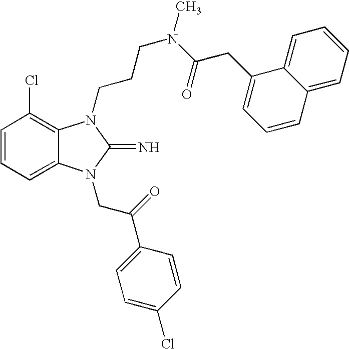 | D45 | 1.84 min (i) | (M + H)⁺559.1, 561.1 |
| 7-Chloro-2- imino-1-(3- methylamino- o-propyl)- 2,3-dihydro- 1H-benzo- imidazole (B, C, D, M) | 5-Methyl- pyrazine-2- carboxylic acid | 2-Bromo- 1-(4- chloro- phenyl)- ethanone | 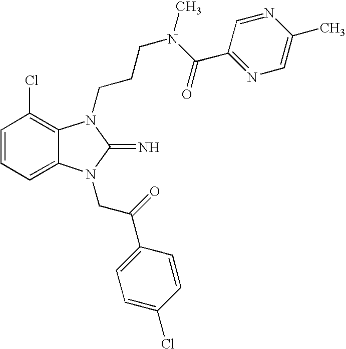 | D46 | 1.51 min (i) | (M + H)⁺511.1, 513.1 |
| 7-Chloro-2- imino-1-(3- methylamino- o-propyl)- 2,3-dihydro- 1H-benzo- imidazole (B, C, D, M) | Tetrahydro- furan-2- carboxylic acid | 2-Bromo- 1-(4- chloro- phenyl)- ethanone | 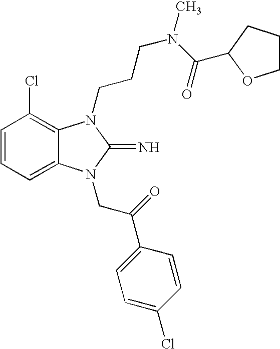 | D47 | 1.58 min (i) | (M + H)⁺489.1, 491.1 |
| 7-Chloro-2- imino-1-(3- methylamino- o-propyl)- 2,3-dihydro- 1H-benzo- imidazole (B, C, D, M) | Tetrahydro- furan-3- carboxylic acid | 2-Bromo- 1-(4- chloro- phenyl)- ethanone | 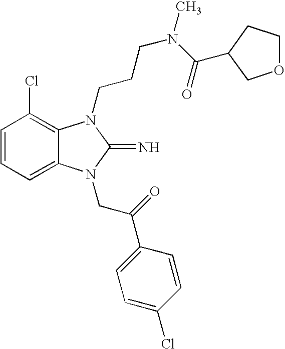 | D48 | 1.52 min (i) | (M + H)⁺489.1, 491.1 |
| 7-Chloro-2- imino-1-(3- methylamino- o-propyl)- 2,3-dihydro- 1H-benzo- imidazole (B, C, D, M) | Cyclo- hexane- carboxylic acid | 2-Bromo- 1-(4- chloro- phenyl)- ethanone | 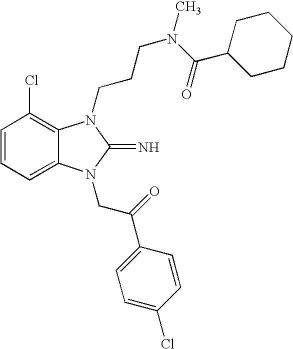 | D49 | 1.76 min (i) | (M + H)⁺501.1, 503.1 |
| 7-Chloro-2- imino-1-(3- methylamino- o-propyl)- 2,3-dihydro- 1H-benzo- imidazole (B, C, D, M) | [(Furan-2- carbonyl)- amino]- acetic acid | 2-Bromo- 1-(4- chloro- phenyl)- ethanone | 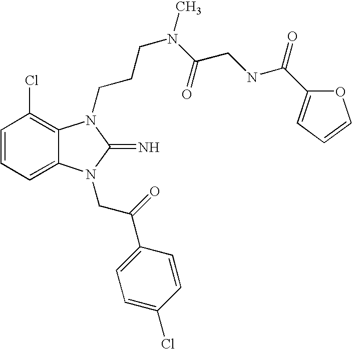 | D50 | 1.54 min (i) | (M + H)⁺542.1, 544.1 |
| 7-Chloro-2- imino-1-(3- methylamino- o-propyl)- 2,3-dihydro- 1H-benzo- imidazole (B, C, D, M) | (Pyrimidin- 2- ylsulfanyl)- acetic acid | 2-Bromo- 1-(4- chloro- phenyl)- ethanone | 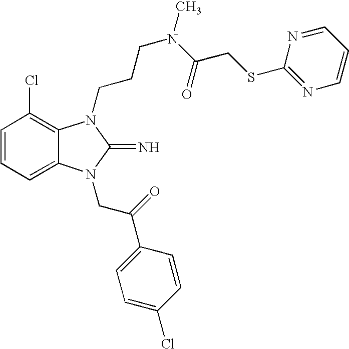 | D51 | 1.58 min (i) | (M + H)⁺543.1, 545.1 |
| 7-Chloro-2- imino-1-(3- methylamino- o-propyl)- 2,3-dihydro- 1H-benzo- imidazole (B, C, D, M) | 3-(2- Chloro- phenyl)- propionic acid | 2-Bromo- 1-(4- chloro- phenyl)- ethanone | 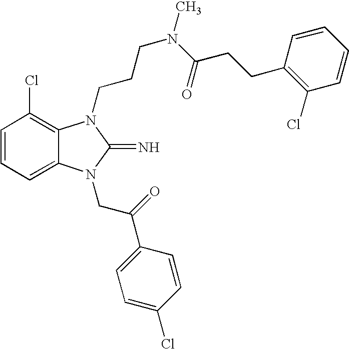 | D52 | 1.82 min (i) | (M + H)⁺557.1, 559.1, 561.1 |
| 7-Chloro-2- imino-1-(3- methylamino- o-propyl)- 2,3-dihydro- 1H-benzo- imidazole (B, C, D, M) | 3-(4- Chloro- phenyl)- propionic acid | 2-Bromo- 1-(4- chloro- phenyl)- ethanone | 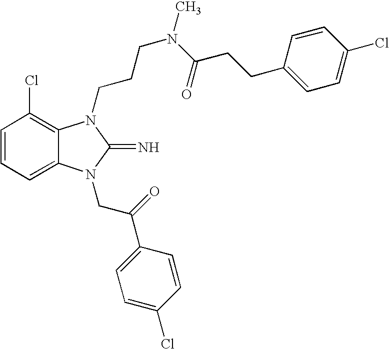 | D53 | 1.86 min (i) | (M + H)⁺557.1, 559.1, 561.1 |
| 7-Chloro-2- imino-1-(3- methylamino- o-propyl)- 2,3-dihydro- 1H-benzo- imidazole (B, C, D, M) | 2-Methyl- cyclo- hexane- carboxylic acid | 2-Bromo- 1-(4- chloro- phenyl)- ethanone | 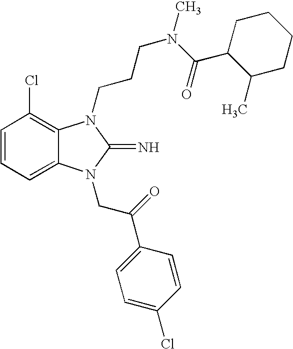 | D54 | 1.97 min (i) | (M + H)⁺515.2, 517.2 |
| 7-Chloro-2- imino-1-(3- methylamino- o-propyl)- 2,3-dihydro- 1H-benzo- imidazole (B, C, D, M) | 4-Methoxy- cyclohexane- carboxylic acid | 2-Bromo- 1-(4- chloro- phenyl)- ethanone | 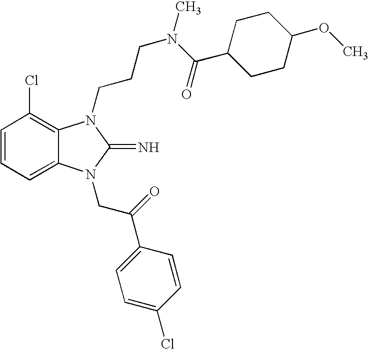 | D55 | 1.73 min (i) | (M + H)⁺531.2, 533.2 |
| 7-Chloro-2- imino-1-(3- methylamino- o-propyl)- 2,3-dihydro- 1H-benzo- imidazole (B, C, D, M) | (Toluene-4- sulfonyl- amino)- acetic acid | 2-Bromo- 1-(4- chloro- phenyl)- ethanone | 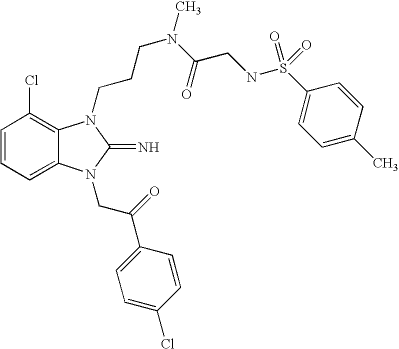 | D56 | 1.73 min (i) | (M + H)⁺602.1, 604.1 |
| 7-Chloro-2- imino-1-(3- methylamino- o-propyl)- 2,3-dihydro- 1H-benzo- imidazole (B, C, D, M) | m-Tolyloxy- acetic acid | 2-Bromo- 1-(4- chloro- phenyl)- ethanone | 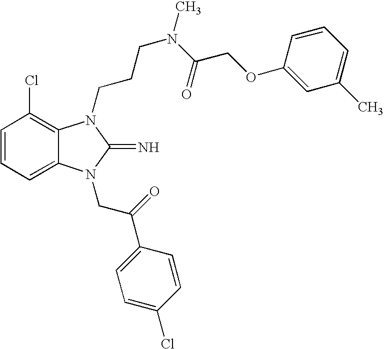 | D57 | 1.78 min (i) | (M + H)⁺539.1, 541.1 |
| 7-Chloro-2- imino-1-(3- methylamino- o-propyl)- 2,3-dihydro- 1H-benzo- imidazole (B, C, D, M) | o-Tolyloxy- acetic acid | 2-Bromo- 1-(4- chloro- phenyl)- ethanone | 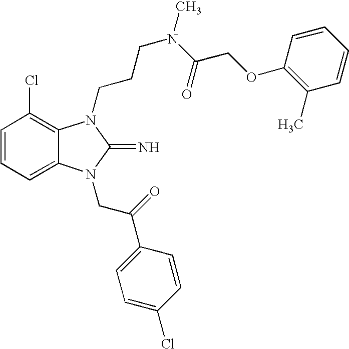 | D58 | 2.45 min (i) | (M + H)⁺539.1, 541.1 |
| 7-Chloro-2- imino-1-(3- methylamino- o-propyl)- 2,3-dihydro- 1H-benzo- imidazole (B, C, D, M) | o-Tolyloxy- acetic acid | 2-Bromo- 1-(4- chloro- phenyl)- ethanone | 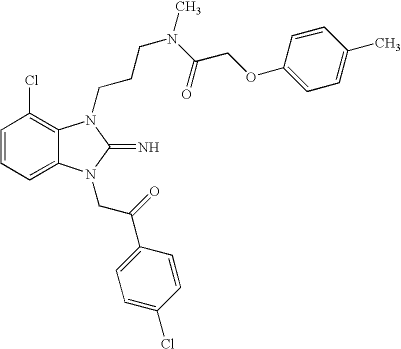 | D59 | 2.54 min (i) | (M + H)⁺239.1, 241.1 |
| 7-Chloro-2- imino-1-(3- methylamino- o-propyl)- 2,3-dihydro- 1H-benzo- imidazole (B, C, D, M) | (4- Benzyloxy- phenyl)- acetic acid | 2-Bromo- 1-(4- chloro- phenyl)- ethanone | 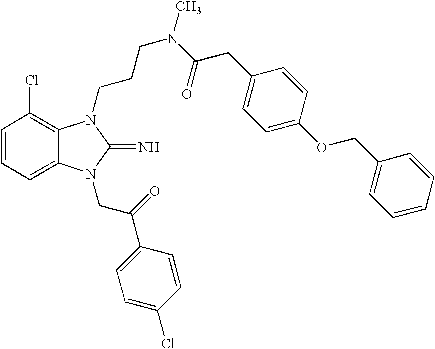 | D60 | 2.25 min (i) | (M + H)⁺615.2, 617.2 |
| 7-Chloro-2- imino-1-(3- methylamino- o-propyl)- 2,3-dihydro- 1H-benzo- imidazole (B, C, D, M) | 4-Phenoxy- benzoic acid | 2-Bromo- 1-(4- chloro- phenyl)- ethanone | 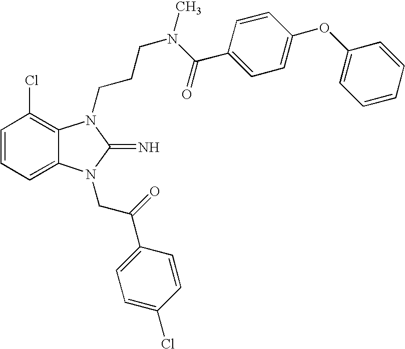 | D61 | 1.97 min (i) | (M + H)⁺587.1, 589.1 |
| 7-Chloro-2- imino-1-(3- methylamino- o-propyl)- 2,3-dihydro- 1H-benzo- imidazole (B, C, D, M) | (3-Trifluoro- methyl- phenyl)- acetic acid | 2-Bromo- 1-(4- chloro- phenyl)- ethanone | 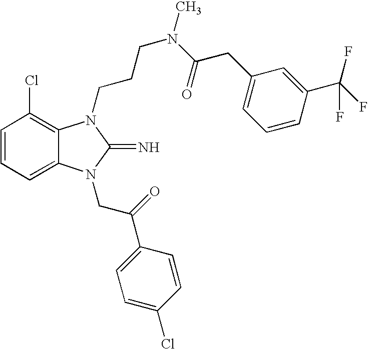 | D62 | 2.63 min (i) | (M + H)⁺575.1, 577.1 |
| 7-Chloro-2- imino-1-(3- methylamino- o-propyl)- 2,3-dihydro- 1H-benzo- imidazole (B, C, D, M) | (4-Trifluoro- methyl- phenyl)- acetic acid | 2-Bromo- 1-(4- chloro- phenyl)- ethanone | 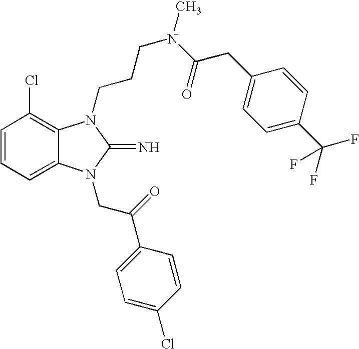 | D63 | 1.86 min (i) | (M + H)⁺577.1, 579.1 |
| 7-Chloro-2- imino-1-(3- methylamino- o-propyl)- 2,3-dihydro- 1H-benzo- imidazole (B, C, D, M) | Cyclo pentane- carboxylic acid | 2-Bromo- 1-(4- chloro- phenyl)- ethanone | 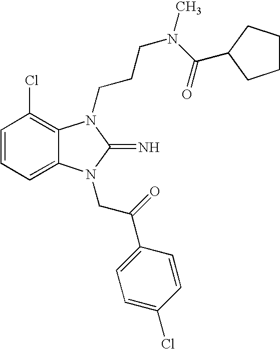 | D64 | 1.78 min (i) | (M + H)⁺487.1, 489.2 |
| 7-Chloro-2- imino-1-(3- methylamino- o-propyl)- 2,3-dihydro- 1H-benzo- imidazole (B, C, D, M) | 3-Thiophen- 2-yl- propionic acid | 2-Bromo- 1-(4- chloro- phenyl)- ethanone | 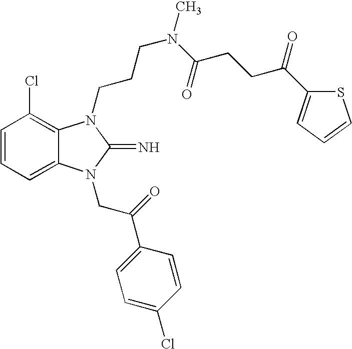 | D65 | 1.70 min (i) | (M + H)⁺557.1, 559.1 |
| 7-Chloro-2- imino-1-(3- methylamino- o-propyl)- 2,3-dihydro- 1H-benzo- imidazole (B, C, D, M) | (3-Methoxy- phenyl)- acetic acid | 2-Bromo- 1-(4- chloro- phenyl)- ethanone | 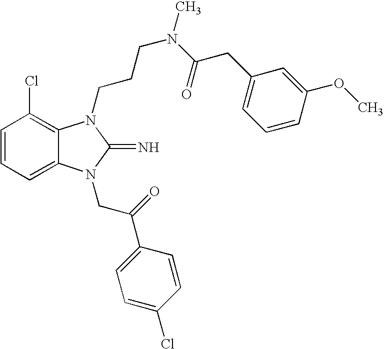 | D66 | 1.75 min (i) | (M + H)⁺539.1, 541.1 |
| 7-Chloro-2- imino-1-(3- methylamino- o-propyl)- 2,3-dihydro- 1H-benzo- imidazole (B, C, D, M) | (4-Methoxy- phenyl)- acetic acid | 2-Bromo- 1-(4- chloro- phenyl)- ethanone | 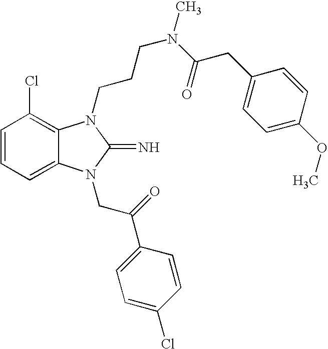 | D67 | 2.43 min (i) | (M + H)⁺539.1, 541.1 |
| 7-Chloro-2- imino-1-(3- methylamino- o-propyl)- 2,3-dihydro- 1H-benzo- imidazole (B, C, D, M) | (3-Chloro- phenyl)- acetic acid | 2-Bromo- 1-(4- chloro- phenyl)- ethanone | 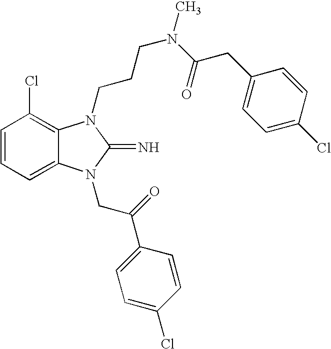 | D68 | 2.48 min (i) | (M + H)⁺543.1, 545.1 |
| 7-Chloro-2- imino-1-(3- methylamino- o-propyl)- 2,3-dihydro- 1H-benzo- imidazole (B, C, D, M) | (2,5- Dimethyl- phenyl)- acetic acid | 2-Bromo- 1-(4- chloro- phenyl)- ethanone | 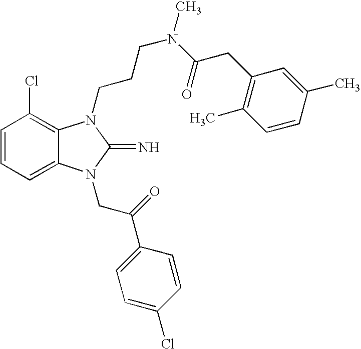 | D69 | 1.92 min (i) | (M + H)⁺537.1, 539.2 |
| 7-Chloro-2- imino-1-(3- methylamino- o-propyl)- 2,3-dihydro- 1H-benzo- imidazole (B, C, D, M) | (2,4,6- Trimethyl- phenyl)- acetic acid | 2-Bromo- 1-(4- chloro- phenyl)- ethanone | 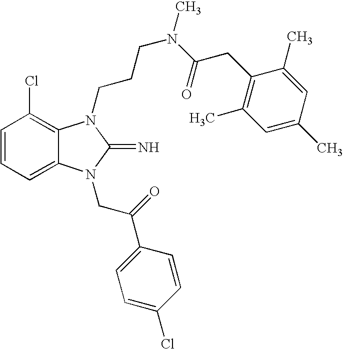 | D70 | 2.18 min (i) | (M + H)⁺551.2, 553.2 |
| 7-Chloro-2- imino-1-(3- methylamino- o-propyl)- 2,3-dihydro- 1H-benzo- imidazole (B, C, D, M) | (3,5- Dimethyl- phenyl)- acetic acid | 2-Bromo- 1-(4- chloro- phenyl)- ethanone | 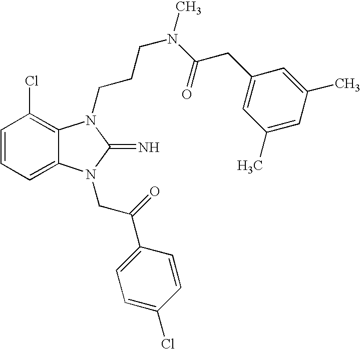 | D71 | 1.88 min (i) | (M + H)⁺537.1, 539.2 |
| 7-Chloro-2- imino-1-(3- methylamino- o-propyl)- 2,3-dihydro- 1H-benzo- imidazole (B, C, D, M) | (2,4- Dimethoxy- phenyl)- acetic acid | 2-Bromo- 1-(4- chloro- phenyl)- ethanone | 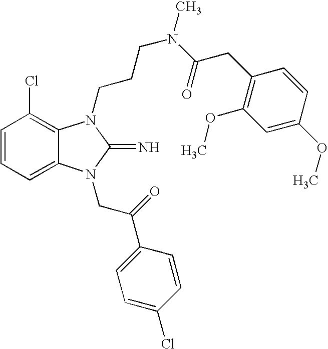 | D72 | 1.77 min (i) | (M + H)⁺569.1, 571.1 |
| 7-Chloro-2- imino-1-(3- methylamino- o-propyl)- 2,3-dihydro- 1H-benzo- imidazole (B, C, D, M) | (2,5- Dimethoxy- phenyl)- acetic acid | 2-Bromo- 1-(4- chloro- phenyl)- ethanone | 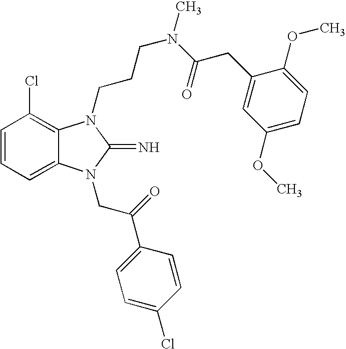 | D73 | 1.74 min (i) | (M + H)⁺569.1, 571.2 |
| 7-Chloro-2- imino-1-(3- methylamino- o-propyl)- 2,3-dihydro- 1H-benzo- imidazole (B, C, D, M) | (3,4- Dimethoxy- phenyl)- acetic acid | 2-Bromo- 1-(4- chloro- phenyl)- ethanone | 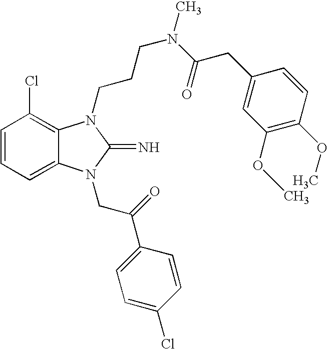 | D74 | 1.64 min (i) | (M + H)⁺569.1, 571.2 |
| 7-Chloro-2- imino-1-(3- methylamino- o-propyl)- 2,3-dihydro- 1H-benzo- imidazole (B, C, D, M) | (3,5- Dimethoxy- phenyl)- acetic acid | 2-Bromo- 1-(4- chloro- phenyl)- ethanone | 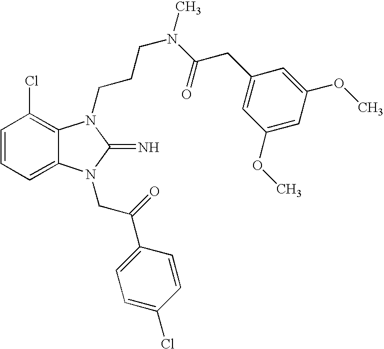 | D75 | 1.78 min (i) | (M + H)⁺569.1, 571.2 |
| 7-Chloro-2- imino-1-(3- methylamino- o-propyl)- 2,3-dihydro- 1H-benzo- imidazole (B, C, D, M) | Benzo[1,3]dioxol-5-yl- acetic acid | 2-Bromo- 1-(4- chloro- phenyl)- ethanone | 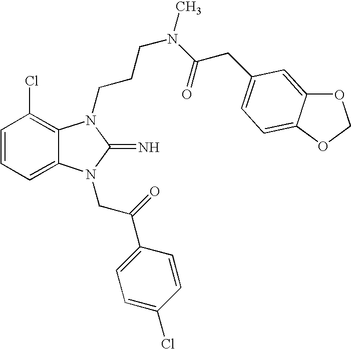 | D76 | 1.72 min (i) | (M + H)⁺553.1, 555.1 |
| 7-Chloro-2- imino-1-(3- methylamino- o-propyl)- 2,3-dihydro- 1H-benzo- imidazole (B, C, D, M) | Naphthalen- 2-yl- acetic acid | 2-Bromo- 1-(4- chloro- phenyl)- ethanone | 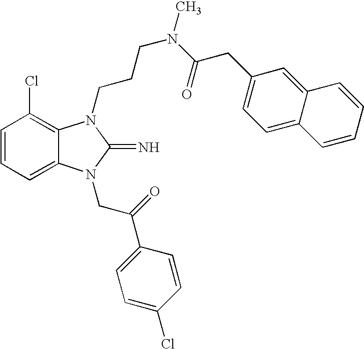 | D77 | 1.87 min (i) | (M + H)⁺559.1, 561.1 |
| 7-Chloro-2- imino-1-(3- methylamino- o-propyl)- 2,3-dihydro- 1H-benzo- imidazole (B, C, D, M) | Diphenyl- acetic acid | 2-Bromo- 1-(4- chloro- phenyl)- ethanone | 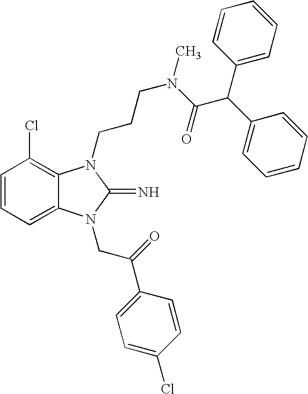 | D78 | 2.08 min (i) | (M + H)⁺585.2, 587.2 |
| 7-Chloro-1- piperidin-3- ylmethyl-1,3- dihydro- benzoimidazol- 2- ylideneamine (B, C, D, M) | 4-Methoxy- benzoic acid | 2-Bromo- 1-(4- chloro- phenyl)- ethanone | 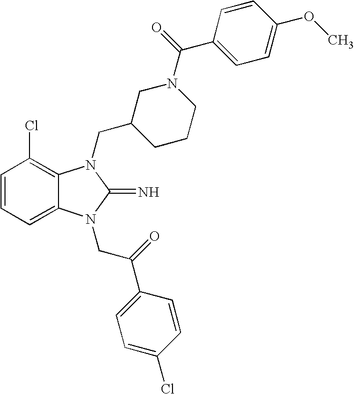 | D79 | 1.75 min (i) | (M + H)⁺551.2, 553.2 |
| 7-Chloro-1- piperidin-3- ylmethyl-1,3- dihydro- benzoimidazol- 2- ylideneamine (B, C, D, M) | 2-Methoxy- benzoic acid | 2-Bromo- 1-(4- chloro- phenyl)- ethanone | 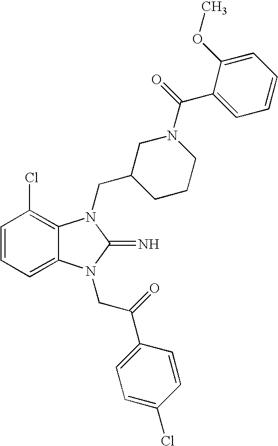 | D80 | 1.77 min (i) | (M + H)⁺551.1, 553.1 |
| 7-Chloro-2- imino-1-(3- methylamino- o-propyl)- 2,3-dihydro- 1H-benzo- imidazole (B, C, D, M) | (2,4- Dimethoxy- phenyl)- acetic acid | 2-Bromo- 1-(4- chloro- phenyl)- ethanone | 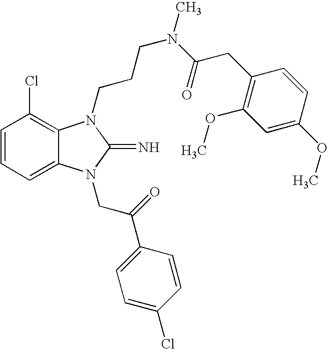 | D81 | 1.74-2.00 min (i) | (M + H)⁺569.1, 571.1 |
| 7-Chloro-2- imino-1-(3- methylamino- o-propyl)- 2,3-dihydro- 1H-benzo- imidazole (B, C, D, M) | Quinoline- 8-carboxylic acid | 2-Bromo- 1-(4- chloro- phenyl)- ethanone | 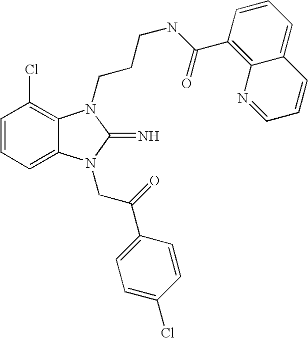 | D82 | 2.34 min (c) | (M + H)⁺532.2, 534.1 |
| 7-Chloro-1- piperidin-3- ylmethyl-1,3- dihydro- benzoimidazol- 2- ylideneamine (B, C, D, M) | Naphthalene- 2-carboxylic acid | 2-Bromo- 1-(4- chloro- phenyl)- ethanone | 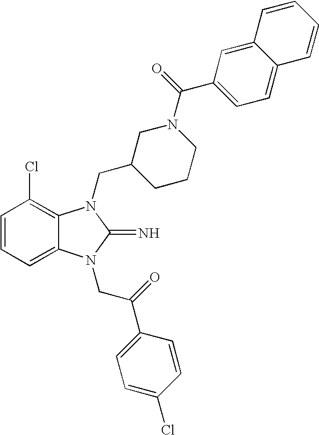 | D83 | 2.55 min (c) | (M + H)⁺571.3, 573.2 |
| 7-Chloro-2- imino-1-(3- methylamino- o-propyl)- 2,3-dihydro- 1H-benzo- imidazole (B, C, D, M) | 3-Methyl- amino- benzoic acid | 2-Bromo- 1-(4- chloro- phenyl)- ethanone | 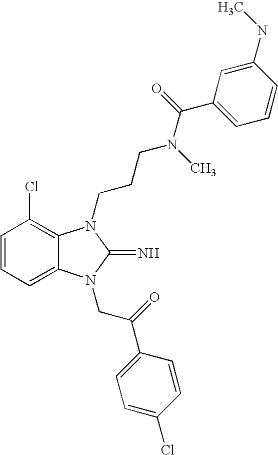 | D84 | 2.28 min (c) | (M + H)⁺524.2, 526.2 |
| 7-Chloro-2- imino-1-(3- methylamino- o-propyl)- 2,3-dihydro- 1H-benzo- imidazole (B, C, D, M) | 3-Phenyl- amino- benzoic acid | 2-Bromo- 1-(4- chloro- phenyl)- ethanone | 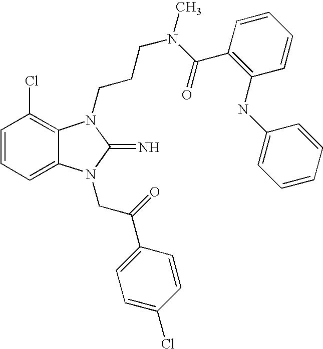 | D85 | 2.63 min (c) | (M + H)⁺586.2, 588.2 |
| 7-Chloro-2- imino-1-(3- methylamino- o-propyl)- 2,3-dihydro- 1H-benzo- imidazole (B, C, D, M) | 2-Dimethyl- amino- benzoic acid | 2-Bromo- 1-(4- chloro- phenyl)- ethanone | 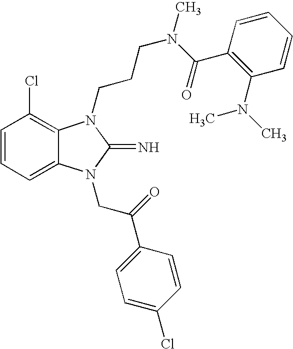 | D86 | 2.23-2.56 min (c) | (M + H)⁺538.6, 540.4 |
| 7-Chloro-2- imino-1-(3- methylamino- o-propyl)- 2,3-dihydro- 1H-benzo- imidazole (B, C, D, M) | 1-Methyl- 1H-indole-2- carboxylic acid | 2-Bromo- 1-(4- chloro- phenyl)- ethanone | 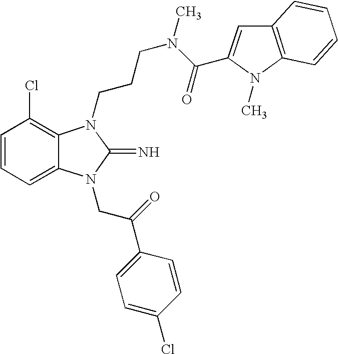 | 2.34- 2.73 min (c) | (M + H)⁺548.3, 550.2 | |
| 7-Chloro-2- imino-1-(3- methylamino- o-propyl)- 2,3-dihydro- 1H-benzo- imidazole (B, C, D, M) | 2,6- Dimethoxy- pyrimidine- 4-carboxylic acid | 2-Bromo- 1-(4- chloro- phenyl)- ethanone | 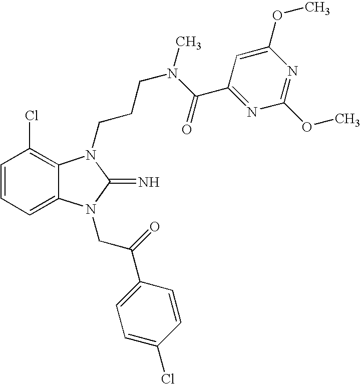 | D88 | 1.83 min (c) | (M + H)⁺557.2, 559.2 |
| 7-Chloro-2- imino-1-(3- methylamino- o-propyl)- 2,3-dihydro- 1H-benzo- imidazole (B, C, D, M) | 2-Pyrrol-1- yl- benzoic acid | 2-Bromo- 1-(4- chloro- phenyl)- ethanone | 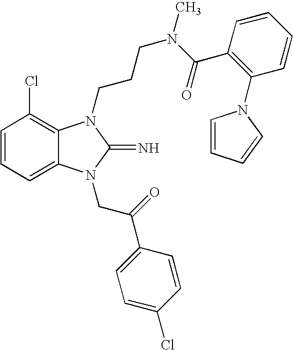 | D89 | 2.38 min (c) | (M + H)⁺560.1, 562.2 |
| 7-Chloro-2- imino-1-(3- methylamino- o-propyl)- 2,3-dihydro- 1H-benzo- imidazole (B, C, D, M) | 2-Methyl- quinoline- 4-carboxylic acid | 2-Bromo- 1-(4- chloro- phenyl)- ethanone | 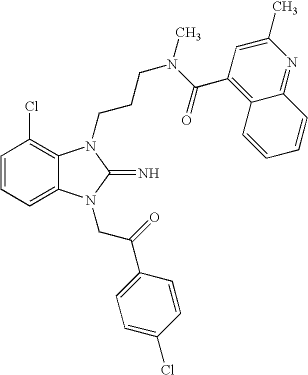 | D90 | 2.04 min (c) | (M + H)⁺560.2, 562.2 |
| 7-Chloro-2- imino-1-(3- methylamino- o-propyl)- 2,3-dihydro- 1H-benzo- imidazole (B, C, D, M) | 4,6- Dimethoxy- pyrimidine- 2-carboxylic acid | 2-Bromo- 1-(4- chloro- phenyl)- ethanone | 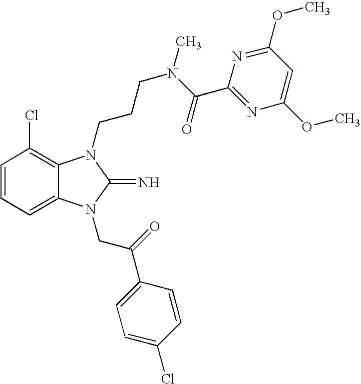 | D91 | 2.06 min (c) | (M + H)⁺557.2, 559.2 |
| 7-Chloro-2- imino-1-(3- methylamino- o-propyl)- 2,3-dihydro- 1H-benzo- imidazole (B, C, D, M) | 3-Phenyl- propionyl chloride | 2-Bromo- 1-(4- bromo- phenyl)- ethanone | 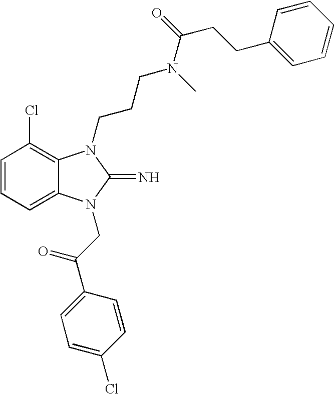 | D92 | 6.25 (a) | 523.19, 525.17 (M + H)⁺ |
| 7-Chloro-2- imino-1-(3- methylamino- o-propyl)- 2,3-dihydro- 1H-benzo- imidazole (B, C, D, M) | Benzo[b]thiophene- 2-carbonyl chloride | 2-Bromo- 1-(4- bromo- phenyl)- ethanone | 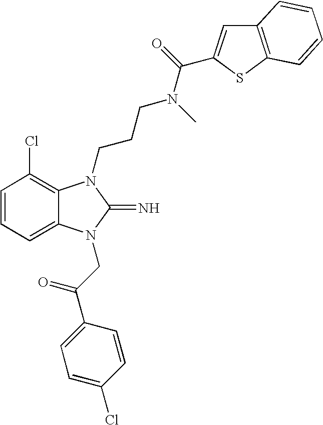 | D93 | 6.36 (a) | 551.21, 553.13 (M + H)⁺ |
| 7-Chloro-2- imino-1-(3- methylamino- o-propyl)- 2,3-dihydro- 1H-benzo- imidazole (B, C, D, M) | Pyrazine-2- carbonyl chloride | 2-Bromo- 1-(4- bromo- phenyl)- ethanone | 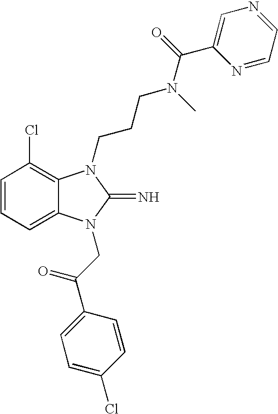 | D94 | 5.41 (a) | 497, 499 (M + H)⁺ |
| 7-Chloro-2- imino-1-(3- methylamino- o-propyl)- 2,3-dihydro- 1H-benzo- imidazole (B, C, D, M) | Quinoxaline- 2-carbonyl chloride | 2-Bromo- 1-(4- bromo- phenyl)- ethanone | 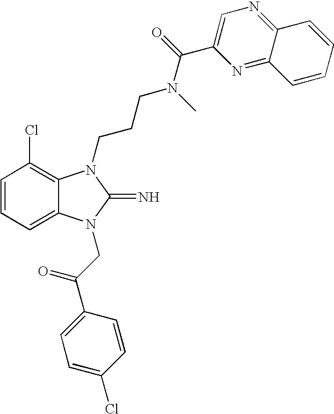 | D95 | 5.84 (a) | 547, 549 (M + H)⁺ |
| 7-Chloro-2- imino-1-(3- methylamino- o-propyl)- 2,3-dihydro- 1H-benzo- imidazole (B, C, D, M) | Phenoxy- acetyl chloride | 2-Bromo- 1-(4- bromo- phenyl)- ethanone | 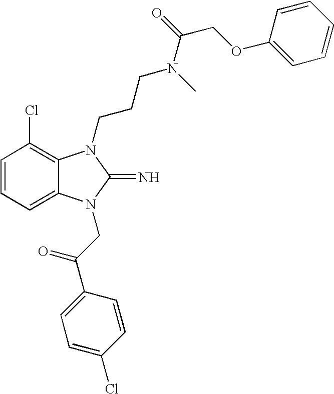 | D96 | 6.05 (a) | 525.13 (M + H)⁺ |
| 7-Chloro-2- imino-1-(3- methylamino- o-propyl)- 2,3-dihydro- 1H-benzo- imidazole (B, C, D, M) | 2,5- Dimethoxy- benzoyl chloride | 2-Bromo- 1-(4- bromo- phenyl)- ethanone | 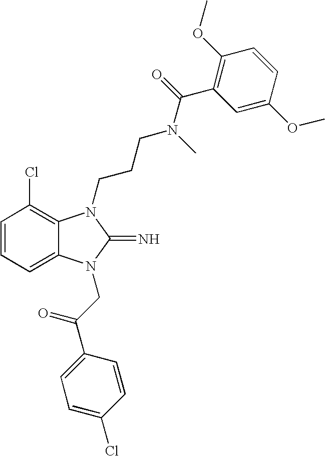 | D97 | 5.94 (a) | 555, 557 (M + H)⁺ |
| 7-Chloro-2- imino-1-(3- methylamino- o-propyl)- 2,3-dihydro- 1H-benzo- imidazole (B, C, D, M) | 2-Fluoro- benzoyl chloride | 2-Bromo- 1-(4- bromo- phenyl)- ethanone | 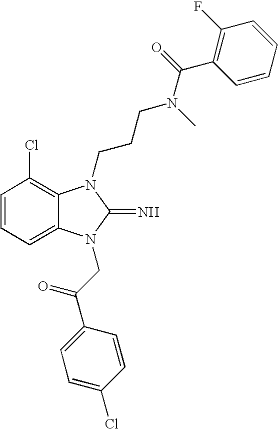 | D98 | 5.98 (a) | 513.1, 515.0 (M + H)⁺ |
| 7-Chloro-2- imino-1-(3- methylamino- o-propyl)- 2,3-dihydro- 1H-benzo- imidazole (B, C, D, M) | 4-Methyl- benzoyl chloride | 2-Bromo- 1-(4- bromo- phenyl)- ethanone | 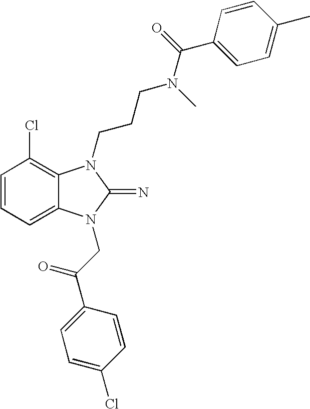 | D99 | 6.11 (a) | 509.1, 511.1 (M + H)⁺ |
| 7-Chloro-2- imino-1-(3- methylamino- o-propyl)- 2,3-dihydro- 1H-benzo- imidazole (B, C, D, M) | 2-Trifluoro- methyl- benzoyl chloride | 2-Bromo- 1-(4- bromo- phenyl)- ethanone | 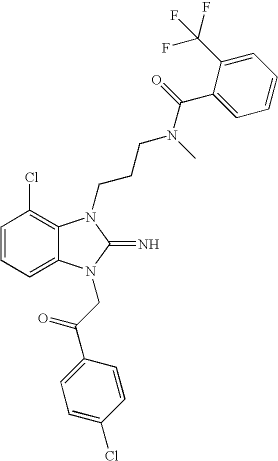 | D100 | 6.28 (a) | 563.1, 565.1 (M + H)⁺ |
| 7-Chloro-2- imino-1-(3- methylamino- o-propyl)- 2,3-dihydro- 1H-benzo- imidazole (B, C, D, M) | Quinoline- 2-carbonyl chloride | 2-Bromo- 1-(4- bromo- phenyl)- ethanone | 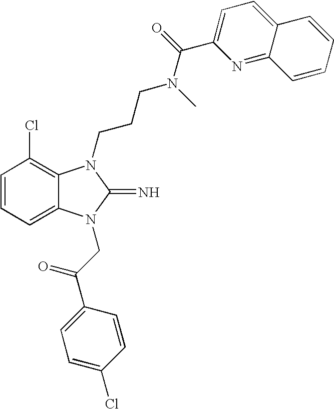 | D101 | 5.96 (a) | 546.1, 548.1 (M + H)⁺ |
| 7-Chloro-2- imino-1-(3- methylamino- o-propyl)- 2,3-dihydro- 1H-benzo- imidazole (B, C, D, M) | Pyrazolo [1,5-a]pyridine-3- chloride | 2-Bromo- 1-(4- bromo- phenyl)- ethanone | 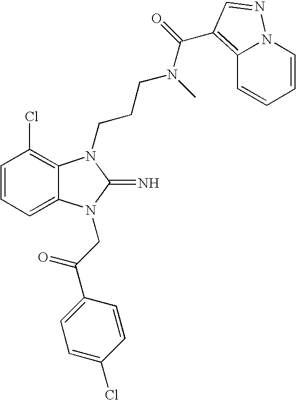 | D102 | 5.68 (a) | 535.13, 538.12 (M + H)⁺ |
| 7-Chloro-2- imino-1-(3- methylamino- o-propyl)- 2,3-dihydro- 1H-benzo- imidazole (B, C, D, M) | Benzofuran- 2-carbonyl chloride | 2-Bromo- 1-(4- bromo- phenyl)- ethanone | 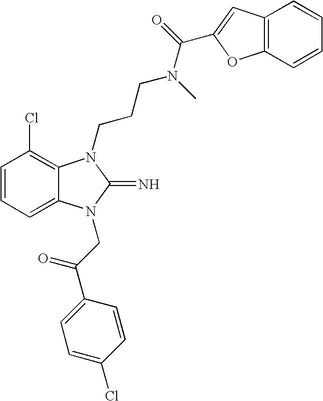 | D103 | 6.24 (a) | 535.12, 538.04 (M + H)⁺ |
| 7-Chloro-2- imino-1-(3- methylamino- o-propyl)- 2,3-dihydro- 1H-benzo- imidazole (B, C, D, M) | 4-{[1- Dimethyl- amino-meth- (E)-ylidene]- sulfamoyl}- benzoyl chloride | 2-Bromo- 1-(4- bromo- phenyl)- ethanone | 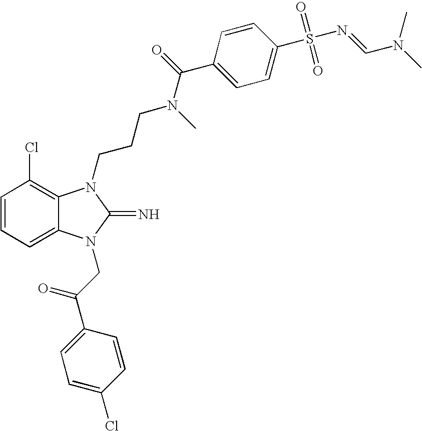 | D104 | 5.52 (a) | 629.12, 631.12, |
| # 632.04 (M + H)⁺ | | | | | | |
| 7-Chloro-2- imino-1-(3- methylamino- o-propyl)- 2,3-dihydro- 1H-benzo- imidazole (B, C, D, M) | Pyridine-2- carbonyl chloride | 2-Bromo- 1-(4- bromo- phenyl)- ethanone | 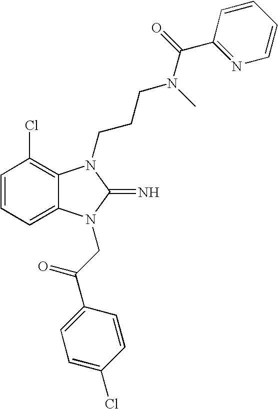 | D105 | 5.51 (a) | 496.11, 498.10, 498.88 (M + H)⁺ |
| 7-Chloro-2- imino-1-(3- methylamino- o-propyl)- 2,3-dihydro- 1H-benzo- imidazole (B, C, D, M) | 2-Trifluoro- methoxy- benzoyl chloride | 2-Bromo- 1-(4- bromo- phenyl)- ethanone | 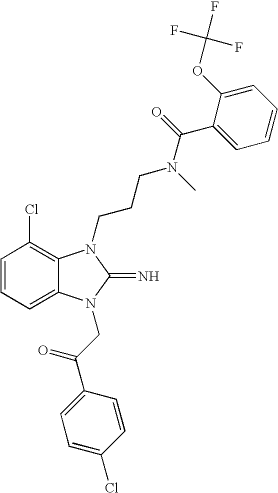 | D106 | 6.28 (a) | 579.10, 581.11, 582.01 (M + H)⁺ |
| 7-Chloro-2- imino-1-(3- methylamino- o-propyl)- 2,3-dihydro- 1H-benzo- imidazole (B, C, D, M) | 2-Ethoxy- benzoyl chloride | 2-Bromo- 1-(4- bromo- phenyl)- ethanone | 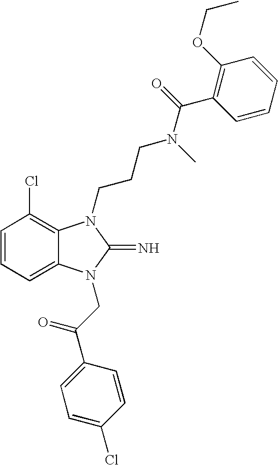 | D107 | 6.11 (a) | 539.14, 541.14, 541.93 (M + H)⁺ |
| 7-Chloro-2- imino-1-(3- methylamino- o-propyl)- 2,3-dihydro- 1H-benzo- imidazole (B, C, D, M) | 3-Dimethyl- amino- benzoyl chloride | 2-Bromo- 1-(4- bromo- phenyl)- ethanone | 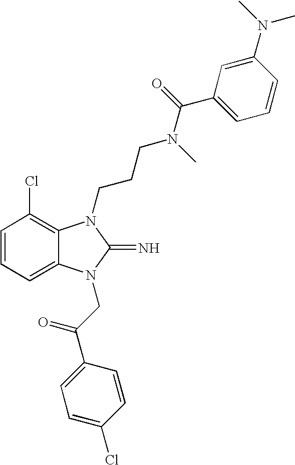 | D108 | 6.11 (a) | 538.17, 540.17, 541.12 (M + H)⁺ |
| 7-Chloro-2- imino-1-(3- methylamino- o-propyl)- 2,3-dihydro- 1H-benzo- imidazole (B, C, D, M) | Phenyl- acetyl chloride | 2-Bromo- 1-(4- bromo- phenyl)- ethanone | 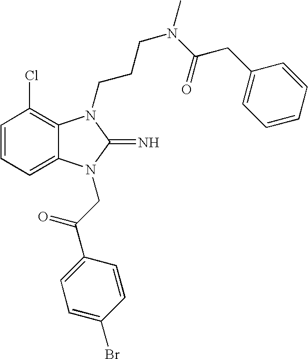 | D109 | 6.03 min (a) | (M + H)⁺553.1, 555.1, 557.0 (2:3:1) |
| 7-Chloro-2- imino-1-(3- methylamino- o-propyl)- 2,3-dihydro- 1H-benzo- imidazole (B, C, D, M) | Benzoyl chloride | 2-Bromo- 1-(4- bromo- phenyl)- ethanone | 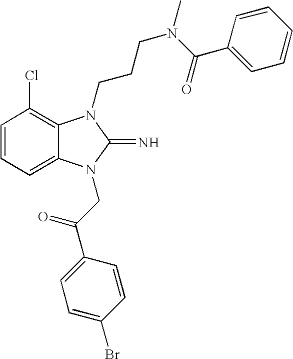 | D110 | | (M + H)⁺539.0, 541.0, 543.0 (2:3:1) |
| 7-Chloro-2- imino-1-(3- methylamino- o-propyl)- 2,3-dihydro- 1H-benzo- imidazole (B, C, D, M) | 4-Chloro- benzoyl chloride | 2-Bromo- 1-(4- bromo- phenyl)- ethanone | 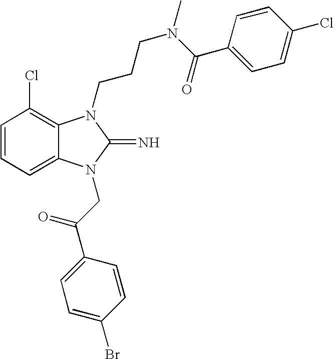 | D111 | 6.16 min (a) | (M + H)⁺573.0, 575.0, 577.0 (2:3:1) |
| 7-Chloro-2- imino-1-(3- methylamino- o-propyl)- 2,3-dihydro- 1H-benzo- imidazole (B, C, D, M) | Benzene- sulfonyl chloride | 2-Bromo- 1-(4- bromo- phenyl)- ethanone | 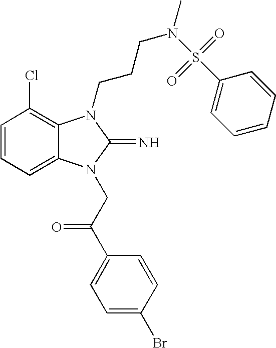 | D112 | 6.36 min (a) | (M + H)⁺575.4, 577.3, 597.3 (2:3:1) |
| 7-Chloro-2- imino-1-(3- methylamino- o-propyl)- 2,3-dihydro- 1H-benzo- imidazole (B, C, D, M) | 2,2- Dimethyl- propionyl chloride | 2-Bromo- 1-(4- chloro- phenyl)- ethanone | 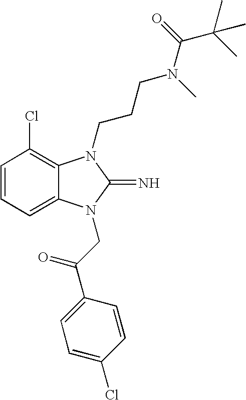 | D113 | 6.25 min (a) | (M + H)⁺475.2, 477.1 (3:2) |
| 7-Chloro-2- imino-1-(3- methylamino- o-propyl)- 2,3-dihydro- 1H-benzo- imidazole (B, C, D, M) | 2-Chloro- benzoyl chloride | 2-Bromo- 1-(4- chloro- phenyl)- ethanone | 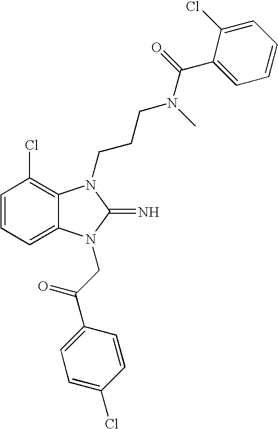 | D114 | 6.24 min (a) | (M + H)⁺529.0, 531.0 (1:1) |
| 7-Chloro-2- imino-1-(3- methylamino- o-propyl)- 2,3-dihydro- 1H-benzo- imidazole (B, C, D, M) | 3-Chloro- benzoyl chloride | 2-Bromo- 1-(4- chloro- phenyl)- ethanone | 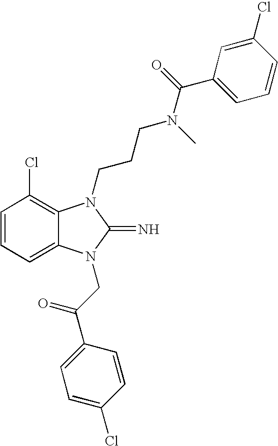 | D115 | 6.21 min (a) | (M + H)⁺529.2, 531.1 (3:2) |
| 7-Chloro-2- imino-1-(3- methylamino- o-propyl)- 2,3-dihydro- 1H-benzo- imidazole (B, C, D, M) | 4-Chloro- benzoyl chloride | 2-Bromo- 1-(4- chloro- phenyl)- ethanone | 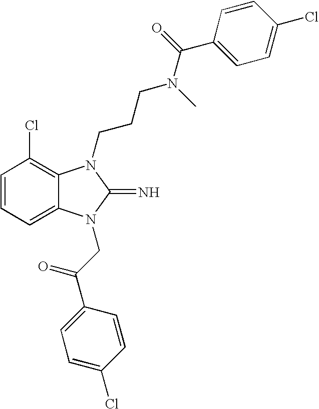 | D116 | 6.21 min (a) | (M + H)⁺529.1, 531.1 (1:1) |
| 7-Chloro-2- imino-1-(3- methylamino- o-propyl)- 2,3-dihydro- 1H-benzo- imidazole (B, C, D, M) | 2-Methoxy- benzoyl chloride | 2-Bromo- 1-(4- chloro- phenyl)- ethanone | 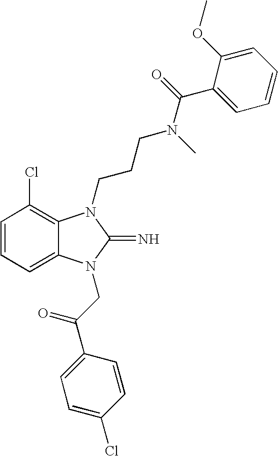 | D117 | 5.97 min (a) | (M + H)⁺525.1, 527.1 (3:2) |
| 7-Chloro-2- imino-1-(3- methylamino- o-propyl)- 2,3-dihydro- 1H-benzo- imidazole (B, C, D, M) | 3-Methoxy- benzoyl chloride | 2-Bromo- 1-(4- chloro- phenyl)- ethanone | 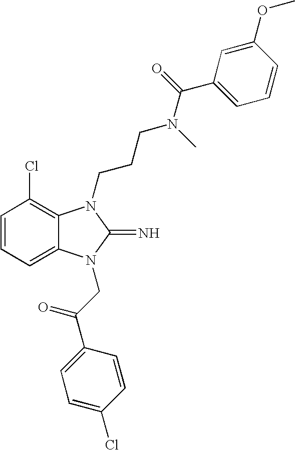 | D118 | 6.04 min (a) | (M + H)⁺525.1, 527.1 (3:2) |
| 7-Chloro-2- imino-1-(3- methylamino- o-propyl)- 2,3-dihydro- 1H-benzo- imidazole (B, C, D, M) | 4-Methoxy- benzoyl chloride | 2-Bromo- 1-(4- chloro- phenyl)- ethanone | 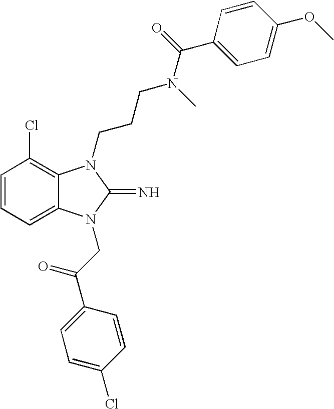 | D119 | 6.00 min (a) | (M + H)⁺525.0, 527.1 (3:2) |
| 7-Chloro-2- imino-1-(3- methylamino- o-propyl)- 2,3-dihydro- 1H-benzo- imidazole (B, C, D, M) | 3-Cyano- benzoyl chloride | 2-Bromo- 1-(4- chloro- phenyl)- ethanone | 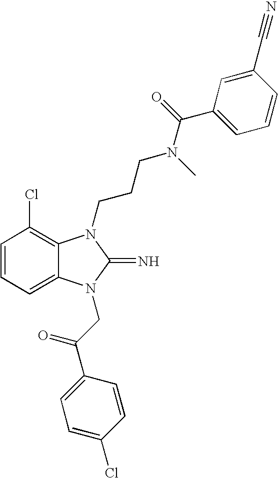 | D120 | 5.82 min (a) | (M + H)⁺520.1, 522.2 (3:2) |
| 7-Chloro-2- imino-1-(3- methylamino- o-propyl)- 2,3-dihydro- 1H-benzo- imidazole (B, C, D, M) | 4-Cyano- benzoyl chloride | 2-Bromo- 1-(4- chloro- phenyl)- ethanone | 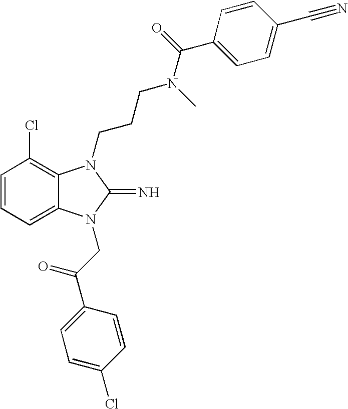 | D121 | 5.79 min (a) | (M + H)⁺520.1, 522.1 (3:2) |
| 2-Imino-1-(3- methylamino- o-propyl)- 2,3-dihydro- 1H-benzo- imidazole (B, C, D, M) | Benzoyl chloride | 2-Bromo- 1-phenyl)- ethanone | 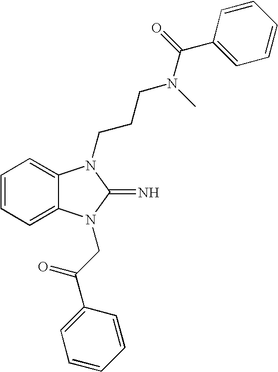 | D122 | 5.45 min (a) | (M + H)⁺427.2 |
| 2- Imino-1-(3- methylamino- propyl)- 2,3-dihydro- 1H-benzo- imidazole (B, C, D, M) | Benzoyl chloride | 2-Bromo- 1-(4- bromo- phenyl)- ethanone | 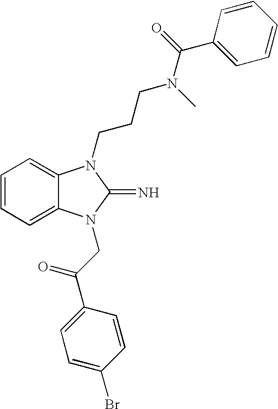 | D123 | 5.83 min (a) | (M + H)⁺505.1, 507.1 (1:1) |
| 2- Imino-1-(3- methylamino- propyl)- 2,3-dihydro- 1H-benzo- imidazole (B, C, D, M) | Benzoyl chloride | 2-Bromo- 1-(4- chloro- phenyl)- ethanone | 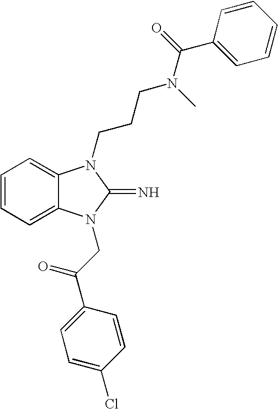 | D124 | 5.78 min (a) | (M + H)⁺461.1, 463.1 (3:1) |
| 7-Chloro-2- imino-1-(3- methylamino- o-propyl)- 2,3-dihydro- 1H-benzo- imidazole (B, C, D, M) | Isocyanato- benzene | 2-Bromo- 1-(4- chloro- phenyl)- ethanone | 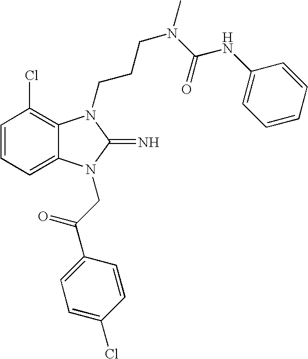 | D125 | 5.91 min (a) | (M + H)⁺510.0, 512.0 (3:2) |
| 7-Chloro-2- imino-1-(3- methylamino- o-propyl)- 2,3-dihydro- 1H-benzo- imidazole (B, C, D, M) | Morpholine- 4-carbonyl chloride | 2-Bromo- 1-(4- chloro- phenyl)- ethanone | 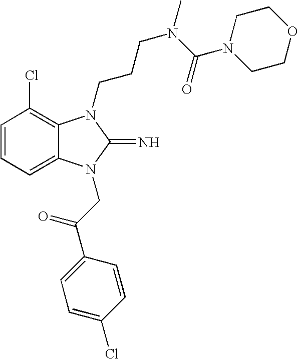 | D126 | 5.55 min (a) | (M + H)⁺504.2, 506.1 (3:2) |
| N-[4-(2- Amino-7- chloro- benzo- imidazol-1- yl)-butyl]- benzamide (B, C, D, M, N, E) | Benzoyl chloride | 2-Bromo- 1-(4- chloro- phenyl)- ethanone | 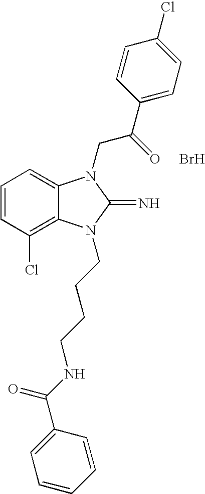 | D127 | 5.94 min (a) | 509.5, 511.5 (M + H)⁺ |
| N-[4-(2- Amino-7- chloro- benzo- imidazol-1- yl)-butyl]- 2-phenyl- acetamide (B, C, D, M, N, E) | Phenyl- acetyl chloride | 2-Bromo- 1-(4- chloro- phenyl)- ethanone | 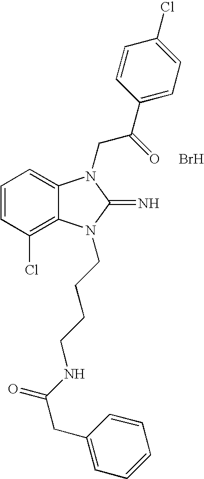 | D128 | 6.08 min (a) | 523.2, 525.1, 526.2 (M + H)⁺ |
| [3-(2-Amino- 7-chloro- benzo- imidazol-1- ylmethyl)- piperidin-1- yl]-phenyl- methanone (B, C, D, M, N, E) | Benzoyl chloride | 2-Bromo- 1-(4- chloro- phenyl)- ethanone | 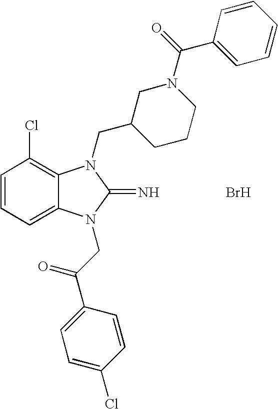 | D129 | 6.01 min (a) | 521.1, 523.2, 525.1 (M + H)⁺ |
| [3-(2-Amino- 7-chloro- benzo- imidazol-1- ylmethyl)- piperidin-1- yl]-phenyl- methanone (B, C, D, M, N, E) | Benzoyl chloride | 2-Bromo- 1-(4- bromo- phenyl)- ethanone | 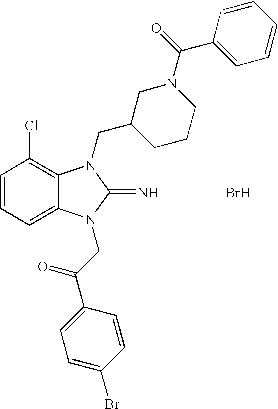 | D130 | 6.07 min (a) | 565.1, 567.1, 568.2, 569.1 (M + H)⁺ |
| N-[4-(2- Amino-7- chloro- benzo- imidazol-1- yl)-butyl]- 2-phenyl- acetamide (B, C, D, M, N, E) | Benzoyl chloride | 2-Bromo- 1-(4- chloro- phenyl)- ethanone | 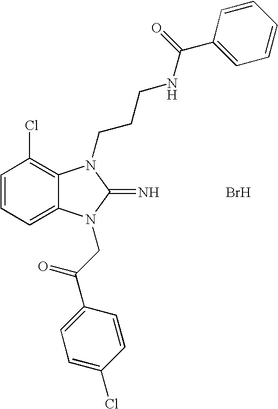 | D131 | 5.86 min (a) | 479.1, 481.1, 482.1, 483.2 (M + H)⁺ |
| N-[4-(2- Amino-7- chloro- benzo- imidazol-1- yl)-butyl]- 2-phenyl- acetamide (B, C, D, M, N, E) | 4-chloro- benzoyl chloride | 2-Bromo- 1-(4- chloro- phenyl)- ethanone | 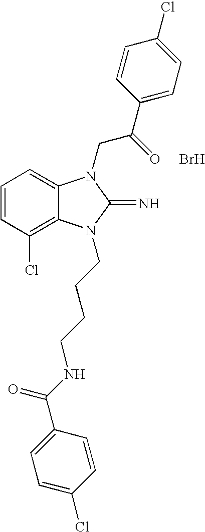 | D132 | 7.13 min (a) | 543.2, 545.1, 547.1 (M + H)⁺ |

General Procedure O: Formation of a N-1,N-3-diacetamide-substituted 2-aminobenzimidazole.

To a solution of an amine in an organic solvent (preferably ether) is added about 0.5 to 5.0 equivalents of an organic base (preferably diisopropylethylamine, preferably 1.0 equivalents) followed by about 0.5 to 2.0 equivalents of chloroacetyl chloride (preferably 1.0 equivalents). The reaction mixture is stirred at about 0° C. to 50° C. (preferably room temperature) for about 4 hours. The reaction mixture is filtered and concentrated in vacuo. The crude reaction mixture is dissolved in an organic solvent (preferably DMF) and about 0.5 to 3.0 equivalents of 1H-benzoimidazol-2-ylamine (preferably 1.0 equivalents) was added. After about 20 hours, an organic solvent (preferably ethyl acetate) is added and the reaction mixture is filtered. The crude product can be purified by chromatography.

Illustration of General Procedure O

### Preparation #13: 2-{2-Imino-3-[(methyl-phenyl-carbamoyl)-methyl]-2,3-dihydro-benzoimidazol-1-yl}—N-methyl-N-phenyl-acetamide

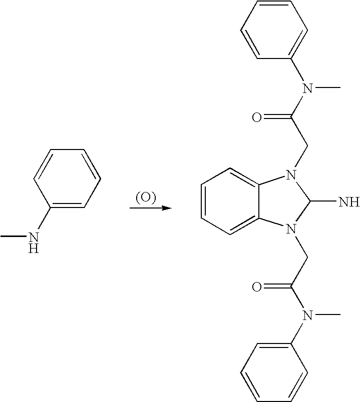

To a solution of methylphenylamine (536 mg, 5.0 mmol) and diisopropylethylamine (565 mg, 5.0 mmol) in diethyl ether (50 mL) at room temperature was added chloroacetyl chloride (565 mg, 5.0 mmol) dropwise. After stirring for about 4 hours the reaction mixture was filtered and concentrated in vacuo. The crude reaction mixture was dissolved in DMF (25 mL) and 1H-benzoimidazol-2-ylamine (666 mg, 5.0 mmol) was added. After stirring for about 20 hours ethyl acetate (50 mL) was added and the reaction mixture was filtered to provide 28 mg of 2-{2-imino-3-[(methyl-phenyl-carbamoyl)-methyl]-2,3-dihydro-benzoimidazol-1-yl}-N-methyl-N-phenyl-acetamide as a white solid. RP-HPLC Rₜ 5.45 min. (table 1, method a). LC/MS (M+H)⁺ 428.2.

TABLE E
Examples prepared using general procedure O

| | | | | | m/z |
|---|---|---|---|---|---|
| | | | | | or ¹H NMR |
| | | | | Rₜ/min | (d₆ DMSO, |
| Starting Material | | Product | Example # | (method) | 400 MHz) |
| 1H- Benzoimidazol-2- ylamine | 2-Chloro-N- methyl-N-phenyl- acetamide | 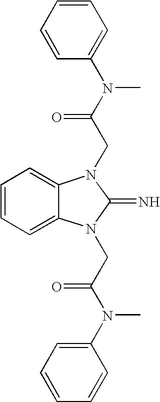 | E1 | 5.48 min (a) | (M + H)⁺428.2 |

General Procedure P: Cross coupling of a 7-Halo-2-aminobenzimidazole. A solution of the 7-halo-benzimidazole (preferably the bromo or chloro), about 0.01 to 1.0 equivalents of a palladium salt (preferably Pd₂(dba)₃, preferably 0.05 equivalents), about 0.01 to 1.0 equivalents of a phosphine ligand (preferably tBu₃PHBF₄, preferably 0.10 equivalents) and about 1 to 5 equivalents of a base (preferably Na₂CO₃, preferably 3 equivalents) in a solvent mixture containing an organic solvent (preferably dioxane) and a protic solvent (preferably water) is degassed by bubbling nitrogen through it for about 5 minutes. In the case of alkynyl coupling partners, about 0.01 to 1.0 equivalents (preferably 0.10 equivalents) of a copper salt (preferably CuI) is also added. About 1 to 10 equivalents of the coupling partner is then added (preferably a boronic acid or boronate, preferably 2 equivalents) and the reaction mixture is heated to about 100° C. After about 15 hours the reaction mixture is cooled to about room temperature, filtered and concentrated to provide the coupled product that can be used directly or purified by chromatography.

Illustration of General Procedure P

### Preparation #14: 1-Methyl-7-vinyl-1H-benzoimidazol-2-ylamine

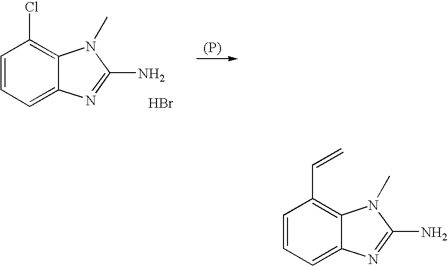

A solution of 7-Chloro-1-methyl-1H-benzoimidazol-2-ylamine (150 mg, 0.57 mmol), Pd₂(dba)₃) (26 mg, 0.029 mmol), tBu₃PHBF₄ (16 mg, 0.057 mmol) and Na₂CO₃(182 mg, 1.7 mmol) in dioxane (2.0 mL) and water (0.5 mL) was degassed by bubbling nitrogen through it for about 5 minutes. 4,4,5,5-Tetramethyl-2-vinyl-[1,3,2]dioxaborolane (194 uL, 1.14 mmol) was then added and the reaction mixture was heated to about 100° C. After about 15 hours the reaction mixture was cooled to about room temperature, filtered and concentrated to provide 247 mg of a crude mixture containing 1-Methyl-7-phenyl-1,3-dihydro-benzoimidazol-2-ylideneamine that was used in subsequent reactions without further purification. RP-HPLC Rₜ 4.38 min. (table 1, method a); m/z: (M+H)⁺ 174.3.

General Procedure Q: Reduction of an alkene. To a solution of an alkene in an organic solvent (preferably ethanol) at about room temperature is added about 0.01 to 1.0 equivalents of palladium on carbon (preferably 0.05 equivalents). Hydrogen gas is bubbled through the solution for about 5 minutes and a hydrogen atmosphere is maintained with a balloon. After about 20 hours the reaction mixture is filtered and concentrated in vacuo. The crude product can be used directly or purified by chromatography.

Illustration of General Procedure Q

### Preparation #15: 1-Methyl-7-pentyl-1H-benzoimidazol-2-ylamine

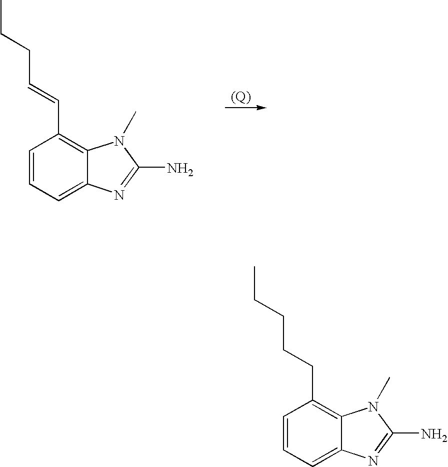

To a solution of 1-Methyl-7-((E)-pent-1-enyl)-1H-benzoimidazol-2-ylamine (135 mg, 0.63 mmol) in ethanol (6.3 mL) at about room temperature was added 10% palladium on carbon (26 mg, 0.24 mmol). Hydrogen gas was bubbled through the solution for about 5 minutes and a hydrogen atmosphere was maintained with a balloon. After about 20 hours the reaction mixture was filtered and concentrated in vacuo to provide 80 mg of 1-Methyl-7-pentyl-1,3-dihydro-benzoimidazol-2-ylideneamine as a white solid. RP-HPLC Rₜ 5.49 min. (table 1, method a); m/z: (M+H)⁺ 218.2.

General Procedure R: Acylation of an amine followed by hydrolysis of a nitrile. To a solution of a 2-aminobenzimidazole containing a tethered secondary amine in an organic solvent (preferably methylene chloride) at about −78° C. to 50° C. (preferably 0° C.) is added about 1.0 to 10.0 equivalents of diisopropylethylamine (preferably 5.0 equivalents) followed by about 0.5 to 2.0 equivalents of a solution of an acylating agent (preferably 1.1 equivalents) in an organic solvent (preferably methylene chloride). After the addition, the reaction mixture is allowed to warm to room temperature and stirred for about 3 hours. The reaction mixture is concentrated in vacuo and the crude mixture is dissolved in a mixture of a protic solvent (preferably water) containing an inorganic base (preferably NaOH) and an organic solvent (preferably dioxane). The reaction mixture is heated at about 25° C. to 150° C. (preferably 80° C.) for about 3 hours. The reaction mixture is allowed to reach room temperature and taken through an aqueous work up. The crude product can be purified by chromatography.

Illustration of General Procedure R

### Preparation #16: N-[3-(2-Amino-7-chloro-benzoimidazol-1-yl)-propyl]—N-methyl-isophthalamide

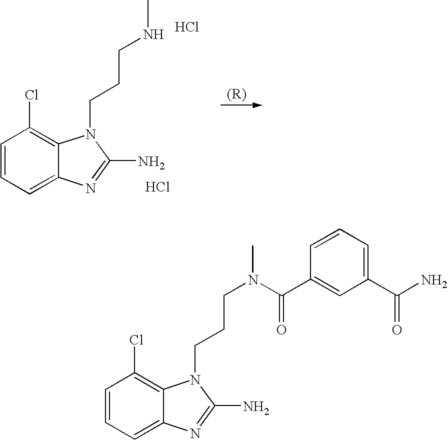

To a solution of 7-Chloro-1-(3-methylamino-propyl)-1H-benzoimidazol-2-ylamine (115 mg, 0.48 mmol) in methylene chloride (5 mL) at about 0° C. was added diisopropylethylamine (0.167 mL, 0.96 mmol) followed by 3-Cyano-benzoyl chloride (80 mg, 0.48 mmol). After the addition the reaction mixture was allowed to warm to room temperature and stirred for about3 hours. The reaction mixture was concentrated in vacuo and the crude mixture was dissolved in a 1:1 mixture of dioxane/2N NaOH (5mL). After stirring for about 3 hours at about 80° C. the reaction mixture was diluted with ethyl acetate and taken through an aqueous work up. Purification by RP-HPLC provided 35 mg of N-[3-(2-Amino-7-chloro-benzoimidazol-1-yl)-propyl]—N-methyl-isophthalamide. RP-HPLC Rₜ 4.39 min. (table 1, method a); m/z: (M+H)⁺ 386.1.

General Procedure S: Acylation of an amine. To a solution of a 2-aminobenzimidazole in an organic solvent (preferably methylene chloride or THF, or mixtures of methylene chloride and THF) at about room temperature is added about 0 to 5 equivalents of an organic base (preferably diisopropylethylamine, preferably 0 or 2 equivalents) followed by an acylating reagent. After completion of the reaction the reaction mixture is taken through an aqueous work up or concentrated in vacuo. The crude product can be purified by chromatography.

Illustration of General Procedure S

### Preparation #17: [1-Methyl-7-vinyl-1,3-dihydro-benzoimidazol-(2E)-ylidene]-carbamic acid benzyl ester

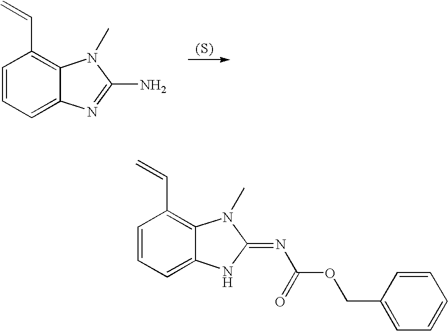

To a solution of 1-Methyl-7-vinyl-1H-benzoimidazol-2-ylamine (600 mg, 3.5 mmol) in a 1:1 mixture of THF and methylene chloride at room temperature was added 1,3-Dioxo-1,3-dihydro-isoindole-2-carboxylic acid benzyl ester (864 mg, 3.5 mmol). After about 24 hours the reaction mixture was concentrated in vacuo. The crude product was purified by chromatography on silica gel (gradient elution 3:7 to 2:3 ethyl acetate:heptane) to provide 200 mg of [1-Methyl-7-vinyl-1,3-dihydro-benzoimidazol-(2E)-ylidene]-carbamic acid benzyl ester as an oil. RP-HPLC Rₜ 6.71 min. (table 1, method a); m/z: (M+H)⁺ 308.0.

TABLE F
Examples prepared using general procedure S

| | | | | m/z |
|---|---|---|---|---|
| | | | | or ¹H NMR |
| | | Example | Rₜ/min | (d₆ DMSO, |
| Starting Material | Product | # | (method) | 400 MHz) |
| 1-(4-Chloro- phenyl)-2-(2- imino-3-methyl- 2,3-dihydro- benzoimidazol- 1-yl)-ethanone (E) | 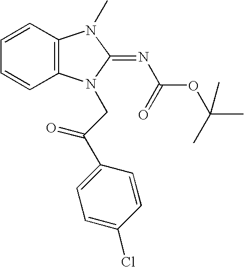 | F1 | 5.9(a) | 400.0 (M + H)⁺ |
| 1-(4-Chloro- phenyl)-2-(2- imino-3-methyl- 2,3-dihydro- benzoimidazol- 1-yl)-ethanone (E) | 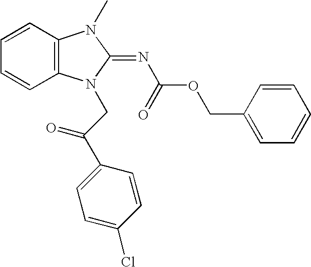 | F2 | 6.0(a) | 434 (M + H)⁺ |
| 1-(4-Chloro- phenyl)-2-(2- imino-3-methyl- 2,3-dihydro- benzoimidazol- 1-yl)-ethanone (E) | 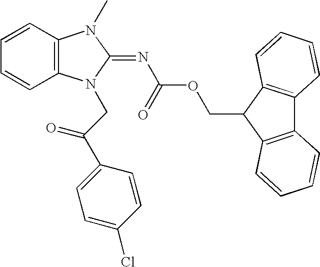 | F3 | 7.4(a) | 522.3 (M + H)⁺ |

General Procedure T: Cyclopropanation of an alkene. To a solution of about 1 to 20 equivalents of diethyl zinc (preferably 10 equivalents) in an organic solvent (preferably methylene chloride) at about −78° C. to about room temperature (preferably 0° C.) is added about 1 to 20 equivalents of an organic acid (preferably trifluroacetic acid, preferably 10 equivalents) in an organic solvent (preferably methylene chloride). After about 10 minutes about 1 equivalent to 20 equivalents of diiodomethane (preferably 10 equivalents) in an organic solvent (preferably methylene chloride) is added. After about 10 minutes the alkene is added in an organic solvent (preferably methylene chloride) and the reaction mixture is allowed to warm to about room temperature. After about 20 hours the reaction is quenched with aqueous acid (preferably 1N HCl) and taken through an aqueous work up. The crude product can be purified by flash chromatography.

Illustration of General Procedure T

### Preparation #18: [7-Cyclopropyl-1-methyl-1,3-dihydro-benzoimidazol-(2E)-ylidene]-carbamic acid benzyl ester

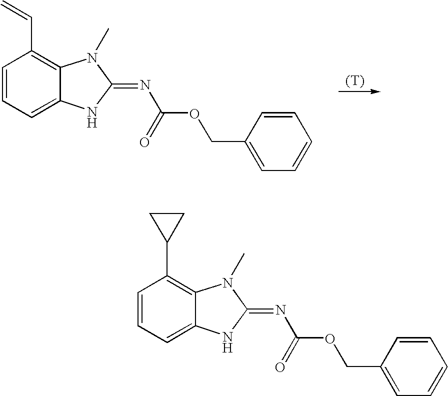

To a 1 M solution of diethyl zinc (2.3 mL, 2.3 mmol) in methylene chloride at about 0° C. was added a solution of trifluroacetic acid (177 uL, 2.3 mmol) in methylene chloride (2.0 mL). After about 10 minutes a solution of diiodomethane (186 uL, 2.3 mmol) in methylene chloride (2.0 mL) was added. After about 10 minutes a solution of [1-Methyl-7-vinyl-1,3-dihydro-benzoimidazol-(2E)-ylidene]-carbamic acid benzyl ester (70 mg, 0.23 mmol) in methylene chloride (2.0 mL) was added and the reaction mixture was allowed to warm to room temperature. After about 20 hours the reaction was quenched with aqueous acid (preferably 1N HCl) and taken through an aqueous work up. The crude product was purified by chromatography on silica gel (eluting with 2:3 ethyl acetate:heptane) to provide 40 mg of [7-Cyclopropyl-1-methyl-1,3-dihydro-benzoimidazol-(2E)-ylidene]-carbamic acid benzyl ester as an oil. RP-HPLC Rₜ 6.87min. (table 1, method a); m/z: (M+H)⁺ 322.1.

General Procedure U: Removal of a CBz group. To a solution of a benzyl carbamate in an organic solvent (preferably methanol) at about room temperature is added about 0.01 to 1.0 equivalents of palladium on carbon (preferably 0.05 equivalents). Hydrogen gas is bubbled through the solution for about 5 minutes and a hydrogen atmosphere is maintained with a balloon. After about 2 hours the reaction mixture is filtered and concentrated in vacuo. The crude product can be used directly or purified by chromatography.

Illustration of General Procedure U

### Preparation #19: 7-Cyclopropyl-1-methyl-1H-benzoimidazol-2-ylamine

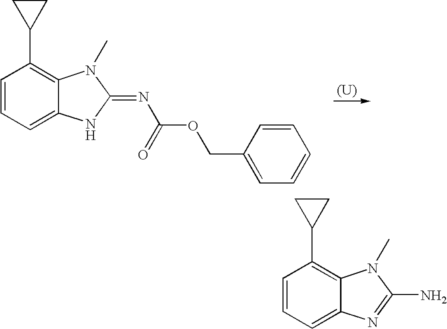

To a solution of [7-Cyclopropyl-1-methyl-1,3-dihydro-benzoimidazol -(2E)-ylidene]-carbamic acid benzyl ester (40 mg, 0.12 mmol) in methanol (2.4 mL) at room temperature was added palladium on carbon (6 mg, 0.006 mmol). Hydrogen gas was bubbled through the solution for about 5 minutes and a hydrogen atmosphere was maintained with a balloon. After about 2 hours the reaction mixture was filtered and concentrated in vacuo to provide 20 mg of 7-Cyclopropyl-1-methyl-1H-benzoimidazol-2-ylamine as an oil. RP-HPLC Rₜ 4.60 min. (table 1, method a); m/z: (M+H)⁺ 188.2.

General Procedure V: Synthesis of a nitro-phenylamino-propionic acid. About 1 to 20 equivalents of the amine (preferably 2.25 equivalents), about 0 to 5 equivalents of an organic base (preferably triethylamine, preferably 2 equivalents) and a halo-nitrobenzene (preferably 1 equivalent) are combined in an organic solvent (preferably ethanol) or neat. The reaction mixture is stirred at about 0° C. to 200° C. (preferably 100° C.) for about 1 to 10 days (preferably 3 days). To the reaction mixture is added and ethereal solvent (preferably diethyl ether) and 1 to 20 equivalents of an aqueous hydroxide salt (preferably NaOH) solution (preferably 2.2 equivalents). The reaction mixture is stirred at about 0° C. to 40° C. (preferably 25° C.) for about 1 to 24 hours (preferably 1 hour). The organic layer is separated and treated with 1 to 10 equivalents of a protic acid (preferably HCl in diethyl ether or glacial acetic acid solution, preferably 2 equivalents) and concentrated in vacuo.

Illustration of General Procedure V

### Preparation #20: 3-(2-Nitro-phenylamino)-propionic acid

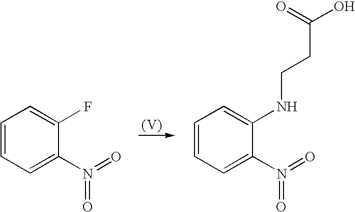

2-Fluoro-nitrobenzene (2.28 g, 16.2 mmol), glycine ethyl ester hydrochloride (2.49 g, 16.2 mmol), triethylamine (1.8 mL, 10.4 mmol), and 81 mL of ethanol were combined and heated to about 80° C. After about 2 days, the reaction mixture was allowed to cool to room temperature and 81 mL of diethyl ether and 36 mL of 1N NaOH solution were added. The resulting mixture was allowed to stir at room temperature for about 1 hour. The organic phase was separated and treated with 30 mL of 1M HCl solution in diethyl ether. After about 1 hour, the solution was concentrated to provide 3.44 g of the desired product as an orange solid that was used in subsequent reactions without further purification. RP-HPLC Rₜ 2.55 min. (table 1, method g); m/z: (M+HCO₂H)⁺ 254.8.

General Procedure W: Conversion of a carboxylic acid to a carboxamide. About 1 to 20 equivalents of the amine (preferably 1.2 equivalents), about 0 to 5 equivalents of an organic base (preferably diisopropylethylamine, preferably 2 equivalents), a peptide coupling reagent (preferably O-benzotriazole-N,N,N′,N′-tetramethyl-uronium-hexafluoro-phosphate), and a carboxylic acid (preferably 1 equivalent) are combined in an organic solvent (preferably methylene chloride). The reaction mixture is stirred at about 0° C. to 45° C. (preferably 25° C.) for about 1 to 72 hours (preferably 4 hours). The reaction mixture is washed with an aqueous basic solution (preferably sodium carbonate) and concentrated in vacuo; alternatively the solution is filtered through celite and concentrated in vacuo.

Illustration of General Procedure W

### Preparation #21: N-Benzyl-N-methyl-3-(2-Nitro-phenylamino)-propionamide

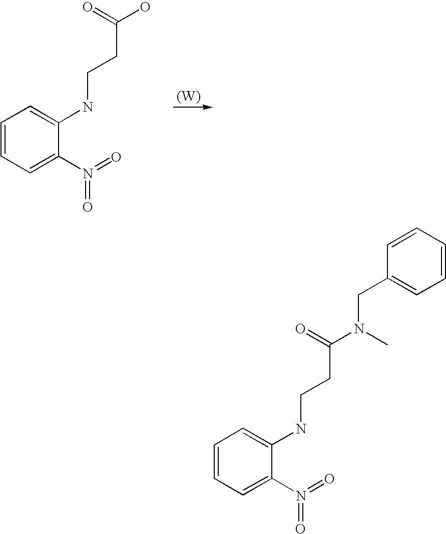

3-(2-Nitro-phenylamino)-propionic acid (0.105 g, 0.500 mmol), N-methyl-benzylamine (0.073 g, 0.60 mmol), diisopropylethylamine (0.17 mL, 1.0 mmol), O-benzotriazole-N,N,N′,N′-tetramethyl-uronium-hexafluoro-phosphate (0.226 g, 0.600 mmol), and 1.25 mL of methylene chloride were combined and stirred at about 25° C. After about 4 hours, the reaction mixture was filtered through a pad of celite with the aid of methylene chloride and concentrated to provide a yellow oil that was used in subsequent reactions without further purification. RP-HPLC Rₜ 2.03 min. (table 1, method d); m/z: (M+H)⁺ 314.2.

General Procedure X: Reduction of an amide to an amine. To a solution of an amide in an organic solvent (preferably THF or diethyl ether) at about room temperature is added about 1 to 10 equivalents of lithium aluminum hydride (preferably about 4 equivalents). After about 2 hours water is added followed by an aqueous solution of NaOH and finally additional water. The resulting slurry is filtered and concentrated in vacuo. The crude product can be purified by chromatography.

Illustration of General Procedure X

### Preparation #22: (4-Bromo-phenyl)-[2-(2-imino-3-methyl-2,3-dihydro-benzoimidazol-1-yl)-ethyl]-amine

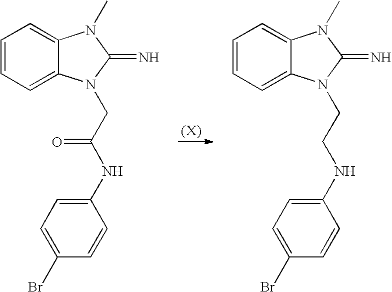

To a solution of N-(4-Bromo-phenyl)-2-(2-imino-3-methyl-2,3-dihydro-benzoimidazol-1-yl)-acetamide (85 mg, 0.22 mmol) in THF (2 mL) at room temperature was added a 2 M solution of lithium aluminum hydride (400 uL, 0.8 mmol). After about 2 hours water (400 uL) was added, followed by a 2N solution of NaOH (400 uL), followed by water (800 uL). The resulting slurry was filtered and concentrated in vacuo. The crude product was purified by RP-HPLC to provide 10 mg of (4-Bromo-phenyl)-[2-(2-imino-3-methyl -2,3-dihydro-benzoimidazol-1-yl)-ethyl]-amine as an oil. RP-HPLC Rₜ 5.48 min. (table 1, method a); m/z: (M+H)⁺ 345.2, 347.2 (1:1).

General Procedure Y: Formation of a cyanoguanidine. To a solution of a diamine in an organic solvent (preferably acetonitrile) is added about 1.0 equivalent to 5 equivalents of diphenyl cyanocarbonimidate (preferably 1 equivalent). The reaction mixture is heated to about 20° C. to 200° C. (preferably 80° C.). Upon completion of the reaction the reaction mixture is cooled to room temperature and the product is isolated by filtration or chromatography.

Illustration of General Procedure Y

### Preparation #23: 1-Methyl-1,3-dihydro-benzoimidazol-(2E)-ylidene-cyanamide

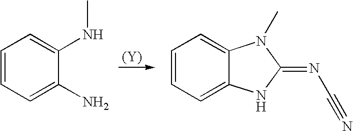

To a solution of N-Methyl-benzene-1, 2 -diamine (283 uL, 2.5 mmol) in acetonitrile (2 mL) was added diphenyl cyanocarbonimidate (596 mg, 2.5 mmol). The reaction mixture was heated to about 80° C. After about 6 hours the reaction mixture was cooled to room temperature and filtered to provide 235 mg of 1-Methyl-1,3-dihydro-benzoimidazol-(2E)-ylidene-cyanamide as a white solid. RP-HPLC Rₜ 4.81 min. (table 1, method a); m/z: (M+H)⁺ 173.2.

General Procedure Z: Ullmann coupling of an aryl bromide. To a solution of an aryl bromide in an organic solvent (preferably DMF) is added a copper salt (preferably copper iodide), followed by an alkoxide base (preferably sodium methoxide in methanol). The solution is heated at about room temperature to about 200° C. (preferably 140° C.) for about 10 minutes. After cooling to about room temperature the reaction mixture is diluted with water and heated at about room temperature to about 200° C. (preferably 150° C.) for about 20 minutes. The crude product is taken through an aqueous work up and can be purified by silica gel chromatography.

Illustration of General Procedure Z

### Preparation #24: 7-Methoxy-1-methyl-1H-benzoimidazol-2-ylamine

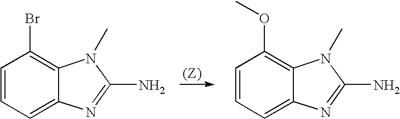

To a solution of 7-Bromo-1-methyl-1,3-dihydro-benzoimidazol-2-ylideneamine hydrobromide (50 mg, 0.16 mmol) in DMF (1.6 mL) was added copper iodide (78 mg, 0.41 mmol), followed by a 3.4 M solution of sodium methoxide in methanol (471 uL, 1.6 mmol). The solution was heated at about room temperature to about 140° C. for about 10 minutes in a single-mode microwave reactor. After cooling to room temperature the reaction mixture was diluted with water and heated at about room temperature to about 150° C. for about 20 minutes. Ethyl acetate and water was added to the crude reaction mixture and the resulting layers were separated. The organic layer was washed with brine, dried (Na₂SO₃), filtered and concentrated in vacuo to provide 20 mg of 7-Methoxy-1-methyl-1H-benzoimidazol-2-ylamine as an oil that was used without further purification. RP-HPLC Rₜ 4.18 min. (table 1, method a); m/z: (M+H)⁺ 178.1.

General Procedure AA: Formation of a protected 2-aminobenzimidazole. To a solution of the 1,3-bis(benzyloxycarbonyl)-1-methyl-2-thiopseudourea in a protic solvent (preferably isopropanol) is added an appropriate diamine (1 equivalent) and p-toluene sulfonic acid monohydrate (0.1 equivalent). After heating to about 0 to 100° C. (preferably 65° C.) for about 16 hours the reaction is concentrated in vacuo. The crude mixture is diluted with an organic solvent (preferably ethyl acetate) and the product was isolated by filtration or obtained through an aqueous work up.

Illustration of General Procedure AA

### Preparation of #25: [7-Chloro-1-(2-dibenzylamino-ethyl)-1H-benzoimidazol-2-yl]-carbamic acid benzyl ester

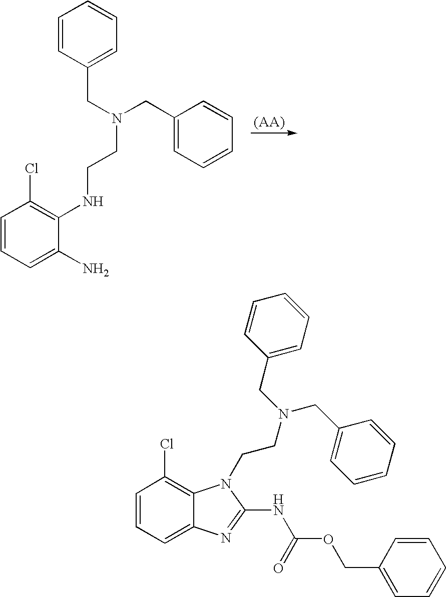

To a solution of 1,3-bis(benzyloxycarbonyl)-methyl-2-thiopseudourea (397 mg, 1.1 mmol) in isopropanol (6 mL) was added 3-Chloro-N2-(2-dibenzylamino-ethyl)-benzene-1, 2 -diamine (405 mg, 1.1 mmol) and p-toluene sulfonic acid (20 mg). The reaction was heated at about 65° C. for about 16 hours. The solvents were removed under vacuum and ethyl acetate was added. The ethyl acetate was washed with 1 N sodium hydroxide. The aqueous layer was extracted with ethyl acetate. The combined ethyl acetate extracts were washed with brine and dried over sodium sulfate. The mixture was filtered and the solvent evaporated to give a mixture of the isopropyl and benzyl carbamates which was used without further isolation in the next reaction. RP-HPLC Rₜ 2.80, 2.88 min. (table 1, method c).

General Procedure BB: Deprotection of a Carbamate Protected 2-Aminobenzimidazole. To a solution of the carbamate protected 2-aminobenzimidazole is added 33% HBr in acetic acid. After stirring at ambient temperature for about 16 hours the reaction is heated at about 25 to 85° C. (preferably 45° C. ) for about 15 to 144 hours (preferably 72 hours). The solvents are removed in vacuo and the residue treated with 10% NaOH. Aqueous work up and trituration with non-polar solvents (preferably ether and heptane) provide the product as a tan solid.

Illustration of General Procedure BB

### Preparation of #26: 7-Chloro-1-(2-dibenzylamino-ethyl)-1H-benzoimidazol-2-ylamine

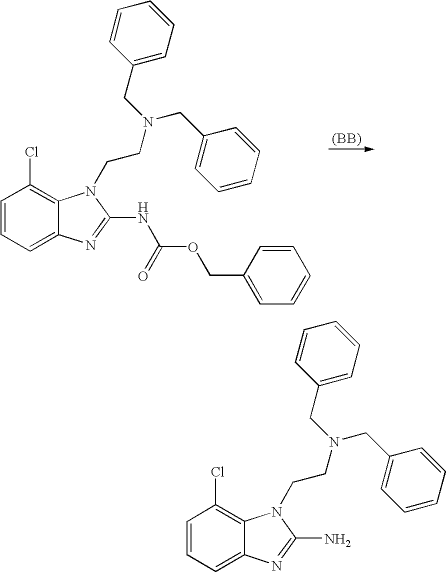

The mixture of benzyl and isopropyl carbamate (1.1 mmol) was dissolved in 33% HBr in acetic acid (10 mL) and stirred at ambient temperature overnight. The reaction was then heated at about 25 to 85° C. (preferably 45° C. ) for about 15 to 144 hours (preferably 72 hours) and the solvent removed in vacuo. The residue was stirred in 10% sodium hydroxide and extracted with ethyl acetate (2x). The combined ethyl acetate extracts were washed with brine, dried over sodium sulfate, filtered, and concentrated. Trituration with ether and heptanes provided the desired 2-aminobenzimidazole (184 mg, 42%) as a tan solid. RP-HPLC Rₜ 7.224 min (table 1, method a).

General Procedure CC: Reductive Amination of an Amine with an Aldehyde.

A suspension of about 1 to 10 equivalents of an amine (preferably 1 equivalent) and about 1 to 10 equivalents of an aldehyde (preferably 1 equivalent) in an organic solvent (for example 1, 2 -dichloroethane, acetonitrile, or methanol, preferably methanol) with about 0.1 to 5 equivalents of an organic acid (preferably 0.1 equivalents of acetic acid) is stirred for about 1 to3 hours (preferably 1 hour) at ambient temperature. To the resulting mixture is added about 1 to 10 equivalents of the reducing agent (preferably sodium borohydride, polymer-bound, preferably about 2.0 equivalents). The reaction is allowed to stir at ambient temperature for about 1 to 24 hours (preferably 18 hours). The resulting mixture is filtered and the filtrate is stirred with about 3 to 10 equivalents of p-toluenesulfonic acid, polymer-bound (preferably 3 equivalents) for about 0.5 to 5 hours (preferably 1 hour). The resulting mixture is then filtered and the scavenger resin is rinsed with methanol. The combined filtrate is discarded. The resin is then washed with 2M solution of ammonia in methanol. The resulting filtrate is concentrated then used directly in the next steps without further purification.

Illustration of General Procedure CC

### Preparation #27: 2-[Methyl-(4-methyl-benzyl)-amino]-ethanol

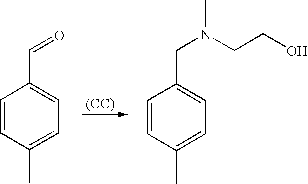

To a suspension of p-tolualdehyde (0.240 g, 2.0 mmol) and 2-(methylamino)-ethanol (0.160 mL, 2.0 mmol) in methanol (10 mL) was added acetic acid (0.011 mL, 0.2 mmol). The resulting mixture was stirred at ambient temperature for about 2 hours. Sodium borohydride, polymer-bound (1.0 g, 4.0 mmol) was added and the reaction was allowed to stir at ambient temperature for 18 hours. The resulting mixture was filtered and the resin rinsed with methanol. The filtrate was stirred with p-toluenesulfonic acid, polymer-bound (1.43 g, 6.0 mmol) for 1.5 hours. The mixture was then filtered and the scavenger resin was again rinsed with methanol. The combined filtrate was discarded and the resin was washed many times with 2M solution of ammonia in methanol. The combined filtrate was concentrated and dried to yield 2-[Methyl-(4-methyl-benzyl)-amino]-ethanol (0.167 g, 0.9 mmol)): RP-HPLC Rₜ 3.8 min (Table 1, Method a), that was used directly in the next step without further purification.

General Procedure DD: Conversion of a primary alcohol to a chloride.

A suspension of an alcohol compound (preferably 1 equivalent) in about 5 to 50 equivalents of thionyl chloride (preferably 20 equivalents) is allowed to stir at temperature of about 25° C. to 80° C. (preferably 70° C.) for about 1 to 10 hours (preferably 1 hour). The result mixture is concentrated in vacuo to afford the product, which is used directly in the next step without further purification.

Illustration of General Procedure DD

### Preparation #28: (2-Chloroethyl)-methyl-(4-methylbenzyl)-amine hydrochloride

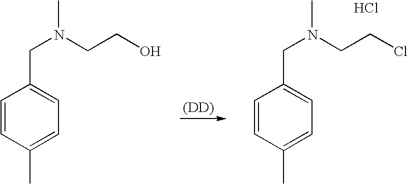

A suspension of 2-[Methyl-(4-methyl-benzyl)-amino]-ethanol (0.167 g, 0.9 mmol) in thionyl chloride (1.5 mL, 20.6 mmol) was heated at about 70° C. for about 1 hour. The reaction was then concentrated in vacuo to yield (2-Chloroethyl)-methyl-(4-methylbenzyl)-amine hydrochloride, (0.218 g, 0.93 mmol): RP-HPLC Rₜ 4.60 min (Table 1, Method a), as crude brown solid.

General Procedure EE: Oxime Formation.

A mixture of a ketone (preferably 1 equivalent) and about 1 to 10 equivalents of hydroxylamine hydrochloride (preferably 5 equivalents) is allowed to stir at about 25° C. to 120° C. (preferably 120° C.) in an organic solvent (for example pyridine, methanol, or water/methanol mixture, preferably pyridine), with or without an acid scavenger (for example MP-carbonate resin), for about 1 to 18 hours (preferably 6 hours). If the acid scavenger is used, the resulting mixture is filtered and the filtrate is concentrated in vacuo. If the acid scavenger is not used, the crude reaction is concentrated in vacuo. The resulting crude material is either purified via chromatography or subjected to extraction with aqueous acid (for example 5% hydrochloric acid solution in water) and organic solvent (for example diethyl ether) to afford the oxime product.

Illustration of General Procedure EE

### Preparation #29: 1-(4-Chloro-2-hydroxyphenyl)ethanone oxime

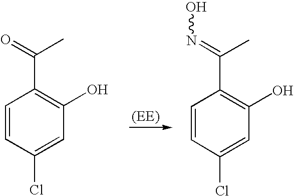

A suspension of 1-(4-Chloro-2-hydroxyphenyl)ethanone (8.7 g, 0.05 mol) and hydroxylamine hydrochloride (17.8 g, 0.25 mol) in pyridine (80 mL) was heated to reflux for about 6 hours. The resulting mixture was concentrated in vacuo. The crude material was taken up in 5% hydrochloric acid solution (200 mL) and extracted with diethyl ether (3×100 mL). The combined organic layer was washed with brine, dried (MgSO₄) and concentrated to yield 1-(4-Chloro-2-hydroxyphenyl)ethanone oxime, (9.7 g, 0.05 mol): RP-HPLC Rₜ 6.13 min (Table 1, Method a), as white solid.

General Procedure FF: Reduction of a carbonyl to a carbanol. To a solution of the carbonyl in an organic solvent (preferably THF or ether) at about room temperature is added about 1 to 10 equivalents of a reducing agent (preferably LAH, preferably 3 equivalents). After completion of the reaction the crude reaction mixture can be quenched with water and taken through an aqueous work up or further diluted with a sodium hydroxide solution and filtered. The crude product can be used directly or purified by chromatography.

Illustration of General Procedure FF

### Preparation #30: (2-Amino-3-methyl-3H-benzoimidazol-4-yl)-methanol

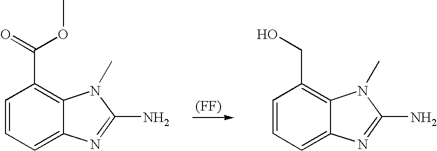

To a solution of (2-Amino-3-methyl-3H-benzoimidazol4-yl)-methanol (150 mg, 0.52 mmol) in THF (10 mL) at room temperature was added a 2.0 M solution of LAH in THF (790 uL, 1.6 mmol). After about 2 hours water (200 uL) was added to the reaction mixture, followed by a solution of 2N NaOH (200 uL), followed by additional water (600 uL). The resulting white granular slurry was filtered and concentrated in vacuo. The crude mixture was dissolved in ethyl acetate and extracted with a solution of 1N HCl. The aqueous layer was made basic with a solution of 2N NaOH and the extracted with ethyl acetate. The organic layer was dried (Na₂SO₄), filtered and concentrated in vacuo to provide 50 mg of (2-Amino-3-methyl-3H-benzoimidazol-4-yl)-methanol as an oil which was used in subsequent reactions without further purification. RP-HPLC Rₜ 2.12 min. (table 1, method a); m/z: (M+H)⁺ 178.2.

What is claimed is:

1. A compound of Formula (I) 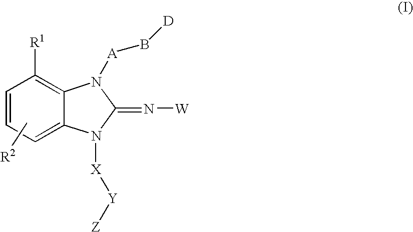
and pharmaceutically acceptable salts, prodrugs and biologically active metabolites thereof wherein
A is selected from the group consisting of a bond, —C(O)—, optionally substituted (C₁-C₆)alkyl and optionally substituted (C₂-C₆)alkenyl;
B is selected from the group consisting of a bond, O, C(O), N(R^{a}), —C(O)—N(R^{a})—, —N(R^{a})—C(O), —CH₂—C(O)—N(R^{a})—, —N(R^{a})—C(O)—CH₂—, —CH₂—N(R^{a})—C(O)—, —C(O)—N(R^{a})—CH₂ and optionally substituted (C₁-C₃)alkyl;
wherein R^{a} is H, CHF₂, (C₁-C₄)alkyl or (C₃-C₆)cycloalkyl;
D is selected from the group consisting of H, halo, OH, CF₃, COOH, (C₁-C₄)alkoxy and dimethylamino; or
D is selected from the optionally substituted group consisting of (C₁-C₆)alkyl, (C₂-C₆)alkenyl, (C₃-C₆)cycloalkyl, —C(O)—OR^{b}, aryl, aryl(C₁-C₄)alkyl, amino, heteroaryl and heterocyclyl;
wherein R^{b} is (C₁-C₄)alkyl, aryl(C₁-C₄)alkyl or aryl;
X is selected from the group consisting of a bond or an optionally substituted (C₁-C₆)alkyl and (C₂-C₄)alkenyl;
Y is selected from the group consisting of a bond, —C(O)—, —NR^{c}, —N(R^{c})—C(O)—, —C(O)—N(R^{c})—, S, optionally substituted (C₃-C₆)alkenyl, —C(O)—N(Q¹)-(CH₂)ₐ, or —N(Q¹)-(CH₂)ₐ or S(O)_{b};
wherein R^{c} is H or (C₁-C₄)alkyl;
wherein Q¹ is H or (C₁-C₄)alkyl;
a is 0, 1 or 2;
b is 1 or 2;
Z is H, or —N(Q²)₂ wherein Q² is (C₁-C₃)alkyl or optionally substituted benzyl; or
Z is selected from the optionally substituted group consisting of (C₂-C₆)alkenyl, (C₁-C₆)alkyl, (C₃-C₆)cycloalkyl, heterocyclyl, aryl, heteroaryl, phenylcarbonylheterocyclyl and phenylcarbonylheteroaryl;
R¹ is selected from the group consisting of H, halo, CF₃, —CH₂—CH₂-Optionally substituted phenyl, —C(O)—OCH₃, (C₁-C₃)alkoxycarbonyl, (C₁-C₂)alkyl-O-phenyl, and (C₁-C₆)alkoxy; or
R¹ is selected from the optionally substituted group consisting of (C₁-C₆)alkyl, (C₂-C₆)alkenyl, (C₃-C₆)cycloalkyl, aryl, aryl(C₁-C₃)alkyl, heteroaryl and heterocyclyl;
R² is one or more substitutents selected from the group consisting of H, CF₃, halo, CN, OCF₃, —C(O)-optionally substituted phenyl, (C₁-C₆)alkoxy and optionally substituted (C₁-C₆)alkyl;
W is H or CN; or
W is selected from the optionally substituted group consisting of (C₁-C₃)alkoxy(C₁-C₃)alkyl, aryl, aryl(C₁-C₄)alkyl, cycloalkyl(C₁-C₄)alkyl, heterocyclyl(C₁-C₄)alkyl, heteroaryl(C₁-C₄)alkyl, —C(O)—(C₁-C₆)alkoxy, —C(O)—NH-phenyl, —C(O)—(C₁-C₆)alkyl, (C₁-C₆)alkyl and —(CH₂)_{d}-Q³;
wherein d is 1, 2, 3 or 4; and
Q³ is selected from the optionally substituted group consisting of (C₃-C₆)cycloalkyl, dimethylamino and phenyl;
provided that the compound of Formula (I) is not 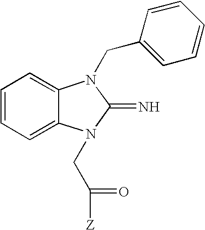
wherein Z is an optionally substituted phenyl;
provided that the compound of Formula (I) is not 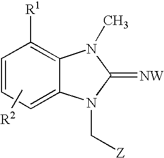
wherein Z is optionally substituted phenyl; and
R¹, R² and W are as defined for Formula (I);
provided that the compound of Formula (I) is not 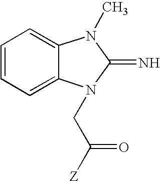
wherein Z is phenyl substituted with OH, t-butyl and —O—CH₂—CH₂—CH₂—CO—NH₂; OH, t-butyl and —O—CH₂—CH₂—CH₂—CN; OH, t-butyl and —OCH₂—CH₂—CH₂—C(O)—NH₂; pyrrolidinyl, t-butyl and —OCH₂—CH₂—CH₂—COOH; pyrrolidinyl, t-butyl and —OCH₂—COOH; or t-butyl and dimethylamino;
provided that the compound of Formula (I) is not 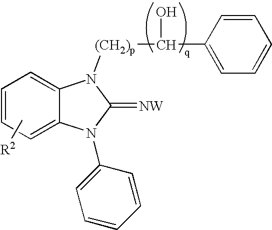
wherein
p is 1 or 2;
q is 0 or 1; and
R² is as defined for Formula (I);
provided that the compound of Formula (I) is not 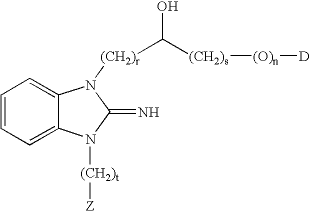
wherein D is CH₃, —CH═CH₂, propyl, butyl, t-butyl, furanyl, naphthyl, optionally substituted thienyl or optionally substituted phenyl;
Z is selected from the group consisting of H, CH₃, CH₂F, ethyl, morpholinyl, dimethylamino, diethylamino, —CH═CH₂, pentyl, dibenzylamino, naphthyl, piperidinyl and optionally substituted phenyl;
n is 0 or 1;
r is 1, 2 or 3;
s is 0, 1 or 2; and
t is 0, 1, 2 or 3;
provided that the compound of Formula (I) is not 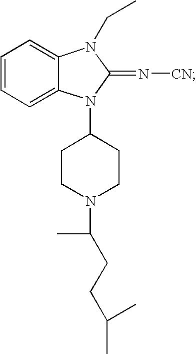
provided that the compound of Formula (I) is not 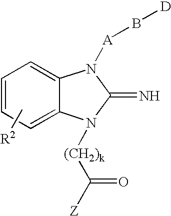
wherein
A-B is a bond or an optionally substituted (C₁-C₅)alkyl;
D is selected from the group consisting of H, COOH, OH, NH₂, propyl, isopropyl, t-butyl, biphenyl, furanyl, pyridinyl, thiazolyl, quinolinyl, morpholinyl, cyclohexyl optionally substituted with NH₂, —C(O)NH₂, COOH or —C(O)—OCH₂CH₃, phenyl optionally substituted with OH, t-butyl and —S(O)₂—CH₃, phenyl substituted with OH and two t-butyl or phenyl substituted with substituted propyl and COOH; or phenyl substituted with a substituent selected from the group consisting of Cl, F, CH₃, CN, COOH, CH₂—CH₂—COOH, —CH₂—C(CH₃)₂—COOH, —CH₂—CH₂—C(O)—O—CH₂—CH₃, —NH—CH₂—COOH, —C(O)—O—CH₂—CH₃, —CH₂—C(O)OH, dimethylamino, —S(O)₂—NH₂, —NH—CH₂—C(O)—NH₂, —NH—CH₂—C(O)—OH, —NH—C(O)—OH, —NH—C(O)—CH₂—CH₃, —NH—CH₂—C(O)—CH₂—CH₃, —NH₂, —CH₂—NH₂, NO₂, one or two OCH₃, —O—CH(CH₃)—C(O)—CH₂—CH₃, —O—CH(CH₃)—C(O)—OH, OH, —O—CH₂—CH₂—CH₃, CF₃, and t-butyl;
k is 1 or 2;
Z is NH₂ or phenyl substituted with OH and two t-butyl; and
R² is H or CF₃;
provided that in the compound of Formula (I) A-B-D and X-Y-Z are not both simultaneously bromobenzyl, —CH₂—CH₂-phenyl, —CH₂—CH₂-bromophenyl, —CH₂—CH₂—CH₂-phenyl or —CH₂—CH₂—CH₂-bromophenyl;
provided that the compound of Formula (I) is not 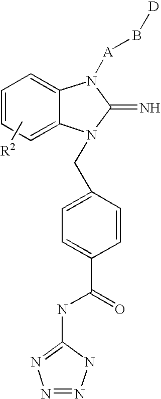
wherein A-B-D is ethyl or isopropyl;
provided that the compound of Formula (I) is not 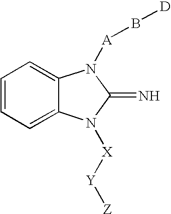
wherein
A is selected from the group consisting of a bond, —CH₂—CH(OH)—CH₂, optionally substituted methyl and optionally substituted ethyl;
B is selected from the group consisting of a bond, —C(O)—, —NH—C(O)— and O;
D is selected from the group consisting of H, OH, COOH, methyl, dimethylamino, furanyl, biphenyl, 3,5-di-t-butyl-4-hydroxyphenyl and phenyl wherein the phenyl is optionally substituted with Br, F, Cl, or —CH₂—OCH₂CH₃;
X is selected from the group consisting of a bond, CH₂ and pentyl;
Y is selected from the group consisting of a bond and —C(O); and
Z is selected from the group consisting of H, OH, butyl, biphenyl, heptyl, and morpholinyl, or
Z is selected from the group consisting of
benzo[1,3]dioxazinyl substituted with two methyls;
benzimidazolyl substituted with methyl and t-butyl;
benz[1,3,4]oxathiazin is optionally substituted with one or more CH₃, t-butyl and oxo;
cyclohexyl substituted with propyl;
indolyl substituted with OCH3
phenyl is optionally substituted with Br, Cl or —C(O)NH-tetrazolyl;
phenyl substituted with OH and two t-butyls;
phenyl is optionally substituted with pyrrolidinyl substituted with two —CH₂—O—C(CH₃)₃;
dihydrobenz[1,4]oxazinyl substituted with CH₃ and t-butyl;
piperazinyl optionally substituted with diphenylmethyl;
piperidinyl substituted with OH and four CH₃;
pyrimidinyl substituted with OH and two t-butyl;
pyrrolidinyl substituted with two —CH₂—O—CH(CH₃)₃;
pyrrolyl substituted with —C(O)—CH(CH₃)₂ and two CH₃;
provided that the compound of Formula (I) is not 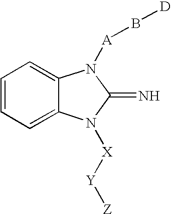
wherein
A is selected from the group consisting of a bond, CH₂, ethyl and propyl;
B is selected from a bond, and —C(O)—NH—CH₂;
D is selected from H, COOH, ethyl, propyl, (C₁-C₂)alkoxy, pentyl, and phenyl wherein the phenyl is optionally substituted with Br, —CH₂—OCH₂CH₃ or —O—CH₂CH(CH₃)₂;
X is selected from the group consisting of a bond, —CH(CH₃), CH₂, —CH₂—CH(OCH₃), —CH(OH), ethyl and pentyl;
Y is selected from the group consisting of a bond, —C(O), —C(O)—NH and NH; and
Z is selected from the group consisting of H, CH₃, ethyl, propyl, butyl, and morpholinyl; or
Z is selected from the group consisting of H, CH₃, CH₂OH, benzyloxy, cyclohexyl substituted with propyl and phenyl substituted with Br, and phenyl substituted with Br and 3,5-di-t-butyl-4-hydroxyphenyl;
provided that the compound of Formula (I) is not 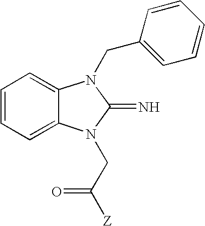
wherein Z is selected from the group consisting of
benz[1,3,4]oxathiazin substituted with t-butyl and two oxo,
benzo[1,4]oxazinyl substituted with one or more CH₃, oxo, t-butyl or —C(O)—CH₃,
benzimidazolyl substituted with CH₃ and t-butyl,
benzo[1,3]dioxazinyl substituted with one or more CH₃,
benzo[1,3]dioxazolyl substituted with one or more CH₃,
benzofuranyl substituted with one or more t-butyl, CH₃, ethyl, NO₂, and oxo,
benzoxazolyl substituted with one or more CH₃, oxo and t-butyl,
biphenyl,
or dihydrobenz[1,4]oxazinyl substituted with two CH₃,
dihydrobenzo[b]thiophenyl substituted with one or more t-butyl or CH₃,
dihydrobenzofuranyl substituted with one or more —N(CH₃)—C(O)—CH₃ or CH₃,
indolyl substituted with one or more Br, CH₃ or —CH₂—C(CH₃)₃,
naphthyl substituted with OH, or
phenyl substituted with one or more OH, CH₃, t-butyl or —CH₂—OCH₃.

2. The compound of claim 1 wherein the compound is 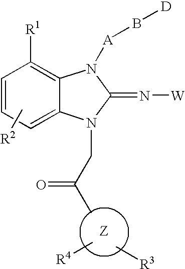
wherein
R¹ is selected from the group consisting of H, Br, Cl, CH₃, —C(O)OCH₃, pyridinyl, OCH₃, (C₂-C₅)alkenyl, phenyl, phenylethyl, biphenyl, imidazolyl, naphthyl, pyrazolyl and optionally substituted (C₁-C₅)alkyl;
Z is selected from the group consisting of benzo[1,3]dioxazolyl, benzo[d]isoxazolyl, 2,3-dihydrobenzo[1,4]dioxine, naphthyl, benzoxazolyl, furanyl, thienyl, phenyl, 4-morpholin-4-yl-phenyl and 4-pyrrolidin-1-yl-phenyl;
R³ is selected from the group consisting of H, Br, Cl, CH₃, CF₃, t-butyl and phenyl;
R⁴ is selected from the group consisting of H, Br, Cl, NO₂, CH₃, CF₃ and phenyl;
R² is selected from the group consisting of H, one or two CH₃, CN, (C₁-C₅)alkoxy, CF₃, OCF₃ and —C(O)-phenyl;
A is selected from the group consisting of a bond or (C₁-C₃)alkyl;
B is selected from the group consisting of a bond, —C(O)—N(R^{a})₂—, —N(R^{a})—C(O)—, C(O) and O;
R¹ is H or (C₁-C₄)alkyl;
D is selected from the group consisting of H, OH, CH₃, COOH, (C₃)alkenyl, (C₂-C₄)alkoxy, (C₃-C₅)cycloalkyl, and dimethylamino, or is selected from the optionally substituted group consisting of morpholinyl, piperidinyl, benzyl, phenyl, piperazinyl, pyridyl, quinolinyl, amino, thienyl, pyridylcarbonyl, phenylcarbonylmorpholinyl, phenylcarbonylpiperizinyl and phenylcarbonylpyrrolidinyl;
W is selected from the group consisting of H, CN, (C₁-C₄)alkyl, —CH₂—CH₂—CH₂OH, CH₂CH₂OH, —CH₂—CH₂—OCH₃, —CH₂-cyclopropyl, benzyl, dimethylaminobutyl, dimethylaminoethyl, dimethylaminopropyl, —C(O)—(C₁-C₂)alky, —CH₂-pyridinyl and —C(O)NH-phenyl wherein the phenyl is substituted with Br.

3. A compound according to claim 2 wherein R¹ is selected from the group consisting of Br, Cl, CH₂OH, CF₃, —C(O)OCH₃, pyridinyl, OCH₃, (C₂-C₅)alkenyl, phenyl, phenylethyl, biphenyl, imidazolyl, naphthyl, pyrazolyl and optionally substituted (C₁-C₅)alkyl.

4. A compound according to claim 2 wherein R¹ is H, Z is biphenyl or Z is phenyl optionally substituted with CN, NO₂, OCHF₂, OCF₃, CF₃, one or more F, one or more OCH₃ or one or more methyl and A-B-D is not benzyl.

5. A compound according to claim 1 wherein the compound is 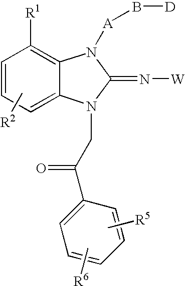
wherein
R¹ is selected from the group consisting of H, —C(O)—OCH₃, Br, Cl, OCH₃, CH₂OH, —C(═CH₂)CH₃, —CH═CH₂, —CH═CH—CH₃, —CH₂CH₂—O-phenyl, —CH₂CH₂CH₂OCH₃, CF₃, phenylethyl, CH₃, ethyl, isopropyl, butyl, propyl and cyclopropyl;
R² is selected from the group consisting of H, Cl, CN, OCH₃, CF₃, CH₃ and —C(O)-phenyl;
A is selected from the group consisting of a bond and optionally substituted (C₁-C₄)alkyl;
B is selected from the group consisting of a bond, —N(R^{a})—C(O)—, —C(O)—N(R^{a})C(O)—, —C(O)N(R^{a})—, C(O) and O;
wherein R^{a} is H or CH₃;
D is selected from the group consisting of H, (C₁-C₂)alkoxy, COOH, optionally substituted (C₁-C₂)alkyl, (C₃-C₆)cycloalkyl, dibenzylamino, thienyl, morpholinyl, optionally substituted benzyl, CF₃, Cl, and optionally substituted phenyl;
wherein the benzyl or the phenyl is optionally substituted with Br, CH₃, NO₂, CF₃ or OCH₃;
W is selected from the group consisting of H, —CH(CH₃)₂ and optionally substituted (C₁-C₄)alkyl;
R⁵ is selected from the group consisting of H, Br, Cl, F, NO₂, OCF₃, OCH₃, ethyl and CH₃;
R⁶ is selected from the group consisting of H, Br, Cl, F, OCH₃, CH₃ and phenyl.

6. A compound according to claim 5 wherein R¹ is selected from the group consisting of —C(O)—OCH₃, Br, Cl, OCH₃, CH₂OH, —C(═CH₂)CH₃, —CH═CH₂, —CH═CH—CH₃, —CH₂CH₂—O-phenyl, —CH₂CH₂CH₂OCH₃, CF₃, phenylethyl, CH₃, ethyl, isopropyl, butyl and propyl.

7. A compound according to claim 5 wherein
R¹ is selected from the group consisting of H, Br, Cl, CF₃, OCH₃, CH₂OH, —C(═CH₂)CH₃, —CH═CH₂, —CH₂═CH—CH₃, —CH₂CH₂—O-phenyl, —CH₂CH₂CH₂OCH₃, CH₃, ethyl, isopropyl, propyl and butyl;
A is a bond or optionally substituted (C₁-C₄)alkyl;
wherein the alkyl is optionally substituted by OH;
B is selected from the group consisting of a bond, —N(CH₃)—C(O), C(O)—N(CH₃), C(O) and O;
D is selected from the group consisting of H, COOH, CH₂OH, (C₁-C₂)alkoxy, cyclopropyl, cyclohexyl, dibenzylamino, phenyl and optionally substituted benzyl;
wherein the benzyl is optionally substituted with CH₃ or NO₂;
W is selected from the group consisting of H, CH₃, ethyl, CH₂CH₂OH and —CH₂CH₂CH₂OH;
R⁵ is selected from the group consisting of H, Br, Cl, OCH₃, ethyl and NO₂; and
R⁶ is selected from the group consisting of H, Br, Cl and OCH₃.

8. A compound according to claim 7 wherein R¹ is selected from the group consisting of Br, Cl, OCH₃, CH₂OH, —C(═CH₂)CH₃, —CH═CH₂, —CH₂═CH—CH₃, CH₃, ethyl, isopropyl and propyl.

9. A compound according to claim 7 wherein
R¹ is selected from the group consisting of H, Br, Cl, CH₂OH, —C(═CH₂)CH₃, —CH═CH₂, —CH₂CH₂—O-phenyl, —CH₂CH₂CH₂OCH₃, CF₃, CH₃, ethyl, isopropyl, butyl and propyl;
A is a bond or (C₁-C₄)alkyl;
B is selected from the group consisting of a bond, C(O), N(CH₃)—C(O), C(O)N(CH₃), and O;
D is selected from the group consisting of H, ethoxy, cyclopropyl, cyclohexyl, dibenzylamino, optionally substituted phenyl and optionally substituted benzyl;
W is H or ethyl; R⁵ is Br or Cl; and R⁶ is H or Cl.

10. A compound according to claim 9 wherein R¹ is Br, Cl, CH₂OH, —C(═CH₂)CH₃, —CH═CH₂, —CH₂CH₂—O-phenyl, —CH₂CH₂CH₂OCH₃, CF₃, CH₃, ethyl, isopropyl, butyl or propyl.

11. A compound according to claim 9 wherein R¹ is selected from the group consisting of H, Br, Cl, CH₂OH, —C(═CH₂)CH₃, CH₃, ethyl, isopropyl and propyl; A is CH₂; B is a bond; D is H; and W is H.

12. A compound according to claim 11 wherein R¹ is selected from the group consisting of H, Cl, CH₃, ethyl, isopropyl, propyl and —C(═CH₂)CH₃.

13. A compound according to claim 12 wherein
R¹ is selected from the group consisting of H, Cl, CH₃, and ethyl.

14. A compound of claim 2 wherein the compound is 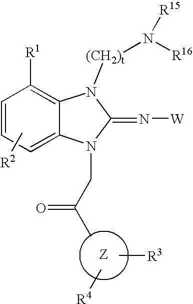
wherein
t is 0, 1, 2 or 3;
Z is phenyl or thienyl;
R¹ is selected from the group consisting of H, Cl and ethyl;
R² is H;
R¹⁵ is selected from the group consisting of H, (C₁-C₂)alkyl, phenyl, benzyl and —C(O)—OC(CH₃)₃;
R¹⁶ is selected from the group consisting of (C₁-C₂)alkyl, (C₃-C₆)cycloalkyl, optionally substituted phenylcarbonyl, optionally substituted benzyl, optionally substituted benzylcarbonyl, methylcarbonyl, and thienylcarbonyl;
R³ is selected from the group consisting of H, Br, Cl and CH₃; and
R⁴ is H or Cl.

15. The compound according to claim 14 wherein Z is phenyl; R¹⁵ is CH₃ or benzyl; R¹⁶ is selected from the group consisting of thienylcarbonyl, benzylcarbonyl, benzyl and cyclohexyl; and R³is selected from the group consisting of Br, Cl and CH₃.

16. The compound according to claim 15 wherein t is 2 or 3; R¹ is H or ethyl; R¹⁵ is CH₃; R¹⁶ is thienylcarbonyl or benzylcarbonyl; and R³ is Cl.

17. The compound of claim 5 wherein the compound is 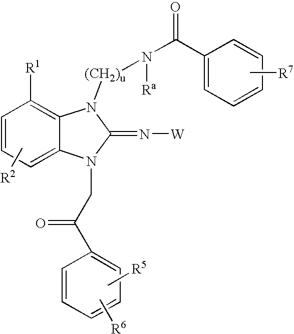
wherein
R¹ is selected from the group consisting of methyl, ethyl and Cl;
R² is H or Cl;
u is 2, 3 or 4;
R⁵ is selected from the group consisting of H, Br, Cl and OCH₃;
R⁶ is selected from the group consisting of H, Cl and OCH₃;
R⁷ is selected from the group consisting of H, CH₃, Cl and F;
R^{a} is H or CH₃; and
W is H.

18. A compound according to claim 1 wherein the compound is 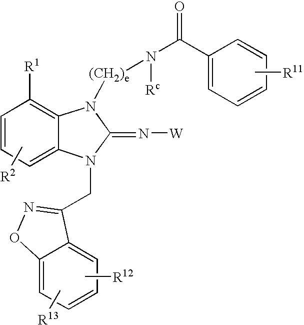
wherein
e is 0, 1 or 2;
R¹¹ is one or more substituents selected from the group consisting of H, CH₃, OH, CN, NO₂, CF₃ CO₂H, CO₂(C₁-C₃)alkyl, and halo;
R¹² and R¹³ are independently selected from the group consisting of H, CH₃, OH, CN, NO₂, CF₃ and halo; and
R^{c} is H, CH₃, NO₂, or CF₃.

19. A compound according to claim 1 wherein the compound is 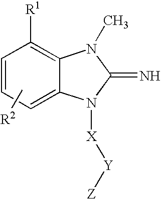
wherein
R¹ is H;
R² is H;
X is CH₂;
Y is S(O) or S; and
Z is phenyl optionally substituted with Cl.
